SEQUENCE LISTING (440 sequences)

SEQ ID NO: 1-2 - skipped (2 entries)

SEQ ID NO: 3 - DNA, 36 bp, Homo sapiens
  source 1..36: mol_type = other DNA; note = CD79A - Interface region of membrane domain
gaaggcattatcctcttgttttgcgcagttgtgccg

SEQ ID NO: 4 - protein, 12 aa, Homo sapiens
  source 1..12: note = CD79A - Interface region of membrane domain
EGIILLFCAVVP

SEQ ID NO: 5 - DNA, 36 bp, Homo sapiens
  source 1..36: mol_type = other DNA; note = CD79B - Interface region of membrane domain
caaactctcttgatcattttgtttatcatcgtccct

SEQ ID NO: 6 - protein, 12 aa, Homo sapiens
  source 1..12: note = CD79B - Interface region of membrane domain
QTLLIILFIIVP

SEQ ID NO: 7 - DNA, 66 bp, Homo sapiens
  source 1..66: mol_type = other DNA; note = CD79A - Membrane domain
ataatcacggccgaaggcattatcctcttgttttgcgcagttgtgccgggcactctgctgctcttt

SEQ ID NO: 8 - protein, 22 aa, Homo sapiens
  source 1..22: note = CD79A - Membrane domain
IITAEGIILLFCAVVPGTLLLF

SEQ ID NO: 9 - DNA, 63 bp, Homo sapiens
  source 1..63: mol_type = other DNA; note = CD79B - Membrane domain
gggattataatgatccaaactctcttgatcattttgtttatcatcgtccctatatttctcctg

SEQ ID NO: 10 - protein, 21 aa, Homo sapiens
  source 1..21: note = CD79B - Membrane domain
GIIMIQTLLIILFIIVPIFLL

SEQ ID NO: 11 - DNA, 333 bp, Homo sapiens
  source 1..333: mol_type = other DNA; note = CD79A - Extracellular domain
ctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccga

SEQ ID NO: 12 - protein, 143 aa, Homo sapiens
  source 1..143: note = CD79A - Extracellular domain
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNR

SEQ ID NO: 13 - DNA, 477 bp, Homo sapiens
  source 1..477: mol_type = other DNA; note = CD79B - Extracellular domain
atggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagac

SEQ ID NO: 14 - protein, 159 aa, Homo sapiens
  source 1..159: note = CD79B - Extracellular domain
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 15 - DNA, 678 bp, Homo sapiens
  source 1..678: mol_type = other DNA; note = CD79A - full length
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgcaaacggtggcagaacgagaaacttggccttgacgcgggagacgagtatgaagacgaaaatttgtacgagggtcttaatctcgacgattgcagtatgtacgaggacattagccgcggacttcaagggacttaccaagatgtaggatcactgaacataggtgacgtgcagttggaaaagcca

SEQ ID NO: 16 - protein, 226 aa, Homo sapiens
  source 1..226: note = CD79A - full length
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 17 - DNA, 687 bp, Homo sapiens
  source 1..687: mol_type = other DNA; note = CD79B - full length
atggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgattcaaaagcaggcatggaggaggaccatacttacgaaggtctggacatagatcaaacggcgacttatgaggacatcgtcactctgcgaacgggggaggttaaatggagtgtaggagaacatcccggacaggaa

SEQ ID NO: 18 - protein, 229 aa, Homo sapiens
  source 1..229: note = CD79B - full length
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 19 - protein, 22 aa, Homo sapiens
  source 1..22: note = mIg - Large motif of interface region of membrane domain
  VARIANT 2..6: note = X can be any amino acid
  VARIANT 8..9: note = X can be any amino acid
  VARIANT 12: note = X can be any amino acid
  VARIANT 14..16: note = X can be any amino acid
  VARIANT 19..21: note = X can be any amino acid
WXXXXXFXXLFXLXXXYSXXXT

SEQ ID NO: 20 - protein, 24 aa, Homo sapiens
  source 1..24: note = mIg - Consensus sequence of interface region of membrane domain
WTTITIFITLFLLSVCYSATVTFF

SEQ ID NO: 21 - protein, 24 aa, Homo sapiens
  source 1..24: note = mIgM - Fragment of membrane domain
WATASTFIVLFLLSLFYSTTVTLF

SEQ ID NO: 22 - protein, 24 aa, Homo sapiens
  source 1..24: note = mIgG1 - Fragment of membrane domain
WTTITIFITLFLLSVCYSATVTFF

SEQ ID NO: 23 - protein, 24 aa, Homo sapiens
  source 1..24: note = mIgG2 - Fragment of membrane domain
WTTITIFITLFLLSVCYSATITFF

SEQ ID NO: 24 - protein, 24 aa, Homo sapiens
  source 1..24: note = mIgG3 - Fragment of membrane domain
WTTITIFITLFLLSVCYSATVTFF

SEQ ID NO: 25 - protein, 24 aa, Homo sapiens
  source 1..24: note = mIgG4 - Fragment of membrane domain
WTTITIFITLFLLSVCYSATVTFF

SEQ ID NO: 26 - protein, 24 aa, Homo sapiens
  source 1..24: note = mIgD - Fragment of membrane domain
WTTLSTFVALFILTLLYSGIVTFI

SEQ ID NO: 27 - protein, 28 aa, Homo sapiens
  source 1..28: note = mIgE - Fragment of membrane domain
WTWTGLCIFAALFLLSVSYSAAITLLMV

SEQ ID NO: 28 - DNA, 129 bp, Homo sapiens
  source 1..129: mol_type = other DNA; note = mIgG1 - Membrane domain
gagctccagcttgaagaaagctgtgccgaggcacaggacggtgagctcgacggattgtggacgaccatcactatattcataactctgtttttgttgtctgtttgctactccgcaaccgtgactttcttt

SEQ ID NO: 29 - protein, 43 aa, Homo sapiens
  source 1..43: note = mIgG1 - Membrane domain
ELQLEESCAEAQDGELDGLWTTITIFITLFLLSVCYSATVTFF

SEQ ID NO: 30 - DNA, 87 bp, Homo sapiens
  source 1..87: mol_type = other DNA; note = mIgM - Membrane domain
gggtttgaaaatctctgggcgacagcatctacatttattgtcctctttctcctctctcttttctatagtacaactgtcacactcttt

SEQ ID NO: 31 - protein, 29 aa, Homo sapiens
  source 1..29: note = mIgM - Membrane domain
GFENLWATASTFIVLFLLSLFYSTTVTLF

SEQ ID NO: 32 - skipped

SEQ ID NO: 33 - DNA, 15 bp, Homo sapiens
  source 1..15: mol_type = other DNA; note = CD16A - Interface region of membrane domain (interaction with FceRIg and CD3z)
ttcgcagtagacact

SEQ ID NO: 34 - protein, 5 aa, Homo sapiens
  source 1..5: note = CD16A - Interface region of membrane domain (interaction with FceRIg and CD3z)
FAVDT

SEQ ID NO: 35 - DNA, 63 bp, Homo sapiens
  source 1..63: mol_type = other DNA; note = CD16A - Membrane domain
gtgtccttttgcctcgtaatggtactgctgttcgcagtagacactggtctttacttttccgta

SEQ ID NO: 36 - protein, 21 aa, Homo sapiens
  source 1..21: note = CD16A - Membrane domain
VSFCLVMVLLFAVDTGLYFSV

SEQ ID NO: 37 - protein, 24 aa, Homo sapiens
  source 1..24: note = mIgA1 membrane domain
WPTTITFLTLFLLSLFYSTALTVT

SEQ ID NO: 38 - protein, 24 aa, Homo sapiens
  source 1..24: note = mIgA2 membrane domain
WPTTITFLTLFLLSLFYSTALTVT

SEQ ID NO: 39 - protein, 74 aa, synthetic construct
  source 1..74: note = CD16 - Motif interface region of extracellular domain (interaction with Ig)
  VARIANT 4..25: note = X can be any amino acid
  VARIANT 27..28: note = X can be any amino acid
  VARIANT 31: note = X can be any amino acid
  VARIANT 34: note = X can be any amino acid
  VARIANT 36..42: note = X can be any amino acid
  VARIANT 46: note = X can be any amino acid
  VARIANT 49..67: note = X can be any amino acid
  VARIANT 69..71: note = X can be any amino acid
  VARIANT 73: note = X can be any amino acid
IGWXXXXXXXXXXXXXXXXXXXXXXWXXTAXHKXTXXXXXXXRKYXHHXXXXXXXXXXXXXXXXXXXRXXXGXK

SEQ ID NO: 40 - DNA, 222 bp, Homo sapiens
  source 1..222: mol_type = other DNA; note = CD16A - Interface region of extracellular domain (interaction with Ig)
ataggatggctgttgttgcaagcgccacgatgggtattcaaggaggaagatccaatacacctgagatgtcactcttggaaaaataccgctcttcataaggttacctacctccagaacggaaaaggcaggaaatacttccaccacaattcagacttttatattcctaaagcgaccttgaaggacagcggtagctacttctgtcggggattgttcggatcaaaa

SEQ ID NO: 41 - protein, 74 aa, Homo sapiens
  source 1..74: note = CD16A - Interface region of extracellular domain (interaction with Ig)
IGWLLLQAPRWVFKEEDPIHLRCHSWKNTALHKVTYLQNGKGRKYFHHNSDFYIPKATLKDSGSYFCRGLFGSK

SEQ ID NO: 42 - DNA, 222 bp, Homo sapiens; identical to SEQ ID NO: 40
  source 1..222: mol_type = other DNA; note = CD16A - Extracellular domain (partial)

SEQ ID NO: 43 - protein, 208 aa, Homo sapiens
  source 1..208: note = CD16A - Extracellular domain
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 44 - DNA, 576 bp, Homo sapiens
  source 1..576: mol_type = other DNA; note = CD16A - Extracellular domain (version 2)
ggtatgcgaaccgaagacctcccgaaggccgtcgtgtttttggagcctcaatggtatagagtcttggaaaaggatagtgtaactttgaaatgtcaaggcgcgtattcaccggaggacaattcaacacaatggtttcataacgagtctctgatctccagtcaagccagctcctatttcatagacgcggctaccgtggatgattccggggaatataggtgtcaaacaaaccttagtacactttctgatccggtccaactcgaagttcatataggatggctgttgttgcaagcgccacgatgggtattcaaggaggaagatccaatacacctgagatgtcactcttggaaaaataccgctcttcataaggttacctacctccagaacggaaaaggcaggaaatacttccaccacaattcagacttttatattcctaaagcgaccttgaaggacagcggtagctacttctgtcggggattgttcggatcaaaaaatgtgtcatctgagacagttaacataacaatcacccagggtctggctgttagcactatatcctctttcttcccgccggggtatcag

SEQ ID NO: 45 - protein, 192 aa, Homo sapiens
  source 1..192: note = CD16A - Extracellular domain (version 2)
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 46 - DNA, 762 bp, Homo sapiens
  source 1..762: mol_type = other DNA; note = CD16A - Full length
atgtggcagcttctgctcccgacagcacttttgcttctcgtatcagcaggtatgcgaaccgaagacctcccgaaggccgtcgtgtttttggagcctcaatggtatagagtcttggaaaaggatagtgtaactttgaaatgtcaaggcgcgtattcaccggaggacaattcaacacaatggtttcataacgagtctctgatctccagtcaagccagctcctatttcatagacgcggctaccgtggatgattccggggaatataggtgtcaaacaaaccttagtacactttctgatccggtccaactcgaagttcatataggatggctgttgttgcaagcgccacgatgggtattcaaggaggaagatccaatacacctgagatgtcactcttggaaaaataccgctcttcataaggttacctacctccagaacggaaaaggcaggaaatacttccaccacaattcagacttttatattcctaaagcgaccttgaaggacagcggtagctacttctgtcggggattgttcggatcaaaaaatgtgtcatctgagacagttaacataacaatcacccagggtctggctgttagcactatatcctctttcttcccgccggggtatcaggtgtccttttgcctcgtaatggtactgctgttcgcagtagacactggtctttacttttccgtaaagaccaatataaggagcagtactcgcgactggaaagaccataagtttaaatggcgcaaagatcctcaggataag

SEQ ID NO: 47 - protein, 254 aa, Homo sapiens
  source 1..254: note = CD16A - Full length
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 48 - protein, 15 aa, synthetic construct
  source 1..15: note = ITAM consensus motif 1, variant 1
  VARIANT 2..3: note = X can be any amino acid
  VARIANT 4: note = L
  VARIANT 5..11: note = X can be any amino acid
  VARIANT 13..14: note = X can be any amino acid
  VARIANT 15: note = L
YXXIXXXXXXXYXXI

SEQ ID NO: 49 - protein, 16 aa, synthetic construct
  source 1..16: note = ITAM consensus motif 1, variant 2
  VARIANT 2..3: note = X can be any amino acid
  VARIANT 4: note = L
  VARIANT 5..12: note = X can be any amino acid
  VARIANT 14..15: note = X can be any amino acid
  VARIANT 16: note = L
YXXIXXXXXXXXYXXI

SEQ ID NO: 50 - protein, 99 aa, synthetic construct
  source 1..99: note = IgG1 - Motif interface region of constant region (interaction with CD16)
  VARIANT 7..31: note = X can be any amino acid
  VARIANT 33: note = X can be any amino acid
  VARIANT 35: note = X can be any amino acid
  VARIANT 37..63: note = X can be any amino acid
  VARIANT 67..93: note = X can be any amino acid
  VARIANT 98: note = X can be any amino acid
LLGGPSXXXXXXXXXXXXXXXXXXXXXXXXXDXSXEXXXXXXXXXXXXXXXXXXXXXXXXXXXNSTXXXXXXXXXXXXXXXXXXXXXXXXXXXALPAXI

SEQ ID NO: 51 - DNA, 297 bp, Homo sapiens
  source 1..297: mol_type = other DNA; note = IgG1 - Interface region of constant region (interaction with CD16)
gatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggag

SEQ ID NO: 52 - protein, 99 aa, Homo sapiens
  source 1..99: note = IgG1 - Interface region of constant region (interaction with CD16)
LLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPI

SEQ ID NO: 53 - DNA, 984 bp, Homo sapiens
  source 1..984: mol_type = other DNA; note = IgG1 - heavy chain constant region
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccg

SEQ ID NO: 54 - protein, 328 aa, Homo sapiens
  source 1..328: note = IgG1 - heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 55 - DNA, 492 bp, Homo sapiens
  source 1..492: mol_type = other DNA; note = CD3zeta - full length
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 56 - protein, 164 aa, Homo sapiens
  source 1..164: note = CD3zeta - full length
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 57 - DNA, 258 bp, Homo sapiens
  source 1..258: mol_type = other DNA; note = FceRIg - full length
atgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 58 - protein, 86 aa, Homo sapiens
  source 1..86: note = FceRIg - full length
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 59 - DNA, 321 bp, Homo sapiens
  source 1..321: mol_type = other DNA; note = Kappa light chain constant region
cgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 60 - protein, 107 aa, Homo sapiens [sequence omitted]

SEQ ID NO: 61 - DNA, 294 bp, Homo sapiens
  source 1..294: mol_type = other DNA; note = IgG1 heavy chain constant domain CH1
aactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtgctagc

SEQ ID NO: 62 - protein, 98 aa, Homo sapiens
  source 1..98: note = IgG1 heavy chain constant domain CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 63 - DNA, 339 bp, Homo sapiens
  source 1..339: mol_type = other DNA; note = IgG1 heavy chain constant domain CH2
ccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 64 - protein, 113 aa, Homo sapiens
  source 1..113: note = IgG1 heavy chain constant domain CH2
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 65 - DNA, 315 bp, Homo sapiens
  source 1..315: mol_type = other DNA; note = IgG1 heavy chain constant domain CH3
gggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccg

SEQ ID NO: 66 - protein, 105 aa, Homo sapiens
  source 1..105: note = IgG1 heavy chain constant domain CH3
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 67 - DNA, 330 bp, Homo sapiens
  source 1..330: mol_type = other DNA; note = IgM heavy chain constant domain CH4
gtcgctcttcatcgtccggatgtctatctcttgcccccagctcgagagcaacttaatctccgtgagagcgcgaccatcacgtgtctcgtcacgggcttctcccccgcagatgttttcgttcaatggatgcaacgcggtcaaccactgagtcctgagaagtatgtgacttctgccccaatgccggaaccacaagcacccggtaggtactttgctcactccatcttgaccgtttccgaggaagagtggaacactggggagacctacacctgtgttgtcgcccacgaagcgttgcccaatcgtgttaccgaacgaacggtcgacaagtccacc

SEQ ID NO: 68 - protein, 110 aa, Homo sapiens
  source 1..110: note = IgM heavy chain constant domain CH4
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 69 - protein, 5 aa, synthetic construct
  source 1..5: note = Glycine-serine linker motif
GGGGS

SEQ ID NO: 70 - protein, 6 aa, synthetic construct
  source 1..6: note = Glycine-serine linker N-terminus
SGGGGS

SEQ ID NO: 71 - DNA, 186 bp, synthetic construct
  source 1..186: mol_type = other DNA; note = Glycine-serine linker
tccggtggaggcgggtctggaggcgggggtagtggcgggggtggaagcgggggtggaggcgggtcgggtggcggaggtagcggaggcggtggaagtggaggcggtggaagtggtggaggaggttctggtggcggaggtagcggtggcggaggtagcggaggcggtggaagtggtggcggaggtagc

SEQ ID NO: 72 - protein, 62 aa, synthetic construct
  source 1..62: note = Glycine-serine linker
SGGGGSGGGGSGGGGSGGGGGSGGGGSGGGGSGGGGSGGGGSGGGGSGGGGSGGGGSGGGGS

SEQ ID NO: 73 - protein, 14 aa, synthetic construct
  source 1..14: note = ITAM consensus motif 1
  VARIANT 2..3: note = X can be any amino acid
  VARIANT 4: note = L
  VARIANT 5..10: note = X can be any amino acid
  VARIANT 12..13: note = X can be any amino acid
  VARIANT 14: note = L
YXXIXXXXXXYXXI

SEQ ID NO: 74 - protein, 20 aa, synthetic construct
  source 1..20: note = ITAM consensus motif 2
  VARIANT 4: note = L
  VARIANT 5..16: note = X can be any amino acid
  VARIANT 18..19: note = X can be any amino acid
  VARIANT 20: note = L
  VARIANT 2..3: note = X can be any amino acid
YXXIXXXXXXXXXXXXYXXI

SEQ ID NO: 75 - DNA, 45 bp, Homo sapiens
  source 1..45: mol_type = other DNA; note = CD3 zeta - ITAM 1
tataacgagctcaatctaggacgaagagaggagtacgatgttttg

SEQ ID NO: 76 - protein, 15 aa, Homo sapiens
  source 1..15: note = CD3 zeta - ITAM 1
YNELNLGRREEYDVL

SEQ ID NO: 77 - DNA, 48 bp, Homo sapiens
  source 1..48: mol_type = other DNA; note = CD3 zeta - ITAM 2
tacaatgaactgcagaaagataagatggcggaggcctacagtgagatt

SEQ ID NO: 78 - protein, 16 aa, Homo sapiens
  source 1..16: note = CD3 zeta - ITAM 2
YNELQKDKMAEAYSEI

SEQ ID NO: 79 - DNA, 45 bp, Homo sapiens
  source 1..45: mol_type = other DNA; note = CD3 zeta - ITAM 3
taccagggtctcagtacagccaccaaggacacctacgacgccctt

SEQ ID NO: 80 - protein, 15 aa, Homo sapiens
  source 1..15: note = CD3 zeta - ITAM 3
YQGLSTATKDTYDAL

SEQ ID NO: 81 - DNA, 255 bp, Homo sapiens
  source 1..255: mol_type = other DNA; note = CD3 zeta - ITAM region
tataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgccctt

SEQ ID NO: 82 - protein, 85 aa, Homo sapiens
  source 1..85: note = CD3 zeta - ITAM region
YNELNLGRREEYDVLDKRRGRDPEMGGKPQRRKNPQEGLYNELQKDKMAEAYSEIGMKGERRRGKGHDGLYQGLSTATKDTYDAL

SEQ ID NO: 83 - DNA, 312 bp, Homo sapiens
  source 1..312: mol_type = other DNA; note = CD3 zeta - Intracellular domain
gcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctaga

SEQ ID NO: 84 - protein, 104 aa, Homo sapiens
  source 1..104: note = CD3 zeta - Intracellular domain
APAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 85 - DNA, 45 bp, Homo sapiens
  source 1..45: mol_type = other DNA; note = FceRIg - ITAM
tatacgggtctgagtacacggaaccaggagacctacgagaccttg

SEQ ID NO: 86 - protein, 15 aa, Homo sapiens
  source 1..15: note = FceRIg - ITAM
YTGLSTRNQETYETL

SEQ ID NO: 87 - DNA, 99 bp, Homo sapiens
  source 1..99: mol_type = other DNA; note = FceRIg - Intracellular domain
gccatcacaagctacgaaaagtctgacggggtgtatacgggtctgagtacacggaaccaggagacctacgagaccttgaaacacgaaaaacctccacaa

SEQ ID NO: 88 - protein, 33 aa, Homo sapiens
  source 1..33: note = FceRIg - Intracellular domain
AITSYEKSDGVYTGLSTRNQETYETLKHEKPPQ

SEQ ID NO: 89 - DNA, 84 bp, Homo sapiens
  source 1..84: mol_type = other DNA; note = mIgG1 - Intracellular domain
aaagtaaaatggatattttcttcagtggtcgatctcaagcagacgatcatcccagactatcgcaatatgatagggcaaggtgcc

SEQ ID NO: 90 - protein, 28 aa, Homo sapiens
  source 1..28: note = mIgG1 - Intracellular domain
KVKWIFSSVVDLKQTIIPDYRNMIGQGA

SEQ ID NO: 91 - DNA, 63 bp, Homo sapiens
  source 1..63: mol_type = other DNA; note = CD79A - ITAM
tatgaagacgaaaatttgtacgagggtcttaatctcgacgattgcagtatgtacgaggacatt

SEQ ID NO: 92 - protein, 15 aa, Homo sapiens
  source 1..15: note = CD79A - ITAM
YEGLNLDDCSMYEDI

SEQ ID NO: 93 - DNA, 45 bp, Homo sapiens
  source 1..45: mol_type = other DNA; note = CD79B - ITAM
tacgaaggtctggacatagatcaaacggcgacttatgaggacatc

SEQ ID NO: 94 - protein, 15 aa, Homo sapiens
  source 1..15: note = CD79B - ITAM
YEGLDIDQTATYEDI

SEQ ID NO: 95 - DNA, 183 bp, Homo sapiens
  source 1..183: mol_type = other DNA; note = CD79A - Intracellular domain
cgcaaacggtggcagaacgagaaacttggccttgacgcgggagacgagtatgaagacgaaaatttgtacgagggtcttaatctcgacgattgcagtatgtacgaggacattagccgcggacttcaagggacttaccaagatgtaggatcactgaacataggtgacgtgcagttggaaaagcca

SEQ ID NO: 96 - protein, 61 aa, Homo sapiens
  source 1..61: note = CD79A - Intracellular domain
RKRWQNEKLGLDAGDEYEDENLYEGLNLDDCSMYEDISRGLQGTYQDVGSLNIGDVQLEKP

SEQ ID NO: 97 - DNA, 147 bp, Homo sapiens
  source 1..147: mol_type = other DNA; note = CD79B - Intracellular domain
ttggataaggacgattcaaaagcaggcatggaggaggaccatacttacgaaggtctggacatagatcaaacggcgacttatgaggacatcgtcactctgcgaacgggggaggttaaatggagtgtaggagaacatcccggacaggaa

SEQ ID NO: 98 - protein, 49 aa, Homo sapiens
  source 1..49: note = CD79B - Intracellular domain
LDKDDSKAGMEEDHTYEGLDIDQTATYEDIVTLRTGEVKWSVGEHPGQE

SEQ ID NO: 99 - DNA, 1197 bp, Homo sapiens
  source 1..1197: mol_type = other DNA; note = IgG1 heavy chain - constant region, membrane domain, and intracellular domain
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccggagctccagcttgaagaaagctgtgccgaggcacaggacggtgagctcgacggattgtggacgaccatcactatattcataactctgtttttgttgtctgtttgctactccgcaaccgtgactttctttaaagtaaaatggatattttcttcagtggtcgatctcaagcagacgatcatcccagactatcgcaatatgatagggcaaggtgcc

SEQ ID NO: 100 - protein, 399 aa, Homo sapiens
  source 1..399: note = IgG1 heavy chain - constant region, membrane domain, and intracellular domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 101 - DNA, 1383 bp, Homo sapiens
  source 1..1383: mol_type = other DNA; note = IgG1 heavy chain - constant region, linker, membrane domain, and intracellular domain
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgtccggtggaggcgggtctggaggcgggggtagtggcgggggtggaagcgggggtggaggcgggtcgggtggcggaggtagcggaggcggtggaagtggaggcggtggaagtggtggaggaggttctggtggcggaggtagcggtggcggaggtagcggaggcggtggaagtggtggcggaggtagcgagctccagcttgaagaaagctgtgccgaggcacaggacggtgagctcgacggattgtggacgaccatcactatattcataactctgtttttgttgtctgtttgctactccgcaaccgtgactttctttaaagtaaaatggatattttcttcagtggtcgatctcaagcagacgatcatcccagactatcgcaatatgatagggcaaggtgcc

SEQ ID NO: 102 - protein, 461 aa, Homo sapiens
  source 1..461: note = IgG1 heavy chain - constant region, linker, membrane domain, and intracellular domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPSGGGGSGGGGSGGGGSGGGGGSGGGGSGGGGSGGGGSGGGGSGGGGSGGGGSGGGGSGGGGSELQLEESCAEAQDGELDGLWTTITIFITLFLLSVCYSATVTFFKVKWIFSSVVDLKQTIIPDYRNMIGQGA

SEQ ID NO: 103 - DNA, 1326 bp, Homo sapiens
  source 1..1326: mol_type = other DNA; note = IgG1 heavy chain - constant region, linker, membrane domain, and intracellular domain
ggaagcgcttctgcgcctacgctctttccactggtttcatgtgagaacagcccttctgatacgtccagcgtagccgttggttgtcttgcccaggatttcttgccagattccattaccttttcctggaaatataagaataattccgatataagttccacccgcgggttcccctctgtcttgcgaggtggtaaatatgccgcaaccagtcaagtccttcttcctagtaaggatgtaatgcaaggcactgacgaacatgtggtctgcaaagtccagcatccgaatggaaacaaggagaagaacgtaccattgcccgtaatcgccgagttgccaccgaaggtgtcagtattcgtgccgccgcgcgatgggttcttcgggaatcctcggaaaagcaaactgatttgtcaagctaccgggtttagtccccggcaaattcaagtctcctggctgcgagaaggaaagcaagtcggaagtggggtgactactgaccaagtccaagctgaagcaaaggaatctggtcccaccacatacaaagtgacttcaacattgacaattaaggaatcagattggctctcacagagcatgttcacgtgtcgagtggaccaccggggtttgacctttcagcaaaacgcaagcagcatgtgtgttccggaccaggacacagccatccgtgtctttgcgattcccccgagctttgcaagcatctttctgaccaaatcaactaaattgacgtgcctggttaccgacctgactacatatgattcagtgactatttcctggacaagacaaaacggtgaggccgtcaagacacatacgaatatatctgaatcccatccgaatgcaaccttctccgctgttggtgaagccagtatttgtgaagatgactggaatagcggcgaacgttttacctgtacagtgactcacaccgatctcccatcaccacttaagcagaccataagtagacccaagggagtcgctcttcatcgtccggatgtctatctcttgcccccagctcgagagcaacttaatctccgtgagagcgcgaccatcacgtgtctcgtcacgggcttctcccccgcagatgttttcgttcaatggatgcaacgcggtcaaccactgagtcctgagaagtatgtgacttctgccccaatgccggaaccacaagcacccggtaggtactttgctcactccatcttgaccgtttccgaggaagagtggaacactggggagacctacacctgtgttgtcgcccacgaagcgttgcccaatcgtgttaccgaacgaacggtcgacaagtccaccgaaggagaagtttccgctgatgaagaa

SEQ ID NO: 104 - protein, 442 aa, Homo sapiens
  source 1..442: note = mIgM - constant region
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTEGEVSADEE

SEQ ID NO: 105 - DNA, 1422 bp, Homo sapiens
  source 1..1422: mol_type = other DNA; note = mIgM - constant region and membrane domain and cytosolic tail
ggaagcgcttctgcgcctacgctctttccactggtttcatgtgagaacagcccttctgatacgtccagcgtagccgttggttgtcttgcccaggatttcttgccagattccattaccttttcctggaaatataagaataattccgatataagttccacccgcgggttcccctctgtcttgcgaggtggtaaatatgccgcaaccagtcaagtccttcttcctagtaaggatgtaatgcaaggcactgacgaacatgtggtctgcaaagtccagcatccgaatggaaacaaggagaagaacgtaccattgcccgtaatcgccgagttgccaccgaaggtgtcagtattcgtgccgccgcgcgatgggttcttcgggaatcctcggaaaagcaaactgatttgtcaagctaccgggtttagtccccggcaaattcaagtctcctggctgcgagaaggaaagcaagtcggaagtggggtgactactgaccaagtccaagctgaagcaaaggaatctggtcccaccacatacaaagtgacttcaacattgacaattaaggaatcagattggctctcacagagcatgttcacgtgtcgagtggaccaccggggtttgacctttcagcaaaacgcaagcagcatgtgtgttccggaccaggacacagccatccgtgtctttgcgattcccccgagctttgcaagcatctttctgaccaaatcaactaaattgacgtgcctggttaccgacctgactacatatgattcagtgactatttcctggacaagacaaaacggtgaggccgtcaagacacatacgaatatatctgaatcccatccgaatgcaaccttctccgctgttggtgaagccagtatttgtgaagatgactggaatagcggcgaacgttttacctgtacagtgactcacaccgatctcccatcaccacttaagcagaccataagtagacccaagggagtcgctcttcatcgtccggatgtctatctcttgcccccagctcgagagcaacttaatctccgtgagagcgcgaccatcacgtgtctcgtcacgggcttctcccccgcagatgttttcgttcaatggatgcaacgcggtcaaccactgagtcctgagaagtatgtgacttctgccccaatgccggaaccacaagcacccggtaggtactttgctcactccatcttgaccgtttccgaggaagagtggaacactggggagacctacacctgtgttgtcgcccacgaagcgttgcccaatcgtgttaccgaacgaacggtcgacaagtccaccgaaggagaagtttccgctgatgaagaagggtttgaaaatctctgggcgacagcatctacatttattgtcctctttctcctctctcttttctatagtacaactgtcacactctttaaggtgaag

SEQ ID NO: 106 - protein, 474 aa, Homo sapiens
  source 1..474: note = mIgM - constant region and membrane domain and cytosolic tail
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTEGEVSADEEGFENLWATASTFIVLFLLSLFYSTTVTLFKVK

SEQ ID NO: 107 - DNA, 240 bp, Homo sapiens
  source 1..240: mol_type = other DNA; note = mIgG1 membrane domain - FceRIg intracellular domain fusion
gagctccagcttgaagaaagctgtgccgaggcacaggacggtgagctcgacggattgtggacgaccatcactatattcataactctgtttttgttgtctgtttgctactccgcaaccgtgactttctttaaagttaaagacgccatcacaagctacgaaaagtctgacggggtgtatacgggtctgagtacacggaaccaggagacctacgagaccttgaaacacgaaaaacctccacaa

SEQ ID NO: 108 - protein, 80 aa, Homo sapiens
  source 1..80: note = mIgG1 membrane domain - FceRIg intracellular domain fusion
ELQLEESCAEAQDGELDGLWTTITIFITLFLLSVCYSATVTFFKVKDAITSYEKSDGVYTGLSTRNQETYETLKHEKPPQ

SEQ ID NO: 109 - DNA, 843 bp, Homo sapiens
  source 1..843: mol_type = other DNA; note = CD79A extracellular and membrane domain_CD3 zeta intracellular domain fusion
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgcaaacggtggcagaacgagaaacttggccttgacgcgcccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 110 - protein, 281 aa, Homo sapiens
  source 1..281: note = CD79A extracellular and membrane domain_CD3 zeta intracellular domain fusion
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 111 - DNA, 864 bp, Homo sapiens
  source 1..864: mol_type = other DNA; note = CD79B extracellular and membrane domain_CD3 zeta intracellular domain fusion
atggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctaga

SEQ ID NO: 112 - protein, 288 aa, Homo sapiens
  source 1..288: note = CD79B extracellular and membrane domain_CD3 zeta intracellular domain fusion
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 113 - DNA, 1224 bp, Homo sapiens
  source 1..1224: mol_type = other DNA; note = mIgG1 constant region and membrane domain_FceRIg intracellular domain fusion
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccggagctccagcttgaagaaagctgtgccgaggcacaggacggtgagctcgacggattgtggacgaccatcactatattcataactctgtttttgttgtctgtttgctactccgcaaccgtgactttctttaaagttaaagacgccatcacaagctacgaaaagtctgacggggtgtatacgggtctgagtacacggaaccaggagacctacgagaccttgaaacacgaaaaacctccacaa

SEQ ID NO: 114 - protein, 408 aa, Homo sapiens
  source 1..408: note = mIgG1 constant region and membrane domain_FceRIg intracellular domain fusion
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKDAITSYEKSDGVYTGLSTRNQETYETLKHEKPPQ

SEQ ID NO: 115 - DNA, 65 bp, synthetic construct
  source 1..65: mol_type = other DNA; note = PR5184
ccatttaccgtaagttatgtaacgcctgcaggttaattaaaggggataacgcaggaaagaacatg

SEQ ID NO: 116 - DNA, 62 bp, synthetic construct
  source 1..62: mol_type = other DNA; note = PR5185
agtaaaacctctacaaatgtggtatggaaatgttaattatggcacttttcggggaaatgtgc

SEQ ID NO: 117 - DNA, 120 bp, synthetic construct
  source 1..120: mol_type = other DNA; note = PR5857
gagctccagcttgaagaaagctgtgccgaggcacaggacggtgagctcgacggattgtggacgaccatcactatattcataactctgtttttgttgtctgtttgctactccgcaaccgtg

SEQ ID NO: 118 - DNA, 120 bp, synthetic construct
  source 1..120: mol_type = other DNA; note = PR5858
ggcaccttgccctatcatattgcgatagtctgggatgatcgtctgcttgagatcgaccactgaagaaaatatccattttactttaaagaaagtcacggttgcggagtagcaaacagacaa

SEQ ID NO: 119 - DNA, 25 bp, synthetic construct
  source 1..25: mol_type = other DNA; note = PR5693
catcgagaaaaccatctccaaagcc

SEQ ID NO: 120 - DNA, 61 bp, synthetic construct
  source 1..61: mol_type = other DNA; note = PR5856
accgtcctgtgcctcggcacagctttcttcaagctggagctccggagacagggagaggctc

SEQ ID NO: 121 - DNA, 23 bp, synthetic construct
  source 1..23: mol_type = other DNA; note = PR5859
gagctccagcttgaagaaagctg

SEQ ID NO: 122 - DNA, 65 bp, synthetic construct
  source 1..65: mol_type = other DNA; note = PR5860
catcaatgtatcttatcatgtctggccagctagctgtacatcaggcaccttgccctatcatattg

SEQ ID NO: 123 - DNA, 21 bp, synthetic construct
  source 1..21: mol_type = other DNA; note = PR8371
agctatgaaaccgagcgagag

SEQ ID NO: 124 - DNA, 23 bp, synthetic construct
  source 1..23: mol_type = other DNA; note = PR8372
agtgttcttgtaggcagaggatg

SEQ ID NO: 125 - DNA, 49 bp, synthetic construct
  source 1..49: mol_type = other DNA; note = PR8563
agcctttggagtacgaaccgtaactcaaatcctgcacaacagattcttc

SEQ ID NO: 126 - DNA, 49 bp, synthetic construct
  source 1..49: mol_type = other DNA; note = PR8564
tcaacatcattcacgccatgtaactcaaatcctgcacaacagattcttc

SEQ ID NO: 127 - DNA, 25 bp, synthetic construct
  source 1..25: mol_type = other DNA; note = PR3894
ttttcttccatttcaggtgtcgtga

SEQ ID NO: 128 - DNA, 66 bp, synthetic construct
  source 1..66: mol_type = other DNA; note = PR8672
attctcctcgacgtcaccgcatgttagcagacttcctctgccctccttgtacagctcgtccatgcc

SEQ ID NO: 129 - DNA, 63 bp, synthetic construct
  source 1..63: mol_type = other DNA; note = PR8673
tctgctaacatgcggtgacgtcgaggagaatcctggcccaatgttgccatcacaactcattgg

SEQ ID NO: 130 - DNA, 63 bp, synthetic construct
  source 1..63: mol_type = other DNA; note = PR8674
cttcaacatcacctgcttgctttagcagagagaagtttgtggcacactctcccctgttgaagc

SEQ ID NO: 131 - DNA, 65 bp, synthetic construct
  source 1..65: mol_type = other DNA; note = PR8675
ctctgctaaagcaagcaggtgatgttgaagaaaaccccgggcctatggactggacctggaggatc

SEQ ID NO: 132 - DNA, 59 bp, synthetic construct
  source 1..59: mol_type = other DNA; note = PR8676
ggttgattatcgataagcttgatatcgttcgaactaggcaccttgccctatcatattgc

SEQ ID NO: 133 - DNA, 58 bp, synthetic construct
  source 1..58: mol_type = other DNA; note = PR8742
atccagaggttgattatcgataagcttgatatcgttatgtctggccagctagctgtac

SEQ ID NO: 134 - DNA, 21 bp, synthetic construct
  source 1..21: mol_type = other DNA; note = PR8700
atctccaaagccaaagggcag

SEQ ID NO: 135 - DNA, 19 bp, synthetic construct
  source 1..19: mol_type = other DNA; note = PR8701
cggagacagggagaggctc

SEQ ID NO: 136 - DNA, 53 bp, synthetic construct
  source 1..53: mol_type = other DNA; note = PR8702
acgcagaagagcctctccctgtctccgtccggtggaggcgggtctggaggcgg

SEQ ID NO: 137 - DNA, 57 bp, synthetic construct
  source 1..57: mol_type = other DNA; note = PR8703
ggcacagctttcttcaagctggagctcgctacctccgccaccacttccaccgcctcc

SEQ ID NO: 138 - DNA, 23 bp, synthetic construct
  source 1..23: mol_type = other DNA; note = PR8704
gagctccagcttgaagaaagctg

SEQ ID NO: 139 - DNA, 29 bp, synthetic construct
  source 1..29: mol_type = other DNA; note = PR8705
catcaatgtatcttatcatgtctggccag

SEQ ID NO: 140 - DNA, 67 bp, synthetic construct
  source 1..67: mol_type = other DNA; note = PR8667
cttcaacatcacctgcttgctttagcagagagaagtttgtggccttgtacaactcgtccatacccag

SEQ ID NO: 141 - DNA, 66 bp, synthetic construct
  source 1..66: mol_type = other DNA; note = PR8667
ctctgctaaagcaagcaggtgatgttgaagaaaaccccgggcctatgcctggtggacccggcgtac

SEQ ID NO: 142 - DNA, 73 bp, synthetic construct
  source 1..73: mol_type = other DNA; note = PR8669
attctcctcgacgtcaccgcatgttagcagacttcctctgccctctggcttttccaactgcacgtcacctatg

SEQ ID NO: 143 - DNA, 20 bp, synthetic construct
  source 1..20: mol_type = other DNA; note = PR6868
cctcagacagtggttcaaag

SEQ ID NO: 144 - DNA, 69 bp, synthetic construct
  source 1..69: mol_type = other DNA; note = PR8867
attctcctcgacgtcaccgcatgttagcagacttcctctgccctccttgtacaactcgtccatacccag

SEQ ID NO: 145 - DNA, 62 bp, synthetic construct
  source 1..62: mol_type = other DNA; note = PR8868
tctgctaacatgcggtgacgtcgaggagaatcctggcccaatgcctggtggacccggcgtac

SEQ ID NO: 146 - DNA, 71 bp, synthetic construct
  source 1..71: mol_type = other DNA; note = PR8869
cttcaacatcacctgcttgctttagcagagagaagtttgtggctggcttttccaactgcacgtcacctatg

SEQ ID NO: 147 - DNA, 64 bp, synthetic construct
  source 1..64: mol_type = other DNA; note = PR8870
ctctgctaaagcaagcaggtgatgttgaagaaaaccccgggccaatggcccgccttgcactttc

SEQ ID NO: 148 - DNA, 59 bp, synthetic construct
  source 1..59: mol_type = other DNA; note = PR8871
caaagtttttttcttccatttcaggtgtcgtgaggaattcgccaccatggtgagcaagg

SEQ ID NO: 149 - DNA, 68 bp, synthetic construct
  source 1..68: mol_type = other DNA; note = PR8875
tctgctaacatgcggtgacgtcgaggagaatcctggcccaatgtggcagcttctgctcccgacagcac

SEQ ID NO: 150 - DNA, 67 bp, synthetic construct
  source 1..67: mol_type = other DNA; note = PR8876
ggttgattatcgataagcttgatatcgttcgaagcgatcgcctacttatcctgaggatctttgcgcc

SEQ ID NO: 151 - DNA, 24 bp, synthetic construct
  source 1..24: mol_type = other DNA; note = PR6702
ctcccgggagctttttgcaaaagc

SEQ ID NO: 152 - DNA, 94 bp, synthetic construct
  source 1..94: mol_type = other DNA; note = PR8982
ggtgtatacgggtctgagtacacggaaccaggagacgtacgagaccttgaaacacgaaaaacctccacaatagtgtacagctagctggccagac

SEQ ID NO: 153 - DNA, 104 bp, synthetic construct
  source 1..104: mol_type = other DNA; note = PR8983
cgtacgtctcctggttccgtgtactcagacccgtatacaccccgtcagacttttcgtagcttgtgatggcgtctttaactttaaagaaagtcacggttgcggag

SEQ ID NO: 154 - DNA, 61 bp, synthetic construct
  source 1..61: mol_type = other DNA; note = PR8862
caaacggtggcagaacgagaaacttggccttgacgcgcccgcgtaccagcagggccagaac

SEQ ID NO: 155 - DNA, 59 bp, synthetic construct
  source 1..59: mol_type = other DNA; note = PR8860
catcacctgcttgctttagcagagagaagtttgtggcgcgagggggcagggcctgcatg

SEQ ID NO: 156 - DNA, 28 bp, synthetic construct
  source 1..28: mol_type = other DNA; note = PR4413
gccacaaacttctctctgctaaagcaag

SEQ ID NO: 157 - DNA, 24 bp, synthetic construct
  source 1..24: mol_type = other DNA; note = PR8857
actgtcatgccatccgtaagatgc

SEQ ID NO: 158 - DNA, 48 bp, synthetic construct
  source 1..48: mol_type = other DNA; note = PR4412
ttgctgatatgcgggggcgtccttatccaacaggagaaatatagggac

SEQ ID NO: 159 - DNA, 26 bp, synthetic construct
  source 1..26: mol_type = other DNA; note = PR9004
gtactcaccagtcacagaaaagcatc

SEQ ID NO: 160 - DNA, 42 bp, synthetic construct
  source 1..42: mol_type = other DNA; note = PR9005
caggccttgccacctagataggcgatcgcttcgaacgatatc

SEQ ID NO: 161 - DNA, 37 bp, synthetic construct
  source 1..37: mol_type = other DNA; note = PR9006
ctcctgttggataaggacgcccccgcatatcagcaag

SEQ ID NO: 162 - DNA, 37 bp, synthetic construct
  source 1..37: mol_type = other DNA; note = PR9007
gttcgaagcgatcgcctatctaggtggcaaggcctgc

SEQ ID NO: 163 - DNA, 18 bp, synthetic construct
  source 1..18: mol_type = other DNA; note = PR9275
ggtgccagggggaagacc

SEQ ID NO: 164 - DNA, 37 bp, synthetic construct
  source 1..37: mol_type = other DNA; note = PR4426
tccatttcaggtgtcgtgagaattcgccaccatggaa

SEQ ID NO: 165 - DNA, 47 bp, synthetic construct
  source 1..47: mol_type = other DNA; note = PR4427
gtgtacggtacaaacccgttaacaacaattctacttgtacagctcgt

SEQ ID NO: 166 - DNA, 829 bp, synthetic construct
  source 1..829: mol_type = other DNA; note = gBlock330
agctatgaaaccgagcgagagaattcgccaccatgtggcagcttctgctcccgacagcacttttgcttctcgtatcagcaggtatgcgaaccgaagacctcccgaaggccgtcgtgtttttggagcctcaatggtatagagtcttggaaaaggatagtgtaactttgaaatgtcaaggcgcgtattcaccggaggacaattcaacacaatggtttcataacgagtctctgatctccagtcaagccagctcctatttcatagacgcggctaccgtggatgattccggggaatataggtgtcaaacaaaccttagtacactttctgatccggtccaactcgaagttcatataggatggctgttgttgcaagcgccacgatgggtattcaaggaggaagatccaatacacctgagatgtcactcttggaaaaataccgctcttcataaggttacctacctccagaacggaaaaggcaggaaatacttccaccacaattcagacttttatattcctaaagcgaccttgaaggacagcggtagctacttctgtcggggattgttcggatcaaaaaatgtgtcatctgagacagttaacataacaatcacccagggtctggctgttagcactatatcctctttcttcccgccggggtatcaggtgtccttttgcctcgtaatggtactgctgttcgcagtagacactggtctttacttttccgtaaagaccaatataaggagcagtactcgcgactggaaagaccataagtttaaatggcgcaaagatcctcaggataagtaggcggccgcgcatcctctgcctacaagaacact

SEQ ID NO: 167 - DNA, 745 bp, synthetic construct
  source 1..745: mol_type = other DNA; note = gBlock331
agctatgaaaccgagcgagaggaattcgccaccatgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgcaaacggtggcagaacgagaaacttggccttgacgcgggagacgagtatgaagacgaaaatttgtacgagggtcttaatctcgacgattgcagtatgtacgaggacattagccgcggacttcaagggacttaccaagatgtaggatcactgaacataggtgacgtgcagttggaaaagccataggcggccgccatcctctgcctacaagaacact

SEQ ID NO: 168 - DNA, 754 bp, synthetic construct
  source 1..754: mol_type = other DNA; note = gBlock332
agctatgaaaccgagcgagaggaattcgccaccatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgattcaaaagcaggcatggaggaggaccatacttacgaaggtctggacatagatcaaacggcgacttatgaggacatcgtcactctgcgaacgggggaggttaaatggagtgtaggagaacatcccggacaggaataggcggccgccatcctctgcctacaagaacact

SEQ ID NO: 169 - DNA, 1729 bp, synthetic construct
  source 1..1729: mol_type = other DNA; note = gBlock339
cgatctgtttccttgacactatggggtttactacaaaaattatatttctttacaatctcgttcttgtttatgcggggtttgatgatcctaggaaagcaattgagctggttcagaaaaggtacggacgcccctgtgactgttccggaggtcaagtgtccgaaccacctagtgacagagtatctcaggtcacatgctctgggaagaccgcgtacctcatgcctgaccagagatggaagtgtaaatccatcccgaaagacacgtcaccgagcggccccctccaagaatgcccgtgcaattcctaccagagctcagttcacagttcatgttacacctcttaccagcagtgcagatccgggaacaaaacgtattacactgccacgctcttgaaaacacaaactgggggaacttccgacgtccaggtgctgggttcaacgaataaactgatacaaagcccatgtaacgggattaaaggccaatctatttgttggagtacgacagcgccaatacacgtatcagatggaggcggtccccttgacaccacacgcattaaaagtgttcaaaggaagcttgaagagattcataaagcactttacccggagttgcagtaccacccgttggcgattcctaaagtccgagataatctgatggtagacgcccagacattgaatatattgaatgccacgtacaatcttcttcttatgtctaacacttccctggtcgatgactgttggctctgtttgaaacttggcccaccgaccccgcttgccattcctaatttcctccttagttatgtaacaagaagtagcgataacattagttgtctcataattcccccattgctggtccagcctatgcagtttagcaattcctcatgcttgttcagtccctcttacaatagtacggaagaaatagatctgggtcacgttgcgttttctaattgcaccagcattactaatgttacaggtccgatatgcgcggtgaatggttccgtcttcctctgtgggaacaatatggcttatacctatttgcccactaactggacaggtttgtgcgtactcgcgactcttcttccggatatagatatcattcctggagacgagcccgttccgattcctgccatcgatcatttcatataccgacctaaacgagccatacaattcattcccttgttggcggggctcggtatcacagccgcattcaccacaggtgccaccggccttggagtctcagttacccaatatactaagctcagcaatcaactgataagcgatgtccaaattttgagctctacaatccaggatcttcaagatcaagtggatagtcttgccgaggtggtcctgcaaaatcggcgcggcttggatctcctcacagccgaacagggaggcatctgtctcgcacttcaggaaaaatgctgtttctatgtgaacaaaagtggtatagttcgcgataaaataaagacactccaggaggaacttgaacggaggcgcaaagaccttgcatctaatccgctgtggaccggccttcaaggcttgctcccctaccttcttccttttctgggacccttgcttactcttttgctgttgttgacaattgggccctgcatctttaatcgcctcgtacagttcgttaaggataggatttctgttgtccaggcccttgtcctcacccagcagtaccaccaattgaagcctttggagtacgaaccgtaactcaaatcctgcacaacaga

SEQ ID NO: 170 - DNA, 1546 bp, synthetic construct
  source 1..1546: mol_type = other DNA; note = gBlock357
tatggattattggggtcaaggtactcttgtcaccgtctcctcaggaagcgcttctgcgcctacgctctttccactggtttcatgtgagaacagcccttctgatacgtccagcgtagccgttggttgtcttgcccaggatttcttgccagattccattaccttttcctggaaatataagaataattccgatataagttccacccgcgggttcccctctgtcttgcgaggtggtaaatatgccgcaaccagtcaagtccttcttcctagtaaggatgtaatgcaaggcactgacgaacatgtggtctgcaaagtccagcatccgaatggaaacaaggagaagaacgtaccattgcccgtaatcgccgagttgccaccgaaggtgtcagtattcgtgccgccgcgcgatgggttcttcgggaatcctcggaaaagcaaactgatttgtcaagctaccgggtttagtccccggcaaattcaagtctcctggctgcgagaaggaaagcaagtcggaagtggggtgactactgaccaagtccaagctgaagcaaaggaatctggtcccaccacatacaaagtgacttcaacattgacaattaaggaatcagattggctctcacagagcatgttcacgtgtcgagtggaccaccggggtttgacctttcagcaaaacgcaagcagcatgtgtgttccggaccaggacacagccatccgtgtctttgcgattcccccgagctttgcaagcatctttctgaccaaatcaactaaattgacgtgcctggttaccgacctgactacatatgattcagtgactatttcctggacaagacaaaacggtgaggccgtcaagacacatacgaatatatctgaatcccatccgaatgcaaccttctccgctgttggtgaagccagtatttgtgaagatgactggaatagcggcgaacgttttacctgtacagtgactcacaccgatctcccatcaccacttaagcagaccataagtagacccaagggagtcgctcttcatcgtccggatgtctatctcttgcccccagctcgagagcaacttaatctccgtgagagcgcgaccatcacgtgtctcgtcacgggcttctcccccgcagatgttttcgttcaatggatgcaacgcggtcaaccactgagtcctgagaagtatgtgacttctgccccaatgccggaaccacaagcacccggtaggtactttgctcactccatcttgaccgtttccgaggaagagtggaacactggggagacctacacctgtgttgtcgcccacgaagcgttgcccaatcgtgttaccgaacgaacggtcgacaagtccaccgaaggagaagtttccgctgatgaagaagggtttgaaaatctctgggcgacagcatctacatttattgtcctctttctcctctctcttttctatagtacaactgtcacactctttaaggtgaagtgatgtacagctagctggccagacatgatcctgcaggttcgaacgatatcaagcttatcgataatcaacctctggattaca

SEQ ID NO: 171 - DNA, 1329 bp, synthetic construct
  source 1..1329: mol_type = other DNA; note = gBlock359
cgaacgctccgagggccgccactccaccggcggcatggacgagctgtacaaggagggcagaggaagtctgctaacatgcggtgacgtcgaggagaatcctggcccaatggcctggatgatgcttctcctcggactccttgcttatggatccggagtcgactctcagtctgccctgactcagccgccctcagtgtctggggccccggggcagagggtctccatctcctgcactgggggcagctccaacttcggggcaggttatgatgtacactggtaccagcagcttccagcaacagcccccaaactcctcatctatggtaacaacaatcggccctcaggggtccctgaccgattctctggctccaagtctggcacctcagcctccctggccatcactgggctccaggctgaggatgagggtgattatttctgccagtcctttgacaccagcctgagtggttggattttcggcggcgggaccaaactgaccgtcctaggtcagcccaaggcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcagccacaaacttctctctgctaaagcaagcaggtgatgttgaagaaaaccccgggcctatggactggacctggaggatcctcttcttggtggcggccgccacaggcgcgcactcccaggtccagcttgtgcagtctggggctgaggtgaggaaccctggggcctcagtgaaggtttcctgcaaggcttctggatacaccttcactagttatgctatccattgggtgcgccaggcccccggacacaggcttgagtgggtgggacggatcaacactgacaacggtaacacaaagtattcacagaagttccatggcagagtcgccctttccagggacacatccgcgagcacaacctacatggacctgagtagcctgaactctgaggacacggctgtctattattgtgcgagagcgttttactattcttcaggcgtaatgtttgactcctggggccagggagccctggtcaccgtctcctcagctagcaccaagggcccatcggtcttccccctggcacc

SEQ ID NO: 172 - DNA, 507 bp, synthetic construct
  source 1..507: mol_type = other DNA; note = gBlock365
aaaaaaatgaagtggaaagccctgttcacagcagccattctccaagctcaactgccaataactgaagctcaatccttcggtttgctggatccaaagctctgttacctgctggacgggattttgtttatttacggagtgatcctgacagctctgttcctgagggtgaaattcagcaggtctgcagatgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatgaaaaaaa

SEQ ID NO: 173 - protein, 32 aa, Homo sapiens
  source 1..32: note = CD79A - leader sequence
MPGGPGVLQALPATIFLLFLLSAVYLGPGCQA

SEQ ID NO: 174 - protein, 111 aa, Homo sapiens
  source 1..111: note = CD79A - Extracellular domain without leader sequence
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNR

SEQ ID NO: 175 - protein, 194 aa, Homo sapiens
  source 1..194: note = CD79A - Full length without leader sequence
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 176 - protein, 28 aa, Homo sapiens
  source 1..28: note = CD79B - leader sequence
MARLALSPVPSHWMVALLLLLSAEPVPA

SEQ ID NO: 177 - protein, 131 aa, Homo sapiens
  source 1..131: note = CD79B - Extracellular domain without leader sequence
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 178 - protein, 201 aa, Homo sapiens
  source 1..201: note = CD79B - Full length without leader sequence
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 179 - DNA, 294 bp, Homo sapiens
  source 1..294: mol_type = other DNA; note = IgG1 heavy chain constant domain CH1, variant
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtt

SEQ ID NO: 180 - DNA, 339 bp, Homo sapiens; identical to SEQ ID NO: 63
  source 1..339: mol_type = other DNA; note = IgG1 heavy chain constant domain CH2, variant

SEQ ID NO: 181 - DNA, 315 bp, Homo sapiens; identical to SEQ ID NO: 65
  source 1..315: mol_type = other DNA; note = IgG1 heavy chain constant domain CH3, variant

SEQ ID NO: 182 - DNA, 102 bp, Homo sapiens
  source 1..102: mol_type = other DNA; note = FceRIg - Intracellular domain, variant
gacgccatcacaagctacgaaaagtctgacggggtgtatacgggtctgagtacacggaaccaggagacctacgagaccttgaaacacgaaaaacctccacaa

SEQ ID NO: 183 - protein, 34 aa, Homo sapiens
  source 1..34: note = FceRIg - Intracellular domain, variant
DAITSYEKSDGVYTGLSTRNQETYETLKHEKPPQ

SEQ ID NO: 184 - protein, 249 aa, Homo sapiens
  source 1..249: note = CD79A extracellular and membrane domain_CD3 zeta intracellular domain fusion, without leader sequence
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 185 - protein, 260 aa, Homo sapiens
  source 1..260: note = CD79B extracellular and membrane domain_CD3 zeta intracellular domain fusion
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 186 - skipped

SEQ ID NO: 187 - protein, 6 aa, Homo sapiens
  source 1..6: note = SLAMF1 - ITSM
TIYAQV

SEQ ID NO: 188 - protein, 13 aa, Homo sapiens
  source 1..13: note = SLAMF1 - intracellular motif
TIYAQVYTVYASV

SEQ ID NO: 189 - protein, 6 aa, Homo sapiens
  source 1..6: note = CD226 - Immunoreceptor tyrosine tail (ITT)-like motif
EDIYVN

SEQ ID NO: 190 - protein, 6 aa, Homo sapiens
  source 1..6: note = CD244 - ITSM 1
TLYSLI

SEQ ID NO: 191 - protein, 6 aa, Homo sapiens
  source 1..6: note = CD244 - ITSM 2
TIYEVI

SEQ ID NO: 192 - protein, 4 aa, Homo sapiens
  source 1..4: note = CD28 - intracellular motif 1
YMNM

SEQ ID NO: 193 - protein, 4 aa, Homo sapiens
  source 1..4: note = CD28 - intracellular motif 2
PYAP

SEQ ID NO: 194 - protein, 14 aa, Homo sapiens
  source 1..14: note = TMIGD2 - proline rich motif 2
PSPRPCPSPRPGHP

SEQ ID NO: 195 - protein, 6 aa, Homo sapiens
  source 1..6: note = SLAMF7 - ITSM 1
TEYDTI

SEQ ID NO: 196 - protein, 6 aa, Homo sapiens
  source 1..6: note = SLAMF7 - ITSM 2
TVYSTV

SEQ ID NO: 197 - protein, 4 aa, Homo sapiens
  source 1..4: note = CD7 - tyrosine motif
YEDM

SEQ ID NO: 198 - protein, 4 aa, Homo sapiens
  source 1..4: note = HCST - YXXM motif
YINM

SEQ ID NO: 199 - protein, 15 aa, Homo sapiens
  source 1..15: note = TYROB - ITAM
YQELQGQRSDVYSDL

SEQ ID NO: 200 - protein, 6 aa, Homo sapiens
  source 1..6: note = SLAMF6 - ITSM 1
TVYASV

SEQ ID NO: 201 - protein, 7 aa, Homo sapiens
  source 1..7: note = SLAMF6 - ITSM 2
ITIYSTI

SEQ ID NO: 202 - protein, 133 aa, Homo sapiens
  source 1..133: note = CD79A extracellular and membrane domain, without leader sequence
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLF

SEQ ID NO: 203 - protein, 152 aa, Homo sapiens
  source 1..152: note = CD79B extracellular and membrane domain, without leader sequence
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLL

SEQ ID NO: 204 - DNA, 126 bp, Homo sapiens
  source 1..126: mol_type = other DNA; note = TNFRSF9 - intracellular domain
aagcgcggccgtaaaaaacttctgtacatttttaagcaacctttcatgcgccccgtgcagaccacacaggaggaggatggatgttcatgcaggttcccggaagaggaagaaggcgggtgtgagctt

SEQ ID NO: 205 - protein, 42 aa, Homo sapiens
  source 1..42: note = TNFRSF9 - intracellular domain
KRGRKKLLYIFKQPFMRPVQTTQEEDGCSCRFPEEEEGGCEL

SEQ ID NO: 206 - DNA, 194 bp, Homo sapiens
  source 1..194: mol_type = other DNA; note = SLAMF1 - intracellular domain
cagctccgccggcgtgggaaaaccaaccactaccaaaccacagtggaaaaaaaaagccttaccatttacgcccaggtacagaagcctggtccgctccagaaaaaactcgactcctttcccgctcaggacccatgcaccacgatctacgtggctgcgacggagcccgtacctgaaagcgtccaagagacaaacag

SEQ ID NO: 207 - protein, 77 aa, Homo sapiens
  source 1..77: note = SLAMF1 - intracellular domain
QLRRRGKTNHYQTTVEKKSLTIYAQVQKPGPLQKKLDSFPAQDPCTTIYVAATEPVPESVQETNSITVYASVTLPES

SEQ ID NO: 208 - DNA, 342 bp, Homo sapiens
  source 1..342: mol_type = other DNA; note = KIR2DL4 - intracellular domain
cgatggtgctccaagaagaaggacgccgctgtaatgaaccaggaacccgctggccacaggacggtgaatagagaagatagcgacgagcaggatcctcaggaggtcacatacgcgcagctggatcattgcatattcacgcagcgcaaaattactggcccttcccagcgaagtaagcgccccagtaccgacaccagcgtttgtatcgagctgcccaatgcagaacctagagctctcagccccgcccacgagcaccattcacaagcgctcatgggctctagtcgcgagaccactgccctttctcagacacaactggcttccagtaatgtgcccgcagccggcatc

SEQ ID NO: 209 - protein, 114 aa, Homo sapiens
  source 1..114: note = KIR2DL4 - intracellular domain
RWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 210 - DNA, 183 bp, Homo sapiens
  source 1..183: mol_type = other DNA; note = CD226
aatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtc

SEQ ID NO: 211 - protein, 61 aa, Homo sapiens
  source 1..61: note = CD226 - intracellular domain
NRRRRRERRDLFTESWDTQKAPNNYRSPISTSQPTNQSMDDTREDIYVNYPTFSRRPKTRV

SEQ ID NO: 212 - DNA, 360 bp, Homo sapiens
  source 1..360: mol_type = other DNA; note = CD244 - intracellular domain
tggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagc

SEQ ID NO: 213 - protein, 120 aa, Homo sapiens
  source 1..120: note = CD244 - intracellular domain
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 214 - DNA, 123 bp, Homo sapiens
  source 1..123: mol_type = other DNA; note = CD28 - intracellular domain
agatcaaaaaggagcagactgcttcactctgactatatgaacatgactccccgtcgccccggccccacacggaagcactatcaaccctacgcccctcctagagattttgccgcgtataggtct

SEQ ID NO: 215 - protein, 41 aa, Homo sapiens
  source 1..41: note = CD28 - intracellular domain
RSKRSRLLHSDYMNMTPRRPGPTRKHYQPYAPPRDFAAYRS

SEQ ID NO: 216 - DNA, 333 bp, Homo sapiens
  source 1..333: mol_type = other DNA; note = TMIGD2 - intracellular domain
ttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggag

SEQ ID NO: 217 - protein, 111 aa, Homo sapiens
  source 1..111: note = TMIGD2 - intracellular domain
FWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 218 - DNA, 264 bp, Homo sapiens
  source 1..264: mol_type = other DNA; note = SLAMF7 - intracellular domain
tggttccttaagcgagaacgtcaggaggagtatatcgaggagaaaaagagggtggacatttgtagagagactccaaacatttgcccgcactccggggagaacacagaatatgacacaatcccccacactaatcggaccattctcaaagaggaccctgccaatactgtctattctacagttgagattcctaagaagatggaaaacccccactcactgctcaccatgcctgacacaccccgtctgtttgcttatgagaatgtaatc

SEQ ID NO: 219 - protein, 88 aa, Homo sapiens
  source 1..88: note = SLAMF7 - intracellular domain
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 220 - DNA, 174 bp, Homo sapiens
  source 1..174: mol_type = other DNA; note = TNFRSF18 - intracellular domain
cagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtg

SEQ ID NO: 221 - protein, 58 aa, Homo sapiens
  source 1..58: note = TNFRSF18 - intracellular domain
QLGLHIWQLRSQCMWPRETQLLLEVPPSTEDARSCQFPEEERGERSAEEKGRLGDLWV

SEQ ID NO: 222 - DNA, 216 bp, Homo sapiens
  source 1..216: mol_type = other DNA; note = CD44 - intracellular domain
aactccagaagacggtgcgggcagaaaaagaagttagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtg

SEQ ID NO: 223 - protein, 72 aa, Homo sapiens
  source 1..72: note = CD44 - intracellular domain
NSRRRCGQKKKLVINSGNGAVEDRKPSGLNGEASKSQEMVHLVNKESSETPDQFMTADETRNLQNVDMKIGV

SEQ ID NO: 224 - DNA, 117 bp, Homo sapiens
  source 1..117: mol_type = other DNA; note = CD7 - intracellular domain
cgaacgcaaatcaaaaaactctgctcttggcgggacaagaattccgcagcttgcgtggtgtatgaagacatgtctcacagtcggtgtaatacactctctagcccgaaccaataccaa

SEQ ID NO: 225 - protein, 39 aa, Homo sapiens
  source 1..39: note = CD7 - intracellular domain
RTQIKKLCSWRDKNSAACVVYEDMSHSRCNTLSSPNQYQ

SEQ ID NO: 226 - DNA, 297 bp, Homo sapiens
  source 1..297: mol_type = other DNA; note = CD84 - intracellular domain
aggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgatt

SEQ ID NO: 227 - protein, 99 aa, Homo sapiens
  source 1..99: note = CD84 - intracellular domain
RLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 228 - DNA, 72 bp, Homo sapiens
  source 1..72: mol_type = other DNA; note = HCST - intracellular domain
ctgtgtgctcgcccacggagaagtccagcccaggaggatggtaaggtttatataaacatgccaggaaggggg

SEQ ID NO: 229 - protein, 24 aa, Homo sapiens
  source 1..24: note = HCST - intracellular domain
LCARPRRSPAQEDGKVYINMPGRG

SEQ ID NO: 230 - DNA, 156 bp, Homo sapiens
  source 1..156: mol_type = other DNA; note = TYROB - intracellular domain
tacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataag

SEQ ID NO: 231 - protein, 52 aa, Homo sapiens
  source 1..52: note = TYROB - intracellular domain
YFLGRLVPRGRGAAEAATRKQRITETESPYQELQGQRSDVYSDLNTQRPYYK

SEQ ID NO: 232 - DNA, 420 bp, Homo sapiens
  source 1..420: mol_type = other DNA; note = FCRL3 - intracellular domain
cattacgctagagcaaggagaaaacccggcggtctgtcagccacgggaacatcttctcacagtcccagcgagtgccaggagccaagttcatccagaccttcacgaattgatccgcaggagcccacccacagcaaacccctcgcgccaatggagctcgagcccatgtacagcaacgtgaaccctggtgattcaaaccccatctactcacagatctggtccattcagcatacaaaagaaaatagtgctaactgtccaatgatgcaccaggagcatgaggagttgaccgtcctgtactctgaactgaagaaaactcacccagatgacagcgcaggtgaggcaagcagccgagggcgcgcacacgaagaggacgacgaagaaaattacgaaaatgtgccacgtgttctgctggcatccgaccac

SEQ ID NO: 233 - protein, 140 aa, Homo sapiens
  source 1..140: note = FCRL3 - intracellular domain
HYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 234 - DNA, 255 bp, Homo sapiens
  source 1..255: mol_type = other DNA; note = SLAMF6 - intracellular domain
ttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtt

SEQ ID NO: 235 - protein, 85 aa, Homo sapiens
  source 1..85: note = SLAMF6 - intracellular domain
LRKRRDSLSLSTQRTQGPAESARNLEYVSVSPTNNTVYASVTHSNRETEIWTPRENDTITIYSTINHSKESKPTFSRATALDNVV

SEQ ID NO: 236 - DNA, 255 bp, Homo sapiens
  source 1..255: mol_type = other DNA; note = TNFRSF13C - intracellular domain
agctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaa

SEQ ID NO: 237 - protein, 85 aa, Homo sapiens
  source 1..85: note = TNFRSF13C - intracellular domain
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 238 - DNA, 1545 bp, synthetic construct
  source 1..1545: mol_type = other DNA; note = CD79A(EC-TM)-4-1BB(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 239 - protein, 514 aa, synthetic construct
  source 1..514: note = CD79A(EC-TM)-4-1BB(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 240 - DNA, 1542 bp, synthetic construct
  source 1..1542: mol_type = other DNA; note = CD79A(EC-TM)-CD28(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 241 - protein, 513 aa, synthetic construct
  source 1..513: note = CD79A(EC-TM)-CD28(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 242 - DNA, 1650 bp, synthetic construct
  source 1..1650: mol_type = other DNA; note = CD79A(EC-TM)-CD150(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctccgccggcgtgggaaaaccaaccactaccaaaccacagtggaaaaaaaaagccttaccatttacgcccaggtacagaagcctggtccgctccagaaaaaactcgactcctttcccgctcaggacccatgcaccacgatctacgtggctgcgacggagcccgtacctgaaagcgtccaagagacaaacagcataactgtgtatgcttccgtcactctgcctgagtccgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 243 - protein, 513 aa, synthetic construct; identical to SEQ ID NO: 241
  source 1..513: note = CD79A(EC-TM)-CD150(CYT)-CD79B(EC-TM)-CD3z(CYT)

SEQ ID NO: 244 - DNA, 1602 bp, synthetic construct
  source 1..1602: mol_type = other DNA; note = CD79A(EC-TM)-CD226(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 245 - protein, 513 aa, synthetic construct; identical to SEQ ID NO: 241
  source 1..513: note = CD79A(EC-TM)-CD226(CYT)-CD79B(EC-TM)-CD3z(CYT)

SEQ ID NO: 246 - DNA, 1716 bp, synthetic construct
  source 1..1716: mol_type = other DNA; note = CD79A(EC-TM)-CD84(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 247 - protein, 571 aa, synthetic construct
  source 1..571: note = CD79A(EC-TM)-CD84(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 248 - DNA, 1779 bp, synthetic construct
  source 1..1779: mol_type = other DNA; note = CD79A(EC-TM)-CD244(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 249 - protein, 592 aa, synthetic construct
  source 1..592: note = CD79A(EC-TM)-CD244(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 250 - DNA, 1536 bp, synthetic construct
  source 1..1536: mol_type = other DNA; note = CD79A(EC-TM)-DR3(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 251 - protein, 511 aa, synthetic construct
  source 1..511: note = CD79A(EC-TM)-DR3(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 252 - DNA, 1839 bp, synthetic construct
  source 1..1839: mol_type = other DNA; note = CD79A(EC-TM)-CD7(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcattacgctagagcaaggagaaaacccggcggtctgtcagccacgggaacatcttctcacagtcccagcgagtgccaggagccaagttcatccagaccttcacgaattgatccgcaggagcccacccacagcaaacccctcgcgccaatggagctcgagcccatgtacagcaacgtgaaccctggtgattcaaaccccatctactcacagatctggtccattcagcatacaaaagaaaatagtgctaactgtccaatgatgcaccaggagcatgaggagttgaccgtcctgtactctgaactgaagaaaactcacccagatgacagcgcaggtgaggcaagcagccgagggcgcgcacacgaagaggacgacgaagaaaattacgaaaatgtgccacgtgttctgctggcatccgaccacgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 253 - protein, 612 aa, synthetic construct
  source 1..612: note = CD79A(EC-TM)-CD7(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDHATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 254 - DNA, 1593 bp, synthetic construct
  source 1..1593: mol_type = other DNA; note = CD79A(EC-TM)-CD357(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 255 - protein, 530 aa, synthetic construct
  source 1..530: note = CD79A(EC-TM)-CD357(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFQLGLHIWQLRSQCMWPRETQLLLEVPPSTEDARSCQFPEEERGERSAEEKGRLGDLWVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 256 - DNA, 1545 bp, synthetic construct
  source 1..1545: mol_type = other DNA; note = CD79A(EC-TM)-FceR1g(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagactgaagattcaggtccggaaagccgcaattacttcatacgaaaaaagcgacggggtttatacaggcctgagcacgcgcaaccaggaaacttacgaaacgctgaagcatgaaaagcccccgcaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 257 - protein, 514 aa, synthetic construct
  source 1..514: note = CD79A(EC-TM)-FceR1g(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLKIQVRKAAITSYEKSDGVYTGLSTRNQETYETLKHEKPPQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 258 - DNA, 1575 bp, synthetic construct
  source 1..1575: mol_type = other DNA; note = CD79A(EC-TM)-DAP12(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 259 - protein, 524 aa, synthetic construct
  source 1..524: note = CD79A(EC-TM)-DAP12(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFYFLGRLVPRGRGAAEAATRKQRITETESPYQELQGQRSDVYSDLNTQRPYYKATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 260 - DNA, 1491 bp, synthetic construct
  source 1..1491: mol_type = other DNA; note = CD79A(EC-TM)-DAP10(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttctgtgtgctcgcccacggagaagtccagcccaggaggatggtaaggtttatataaacatgccaggaaggggggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 261 - protein, 496 aa, synthetic construct
  source 1..496: note = CD79A(EC-TM)-DAP10(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFLCARPRRSPAQEDGKVYINMPGRGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 262 - DNA, 1674 bp, synthetic construct
  source 1..1674: mol_type = other DNA; note = CD79A(EC-TM)-TNFRSF13C(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 263 - protein, 557 aa, synthetic construct
  source 1..557: note = CD79A(EC-TM)-TNFRSF13C(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFSWRRRQRRLRGASSAEAPDGDKDAPEPLDKVIILSPGISDATAPAWPPPGEDPGTTPPGHSVPVPATELGSTELVTTKTAGPEQQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 264 - DNA, 1761 bp, synthetic construct
  source 1..1761: mol_type = other DNA; note = CD79A(EC-TM)-CD158d(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgatggtgctccaagaagaaggacgccgctgtaatgaaccaggaacccgctggccacaggacggtgaatagagaagatagcgacgagcaggatcctcaggaggtcacatacgcgcagctggatcattgcatattcacgcagcgcaaaattactggcccttcccagcgaagtaagcgccccagtaccgacaccagcgtttgtatcgagctgcccaatgcagaacctagagctctcagccccgcccacgagcaccattcacaagcgctcatgggctctagtcgcgagaccactgccctttctcagacacaactggcttccagtaatgtgcccgcagccggcatcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 265 - protein, 586 aa, synthetic construct
  source 1..586: note = CD79A(EC-TM)-CD158d(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 266 - DNA, 1635 bp, synthetic construct
  source 1..1635: mol_type = other DNA; note = CD79A(EC-TM)-CD44(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 267 - protein, 544 aa, synthetic construct
  source 1..544: note = CD79A(EC-TM)-CD44(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNSRRRCGQKKKLVINSGNGAVEDRKPSGLNGEASKSQEMVHLVNKESSETPDQFMTADETRNLQNVDMKIGVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 268 - DNA, 1674 bp, synthetic construct
  source 1..1674: mol_type = other DNA; note = CD79A(EC-TM)-NTB-A(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 269 - protein, 557 aa, synthetic construct
  source 1..557: note = CD79A(EC-TM)-NTB-A(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFLRKRRDSLSLSTQRTQGPAESARNLEYVSVSPTNNTVYASVTHSNRETEIWTPRENDTITIYSTINHSKESKPTFSRATALDNVVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 270 - DNA, 1683 bp, synthetic construct
  source 1..1683: mol_type = other DNA; note = CD79A(EC-TM)-CD319(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggttccttaagcgagaacgtcaggaggagtatatcgaggagaaaaagagggtggacatttgtagagagactccaaacatttgcccgcactccggggagaacacagaatatgacacaatcccccacactaatcggaccattctcaaagaggaccctgccaatactgtctattctacagttgagattcctaagaagatggaaaacccccactcactgctcaccatgcctgacacaccccgtctgtttgcttatgagaatgtaatcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 271 - protein, 560 aa, synthetic construct
  source 1..560: note = CD79A(EC-TM)-CD319(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWFLKRERQEEYIEEKKRVDICRETPNICPHSGENTEYDTIPHTNRTILKEDPANTVYSTVEIPKKMENPHSLLTMPDTPRLFAYENVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 272 - DNA, 1752 bp, synthetic construct
  source 1..1752: mol_type = other DNA; note = CD79A(EC-TM)-CD28H(CYT)-CD79B(EC-TM)-CD3z(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttggataaggacgcccccgcatatcagcaaggacagaatcaactgtacaatgaactgaatctgggccgacgcgaagaatatgacgtgcttgataaacggagaggtagagatcccgagatgggtgggaagcctcagcgtagaaagaatccccaagagggactgtacaatgagctgcagaaagataagatggccgaggcttacagcgaaattggcatgaaaggtgagcggcggagaggaaagggacacgacggattgtaccagggtctcagcacagctaccaaagatacgtacgatgccttgcatatgcaggccttgccacctagatag

SEQ ID NO: 273 - protein, 583 aa, synthetic construct
  source 1..583: note = CD79A(EC-TM)-CD28H(CYT)-CD79B(EC-TM)-CD3z(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEEATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 274 - DNA, 1566 bp, synthetic construct
  source 1..1566: mol_type = other DNA; note = CD79A(EC-TM)-BAFF(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 275 - protein, 521 aa, synthetic construct
  source 1..521: note = CD79A(EC-TM)-BAFF(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFSWRRRQRRLRGASSAEAPDGDKDAPEPLDKVIILSPGISDATAPAWPPPGEDPGTTPPGHSVPVPATELGSTELVTTKTAGPEQQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 276 - DNA, 1605 bp, synthetic construct
  source 1..1605: mol_type = other DNA; note = CD79A(EC-TM)-BAFF(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 277 - protein, 534 aa, synthetic construct
  source 1..534: note = CD79A(EC-TM)-BAFF(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFSWRRRQRRLRGASSAEAPDGDKDAPEPLDKVIILSPGISDATAPAWPPPGEDPGTTPPGHSVPVPATELGSTELVTTKTAGPEQQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 278 - DNA, 1683 bp, synthetic construct
  source 1..1683: mol_type = other DNA; note = CD79A(EC-TM)-BAFF(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 279 - protein, 560 aa, synthetic construct
  source 1..560: note = CD79A(EC-TM)-BAFF(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFSWRRRQRRLRGASSAEAPDGDKDAPEPLDKVIILSPGISDATAPAWPPPGEDPGTTPPGHSVPVPATELGSTELVTTKTAGPEQQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 280 - DNA, 1566 bp, synthetic construct
  source 1..1566: mol_type = other DNA; note = CD79A(EC-TM)-CD44(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaactccagaagacggtgcgggcagaaaaagaagttagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 281 - protein, 521 aa, synthetic construct
  source 1..521: note = CD79A(EC-TM)-CD44(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNSRRRCGQKKKLVINSGNGAVEDRKPSGLNGEASKSQEMVHLVNKESSETPDQFMTADETRNLQNVDMKIGVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 282 - DNA, 1644 bp, synthetic construct
  source 1..1644: mol_type = other DNA; note = CD79A(EC-TM)-CD44(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaactccagaagacggtgcgggcagaaaaagaagttagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 283 - protein, 547 aa, synthetic construct
  source 1..547: note = CD79A(EC-TM)-CD44(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNSRRRCGQKKKLVINSGNGAVEDRKPSGLNGEASKSQEMVHLVNKESSETPDQFMTADETRNLQNVDMKIGVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 284 - DNA, 1683 bp, synthetic construct
  source 1..1683: mol_type = other DNA; note = CD79A(EC-TM)-NTB-A(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 285 - protein, 560 aa, synthetic construct
  source 1..560: note = CD79A(EC-TM)-NTB-A(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFLRKRRDSLSLSTQRTQGPAESARNLEYVSVSPTNNTVYASVTHSNRETEIWTPRENDTITIYSTINHSKESKPTFSRATALDNVVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 286 - DNA, 1638 bp, synthetic construct
  source 1..1638: mol_type = other DNA; note = CD79A(EC-TM)-CD226(CYT)-CD79B(EC-TM)-CD244(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagctag

SEQ ID NO: 287 - protein, 545 aa, synthetic construct
  source 1..545: note = CD79A(EC-TM)-CD226(CYT)-CD79B(EC-TM)-CD244(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 288 - DNA, 1395 bp, synthetic construct
  source 1..1395: mol_type = other DNA; note = CD79A(EC-TM)-CD226(CYT)-CD79B(EC-TM)-CD7(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcgaacgcaaatcaaaaaactctgctcttggcgggacaagaattccgcagcttgcgtggtgtatgaagacatgtctcacagtcggtgtaatacactctctagcccgaaccaataccaatag

SEQ ID NO: 289 - protein, 464 aa, synthetic construct
  source 1..464: note = CD79A(EC-TM)-CD226(CYT)-CD79B(EC-TM)-CD7(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRTQIKKLCSWRDKNSAACVVYEDMSHSRCNTLSSPNQYQ

SEQ ID NO: 290 - DNA, 1452 bp, synthetic construct
  source 1..1452: mol_type = other DNA; note = CD79A(EC-TM)-CD226(CYT)-CD79B(EC-TM)-CD357(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtgtag

SEQ ID NO: 291 - protein, 483 aa, synthetic construct
  source 1..483: note = CD79A(EC-TM)-CD226(CYT)-CD79B(EC-TM)-CD357(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLQLGLHIWQLRSQCMWPRETQLLLEVPPSTEDARSCQFPEEERGERSAEEKGRLGDLWV

SEQ ID NO: 292 - DNA, 1434 bp, synthetic construct
  source 1..1434: mol_type = other DNA; note = CD79A(EC-TM)-CD226(CYT)-CD79B(EC-TM)-DAP12(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataagtag

SEQ ID NO: 293 - protein, 477 aa, synthetic construct
  source 1..477: note = CD79A(EC-TM)-CD226(CYT)-CD79B(EC-TM)-DAP12(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLYFLGRLVPRGRGAAEAATRKQRITETESPYQELQGQRSDVYSDLNTQRPYYK

SEQ ID NO: 294 - DNA, 1350 bp, synthetic construct
  source 1..1350: mol_type = other DNA; note = CD79A(EC-TM)-CD226(CYT)-CD79B(EC-TM)-DAP10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 295 - protein, 449 aa, synthetic construct
  source 1..449: note = CD79A(EC-TM)-CD226(CYT)-CD79B(EC-TM)-DAP10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 296 - DNA, 1533 bp, synthetic construct
  source 1..1533: mol_type = other DNA; note = CD79A(EC-TM)-CD226(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 297 - protein, 510 aa, synthetic construct
  source 1..510: note = CD79A(EC-TM)-CD226(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 298 - DNA, 1494 bp, synthetic construct
  source 1..1494: mol_type = other DNA; note = CD79A(EC-TM)-CD226(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 299 - protein, 497 aa, synthetic construct
  source 1..497: note = CD79A(EC-TM)-CD226(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 300 - DNA, 1533 bp, synthetic construct
  source 1..1533: mol_type = other DNA; note = CD79A(EC-TM)-CD226(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 301 - protein, 510 aa, synthetic construct
  source 1..510: note = CD79A(EC-TM)-CD226(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 302 - DNA, 1611 bp, synthetic construct
  source 1..1611: mol_type = other DNA; note = CD79A(EC-TM)-CD226(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 303 - protein, 536 aa, synthetic construct
  source 1..536: note = CD79A(EC-TM)-CD226(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 304 - DNA, 1710 bp, synthetic construct
  source 1..1710: mol_type = other DNA; note = CD79A(EC-TM)-CD244(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 305 - protein, 569 aa, synthetic construct
  source 1..569: note = CD79A(EC-TM)-CD244(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 306 - DNA, 1527 bp, synthetic construct
  source 1..1527: mol_type = other DNA; note = CD79A(EC-TM)-CD244(CYT)-CD79B(EC-TM)-Dap10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 307 - protein, 508 aa, synthetic construct
  source 1..508: note = CD79A(EC-TM)-CD244(CYT)-CD79B(EC-TM)-Dap10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 308 - DNA, 1545 bp, synthetic construct
  source 1..1545: mol_type = other DNA; note = CD79A(EC-TM)-CD7(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 309 - protein, 514 aa, synthetic construct
  source 1..514: note = CD79A(EC-TM)-CD7(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 310 - DNA, 1284 bp, synthetic construct
  source 1..1284: mol_type = other DNA; note = CD79A(EC-TM)-CD7(CYT)-CD79B(EC-TM)-Dap10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 311 - protein, 427 aa, synthetic construct
  source 1..427: note = CD79A(EC-TM)-CD7(CYT)-CD79B(EC-TM)-Dap10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 312 - DNA, 1422 bp, synthetic construct
  source 1..1422: mol_type = other DNA; note = CD79A(EC-TM)-BAFF(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 313 - protein, 473 aa, synthetic construct
  source 1..473: note = CD79A(EC-TM)-BAFF(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFSWRRRQRRLRGASSAEAPDGDKDAPEPLDKVIILSPGISDATAPAWPPPGEDPGTTPPGHSVPVPATELGSTELVTTKTAGPEQQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 314 - DNA, 1575 bp, synthetic construct
  source 1..1575: mol_type = other DNA; note = CD79A(EC-TM)-CD226(CYT)-CD79B(EC-TM)-CD84(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgatttag

SEQ ID NO: 315 - protein, 524 aa, synthetic construct
  source 1..524: note = CD79A(EC-TM)-CD226(CYT)-CD79B(EC-TM)-CD84(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFNRRRRRERRDLFTESWDTQKAPNNYRSPISTSQPTNQSMDDTREDIYVNYPTFSRRPKTRVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 316 - DNA, 1572 bp, synthetic construct
  source 1..1572: mol_type = other DNA; note = CD79A(EC-TM)-CD244(CYT)-CD79B(EC-TM)-CD7(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcgaacgcaaatcaaaaaactctgctcttggcgggacaagaattccgcagcttgcgtggtgtatgaagacatgtctcacagtcggtgtaatacactctctagcccgaaccaataccaatag

SEQ ID NO: 317 - protein, 523 aa, synthetic construct
  source 1..523: note = CD79A(EC-TM)-CD244(CYT)-CD79B(EC-TM)-CD7(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRTQIKKLCSWRDKNSAACVVYEDMSHSRCNTLSSPNQYQ

SEQ ID NO: 318 - DNA, 1629 bp, synthetic construct
  source 1..1629: mol_type = other DNA; note = CD79A(EC-TM)-CD244(CYT)-CD79B(EC-TM)-CD357(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtgtag

SEQ ID NO: 319 - protein, 542 aa, synthetic construct
  source 1..542: note = CD79A(EC-TM)-CD244(CYT)-CD79B(EC-TM)-CD357(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLQLGLHIWQLRSQCMWPRETQLLLEVPPSTEDARSCQFPEEERGERSAEEKGRLGDLWV

SEQ ID NO: 320 - DNA, 1611 bp, synthetic construct
  source 1..1611: mol_type = other DNA; note = CD79A(EC-TM)-CD244(CYT)-CD79B(EC-TM)-DAP12(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataagtag

SEQ ID NO: 321 - protein, 536 aa, synthetic construct
  source 1..536: note = CD79A(EC-TM)-CD244(CYT)-CD79B(EC-TM)-DAP12(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLYFLGRLVPRGRGAAEAATRKQRITETESPYQELQGQRSDVYSDLNTQRPYYK

SEQ ID NO: 322 - DNA, 1671 bp, synthetic construct
  source 1..1671: mol_type = other DNA; note = CD79A(EC-TM)-CD244(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 323 - protein, 556 aa, synthetic construct
  source 1..556: note = CD79A(EC-TM)-CD244(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 324 - DNA, 1710 bp, synthetic construct
  source 1..1710: mol_type = other DNA; note = CD79A(EC-TM)-CD244(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 325 - protein, 569 aa, synthetic construct
  source 1..569: note = CD79A(EC-TM)-CD244(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 326 - DNA, 1788 bp, synthetic construct
  source 1..1788: mol_type = other DNA; note = CD79A(EC-TM)-CD244(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagcgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 327 - protein, 595 aa, synthetic construct
  source 1..595: note = CD79A(EC-TM)-CD244(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 328 - DNA, 1386 bp, synthetic construct
  source 1..1386: mol_type = other DNA; note = CD79A(EC-TM)-CD7(CYT)-CD79B(EC-TM)-CD357(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtgtag

SEQ ID NO: 329 - protein, 461 aa, synthetic construct
  source 1..461: note = CD79A(EC-TM)-CD7(CYT)-CD79B(EC-TM)-CD357(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLQLGLHIWQLRSQCMWPRETQLLLEVPPSTEDARSCQFPEEERGERSAEEKGRLGDLWV

SEQ ID NO: 330 - DNA, 1368 bp, synthetic construct
  source 1..1368: mol_type = other DNA; note = CD79A(EC-TM)-CD7(CYT)-CD79B(EC-TM)-DAP12(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataagtag

SEQ ID NO: 331 - protein, 455 aa, synthetic construct
  source 1..455: note = CD79A(EC-TM)-CD7(CYT)-CD79B(EC-TM)-DAP12(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLYFLGRLVPRGRGAAEAATRKQRITETESPYQELQGQRSDVYSDLNTQRPYYK

SEQ ID NO: 332 - DNA, 1467 bp, synthetic construct
  source 1..1467: mol_type = other DNA; note = CD79A(EC-TM)-CD7(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 333 - protein, 488 aa, synthetic construct
  source 1..488: note = CD79A(EC-TM)-CD7(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 334 - DNA, 1428 bp, synthetic construct
  source 1..1428: mol_type = other DNA; note = CD79A(EC-TM)-CD7(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 335 - protein, 475 aa, synthetic construct
  source 1..475: note = CD79A(EC-TM)-CD7(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 336 - DNA, 1467 bp, synthetic construct
  source 1..1467: mol_type = other DNA; note = CD79A(EC-TM)-CD7(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcgaacgcaaatcaaaaaactctgctcttggcgggacaagaattccgcagcttgcgtggtgtatgaagacatgtctcacagtcggtgtaatacactctctagcccgaaccaataccaagccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 337 - protein, 488 aa, synthetic construct
  source 1..488: note = CD79A(EC-TM)-CD7(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRTQIKKLCSWRDKNSAACVVYEDMSHSRCNTLSSPNQYQATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 338 - DNA, 1545 bp, synthetic construct; identical to SEQ ID NO: 308
  source 1..1545: mol_type = other DNA; note = CD79A(EC-TM)-CD7(CYT)-CD79B(EC-TM)-CD28H(CYT)

SEQ ID NO: 339 - protein, 514 aa, synthetic construct; identical to SEQ ID NO: 309
  source 1..514: note = CD79A(EC-TM)-CD7(CYT)-CD79B(EC-TM)-CD28H(CYT)

SEQ ID NO: 340 - DNA, 1425 bp, synthetic construct
  source 1..1425: mol_type = other DNA; note = CD79A(EC-TM)-CD357(CYT)-CD79B(EC-TM)-DAP12(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataagtag

SEQ ID NO: 341 - protein, 474 aa, synthetic construct
  source 1..474: note = CD79A(EC-TM)-CD357(CYT)-CD79B(EC-TM)-DAP12(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFQLGLHIWQLRSQCMWPRETQLLLEVPPSTEDARSCQFPEEERGERSAEEKGRLGDLWVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLYFLGRLVPRGRGAAEAATRKQRITETESPYQELQGQRSDVYSDLNTQRPYYK

SEQ ID NO: 342 - DNA, 1341 bp, synthetic construct
  source 1..1341: mol_type = other DNA; note = CD79A(EC-TM)-CD357(CYT)-CD79B(EC-TM)-DAP10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 343 - protein, 446 aa, synthetic construct
  source 1..446: note = CD79A(EC-TM)-CD357(CYT)-CD79B(EC-TM)-DAP10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFQLGLHIWQLRSQCMWPRETQLLLEVPPSTEDARSCQFPEEERGERSAEEKGRLGDLWVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 344 - DNA, 1524 bp, synthetic construct
  source 1..1524: mol_type = other DNA; note = CD79A(EC-TM)-CD357(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 345 - protein, 507 aa, synthetic construct
  source 1..507: note = CD79A(EC-TM)-CD357(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFQLGLHIWQLRSQCMWPRETQLLLEVPPSTEDARSCQFPEEERGERSAEEKGRLGDLWVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 346 - DNA, 1485 bp, synthetic construct
  source 1..1485: mol_type = other DNA; note = CD79A(EC-TM)-CD357(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 347 - protein, 494 aa, synthetic construct
  source 1..494: note = CD79A(EC-TM)-CD357(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFQLGLHIWQLRSQCMWPRETQLLLEVPPSTEDARSCQFPEEERGERSAEEKGRLGDLWVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 348 - DNA, 1323 bp, synthetic construct
  source 1..1323: mol_type = other DNA; note = CD79A(EC-TM)-DAP12(CYT)-CD79B(EC-TM)-DAP10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 349 - protein, 440 aa, synthetic construct
  source 1..440: note = CD79A(EC-TM)-DAP12(CYT)-CD79B(EC-TM)-DAP10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFYFLGRLVPRGRGAAEAATRKQRITETESPYQELQGQRSDVYSDLNTQRPYYKATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 350 - DNA, 1506 bp, synthetic construct
  source 1..1506: mol_type = other DNA; note = CD79A(EC-TM)-DAP12(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 351 - protein, 501 aa, synthetic construct
  source 1..501: note = CD79A(EC-TM)-DAP12(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFYFLGRLVPRGRGAAEAATRKQRITETESPYQELQGQRSDVYSDLNTQRPYYKATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 352 - DNA, 1467 bp, synthetic construct
  source 1..1467: mol_type = other DNA; note = CD79A(EC-TM)-DAP12(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 353 - protein, 488 aa, synthetic construct
  source 1..488: note = CD79A(EC-TM)-DAP12(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFYFLGRLVPRGRGAAEAATRKQRITETESPYQELQGQRSDVYSDLNTQRPYYKATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 354 - DNA, 1506 bp, synthetic construct
  source 1..1506: mol_type = other DNA; note = CD79A(EC-TM)-DAP12(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 355 - protein, 501 aa, synthetic construct
  source 1..501: note = CD79A(EC-TM)-DAP12(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFYFLGRLVPRGRGAAEAATRKQRITETESPYQELQGQRSDVYSDLNTQRPYYKATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 356 - DNA, 1584 bp, synthetic construct
  source 1..1584: mol_type = other DNA; note = CD79A(EC-TM)-DAP12(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctcttttacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataaggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 357 - protein, 527 aa, synthetic construct
  source 1..527: note = CD79A(EC-TM)-DAP12(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFYFLGRLVPRGRGAAEAATRKQRITETESPYQELQGQRSDVYSDLNTQRPYYKATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 358 - DNA, 1383 bp, synthetic construct
  source 1..1383: mol_type = other DNA; note = CD79A(EC-TM)-DAP10(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttctgtgtgctcgcccacggagaagtccagcccaggaggatggtaaggtttatataaacatgccaggaaggggggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 359 - protein, 460 aa, synthetic construct
  source 1..460: note = CD79A(EC-TM)-DAP10(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFLCARPRRSPAQEDGKVYINMPGRGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 360 - DNA, 1422 bp, synthetic construct
  source 1..1422: mol_type = other DNA; note = CD79A(EC-TM)-DAP10(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttctgtgtgctcgcccacggagaagtccagcccaggaggatggtaaggtttatataaacatgccaggaaggggggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 361 - protein, 473 aa, synthetic construct
  source 1..473: note = CD79A(EC-TM)-DAP10(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFLCARPRRSPAQEDGKVYINMPGRGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 362 - DNA, 1500 bp, synthetic construct
  source 1..1500: mol_type = other DNA; note = CD79A(EC-TM)-DAP10(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttctgtgtgctcgcccacggagaagtccagcccaggaggatggtaaggtttatataaacatgccaggaaggggggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 363 - protein, 499 aa, synthetic construct
  source 1..499: note = CD79A(EC-TM)-DAP10(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFLCARPRRSPAQEDGKVYINMPGRGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 364 - DNA, 1344 bp, synthetic construct
  source 1..1344: mol_type = other DNA; note = CD79A(EC-TM)-41BB(CYT)-CD79B(EC-TM)-CD28(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagatcaaaaaggagcagactgcttcactctgactatatgaacatgactccccgtcgccccggccccacacggaagcactatcaaccctacgcccctcctagagattttgccgcgtataggtcttag

SEQ ID NO: 365 - protein, 447 aa, synthetic construct
  source 1..447: note = CD79A(EC-TM)-41BB(CYT)-CD79B(EC-TM)-CD28(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRSKRSRLLHSDYMNMTPRRPGPTRKHYQPYAPPRDFAAYRS

SEQ ID NO: 366 - DNA, 1404 bp, synthetic construct
  source 1..1404: mol_type = other DNA; note = CD79A(EC-TM)-41BB(CYT)-CD79B(EC-TM)-CD266(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtctag

SEQ ID NO: 367 - protein, 467 aa, synthetic construct
  source 1..467: note = CD79A(EC-TM)-41BB(CYT)-CD79B(EC-TM)-CD266(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNRRRRRERRDLFTESWDTQKAPNNYRSPISTSQPTNQSMDDTREDIYVNYPTFSRRPKTRV

SEQ ID NO: 368 - DNA, 1518 bp, synthetic construct
  source 1..1518: mol_type = other DNA; note = CD79A(EC-TM)-41BB(CYT)-CD79B(EC-TM)-CD84(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgatttag

SEQ ID NO: 369 - protein, 505 aa, synthetic construct
  source 1..505: note = CD79A(EC-TM)-41BB(CYT)-CD79B(EC-TM)-CD84(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 370 - DNA, 1581 bp, synthetic construct
  source 1..1581: mol_type = other DNA; note = CD79A(EC-TM)-41BB(CYT)-CD79B(EC-TM)-CD244(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagctag

SEQ ID NO: 371 - protein, 526 aa, synthetic construct
  source 1..526: note = CD79A(EC-TM)-41BB(CYT)-CD79B(EC-TM)-CD244(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 372 - DNA, 1338 bp, synthetic construct
  source 1..1338: mol_type = other DNA; note = CD79A(EC-TM)-41BB(CYT)-CD79B(EC-TM)-CD7(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcgaacgcaaatcaaaaaactctgctcttggcgggacaagaattccgcagcttgcgtggtgtatgaagacatgtctcacagtcggtgtaatacactctctagcccgaaccaataccaatag

SEQ ID NO: 373 - protein, 445 aa, synthetic construct
  source 1..445: note = CD79A(EC-TM)-41BB(CYT)-CD79B(EC-TM)-CD7(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRTQIKKLCSWRDKNSAACVVYEDMSHSRCNTLSSPNQYQ

SEQ ID NO: 374 - DNA, 1395 bp, synthetic construct
  source 1..1395: mol_type = other DNA; note = CD79A(EC-TM)-41BB(CYT)-CD79B(EC-TM)-CD357(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtgtag

SEQ ID NO: 375 - protein, 464 aa, synthetic construct
  source 1..464: note = CD79A(EC-TM)-41BB(CYT)-CD79B(EC-TM)-CD357(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLQLGLHIWQLRSQCMWPRETQLLLEVPPSTEDARSCQFPEEERGERSAEEKGRLGDLWV

SEQ ID NO: 376 - DNA, 1377 bp, synthetic construct
  source 1..1377: mol_type = other DNA; note = CD79A(EC-TM)-41BB(CYT)-CD79B(EC-TM)-DAP12(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataagtag

SEQ ID NO: 377 - protein, 458 aa, synthetic construct
  source 1..458: note = CD79A(EC-TM)-41BB(CYT)-CD79B(EC-TM)-DAP12(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLYFLGRLVPRGRGAAEAATRKQRITETESPYQELQGQRSDVYSDLNTQRPYYK

SEQ ID NO: 378 - DNA, 1293 bp, synthetic construct
  source 1..1293: mol_type = other DNA; note = CD79A(EC-TM)-41BB(CYT)-CD79B(EC-TM)-DAP10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 379 - protein, 430 aa, synthetic construct
  source 1..430: note = CD79A(EC-TM)-41BB(CYT)-CD79B(EC-TM)-DAP10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 380 - DNA, 1476 bp, synthetic construct
  source 1..1476: mol_type = other DNA; note = CD79A(EC-TM)-41BB(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 381 - protein, 491 aa, synthetic construct
  source 1..491: note = CD79A(EC-TM)-41BB(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 382 - DNA, 1437 bp, synthetic construct
  source 1..1437: mol_type = other DNA; note = CD79A(EC-TM)-41BB(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 383 - protein, 478 aa, synthetic construct
  source 1..478: note = CD79A(EC-TM)-41BB(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 384 - DNA, 1476 bp, synthetic construct
  source 1..1476: mol_type = other DNA; note = CD79A(EC-TM)-41BB(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 385 - protein, 478 aa, synthetic construct; identical to SEQ ID NO: 383
  source 1..478: note = CD79A(EC-TM)-41BB(CYT)-CD79B(EC-TM)-NTB-A(CYT)

SEQ ID NO: 386 - DNA, 1554 bp, synthetic construct
  source 1..1554: mol_type = other DNA; note = CD79A(EC-TM)-41BB(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaagcgcggccgtaaaaaacttctgtacatttttaagcaacctttcatgcgccccgtgcagaccacacaggaggaggatggatgttcatgcaggttcccggaagaggaagaaggcgggtgtgagcttgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 387 - protein, 517 aa, synthetic construct
  source 1..517: note = CD79A(EC-TM)-41BB(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFKRGRKKLLYIFKQPFMRPVQTTQEEDGCSCRFPEEEEGGCELATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 388 - DNA, 1401 bp, synthetic construct
  source 1..1401: mol_type = other DNA; note = CD79A(EC-TM)-CD28(CYT)-CD79B(EC-TM)-CD266(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaatcggcggcgacggagagaacgcagagacctctttactgaatcatgggacacacaaaaagcaccaaataactatcggtcacccatatcaaccagccaaccgactaaccagtccatggatgatacccgggaagacatatatgttaactatcccaccttttcacgacggcctaagacaagggtctag

SEQ ID NO: 389 - protein, 466 aa, synthetic construct
  source 1..466: note = CD79A(EC-TM)-CD28(CYT)-CD79B(EC-TM)-CD266(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNRRRRRERRDLFTESWDTQKAPNNYRSPISTSQPTNQSMDDTREDIYVNYPTFSRRPKTRV

SEQ ID NO: 390 - DNA, 1515 bp, synthetic construct
  source 1..1515: mol_type = other DNA; note = CD79A(EC-TM)-CD28(CYT)-CD79B(EC-TM)-CD84(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgatttag

SEQ ID NO: 391 - protein, 504 aa, synthetic construct
  source 1..504: note = CD79A(EC-TM)-CD28(CYT)-CD79B(EC-TM)-CD84(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 392 - DNA, 1578 bp, synthetic construct
  source 1..1578: mol_type = other DNA; note = CD79A(EC-TM)-CD28(CYT)-CD79B(EC-TM)-CD244(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagctag

SEQ ID NO: 393 - protein, 525 aa, synthetic construct
  source 1..525: note = CD79A(EC-TM)-CD28(CYT)-CD79B(EC-TM)-CD244(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 394 - DNA, 1335 bp, synthetic construct
  source 1..1335: mol_type = other DNA; note = CD79A(EC-TM)-CD28(CYT)-CD79B(EC-TM)-CD7(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcgaacgcaaatcaaaaaactctgctcttggcgggacaagaattccgcagcttgcgtggtgtatgaagacatgtctcacagtcggtgtaatacactctctagcccgaaccaataccaatag

SEQ ID NO: 395 - protein, 444 aa, synthetic construct
  source 1..444: note = CD79A(EC-TM)-CD28(CYT)-CD79B(EC-TM)-CD7(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRTQIKKLCSWRDKNSAACVVYEDMSHSRCNTLSSPNQYQ

SEQ ID NO: 396 - DNA, 1392 bp, synthetic construct
  source 1..1392: mol_type = other DNA; note = CD79A(EC-TM)-CD28(CYT)-CD79B(EC-TM)-CD357(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtgtag

SEQ ID NO: 397 - protein, 463 aa, synthetic construct
  source 1..463: note = CD79A(EC-TM)-CD28(CYT)-CD79B(EC-TM)-CD357(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLQLGLHIWQLRSQCMWPRETQLLLEVPPSTEDARSCQFPEEERGERSAEEKGRLGDLWV

SEQ ID NO: 398 - DNA, 1374 bp, synthetic construct
  source 1..1374: mol_type = other DNA; note = CD79A(EC-TM)-CD28(CYT)-CD79B(EC-TM)-DAP12(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataagtag

SEQ ID NO: 399 - protein, 457 aa, synthetic construct
  source 1..457: note = CD79A(EC-TM)-CD28(CYT)-CD79B(EC-TM)-DAP12(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLYFLGRLVPRGRGAAEAATRKQRITETESPYQELQGQRSDVYSDLNTQRPYYK

SEQ ID NO: 400 - DNA, 1290 bp, synthetic construct
  source 1..1290: mol_type = other DNA; note = CD79A(EC-TM)-CD28(CYT)-CD79B(EC-TM)-DAP10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 401 - protein, 429 aa, synthetic construct
  source 1..429: note = CD79A(EC-TM)-CD28(CYT)-CD79B(EC-TM)-DAP10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 402 - DNA, 1473 bp, synthetic construct
  source 1..1473: mol_type = other DNA; note = CD79A(EC-TM)-CD28(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 403 - protein, 490 aa, synthetic construct
  source 1..490: note = CD79A(EC-TM)-CD28(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 404 - DNA, 1434 bp, synthetic construct
  source 1..1434: mol_type = other DNA; note = CD79A(EC-TM)-CD28(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 405 - protein, 477 aa, synthetic construct
  source 1..477: note = CD79A(EC-TM)-CD28(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 406 - DNA, 1473 bp, synthetic construct
  source 1..1473: mol_type = other DNA; note = CD79A(EC-TM)-CD28(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 407 - protein, 490 aa, synthetic construct
  source 1..490: note = CD79A(EC-TM)-CD28(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 408 - DNA, 1551 bp, synthetic construct
  source 1..1551: mol_type = other DNA; note = CD79A(EC-TM)-CD28(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttagatcaaaaaggagcagactgcttcactctgactatatgaacatgactccccgtcgccccggccccacacggaagcactatcaaccctacgcccctcctagagattttgccgcgtataggtctgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 409 - protein, 516 aa, synthetic construct
  source 1..516: note = CD79A(EC-TM)-CD28(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRSKRSRLLHSDYMNMTPRRPGPTRKHYQPYAPPRDFAAYRSATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 410 - DNA, 1752 bp, synthetic construct
  source 1..1752: mol_type = other DNA; note = CD79A(EC-TM)-CD84(CYT)-CD79B(EC-TM)-CD244(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtggaggcgcaaacgaaaagagaaacaatcagaaacatcccccaaggagttcttgactatttacgaggacgtgaaagaccttaaaactaggaggaaccacgaacaggagcaaacatttcccggcggcggctccacgatctatagtatgattcagtcacagagttccgcacctacaagccaagagccggcgtatactctgtactcccttattcagccctcaaggaagtccggatctcgtaagcgaaaccattctccctcattcaactctacaatttacgaggtgatcgggaagagtcaacctaaggcacagaatccagccagactcagtaggaaagagctggaaaattttgacgtgtatagctag

SEQ ID NO: 411 - protein, 583 aa, synthetic construct
  source 1..583: note = CD79A(EC-TM)-CD84(CYT)-CD79B(EC-TM)-CD244(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 412 - DNA, 1509 bp, synthetic construct
  source 1..1509: mol_type = other DNA; note = CD79A(EC-TM)-CD84(CYT)-CD79B(EC-TM)-CD7(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcgaacgcaaatcaaaaaactctgctcttggcgggacaagaattccgcagcttgcgtggtgtatgaagacatgtctcacagtcggtgtaatacactctctagcccgaaccaataccaatag

SEQ ID NO: 413 - protein, 502 aa, synthetic construct
  source 1..502: note = CD79A(EC-TM)-CD84(CYT)-CD79B(EC-TM)-CD7(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLRTQIKKLCSWRDKNSAACVVYEDMSHSRCNTLSSPNQYQ

SEQ ID NO: 414 - DNA, 1566 bp, synthetic construct
  source 1..1566: mol_type = other DNA; note = CD79A(EC-TM)-CD84(CYT)-CD79B(EC-TM)-CD357(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtgtag

SEQ ID NO: 415 - protein, 521 aa, synthetic construct
  source 1..521: note = CD79A(EC-TM)-CD84(CYT)-CD79B(EC-TM)-CD357(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLQLGLHIWQLRSQCMWPRETQLLLEVPPSTEDARSCQFPEEERGERSAEEKGRLGDLWV

SEQ ID NO: 416 - DNA, 1548 bp, synthetic construct
  source 1..1548: mol_type = other DNA; note = CD79A(EC-TM)-CD84(CYT)-CD79B(EC-TM)-DAP12(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgtacttcctcggccgactggtccctaggggcagaggggctgccgaggcggctacacggaaacagcgcataactgaaactgaaagtccctaccaggagctccagggtcagagatccgacgtatacagtgatctgaatacgcagcgaccatactataagtag

SEQ ID NO: 417 - protein, 515 aa, synthetic construct
  source 1..515: note = CD79A(EC-TM)-CD84(CYT)-CD79B(EC-TM)-DAP12(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLYFLGRLVPRGRGAAEAATRKQRITETESPYQELQGQRSDVYSDLNTQRPYYK

SEQ ID NO: 418 - DNA, 1464 bp, synthetic construct
  source 1..1464: mol_type = other DNA; note = CD79A(EC-TM)-CD84(CYT)-CD79B(EC-TM)-DAP10(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgctgtgtgctcgcccacggagaagtccagcccaggaggatggtaaggtttatataaacatgccaggaagggggtag

SEQ ID NO: 419 - protein, 487 aa, synthetic construct
  source 1..487: note = CD79A(EC-TM)-CD84(CYT)-CD79B(EC-TM)-DAP10(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLCARPRRSPAQEDGKVYINMPGRG

SEQ ID NO: 420 - DNA, 1647 bp, synthetic construct
  source 1..1647: mol_type = other DNA; note = CD79A(EC-TM)-CD84(CYT)-CD79B(EC-TM)-BAFF(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgagctggcggaggcgccagcgccgcttgcggggggcgtcttccgccgaggccccagacggcgacaaggatgcacccgagcctctcgacaaggtgatcattttgtctcccggaatttcagacgcaaccgcaccagcatggccacctcctggtgaggacccagggacaactcccccaggacattccgtacccgtaccagcaacagaattaggctccacagagttagtgaccactaaaaccgccggccctgaacaacaatag

SEQ ID NO: 421 - protein, 548 aa, synthetic construct
  source 1..548: note = CD79A(EC-TM)-CD84(CYT)-CD79B(EC-TM)-BAFF(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 422 - DNA, 1608 bp, synthetic construct
  source 1..1608: mol_type = other DNA; note = CD79A(EC-TM)-CD84(CYT)-CD79B(EC-TM)-CD44(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgaactccagaagacggtgcgggcaaaaaaaaaaattagttatcaatagcggcaacggagctgtggaggatcgcaagccctctgggctgaacggggaggctagcaaaagtcaggaaatggtacatctggtaaataaagagtccagtgaaactccagatcagttcatgaccgccgatgaaactcgcaatctgcaaaatgttgacatgaaaatcggggtgtag

SEQ ID NO: 423 - protein, 535 aa, synthetic construct
  source 1..535: note = CD79A(EC-TM)-CD84(CYT)-CD79B(EC-TM)-CD44(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLNSRRRCGQKKKLVINSGNGAVEDRKPSGLNGEASKSQEMVHLVNKESSETPDQFMTADETRNLQNVDMKIGV

SEQ ID NO: 424 - DNA, 1647 bp, synthetic construct
  source 1..1647: mol_type = other DNA; note = CD79A(EC-TM)-CD84(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 425 - protein, 548 aa, synthetic construct
  source 1..548: note = CD79A(EC-TM)-CD84(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 426 - DNA, 1725 bp, synthetic construct
  source 1..1725: mol_type = other DNA; note = CD79A(EC-TM)-CD84(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttaggctgttcaaaaggaggcaaggccgcatctttcctgagggcagctgcctgaataccttcactaaaaatccttatgcagcgtccaagaagacgatatatacctatattatggccagtaggaatacacagccggccgagtccaggatctacgacgagatattacaatctaaggttcttccaagtaaagaggaaccagtgaataccgtttattccgaagtgcaatttgctgacaagatgggaaaagcttcaacccaagactccaagccacccggcaccagcagctatgagatcgtgattgccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 427 - protein, 574 aa, synthetic construct
  source 1..574: note = CD79A(EC-TM)-CD84(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVIATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 428 - DNA, 1524 bp, synthetic construct
  source 1..1524: mol_type = other DNA; note = CD79A(EC-TM)-CD357(CYT)-CD79B(EC-TM)-NTB-A(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttgaggaaacgccgtgacagtctcagcctgtcaactcagagaacacagggtccagctgaatccgcacggaacctggagtacgtgtcagtgagcccaaccaataacacagtctacgcttctgtcacccatagtaatcgtgagactgagatttggactccacgtgagaacgacaccatcaccatttactccacgatcaatcactctaaggagtccaagccgacattctctagagccaccgccctggacaacgtagtttag

SEQ ID NO: 429 - protein, 507 aa, synthetic construct
  source 1..507: note = CD79A(EC-TM)-CD357(CYT)-CD79B(EC-TM)-NTB-A(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFQLGLHIWQLRSQCMWPRETQLLLEVPPSTEDARSCQFPEEERGERSAEEKGRLGDLWVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLRKRRDSLSLSTQRTQGPAESARNLEYVSVSPTNNTVYASVTHSNRETEIWTPRENDTITIYSTINHSKESKPTFSRATALDNVV

SEQ ID NO: 430 - DNA, 1602 bp, synthetic construct
  source 1..1602: mol_type = other DNA; note = CD79A(EC-TM)-CD357(CYT)-CD79B(EC-TM)-CD28H(CYT)
atgcctggtggacccggcgtactgcaagcgttgccagctaccatctttctgctttttcttttgtcagctgtctatcttggtccaggttgccaagctctctggatgcacaaggttccagcctcacttatggtctcactgggagaagacgcgcatttccaatgcccacataattcttcaaacaacgccaacgtaacctggtggcgcgtccttcacggaaactatacctggccgcccgagttcctcggcccaggcgaagatcctaacggtacgctcataatccagaatgttaataaatcccacggtgggatttacgtttgcagagtgcaggaaggtaatgaaagctaccagcaaagctgcggaacctaccttagggtccggcagccgcccccaagacccttcctcgatatgggtgagggaacaaagaaccgaataatcacggccgaaggcattatcctcttgttttgcgcagttgtgccgggcactctgctgctctttcagctggggttgcacatttggcagttacgatcacaatgtatgtggccccgggagacccagctgctcctggaggttcccccttctactgaggatgcacgatcttgccagtttccagaagaagagaggggcgaacgatccgccgaggaaaaaggacgcctcggcgacctgtgggtggccacaaacttctctctgctaaagcaagcaggtgatgttgaagaaaaccccgggccaatggcccgccttgcactttccccggtgccaagtcattggatggtggccctgctgctgttgctcagcgcagaacctgtccctgcggcgaggagtgaagatcgctatcggaatcctaaaggaagcgcgtgctctagaatttggcaatctccccgatttattgcaagaaagcggggctttacagtcaaaatgcattgttacatgaatagtgcatctgggaacgtatcttggttgtggaaacaagaaatggacgaaaaccctcagcagttgaaacttgaaaaagggagaatggaagaaagtcaaaacgaaagtcttgcaacactcactatccaagggattaggtttgaggataacggcatttacttctgtcagcagaaatgtaataatacgagtgaagtataccaagggtgcggtacagagctgagagttatgggcttttctaccctggcccaactcaaacagcggaataccttgaaagacgggattataatgatccaaactctcttgatcattttgtttatcatcgtccctatatttctcctgttctggggcagacggagctgtcaacaacgggatagcggtaattcaccaggcaatgccttctattccaatgttttgtaccggccacgaggcgcccctaagaagagcgaagactgttccggtgagggaaaagaccagagaggccaaagtatctatagtacttctttccctcagcccgcgcccaggcagccccacctggctagccggccttgtcccagtccaagaccttgcccttcaccaaggcccgggcaccccgtttcaatggttagggtatccccacgcccatccccaacacaacaaccacgcccaaaggggtttccaaaggtgggcgaggagtag

SEQ ID NO: 431 - protein, 533 aa, synthetic construct
  source 1..533: note = CD79A(EC-TM)-CD357(CYT)-CD79B(EC-TM)-CD28H(CYT)
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFQLGLHIWQLRSQCMWPRETQLLLEVPPSTEDARSCQFPEEERGERSAEEKGRLGDLWVATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 432 - DNA, 2397 bp, synthetic construct
  source 1..2397: mol_type = other DNA; note = mIgG/CD20-FceRIg
atgttgccatcacaactcattgggtttctgctgctctgggttccagctagccgcggtcaaattgtgctctcccagtcccctgcaatcctgagcgcatctcccggcgagaaagtgactatgacctgtcgggcctccagtagcgtcagctatatccattggttccagcagaaacccggcagcagccctaagccttggatttacgcaacatctaatttagcctccggagtgcctgtgaggtttagcggctctggctccggcacatcatacagccttacaattagcagagtggaggccgaggacgccgccacatattactgtcagcaatggacatctaacccgcctacttttggagggggaaccaagctggaaataaagcgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgccacaaacttctctctgctaaagcaagcaggtgatgttgaagaaaaccccgggcctatggactggacctggaggatcctcttcttggtggcggccgccacaggcgcgcactcccaagtgcagttgcaacagcccggagcggaactcgtcaaacctggcgcaagcgtaaagatgtcatgcaaggcctcaggttataccttcacaagttataatatgcattgggtgaagcagaccccagggaggggtctggagtggatcggggccatctatcctggtaacggagatacaagctataaccagaaattcaagggcaaggccactctgacggcggacaagagctcatccacagcttatatgcagctgtcctctctcacatccgaagactccgcagtctactactgcgcgcgctccacttattatggtggggattggtatttcaacgtgtggggggccggcactacagtgacggtgtctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccggagctccagcttgaagaaagctgtgccgaggcacaggacggtgagctcgacggattgtggacgaccatcactatattcataactctgtttttgttgtctgtttgctactccgcaaccgtgactttctttaaagttaaagacgccatcacaagctacgaaaagtctgacggggtgtatacgggtctgagtacacggaaccaggagacctacgagaccttgaaacacgaaaaacctccacaa

SEQ ID NO: 433 - protein, 799 aa, synthetic construct
  source 1..799: note = mIgG/CD20-FceRIg
MLPSQLIGFLLLWVPASRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECATNFSLLKQAGDVEENPGPMDWTWRILFLVAAATGAHSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKDAITSYEKSDGVYTGLSTRNQETYETLKHEKPPQ

SEQ ID NO: 434 - DNA, 2397 bp, synthetic construct
  source 1..2397: mol_type = other DNA; note = mIgG/CD19.1-FceRIg
atgttgccatcacaactcattgggtttctgctgctctgggttccagctagccgcggtgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgccacaaacttctctctgctaaagcaagcaggtgatgttgaagaaaaccccgggcctatggactggacctggaggatcctcttcttggtggcggccgccacaggcgcgcactccgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccggagctccagcttgaagaaagctgtgccgaggcacaggacggtgagctcgacggattgtggacgaccatcactatattcataactctgtttttgttgtctgtttgctactccgcaaccgtgactttctttaaagttaaagacgccatcacaagctacgaaaagtctgacggggtgtatacgggtctgagtacacggaaccaggagacctacgagaccttgaaacacgaaaaacctccacaa

SEQ ID NO: 435 - protein, 799 aa, synthetic construct
  source 1..799: note = mIgG/CD19.1-FceRIg
MLPSQLIGFLLLWVPASRGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECATNFSLLKQAGDVEENPGPMDWTWRILFLVAAATGAHSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKDAITSYEKSDGVYTGLSTRNQETYETLKHEKPPQ

SEQ ID NO: 436 - DNA, 2412 bp, synthetic construct
  source 1..2412: mol_type = other DNA; note = mIgG/CD19.2-FceRIg
atgttgccatcacaactcattgggtttctgctgctctgggttccagctagccgcggtgaaatcgtgctcacccaaagcccggacttccagtctgtcacaccaaaggagaaagtgacaatcacctgtagggctagtgaaagcgtagatacatttggcatatcttttatgaattggttccagcagaaacctgatcagagccctaaactcctgattcatgaggcgagcaaccaaggatccggagtgccatcccggttttcaggttccggaagtggaactgacttcacattgaccatcaactctctcgaggccgaggacgctgcaacctactactgccagcaatctaaggaggttccattcactttcggaggcgggactaaagtcgagattaagcgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgccacaaacttctctctgctaaagcaagcaggtgatgttgaagaaaaccccgggcctatggactggacctggaggatcctcttcttggtggcggccgccacaggcgcgcactccgaggtgcagctggtggagagcggcggcggcctggtgcagcccggcggcagcctgagactgagctgcgccgccagcggcttcaccttcagcagcagctggatgaactgggtgagacaggcccccggcaagggcctggagtgggtgggcagaatctaccccggcgacggcgacaccaactacaacgtgaagttcaagggcagattcaccatcagcagagatgacagcaagaacagcctgtacctgcagatgaacagcctgaagaccgaggacaccgccgtgtactactgcgccagaagcggcttcatcaccaccgtgagagacttcgactactggggccagggcaccctggtgaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccggagctccagcttgaagaaagctgtgccgaggcacaggacggtgagctcgacggattgtggacgaccatcactatattcataactctgtttttgttgtctgtttgctactccgcaaccgtgactttctttaaagttaaagacgccatcacaagctacgaaaagtctgacggggtgtatacgggtctgagtacacggaaccaggagacctacgagaccttgaaacacgaaaaacctccacaa

SEQ ID NO: 437 - protein, 804 aa, synthetic construct
  source 1..804: note = mIgG/CD19.2-FceRIg
MLPSQLIGFLLLWVPASRGEIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECATNFSLLKQAGDVEENPGPMDWTWRILFLVAAATGAHSEVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKDAITSYEKSDGVYTGLSTRNQETYETLKHEKPPQ

SEQ ID NO: 438 - skipped

SEQ ID NO: 439 - DNA, 57 bp, synthetic construct
  source 1..57: mol_type = other DNA; note = 2A
gccacaaacttctctctgctaaagcaagcaggtgatgttgaagaaaaccccgggcca

SEQ ID NO: 440 - protein, 19 aa, synthetic construct
  source 1..19: note = 2A
ATNFSLLKQAGDVEENPGP

(19) United States
(12) Patent Application Publication
(10) Pub. No.: US 2025/0152713 A1
(43) Pub. Date: May 15, 2025

(54) MULTICHAIN ANTIGEN-SPECIFIC RECEPTORS FOR CELL-BASED IMMUNOTHERAPY

(71) Applicant: ETH ZURICH, Zurich (CH)

(72) Inventors: Fabian Enrik Trick, Basel (CH); Yaakov Benenson, Binningen (CH)

(73) Assignee: ETH ZURICH, Zurich (CH)

(21) Appl. No.: 18/722,822

(22) PCT Filed: Dec. 23, 2022
(86) PCT No.: PCT/EP2022/087835

§ 371 Date: Jun. 21, 2024

(30) Foreign Application Priority Data
Dec. 24, 2021 (EP) 21217757.0

(51) Int. Cl. A61K 40/42; A61K 40/15; A61K 40/30; C07K 14/735; C07K 16/32

(52) U.S. Cl. CPC A61K 40/4205; A61K 40/15; A61K 40/30; C07K 14/70535; C07K 16/32; C07K 2317/24; C07K 2317/33; C07K 2317/732

(57) ABSTRACT

The present invention is in the field of cell-based immunotherapies. In particular, the invention provides a modified cell comprising a first and second polypeptide forming an antigen-binding site at the external side of the cell, and a polypeptide comprising a signaling domain, wherein, upon binding of the antigen-binding site to a corresponding antigen, the signaling domain triggers a process in the cell that enables the cell to promote death of a target cell comprising said antigen on the cell surface. The invention also provides medical uses of the modified cell, in particular for use in the treatment of diseases. Furthermore, the invention provides a kit comprising at least one nucleic acid molecule encoding said polypeptides, and methods for producing the modified cells of the invention. In addition, the invention provides chimeric polypeptides and nucleic acid molecules encoding chimeric polypeptides.

The present invention is in the field of cell-based immunotherapies. In particular, the invention provides a modified cell comprising a first and second polypeptide forming an antigen-binding site at the external side of the cell, and a polypeptide comprising a signaling domain, wherein, upon binding of the antigen-binding site to a corresponding antigen, the signaling domain triggers a process in the cell that enables the cell to promote death of a target cell comprising said antigen on the cell surface. The invention also provides medical uses of the modified cell, in particular for use in the treatment of diseases. Furthermore, the invention provides a kit comprising at least one nucleic acid molecule encoding said polypeptides, and methods for producing the modified cells of the invention. In addition, the invention provides chimeric polypeptides and nucleic acid molecules encoding chimeric polypeptides.

Cell therapies harness genetically modified or un-modified immune cells to target or re-target pathogenic cells, e.g., cancerous cells. Most of these treatments depend on receptors, e.g., chimeric antigen receptors (CARs), redirecting the immune cells towards their target cells. These CARs are typically transduced into T cells, resulting in so-called CAR T cells.

The general idea of recombinant, chimeric receptors for immune cells that confer antigen specificity via an extracellular binding domain (i.e., a single-chain variable fragment (scFv)) and trigger immune signaling via cytoplasmic signaling motifs, has been the basis for the development of CARs since the early days of the field. The design of such receptors initially started with the effort to reprogram T cell receptor (TCR) specificity (Kuwana, et al. 1987) and later moved towards single-chain variants, all the way to clinically-approved CAR-T cell therapies (Jayaraman, et al. 2020). In particular, single chain receptors offered reduced complexity in their application to cells. In Eshhar's ‘T-bodies’, the first implementation of a CAR, the scFv was directly fused to the CD3ζ (CD3 zeta, or CD247) chain of the T cell receptor complex (Eshhar, et al. 1993; Gross, et al. 1989; Yáñez-Muñoz and Grupp 2018). Since then many co-activating, hinge, and transmembrane domains were added or exchanged, to further enhance effector cell expansion, persistence, and anti-tumor activity (Weinkove, et al. 2019). However, the underlying single-chain receptor architecture featuring antigen targeting scFvs, one hinge domain, a single transmembrane domain, one or two co-factor domains, and CD3ζ, has barely evolved (Jayaraman, et al. 2020).

Thus, CAR architecture is still based on a single gene construct coding for different protein domains from multiple sources, resulting in a single chain chimeric receptor. The most common denominators of CARs are their N- and C-terminal domains. Usually, CARs comprise an N-terminal single chain variable fragment (scFv) as the target antigen binder and a C-terminal CD3 zeta chain as the cell activating signaling domain. There are no defined standards yet for the connecting spacer, hinge, transmembrane, and co-stimulatory domains in CARs. Although it was shown that the CAR approach yields efficacious treatments in terminal lymphoma and myeloma patients, it still has, at least, three underlying problems.

First, in a modularity sense, each exchange of a domain requires cumbersome construct optimization, as it might lead to loss of receptor surface-localization or function. In particular, in current CAR designs, scFvs and hinge domains need to be adjusted to switch the target epitope. This has the disadvantage that a fully new protein has to be created which often leads to expression issues (Jayaraman, et al. 2020). Furthermore, since the hinge domain is also associated with CAR activity, there has been extensive research on identifying good hinge domains, as well as their length. It has been found that shorter hinge domains tend to have higher activation potential. However, short hinge domains need to be adjusted for each target, as they might hinder the scFv from reaching a membrane-proximal epitope.

Second, mispairing of scFvs may crosslink multiple receptors on the effector cell surface and lead to undesired tonic signaling in CAR-T cells (Zajc, et al. 2021). In particular, the predisposition of scFv-based receptors to cross-assemble limits the potential cell surface density of functional protein. This results in a low efficacy and can lead to effector cell exhaustion despite good receptor expression levels.

Third, compared to signaling domains in natural receptors, the in-line arrangement of co-stimulatory and activating domains in current chimeric antigen receptors changes the plasma membrane distance of these domains. This is known to negatively influence the activity of juxtamembrane signaling domains and has been associated with poor functionality. Furthermore, the suboptimal plasma membrane distance of signaling domains in single chain multi activation domain receptors that have to be placed in a continuous in-line arrangement, limits the maximal multiplicity of ITAMs. For example, only to up 6 ITAMs have been used so far, and only in case of CAR-dimers. (Feucht, et al. 2019; Jayaraman, et al. 2020; Zajc, et al. 2021). In almost all current CAR approaches only one transmembrane domain is used bearing only one slot for a signaling/activation domain. In some cases, up to two co-stimulatory domains (CD28, 41BB, OX40) and one activating domain (CD3z or FceRIg) have been combined in CARs (Pfefferle and Huntington 2020). However, the stacking of domains in such third-generation CARs more or less failed, as there was no significant increase in cytotoxic activity (Huang, et al. 2020). In one case, two activation domains (DAP10 and CD3z) have been combined in one ligand CAR, which, however, cannot be easily adapted to another target epitope. Furthermore, the DAP10 cannot be exchanged (Chang, et al. 2013).

Furthermore, the CAR constructs of the prior art are usually delivered to T cells and are not optimized for other cell types.

Natural Killer (NK) cells are another class of cytotoxic cells which have better safety properties compared to T cells, and which can be deployed in an allogeneic fashion. Natural killer cells play a pivotal role in immune surveillance via recognition and elimination of cancerous, virally infected, and other disease cells (Abel, et al. 2018; Correia, et al. 2021; Iannello and Raulet 2013; Pech, et al. 2019; Vivier, et al. 2008). NK cells belong to the innate immune system, and therefore they do not bear antigen-specific receptors. Hence, NK cells are intrinsically non-specific and cannot specifically recognize a target antigen. Unlike antigen-specific cytotoxic cells such as CD8⁺ T cells, NK cells meticulously integrate multiple signals from antigen-unspecific activating and inactivating receptors. The majority of these unspecific receptors have to rely on synergistic co-activation to induce a signal sufficient to trigger a cytotoxic response in NK cells (Bryceson, et al. 2006).

However, CD16, a low affinity receptor for the fragment crystallizable (Fc) region of Immunoglobulin γ (IgG) expressed on a subset of NK cells, functions on its own to form the link between the adaptive and the innate immune responses, endowing NK cells with antibody-supported antigen specificity (Cooper, et al. 2001). This link is formed via the engagement of CD16 with a soluble IgG antibody bound to its antigen on a cell surface, which induces antibody-dependent cellular cytotoxicity (ADCC) (Capuano, et al. 2021; Nimmerjahn and Ravetch 2006; Wang, et al. 2015b) through NK cell activation, polarization, and degranulation. The induction of ADCC in NK cells with monoclonal antibodies contributes to the antibody efficacy (Muntasell, et al. 2017). For example, some of the antitumor efficacy of monoclonal IgG antibodies like Trastuzumab/Herceptin is due to ADCC (Lee, et al. 2020; Liang, et al. 2018). One of the initial steps in this process involves the signaling domains of CD16-associated CD247 (CD3ζ (CD3 zeta)) and FcεRIγ (FceRIg), which comprise immunoreceptor tyrosine-based activation motifs (ITAM). The phosphorylation of the ITAMs leads to a strong activation of NK cells that tilts the scale between activating and inactivating signals in favor of NK cell activation and their resulting cytotoxicity (Blezquez-Moreno, et al. 2017).

The ability to activate antigen-specific cytotoxicity of NK cells has been exploited for clinical research and applications. For instance, the combination of NK-92 cells expressing a high-affinity mutant of CD16 with PDL1-targeting Avelumab was used to treat Merkel cell carcinoma in clinical trials (Park, et al. 2021).

However, triggering ADCC against a tumor using NK cells infusion is a three-component process involving the NK cells, the soluble antibody, and the tumors. Consequently, its efficacy is limited by the availability of the NK cells and the soluble antibody at the tumor site. In particular, the contact of a soluble antibody with its target antigen on a target relies on the passive process of diffusion, which can severely hamper ADCC efficiency. Indeed, it has been shown that ADCC using a combination of NK cells and soluble antibody against solid tumors was still outperformed by T cells carrying an antigen specific receptor (Sz66r, et al. 2020) due to insufficient availability of soluble antibody within the tumor (Thurber, et al. 2008).

In some studies, CARs used in the context of T cells have been also applied to NK cells. However, these CARs are not optimized for NK cells and they may not be the best way to augment NK cells with antigen specificity. Indeed, the low efficacy of modified NK cells is often cited as the obstacle to their widespread acceptance as a therapeutic modality.

Further approaches for CAR-cell-based immunotherapies have been developed. For example, a CAR-like single chain receptor has been employed in B cells, whose purpose was to trigger B cell expansion upon the encounter of a cognate antigen (Pesch, et al. 2019). Furthermore, a CAR-based multi-chain receptor has been engineered that links a signaling-deficient CAR via a transmembrane domain to endogenous signaling chains, to utilize their signaling capacity (Wang, et al. 2015a). However, many or all the limitations and drawbacks of the CARs described above still apply to these studies.

Furthermore, the reprogramming of T cell specificity via modifications of their TCR is still a venue pursued for the development of T cell therapeutics. The latter approaches range from modifying TCRs with binding domain candidates identified in mice with fully diverse human TCRαβ repertoire (Li, et al. 2010), to scFv-decorated TCRs (TRuCs) (Baeuerle, et al. 2019). However, only the antigen binding moiety is engineered, while the signaling domains remain the endogenous TCR components. Moreover, retaining the endogenous αβ or γδ TCR antigen-binding domains in T cells augmented with engineered binding domains, can result in cross talk and lead to Graft versus Host disease (GvHd) (Bendle, et al. 2010).

Thus, there is a need for improved means and methods for cell-based immunotherapies.

In particular, the present invention relates to the embodiments as characterized in the claims and as described herein below.

Accordingly, the invention relates to a modified mammalian cell comprising the following (I) and (II):
(I) a first and second polypeptide, each comprising a variable region, wherein the variable region of the first polypeptide and the variable region of the second polypeptide form an antigen-binding site at the external side of the cell, wherein the first polypeptide further comprises a membrane domain located within the membrane of the cell, and wherein the first and second polypeptide are not, preferably do not comprise:
   (i) an alpha and beta chain of a T cell receptor (TCR), or
   (ii) a gamma and delta chain of a TCR; and
(II) at least one polypeptide, e.g. said first polypeptide and/or at least one further polypeptide, comprising an intracellular domain containing at least one signaling domain; and
wherein, upon binding of the antigen-binding site to a corresponding antigen, at least one of the signaling domains triggers a process in the cell that enables the cell to promote death of a target cell comprising said antigen on the cell surface.

In particular, said first polypeptide, said second polypeptide and (when present) said at least one further polypeptide comprising an intracellular domain containing at least one signaling domain (e.g. a third CD79A-like and/or a fourth CD79B-like polypeptide) form a multi-chain antigen receptor (i.e a multi-chain antigen-specific receptor) according to the invention as described herein.

Furthermore, said multi-chain antigen receptor may comprise one or more further polypeptides that bind to or interact with said polypeptides as described herein in context of the present invention, e.g. a CD16-like polypeptide.

In particular, the term “multi-chain” refers to the presence of multiple polypeptides (i.e. multiple amino acid chains).

Sometimes, the multi-chain antigen receptors of the present invention are briefly called “antigen receptor” or “antigen-specific receptor” herein.

Sometimes, in particular when at least one further polypeptide as described herein binds to or interacts with said first and said second polypeptide, the multi-chain antigen receptor is also considered as a multi-chain antigen receptor complex herein and in context of the present invention, e.g., a BCR-like complex.

Herein and in context of the present invention, one or more of the polypeptides comprising an intracellular domain containing at least one signaling domain (e.g. said first polypeptide, a third CD79A-like polypeptide and/or a fourth CD79B-like polypeptide) may be a chimeric polypeptide as described herein, e.g. wherein the extracellular and membrane domains are derived from one protein (e.g. CD79A or CD79B) and the intracellular domain is derived from another protein (e.g. CD3zeta). In some instances, multi-chain antigen receptors of the present invention are also called “Antigen-specific Synthetic Immunoglobulin-based Multi-chain receptors (ASIMut Receptors)” herein and in context of the present invention. As described herein and as illustrated in the present Examples, the multi-chain antigen receptors of the present invention can be highly modular and versatile and thus may be also considered as a platform herein and in context of the present invention, e.g., an ASIMut platform.

Exemplary modified mammalian cells according to the invention are the modified NK cells that are described and illustrated in the appended Examples. An exemplary first and second polypeptide according to the present invention is the membrane-bound anti-Her2 antibody that is expressed on the surface of the NK cells described and illustrated in the appended Examples. Accordingly, corresponding exemplary target cells are the Her-2 expressing target cells employed in the appended Examples. Furthermore, an exemplary polypeptide comprising an intracellular domain containing at least one signaling domain is the CD79A or CD79B polypeptide employed in the appended Examples. Exemplary polypeptides comprising an intracellular domain containing at least one signaling domain may be further the CD3 zeta and/or FceRIg polypeptides employed in the appended Examples, in particular, when the modified cell further comprises a CD16-like polypeptide which is exemplified by the CD16 polypeptide employed in the appended Examples. Exemplary intracellular domains or signaling domains are the CD3 zeta, FceRIG, CD79A, CD79B or IgG1 intracellular or signaling domains employed in the appended Examples. An exemplary process that enables the modified cell to promote death of a target cell is the cis-ADCC, as illustrated in the appended Examples. The modularity and versatility of the multi-chain antigen receptors of the present invention is shown, e.g., in Example 5 and 6. Exemplary chimeric polypeptides than can be used, e.g., in the multi-chain antigen receptors of the present invention, are also shown in these Examples.

However, the present invention is in no way limited to the appended Examples or these exemplary cells, polypeptides, domains or processes.

The invention is, at least partly, based on the surprising finding that an antibody, e.g. an IgG1 or IgM, can be also expressed on the surface of other mammalian cells than B cells, e.g. natural killer (NK) cells or cancer cells such as HeLa cells, even in the absence of CD79, as illustrated in the appended Examples. Furthermore, the inventors surprisingly found that NK cells expressing an antibody on the cell surface, e.g. against Her-2, were able to eliminate Her-2 positive target cells (FIG. 2h). This suggests that expression of a membrane-bound antibody in NK cells induced antibody dependent cellular cytotoxicity (ADCC) against target cells in cis, i.e. in the absence of a soluble antibody (see, e.g, FIG. 1b). Remarkably and completely unexpectedly, NK cells which expressed an anti-Her2 antibody on the cell surface but lacked CD16, still eliminated up to 27% of Her2 positive target cells (FIG. 2h, i), indicating that the membrane-bound antibody may even function on its own.

Furthermore, it has been surprisingly found that the cis-ADCC induced by the expression of a membrane-bound anti-Her2 antibody in CD16-expressing NK cells was similarly effective in killing target cells than canonical trans-ADCC induced by addition of the corresponding soluble anti-Her2 antibody to control NK cells not expressing any antibody (compare FIGS. 2g and j).

This further shows that, in the context of the present invention, mammalian cells such as NK cells can be endowed with selective cytotoxicity against antigen-expressing target cells. Furthermore, this demonstrates that the immobilization of an antibody on the surface of mammalian cells such as NK cells (FIG. 1b) circumvents the need for a soluble antibody to make contact with its target antigen and induce ADCC in trans (FIG. 1a). In particular, inducing ADCC in cis according to the present invention (FIG. 1b) can greatly increase the ADCC efficiency in many therapeutic applications, e.g. in the treatment of tumors, at least, because the ADCC does not rely on the diffusion of a soluble antibody which is often a limiting factor.

Yet, the invention is not limited to the use of antibodies but also encompasses, inter alia, the use of proteins which share some similarities with antibodies, as described herein, in particular, in the context of the first and second polypeptide of the invention. Preferably, the proteins used in the context of the present invention for antigen-binding comprise at least two polypeptides each comprising a part of the antigen-binding site, wherein at least one of them has a membrane domain. In other words, the use of antigen-binding sites which are split into at least two, e.g. two, polypeptides, e.g. similarly as in antibodies, is preferred in the context of the invention.

The use of multichain antigen-binding sites and multichain antigen receptors, for example, those formed by or comprising the first and second polypeptide of the present invention, e.g. as in an antibody (immunoglobulin), allows to overcome many drawbacks associated with single-chain chimeric antigen receptors (CARs).

For example, the use of full-length membrane bound antibodies or antibody-like multichain proteins as or in antigen receptors may avoid undesired clustering of the antigen receptors on the cell surface and tonic signaling as often observed for CARs, and allow more robust expression of the antigen-receptors in mammalian cells. In particular, full-length immunoglobulins are unable to mispair. Moreover, an immunoglobulin appears to be already perfect in length for cell activation, as it has been designed for this purpose by nature. Thus, multichain antigen receptors which resemble or comprise immunoglobulins (antibodies), in particular those formed by or comprising the first and second polypeptide according to the invention, may be advantageous for activating mammalian cells in cis for this additional reason.

Furthermore, in particular due to the high modularity, an antigen receptor according to the invention can be built in a straightforward fashion on the basis of a preexisting monoclonal antibody. In particular, as illustrated in Example 6 and FIG. 13, the antigen-binding site, which can be formed, e.g., by the CDRs of the heavy and light chains of an antibody, may be easily switched and/or adapted to a target antigen, while the remaining parts of the antigen-receptor protein can be left unchanged. Furthermore, as illustrated in the appended Examples, also non-antigen binding parts of a polypeptide involved in antigen-binding (for example the first polypeptide of the present invention) e.g. a polypeptide which resembles the heavy chain of a membrane-bound antibody, can be easily switched and/or adapted for specific purposes. For example, as demonstrated in the appended Examples, the non-variable region of the heavy chain of a membrane-bound IgG1 could be replaced by the non-variable region of the heavy chain of a membrane-bound IgM without adversely affecting the killing of target cells, e.g., when chimeric CD79-CD3 zeta polypeptides were co-expressed in the NK cells (see, e.g., FIG. 4). Furthermore, by using antigen-binding polypeptides derived from different immunoglobulin classes, it is possible to couple (e.g., IgG1) or decouple (e.g., IgM) the antigen receptor response from other endogenous receptors such as CD16. Furthermore, it is possible, in the context of the present invention, to exchange a part which resembles the constant region of an IgG antibody with a less or non-immunogenic constant region derived from IgM.

Thus, the multichain antigen receptors according to the invention, i.e. formed by or comprising the first and second polypeptide of the invention, can provide a greater modularity and adaptability than conventional CARs. In addition, the use of multichain antigen receptors according to the invention, which may form dimers, multimers and/or complexes, and which may have several membrane and intracellular domains, allows to add intracellular signaling or activation domains in parallel, i.e. in different polypeptide chains of the antigen receptor, and not only in series (“in-line”) within one polypeptide chain as in conventional CARs. As described herein, the use of multiple signaling or activation domains in parallel avoids undesired issues due to suboptimal distances of the domains from the cell membrane, which may be associated with insufficient or undesired activation of signaling pathways, as has been observed with CARs (Feucht, et al. 2019)). Therefore, the multichain antigen receptors or antigen receptor complexes according to the invention can have higher efficacy than conventional CARs, at least, because multiple signaling domains and/or co-stimulatory domains can be arranged in an optimal manner, e.g., in parallel and with an optimal distance and orientation to the cell membrane. Moreover, many more active motifs such as ITAMs can be included in multichain antigen receptor complexes according to the invention compared to CARs of the prior art. By increasing the number of ITAMs per antigen receptor, it is possible to lower the activation threshold for cytotoxic cells for a given concentration of target epitopes.

Furthermore, the multichain antigen receptors according to the invention, i.e. those formed by or comprising the first and second polypeptide of the invention, may interact with further polypeptides in mammalian cells, for example CD16, as illustrated in the appended Examples.

In addition, it has been found by the inventors that the co-expression of CD79 increased the surface expression of an antibody in mammalian cells, e.g., NK cells or cancer cells such as HeLa cells, as illustrated in the appended Examples. Surprisingly, as illustrated in the appended Examples, the NK cells were effective in killing target cells, when the antibody was expressed in combination with CD79 in the NK cells. Without being bound by theory, the antibody may have formed a multimeric B cell receptor (BCR)-like complex in these mammalian cells.

Thus, the antigen receptors according to the invention, i.e. the first and second polypeptide, may form BCR-like complexes with CD79 also in mammalian cells, e.g. cytotoxic cells, which are not B cells. The combination with CD79A and/or CD79B or polypeptides which resemble CD79 at least in some aspects, in particular CD79A-like and/or CD79B-like polypeptides as described herein, has the further advantage that even more signaling domains can be present within the multichain antigen receptor complexes. Again, the signaling domains can be added in parallel in the intracellular domains of different polypeptides (e.g. a polypeptide that is involved in antigen-binding such as the first polypeptide of the invention and CD79-like polypeptides such as the third and/or fourth polypeptides of the invention), and not only in series within one polypeptide chain as in conventional CARs.

While the extracellular and membrane domain of a polypeptide that is involved in antigen-binding, e.g. the first polypeptide of the invention, may resemble the extracellular and membrane domain of the heavy chain of a membrane-bound antibody, and the extracellular and membrane domains of the CD79-like polypeptides may resemble the extracellular and membrane domains of CD79A and/or CD79B, the intracellular domains of these antibody heavy chain-like polypeptide or CD79-like polypeptides may be completely different. Thus, in certain embodiments, polypeptides of the present invention, e.g. the first, third and/or fourth polypeptide, may be considered as chimeric polypeptides. For example, the intracellular domains of such chimeric polypeptides according to the invention, may resemble the intracellular domains and/or ITAMs of CD3 zeta and/or FceRIg. Thus, the multichain antigen receptors according to the invention may comprise chimeric polypeptides, e.g., wherein the extracellular and/or membrane domains are derived from a different protein (e.g. an antibody or CD79) and the intracellular domain is derived from another protein (e.g. CD3 zeta or FceRIg).

Very surprisingly, as illustrated in the appended Examples, the inventors have further found that NK cells expressing a membrane-bound anti-Her-2 antibody, e.g. an IgG1 or IgM, and chimeric CD79-CD3ζ (CD3 zeta) polypeptides which comprised the extracellular and membrane domains of CD79A or CD79B and the intracellular domain of CD3 zeta (i.e. CD3 zeta ITAMs), killed Her-2 expressing target cells much more efficiently, i.e. with much higher efficiency, than canonical trans-ADCC induced by the addition of a corresponding soluble anti-Her2 antibody to NK cells which expressed CD16 but not any antibody (see, e.g., FIGS. 4c, 4i, and 2g and Example 5). Notably, the presence of CD16 in the NK cells harboring the antibody and the CD79-CD3 zeta chimeric polypeptides was not required to achieve this outstanding and improved target cell lysis efficiency. Moreover, this demonstrates that chimeric polypeptides according to the present invention, e.g., polypeptides comprising an extracellular and/or membrane domain resembling the extracellular and/or membrane domains of CD79A or CD79B and the intracellular domain of CD3 zeta, can further improve the multichain antigen receptors according to the invention, and may be particularly useful for killing target cells.

Also very surprisingly, the inventors have further found that NK cells expressing a membrane-bound chimeric anti-Her-2 antibody-like protein which comprised the extracellular and membrane domains of an membrane bound antibody and the intracellular domain of FceRIg killed Her-2 expressing target cells much more efficiently than canonical trans-ADCC induced by the addition of a corresponding soluble anti-Her2 antibody to NK cells which expressed CD16 but not any antibody (see, e.g., FIGS. 5c, 2g and 3i, and Example 5). Remarkably, this chimeric antibody was well expressed on the surface of NK cells and killed the target cells efficiently even in the absence of CD16 and CD79.

Further very surprisingly, the inventors found that combined expression of the chimeric antibody-FceRIg polypeptide and chimeric CD79-CD3 zeta polypeptides even further increased the surface expression of the antibody and the efficiency of the killing process such that 96% of the target cells got killed at an NK cell:target cell ratio of 10:1 (see, e.g., FIG. 5d-f, and Example 5). This response strongly exceeded the responses achieved by canonical trans-ADCC. This further demonstrates that a BCR-like complex domains according to the invention comprising CD3ζ (CD3 zeta) and/or FcεRIγ (FceRIg) signaling can endow mammalian cells with very high levels of antigen-specific cytotoxicity against antigen-expressing cells.

Moreover, as illustrated in Example 6 and FIG. 13, it has been confirmed that the antigen-specificity of the multi-chain antigen receptors of the present invention, e.g. antigen receptors comprising a chimeric antibody-FceRIg polypeptide and chimeric CD79-CD3 zeta polypeptides, can be easily switched in order to kill different target cells. For example, it has been found that a multi-chain antigen receptor containing a CD19 or CD20 binding site very efficiently killed CD19 and CD20 expressing Raji tumor cells.

However, the intracellular domain can be also derived from the same polypeptide as the extracellular and/or membrane domains, e.g., the entire polypeptide may resemble a heavy chain of a membrane-bound antibody, or CD79A or CD79B. As already mentioned above, and as illustrated in the appended Examples, non-chimeric antibodies alone, or in combination with CD16 and/or non-chimeric CD79 proteins also achieved a considerable efficiency of killing of target cells.

Furthermore, as illustrated in the appended Examples, the inventors further surprisingly found that the introduction of a flexible peptide linker at the membrane-proximal part of the extracellular domain significantly increased the antibody surface expression in NK cells and resulted in improved lysis of target cells compared to antibodies without such a linker, in particular, in the presence of CD16 or CD16 and CD79 (Example 4). Remarkably and unexpectedly, the tethering of an antibody to the cell membrane by means of a flexible linker in combination with CD16 or CD16 and CD79 eliminated target cells much more efficiently than canonical ADCC, and about as efficiently as a membrane-bound antibody (without linker) in combination with chimeric CD79-CD3 zeta polypeptides carrying ITAMs from CD3 zeta.

This demonstrates again that the multichain receptor architecture according to the invention exhibits superior cytotoxicity, further highlighting the advantages of synergizing between multiple extracellular and intracellular domains that are placed at an optimal distance from, and/or orientation with respect to each other and the plasma membrane.

Overall, the findings of the inventors show that the antigen receptors or antigen receptor complexes according to the invention, which may be also called Antigen-specific Synthetic Immunoglobulin-based Multi-chain receptors (ASIMut Receptors), exhibit high surface expression and induce strong ADCC against target cells in an antigen specific manner. Thus, the present invention further provides a flexible, programmable framework and platform for the development of further therapeutic antigen-specific mammalian cells such as NK cells. Furthermore, the invention provides a new class of mammalian cell (e.g. NK cell)-based cell therapies that may supplement or even outperform the existing antigen-specific cell therapies, e.g. CAR-T cell therapies, for the treatment of cancer and other diseases. Moreover, the use of modified mammalian cells according to the invention, e.g. NK cells, for therapy may have further advantages, such as the possibility to employ allogeneic cell material due to the lack of GvHd (Xie, et al. 2020).

Accordingly, as described herein, the present invention relates to a modified mammalian cell comprising the following (I) and (II):
(I) a first and second polypeptide, each comprising a variable region,
   wherein the variable region of the first polypeptide and the variable region of the second polypeptide form an antigen-binding site at the external side of the cell, and
   wherein the first polypeptide further comprises a membrane domain located within the membrane of the cell, and
(II) at least one polypeptide comprising an intracellular domain containing at least one signaling domain, e.g. said first polypeptide and/or at least one further polypeptide.

In particular, said first and second polypeptide are not: (i) an alpha and beta chain of a T cell receptor (TCR), or (ii) a gamma and delta chain of a TCR. In particular, when the modified cell is a T cell, it comprises the polypeptides as defined in (I) and (II) above in addition to or instead of any conventional, e.g., endogenous, T cell receptors.

In particular herein, the modified cell of the invention is able to promote death of a target cell comprising an antigen on the cell surface to which said antigen-binding site can bind. In particular, herein and in context of the present invention, at least one of said signaling domains triggers a process in the cell that enables the cell to promote death of a target cell comprising said antigen on the cell surface upon binding of the antigen-binding site to a corresponding antigen, e.g. on the surface of said target cell. Preferably, herein and in context of the present invention, the modified cell is not a B cell that is required to interact with another immune cell type to promote death of the target cell. In some embodiments, the modified cell of the invention is not a B cell.

The modified cell, as used herein and in the context of the invention, is a mammalian cell. The modified mammalian cell according to the present invention is not limited to any particular cell type or mammalian species. Preferably, the mammalian cell is a human cell, but it may be also a cell from another mammalian species, e.g. mouse, rat, hamster, monkey, horse, pig, cow or sheep, etc. In some embodiments of the invention, the modified cell is not a HEK393 cell.

Preferably herein, and in the context of the present invention, the modified cell is a cytotoxic lymphocyte, in particular a natural killer (NK) cell or a T cell, most preferably a NK cell. The T cell may be a CD8+ T cell such as a cytotoxic T cell, or a CD4+ T cell such as a helper T cell or a regulatory T cell. Preferably, the T cell is a CD8+ T cell, preferably a cytotoxic CD8+ T cell.

Furthermore, the modified cell of the invention may be a primary cell or a cell line, as commonly understood in the art. Furthermore, the modified cell of the invention may be derived from cord blood or peripheral blood, obtained by differentiation of a pluripotent cell, e.g. an induced pluripotent stem cell, or obtained by reprogramming of another cell type.

A polypeptide, as used herein, comprises or refers to an amino acid chain, which preferably is at least 5, 10, 15, 20, 30, 40, 50, 100, 200, 300, 400, 500, 600, 700, 800, 900, 1000, 2000, 3000, 4000, 5000 or 10000, preferably at least 50, 100, 200, 300, 400 or 500, more preferably at least 200, 300, 400 or 500 amino acids in length. Furthermore, the polypeptide is preferably less than 50000, 20000, 10000, 5000, 4000, 3000, 2000, or 1000 amino acids in length. A polypeptide may be also called a protein herein, and a protein may be called a polypeptide, because a polypeptide, as used herein, can be considered a single chain protein. However, in contrast to a polypeptide, a protein can also refer to a complex of multiple polypeptides.

Unless explicitly indicated otherwise, generally herein and in the context of the present invention, different polypeptides refer to separate amino acid chains. However, when it is explicitly indicated that two or more polypeptides are covalently linked, e.g., via peptide bonds, these two or more polypeptides form one amino acid chain, and thus may exceptionally be considered one polypeptide.

Preferably, and in most aspects of the present invention, the modified mammalian cell comprises a first and second polypeptide, each comprising a variable region, wherein the variable region of the first polypeptide and the variable region of the second polypeptide form an antigen-binding site at the external side of the cell, and wherein the first polypeptide further comprises a membrane domain located within the membrane of the cell. Furthermore, the first polypeptide may comprise an intracellular domain. The second polypeptide may also comprise a membrane domain, and optionally an intracellular domain, or it may be located in its entirety at the external side of the cell. Preferably, the first and second polypeptide are expressed in the modified mammalian cell from one or more nucleic acids. Thus, the modified cell may comprise one or more nucleic acid molecules, from which the first and second polypeptide are expressed.

Hence, the modified mammalian cell of the invention comprises preferably a multichain antigen-binding site, wherein one part of the antigen-binding site is comprised in the variable region of the first polypeptide and the other part of the antigen-binding site is comprised in the variable region of the second polypeptide.

The amino acid sequence of a variable region, as used herein and in context of the invention, depends on the antigen-specificity. A variable region, as used herein, may correspond to the variable region of an antibody, but it is not limited thereto. For example, it may comprise only one or more CDRs of a variable region of an antibody, and/or other sequences which allow a specific binding to an antigen.

As used herein, and in the context of the invention, the antigen-binding site can specifically bind to an antigen or epitope, as commonly understood in the art. Preferably, the first and second polypeptide according to the invention, which may be considered an antigen receptor, bind via their common antigen-binding site to an antigen with a similar strength and/or with a similar specificity as antibodies, preferably monoclonal antibodies that are used for therapeutic and/or detection purposes. For example, the first and second polypeptide may bind an antigen with a dissociation constant (KD) of 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or 10⁻¹³ M, or less, preferably 10⁻⁶ M, 10⁻⁷ M, 10⁻⁸ M, 10⁻⁹ M, 10⁻¹⁰ M, 10⁻¹¹ M, or 10⁻¹² or less.

In particular, herein and in context of the invention, the first and second polypeptide form or are part of an antigen receptor or antigen receptor complex, i.e. a multichain antigen receptor or multichain antigen receptor complex, as described herein. The terms “antigen receptor” and “antigen-specific receptor” may be used interchangeably herein.

A membrane domain, as used herein, may be, but is not limited to, a transmembrane domain, as commonly understood in the art. The membrane of a cell, as used herein, refers to the plasma membrane of the cell, as commonly understood in the art.

The modified mammalian cell according to the invention is not an unmodified B cell or an unmodified T cell.

While the modified cell of the invention may be a modified T cell, it does not only comprise a conventional T cell receptor (TCR) as only antigen receptor. In particular, a modified T cell of the present invention comprises the first and second polypeptide described herein instead of or in addition to a TCR.

Thus, preferably herein, and in most aspects of the invention, the first and second polypeptide are not (i) an alpha and beta chain of a TCR, or (ii) a gamma and delta chain of a TCR. More preferably, the first and second polypeptide do not comprise (i) an alpha and beta chain of a TCR, or (ii) a gamma and delta chain of a TCR. In particular, the first polypeptide does not comprise an alpha chain of a TCR and the second polypeptide does not comprise a beta chain of a TCR or vice versa, and/or the first polypeptide does not comprise a gamma chain of a TCR and the second polypeptide does not comprise a delta chain of a TCR or vice versa. In particular, the first and second polypeptide described herein do not form a conventional T cell receptor, as commonly understood in the art. Furthermore, the person skilled in the art can easily recognize whether a polypeptide is an alpha, beta, gamma or delta chain of a conventional TCR based on common general knowledge and using common sense, even when the alpha, beta, gamma or delta chain of a TCR has been slightly modified. However, the first and second peptide according to the invention may be or comprise considerably modified alpha, beta, gamma or delta chains of a TCR, e.g., chimeric polypeptides and/or polypeptides which have a considerably different functionality than unmodified alpha, beta, gamma or delta chains.

Furthermore, the modified cell according to the invention comprises at least one polypeptide which comprises an intracellular domain containing at least one signaling domain, as described herein. A signaling domain may be comprised in the first polypeptide according to the invention and/or in another polypeptide, e.g. a third and/or fourth polypeptide, contained in the modified cell. Thus, said at least one polypeptide which comprises an intracellular domain containing at least one signaling domain, may comprise the first polypeptide, as described herein, and/or at least one other polypeptide, e.g. the third and/or fourth polypeptide, as described herein.

A signaling domain, as used herein, which is also sometimes called an “activation domain” herein, has the capacity to trigger or enhance a process in a mammalian cell, e.g., the modified cell of the invention, that enables the cell to promote death of a target cell. A target cell, as used herein and in the context of the invention, is a cell, preferably a mammalian cell, e.g. from the same species as the modified cell of the invention, which comprises an antigen to which the antigen-binding site of the modified cell according to the invention can bind, i.e. specifically bind, as described herein. In particular, the target cell expresses or displays said antigen on the cell surface.

In particular, in the context of the present invention, upon binding of the antigen-binding site to a corresponding antigen, as described herein, a signaling domain triggers or enhances a process in the modified cell of the invention that enables the cell to promote death of a target cell comprising said antigen on the cell surface. In particular, the modified cell of the invention kills the target cell upon binding to the antigen, e.g. when the antigen-binding site binds or has bound an antigen on the cell surface of the target cell. Preferably, the signaling domain does not trigger or enhance said death promoting process in the cell, when the antigen-binding site has not bound or does not bind to its cognate antigen. At least, said death promoting process is triggered or enhanced to a much greater extent when the antigen-binding site binds or has bound an antigen. For example, a target cell may be killed when the modified cell of the invention binds an antigen on the surface of the target cell via the antigen-binding site formed by the first and second polypeptide according to the invention.

The modified cell may promote the death of a target cell upon binding of the antigen-binding site to an antigen according to the invention in a direct or indirect manner, preferably in a direct manner.

Thus, the promotion of the death of a target cell upon binding of the first and second polypeptide, i.e. an antigen receptor of the invention, to an antigen, in particular on the surface of the target cell, can be, at least in some aspects, similar to the antibody-dependent cellular cytotoxicity (ADCC), in particular, the cis-ADCC, as described herein and as illustrated in the appended Examples, although the first and second polypeptide can be different from a conventional antibody. In particular, the promotion of the death of a target cell may be similar to ADCC, at least in some aspects, i.e. cis-ADCC, as described herein and as illustrated in the appended Examples, when CD16 is involved.

Without being bound by theory, the modified cell of the invention may bind to an antigen on the surface of a target cell via the antigen-binding site, wherein an immunological synapse is formed. Considering a kinetic segregation model, as is discussed in the art, upon binding of the antigen, phosphatases in the modified cell which stick out into the extracellular space and which constantly dephosphorylate the signaling domains, e.g. the ITAMs comprised in the signaling domains, may be deplaced, i.e. separated from phosphatases, and, as a consequence, the signaling domains may get phosphorylated. When the signaling domains are phosphorylated, a process may be triggered or enhanced in the modified which enables the modified cell to kill the target cell, e.g. via a cis-ADCC-like mechanism. It is therefore important that the modified cell comprises an antigen-binding site at the external side of the cell, and at least one signaling domain, i.e. at the internal side of the cell. However, it is less important, whether the signaling domains are comprised in the same polypeptides which form the antigen-binding site, or whether they are comprised in other polypeptides in the modified cell. For example, the first and second polypeptides which form the antigen-binding site, and other polypeptides carrying signaling domains may get together in a membrane raft in the modified cell, with or without direct contact.

Preferably, the modified cell of the invention is not required to interact with another immune cell type to the promote death of a target cell. In other words, the modified cell of the invention preferably kills the target cell in a direct manner, e.g., by means of a process which is, at least in some aspects, similar to the cis-ADCC described herein and illustrated in the appended Examples. Furthermore, the modified cell of the invention may be also considered an effector cell as described herein and as illustrated in the appended Examples.

As well known in the art, conventional B cells cannot promote the death of a target cell, at least not without the requirement to interact with another immune cell type. In particular, conventional B cells cannot kill a target cell in a direct manner.

Hence, in certain embodiments of the invention, the modified cell is not a B cell that is required to interact with another immune cell type to promote death of a target cell. However, the modified cell of the invention may be a B cell which has been modified according to the present invention, for example, a B cell which has the capacity to promote the death of a target cell without the requirement to interact with another immune cell type, e.g., in a cis-ADCC like fashion as described herein. Furthermore, the modified cell of the invention may be a B cell which comprises instead of or in addition to an unmodified or conventional membrane-bound antibody, the first and second polypeptide and/or an antigen receptor according to the present invention, wherein said first and second polypeptide and said antigen receptor are different from a conventional membrane-bound antibody e.g., a membrane-bound antibody of unmodified B cells.

Furthermore, the modified cell of the invention may be a B cell which comprises an antigen receptor complex, e.g. a BCR-like complex, according to the invention, e.g., comprising the first and second polypeptide, and the third CD79A-like and/or fourth CD79B-like polypeptide as described herein, wherein said antigen receptor complex is different from a conventional BCR, e.g. a BCR of unmodified B cells. For example, in the modified B cell of the invention, at least one or all components of the endogenous BCR may be missing, e.g. the genes for the membrane-bound antibody, and/or CD79A or CD79B may have been knocked out, and at least one nucleic acid encoding the first and second polypeptide, and/or the CD79A-like third and/or CD79B-like fourth polypeptide of the invention, as described herein, may have been introduced.

Furthermore, the modified B cell of the invention may be a B cell which further comprises CD16 and/or the fifth polypeptide of the invention, as described herein.

Furthermore, the modified cell of the invention, e.g., the modified B cell of the invention, may comprise a chimeric antigen-binding polypeptide, or a chimeric CD79 peptide, as described herein.

Yet, in some embodiments of the invention, the mammalian cell is not a B cell.

Herein, and in the context of the present invention, the variable regions of the first and second polypeptide may contain at least one, preferably at least three, preferably all, complementary determining region(s) (CDR) of an antibody. Preferably the first and second polypeptide contain each at least one, preferably three, CDR(s) of the antibody.

In particular, the first polypeptide may contain the complementary determining regions (CDRs) of a heavy chain of an antibody, i.e., CDR-H1, CDR-H2 and CDR-H3, and/or the variable region of the second polypeptide may contain the CDRs of a light chain of said antibody, i.e., CDR-L1, CDR-L2 and CDR-L3.

However, it is also possible that the first polypeptide contains the complementary determining regions (CDRs) of a light chain of an antibody, i.e., CDR-L1, CDR-L2 and CDR-L3, and/or the variable region of the second polypeptide contains the CDRs of a heavy chain of said antibody, i.e., CDR-H1, CDR-H2 and CDR-H3.

Furthermore, the first polypeptide according to the invention may comprise the variable region of a heavy chain of the antibody, and/or the second polypeptide may comprise the variable region of a light chain of the antibody.

It is well known in the art that the complementary determining regions (CDRs) determine the binding specificity of an antibody. The CDR regions of an antibody or Ig-derived region, may be determined as described in Kabat (1991), Sequences of Proteins of Immunological Interest, 5th edit., NIH Publication no. 91-3242 U.S. Department of Health and Human Services, and/or Chothia (1987), J. Mol. Biol. 196, 901-917; and Chothia (1989) Nature, 342, 877-883. The CDRs as provided herein above were determined by the Kabat system. Due to its wide-spread use and reliability, the Kabat numbering system may be preferred.

Suitable methods to determine the sequence of an antibody, e.g. a monoclonal antibody are also readily available in the art.

The variable regions of an antibody may be grouped into the CDRs and the framework regions (FRs), in particular by using the Kabat or Chothia numbering schemes.

Thus, the CDRs or variable regions of an antibody can be readily determined by methods known in the art, e.g. by employing the Kabat system.

The terms “antibody” and “immunoglobulin” are used interchangeably herein, and as commonly understood in the art. Preferably, an antibody, as used herein, is a monoclonal antibody. The antibody may be also a CDR grafted antibody, a chimeric antibody, a humanized antibody, or a fully human antibody.

The antibody according to the present invention, i.e. in the context of CDRs and/or variable regions of an antibody, is not limited to any particular antibody. In fact, any existing or future antibody may be used in the context of the present invention. In particular, the first and/or second polypeptide of the invention can comprise CDRs and/or variable regions from any existing or future antibody. The selection of the CDRs or variable regions of an antibody depends primarily on the antigen that is to be recognized, and/or the target cell that is to be killed. For example, as illustrated in the appended Examples, an anti-Her-2 antibody, i.e. derived from Trastuzumab/Herceptin, has been expressed on the surface of a modified cell according to the invention to kill Her-2 expressing target cells. However, the invention is in no way limited to an anti-Her-2 antibody, or Her-2 expressing target cells. It has been further confirmed in Example 6 and FIG. 13 that the CDRs or variable region of Trastuzumab in a multi-chain antigen receptor of the present invention can be readily replaced by the CDRs or the variable region of another antibody, e.g. an anti-CD19.1 antibody (i.e. FMC63), an anti-CD19.2 antibody (i.e. inebilizumab) or an anti-CD20 antibody (i.e. Rituximab). Indeed, it has been found that effector cells expressing such a modified multi-chain antigen receptor efficiently killed CD19 and CD20 expressing target cells (e.g. Raji) cells.

Further suitable antibodies, that may be used the context of the present invention may be, inter alia:
3F8, Abagovomab, Abciximab, Abituzumab, Abrezekimab, Abrilumab, Actoxumab, Adalimumab, Adecatumumab, Aducanumab, Afasevikumab, Afelimomab, Alacizumab pegol, Alemtuzumab, Alirocumab, Altumomab pentetate, Amatuximab, Amivantamab, Anatumomab mafenatox, Andecaliximab, Anetumab ravtansine, Anifrolumab, Ansuvimab, Anrukinzumab (=IMA-638), Apolizumab, Aprutumab ixadotin, Arcitumomab, Ascrinvacumab, Aselizumab, Atezolizumab, Atidortoxumab, Atinumab, Atoltivimab, Atoltivimab/maftivimab/odesivimab, Atorolimumab, Avelumab, Azintuxizumab vedotin, Bamlanivimab, Bapineuzumab, Basiliximab, Bavituximab, BCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, Birtamimab, Bivatuzumab, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Casirivimab, Capromab, Carlumab, Carotuximab, Catumaxomab, cBR96-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Dinutuximab beta, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epcoritamab, Epitumomab cituxetan, Epratuzumab, Eptinezumab, Erenumab, Erlizumab, Ertumaxomab, Etaracizumab, Etesevimab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, lanalumab, Ibalizumab, Sintilimab, Ibritumomab tiuxetan, Icrucumab, Idarucizumab, Ifabotuzumab, Igovomab, Iladatuzumab vedotin, Imalumab, Imaprelimab, Imciromab, Imdevimab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Regdanvimab, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Sotrovimab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Talquetamab, Tamtuvetmab, Tanezumab, Taplitumomab paptox, Tarextumab, Tavolimab, Teclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, tiragolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine, TRBSO7, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab, Zolbetuximab, or Zolimomab aritox.

Furthermore, the present invention relates to a kit comprising one or more nucleic acid molecules encoding the first and the second polypeptides, and optionally at least one further polypeptide, according to the present invention.

Accordingly, the present invention further relates to a kit comprising one or more nucleic acid molecules, wherein said nucleic acid molecule(s) comprise the following (I) and (II):
(I) a first coding sequence encoding a first polypeptide, and a second coding sequence encoding a second polypeptide,
   wherein the first and second polypeptide each comprise a variable region, and the first polypeptide further comprises a membrane domain,
   wherein the variable region of the first polypeptide and the variable region of the second polypeptide are able to form an antigen-binding site when expressed together in a modified mammalian cell,
   and wherein the first and second polypeptide are not, preferably do not comprise:
   (i) an alpha and beta chain of a T cell receptor (TCR), or
   (ii) a gamma and delta chain of a TCR; and
(II) at least one coding sequence encoding at least one polypeptide, e.g. said first polypeptide and/or at least one further polypeptide, comprising an intracellular domain containing at least one signaling domain; and
wherein, in a modified cell comprising the first and second polypeptide and at least one polypeptide comprising an intracellular domain containing at least one signaling domain, upon binding of the antigen-binding site to a corresponding antigen, at least one of the signaling domains is able to trigger a process in the cell that enables the cell to promote death of a target cell comprising said antigen on the cell surface.

As regards, e.g., the first and second polypeptide, the antigen-binding site, the modified cell, the intracellular domain, the signaling domain, the antigen, the target cell and the death promoting process, the same applies to the kit of the invention, as described herein in the context of the modified cell of the invention.

Herein, and in the context of the present invention, the first polypeptide may comprise an intracellular domain containing at least one signaling domain, as described herein. Furthermore, the modified cell of the invention may comprise a third and/or a fourth polypeptide, wherein at least one of the third and fourth polypeptides comprises an intracellular domain containing at least one signaling domain, as described herein. Preferably, each of the third and fourth polypeptide comprises an intracellular domain containing at least one signaling domain. Furthermore, the third and/or fourth polypeptide comprising an intracellular domain containing at least one signaling domain may be able to interact with and/or bind to the first polypeptide, i.e., in the modified cell of the invention.

Furthermore, the modified cell may comprise a fifth polypeptide, wherein the fifth polypeptide is able to interact with and/or bind to the first polypeptide, and the third and/or fourth polypeptide comprising an intracellular domain containing at least one signaling domain as described herein, i.e., in the modified cell of the invention.

Accordingly, the nucleic acid molecule(s), e.g. in the context of the kit of the invention, may comprise a third coding sequence encoding a third polypeptide and/or a fourth coding sequence encoding a fourth polypeptide, as described herein, e.g. in the context of the modified cell of the invention. Furthermore, the nucleic acid molecule(s), e.g. in the context of the kit may further comprise a fifth coding sequence encoding the fifth polypeptide, as described herein, e.g. in the context of the modified cell of the invention.

In the context of the invention, the first polypeptide may be considered as an antibody heavy chain-like polypeptide, and the second polypeptide may be considered as an antibody light chain-like polypeptide. Furthermore, the first and second polypeptide together may be considered an antibody-like protein.

Furthermore, the third polypeptide may be considered as a CD79A-like polypeptide, and the fourth polypeptide may be considered as a CD79B-like polypeptide. Furthermore, the fifth polypeptide may be considered as a CD16-like polypeptide. However, in some embodiments of the invention, i.e. when the fifth CD16-like polypeptide is involved, the third polypeptide may be considered as a CD3 zeta-like polypeptide and/or the fourth polypeptide may be considered as a FceRIg-like polypeptide.

In particular, as described herein, the first and second polypeptide, optionally in combination with the third, fourth and/or fifth polypeptide as described herein in context of the present invention, form a multi-chain antigen receptor according to the present invention and as described herein.

Therefore, the first and second polypeptide, in combination with at least one further polypeptide, e.g. the third and/or fourth polypeptide or the fifth polypeptide, according to the invention, may be also considered as an antigen receptor complex, or simply an antigen-receptor. However, the first and second polypeptide may also form an antigen-receptor of the invention by themselves. The combination of the first and second polypeptide with a third CD79A-like and/or a fourth CD79B-like polypeptide may be further considered as a BCR-like protein or BCR-like complex.

In general, herein, and in the context of the present invention, a polypeptide may comprise an amino acid sequence that has at least 10%, 20%, 30%, 40%, 50%, 60%, 70%, 80%, 90% or 100%, preferably at least 80%, 85%, 90%, 95% or 100%, preferably at least 90%, 91%, 92%, 93%, 94%, 95%, 96%, 97%, 98% or 99%, sequence identity to an amino acid sequence set forth in a certain SEQ ID NO. In general, the higher the % of the sequence identity, the more preferred the amino acid sequence is. For example, a sequence that has at least 90% sequence identity to the sequence set forth in SEQ ID NO: 4, may be more preferred than a sequence that has at least 50% sequence identity to the sequence set forth in SEQ ID NO: 4. Furthermore, a sequence which has 100% sequence identity to SEQ ID NO: 4, i.e. the sequence set forth in SEQ ID NO: 4 itself, may be the most preferred one in this example. A similar logic applies to essentially all amino acid sequences herein and in the context of the present invention which are defined by a sequence identity to an amino acid sequence set forth in a certain SEQ ID NO. However, it should be noted that the invention is in no way limited to high sequence identities, but any sequence identity, e.g. as just described above, may be considered.

The term “sequence identity”, as used herein, and in the context of the present invention, has essentially the same meaning, as commonly used and understood by the person skilled in the art.

In particular, herein, the term “sequence identity” is used to describe the sequence relationships between two or more amino acid sequences, proteins (or fragments thereof), or polypeptides (or fragments thereof). The term can be understood in the context of and in conjunction with the terms including: (a) reference sequence, (b) comparison window, (c) sequence identity, (d) percentage of sequence identity, and (e) substantial identity or “homologous”, as described in the following.

In particular, a “reference sequence”, e.g. a sequence as set forth in a certain SEQ ID NO., is a defined sequence used as a basis for sequence comparison.

In particular, a “comparison window” includes reference to a contiguous and specified segment of an amino acid sequence/polypeptide sequence/protein sequence, wherein the amino acid sequence/polypeptide sequence/protein sequence may be compared to a reference sequence. The portion of the amino acid sequence/polypeptide sequence/protein sequence in the comparison window may comprise additions, substitutions, or deletions (i.e., gaps) compared to the reference sequence (which does not comprise additions, substitutions, or deletions) for optimal alignment of the two sequences. For example, the comparison window may be about 20, 50, 100 or 200 amino acid residues in length or longer. Those of skill in the art understand that to avoid a misleadingly high similarity to a reference sequence due to inclusion of gaps in the polynucleotide or polypeptide sequence a gap penalty may be introduced and subtracted from the number of matches.

Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wisc., USA; the CLUSTAL program is well described by Higgins and Sharp (1988) Gene 73: 237-244; Corpet et al. (1988) Nucleic Acids Research 16:881-90; Huang, et al. (1992) Computer Applications in the Biosciences, 8:1-6; and Pearson, et al. (1994) Methods in Molecular Biology, 24:7-331. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future and can be used with the present invention.

Unless otherwise stated, sequence identity/similarity values provided herein may refer to the value obtained using the BLAST 2.0 suite of programs, or their successors, using default parameters. Altschul et al. (1997) Nucleic Acids Res, 2:3389-3402. It is to be understood that default settings of these parameters can be readily changed as needed in the future. Evidently, for comparison of amino acid sequences/protein sequences/polypeptide sequences, an algorithm/program directed to the alignment of amino acid sequences/protein sequences/polypeptide sequences should be used, e.g. BLASTP. As those ordinary skilled in the art will understand, BLAST searches assume that proteins or nucleic acids can be modeled as random sequences. However, many real proteins and nucleic acids comprise regions of nonrandom sequences which may be homopolymeric tracts, short-period repeats, or regions enriched in one or more amino acids or nucleic acids. Such low-complexity regions may be aligned between unrelated proteins even though other regions of the protein or nucleic acid are entirely dissimilar. A number of low-complexity filter programs can be employed to reduce such low-complexity alignments. For example, the SEG (Wooten et al. (1993) Comput. Chem. 17:149-163) and XNU (Claverie et al. (1993) Comput. Chem. 17:191-1) low-complexity filters can be employed alone or in combination.

“Sequence identity” in the context of two polypeptide/protein sequences includes, in particular, reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions may be said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

“Percentage of sequence identity” refers, in particular, to the value determined by comparing two optimally aligned sequences over a comparison window, wherein the portion of the amino acid/peptide/protein sequence in the comparison window may comprise additions, substitutions, or deletions (i.e., gaps) as compared to the reference sequence (which does not comprise additions, substitutions, or deletions) for optimal alignment of the two sequences. The percentage may be calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

Furthermore, the third polypeptide and/or the fourth polypeptide of the invention may comprise a membrane domain, as described herein. In particular, the membrane domain is located within the membrane of the modified cell of the invention. Preferably, each of the third and fourth polypeptide comprises a membrane domain.

The membrane domain of the third, i.e. CD79A-like, polypeptide, according to the invention may comprise the sequence motif “E-X₍₁₀₎-P” (i.e. EXXXXXXXXXXP), or a sequence that has at least 80% sequence identity to SEQ ID NO: 1. Furthermore, the membrane domain of the fourth, i.e. CD79B-like polypeptide may comprise the sequence motif “Q-X₍₁₀₎-P” (i.e. QXXXXXXXXXXP), or a sequence that has at least 80% sequence identity to SEQ ID NO: 2.

Furthermore, the membrane domain of the third, i.e. CD79A-like, polypeptide may comprise a sequence that has at least 50% sequence identity to the sequence set forth in SEQ ID NO: 4. Furthermore, the membrane domain of the fourth, i.e. CD79B-like, polypeptide may comprise a sequence that has at least 50% sequence identity to the sequence set forth in SEQ ID NO: 6. Furthermore, the membrane domain of the third, i.e. CD79A-like, polypeptide may comprise a sequence that has at least 50% sequence identity to the membrane domain of a CD79A protein and/or the sequence set forth in SEQ ID NO: 8. Furthermore, the membrane domain of the fourth, i.e. CD79B-like polypeptide may comprise a sequence that has at least 50% sequence identity to the membrane domain of a CD79B protein and/or the sequence set forth in SEQ ID NO: 10.

Evidently, herein and in context of the present invention, a certain domain or combination of domains (e.g. a membrane domain, an extracellular domain or an intracellular domain, or combinations thereof) that is defined by several larger and smaller sequences and/or sequence motifs derived from the same protein or corresponding domain(s) thereof may contain these sequences and/or motifs in an overlapping (i.e. nested) manner. In particular, the larger sequence normally contains the smaller sequence in such a case. Herein, a certain domain or combination of domains (e.g. a membrane domain, an extracellular domain or an intracellular domain or combinations thereof) may be described in preferred or more preferred ways by sequences and/or motifs derived from the same protein or corresponding domain(s) thereof or corresponding sequences (defined by a certain % sequence identity). In particular, both a higher sequence identity and a larger overlap with the reference protein or domain(s), as well as the presence of important motifs, may indicate a higher level of preference. With respect to the level of preference, in particular the similarity (structurally and functionally) to a reference protein (e.g. CD79A) or the corresponding reference domain thereof (e.g. the membrane domain of CD79A, or the combination of the extracellular domain and the membrane domain of CD79A) may be considered.

In an illustrative example, a membrane domain of the CD79A-like third polypeptide may comprise (i) a sequence that has at least 50% sequence identity to the sequence set forth in SEQ ID NO: 8 (which may be considered as a membrane domain derived from a CD79A protein), (ii) a sequence that has at least 50% sequence identity to the sequence set forth in SEQ ID NO: 4 (which may be considered as a part of a membrane domain derived from a CD79A) and (iii) the sequence motif “E-X₍₁₀₎-P”. In this example, the sequence defined in (ii) is contained, in particular, within the larger sequence defined in (i). Furthermore, the sequence motif “E-X₍₁₀₎-P” is, in particular, present within the sequences defined in (i) and (ii) such that the “E” and “P” are present in the sequences defined in (i) and (ii) and the positions of the “E” and the “P” are defined by the 10 “X” in between them.

A similar logic can be also applied to other domains, e.g., membrane domains, constant domains and intracellular domains and combinations thereof as described herein and in context of the present invention.

Furthermore, the third polypeptide and/or the fourth polypeptide according to the invention may comprise an extracellular domain. In particular, the extracellular domain is located at the external side of the modified cell of the invention. Preferably, each of the third and fourth polypeptide comprises an extracellular domain.

The extracellular domain of the third, i.e. CD79A-like, polypeptide may comprise a sequence that has at least 50% sequence identity to the extracellular domain of a CD79A protein and/or the sequence set forth in SEQ ID NO: 12 or 174, e.g. a sequence that has at least about 70% sequence identity to SEQ ID NO: 12. Furthermore, the extracellular domain of the fourth, i.e. CD79B-like, polypeptide may comprise a sequence that has at least 50% sequence identity to the extracellular domain of a CD79B protein and/or the sequence set forth in SEQ ID NO: 14 or 177, e.g. a sequence that has at least about 70%, preferably at least 80%, sequence identity to SEQ ID NO: 14.

Furthermore, the third, i.e. CD79A-like, polypeptide may comprise a sequence that has at least 50% sequence identity to a CD79A protein and/or the sequence set forth in SEQ ID NO: 16 or 175, e.g. a sequence that has at least 80% sequence identity to SEQ ID NO: 16. Furthermore, the fourth, i.e. CD79B-like, polypeptide may comprise a sequence that has at least 50% sequence identity to a CD79B protein, and/or the sequence set forth in SEQ ID NO: 18 or 178, e.g. a sequence that has at least 80% sequence identity to SEQ ID NO: 18.

Preferably, the third, i.e. CD79A-like, and fourth, i.e. CD79B-like, polypeptide are able to interact with and/or bind to each other, i.e., in the modified cell of the invention.

In particular, herein and in context of the present invention, it is not necessary that CD79A or the extracellular domain of CD79A, e.g. as shown in SEQ ID NO: 16 or 12 or as contained in SEQ ID NO: 110, or a corresponding sequence (e.g. having about 50% sequence identity thereto) comprises a leader sequence, e.g., as shown in SEQ ID NO: 173 (i.e. MPGGPGVLQALPATIFLLFLLSAVYLGPGCQA). Therefore, the leader sequence, i.e. SEQ ID NO: 173 or a sequence corresponding thereto, may be omitted in SEQ ID NO: 12, 16 and 110 or corresponding sequences, e.g., as shown in SEQ ID NO: 174, 175, and 184, respectively.

For example, it is further possible that a sequence having about 70%, 75% or about 80% sequence identity to SEQ ID NO: 12 does not or essentially not contain a leader sequence (e.g. a leader sequence as shown in SEQ ID NO: 173).

Accordingly, the extracellular domain and the membrane domain of the third, i.e. CD79A-like, polypeptide may comprise a sequence that has at least 50% sequence identity to the sequence set forth in SEQ ID NO: 202.

Similarly, herein and in context of the present invention, it is, in particular, not necessary that CD79B or the extracellular domain of CD79B, e.g. as shown in SEQ ID NO: 18 or 14 or as contained in SEQ ID NO: 112, or a corresponding sequence (e.g. having about 50% sequence identity thereto) comprises a leader sequence, e.g., as shown in SEQ ID NO: 176 (i.e. MARLALSPVPSHWMVALLLLLSAEPVPA). Therefore, the leader sequence, i.e. SEQ ID NO: 176 or a sequence corresponding thereto, may be omitted in SEQ ID NO: 14, 18 and 112 or corresponding sequences, e.g., as shown in SEQ ID NO: 177, 178, and 185, respectively.

For example, it is further possible that a sequence having about 80% sequence identity to SEQ ID NO: 14 does not or essentially not contain a leader sequence (e.g. a leader sequence as shown in SEQ ID NO: 176).

Accordingly, the extracellular domain and the membrane domain of the fourth, i.e. CD79B-like, polypeptide may comprise a sequence that has at least 50% sequence identity to the sequence set forth in SEQ ID NO: 203.

It has been previously determined that the amino acid sequence motif “YS” in the membrane domain of a membrane-bound antibody is sufficient for the interaction of the membrane-bound antibody with CD79 in the context of a B cell receptor; Gottwick (2019), PNAS, 116 (27).

Hence, herein and in the context of the present invention, the membrane domain of the first polypeptide may comprise the sequence motif “YS”. Furthermore, the membrane domain of the first polypeptide may comprise the larger sequence motif “WXXXXXFXXLFXLXXXYSXXXT” (SEQ ID NO: 19), or a sequence that has at least 80% sequence identity to SEQ ID NO: 19. Furthermore, the membrane domain of the first polypeptide may comprises a sequence that has at least 50% sequence identity to the membrane domain of a membrane-bound immunoglobulin and/or a sequence set forth in SEQ ID NO: 20, 21, 22, 23, 24, 25, 26, 27, 29, 31, 37 or 38, preferably SEQ ID NO: 20, 21, 22, 29 or 31.

Furthermore, the first polypeptide according to the invention may further comprise a constant region, as described herein. In particular, the constant region is located at the external side of the modified cell of the invention. The constant region of the first polypeptide, as described herein, may be a constant region of an antibody, a Fc-fragment of an antibody or derived from the constant region or Fc-fragment of an antibody, as commonly understood in the art, however, the constant region of the first polypeptide is not limited thereto.

Furthermore, the membrane domain and/or constant region of the first polypeptide according to the invention may be able to interact with and/or bind to the membrane domain and/or extracellular domain of at least one polypeptide selected from the group consisting of: a CD79A protein, a CD79B protein, the third, i.e. CD79A-like, polypeptide and the fourth, i.e. CD79B-like, polypeptide according to the invention. Furthermore, the membrane domain and/or extracellular domain of the third, i.e. CD79A-like, polypeptide of the invention and/or the membrane domain and/or extracellular domain of the fourth, i.e. CD79B-like polypeptide of the invention may be able to interact with and/or bind to the membrane domain and/or constant region of at least one polypeptide selected from the group consisting of: a membrane-bound immunoglobulin and the first polypeptide of the invention. Preferably, the membrane domain of the first polypeptide is able to interact with and/or bind to the membrane domain of at least one polypeptide selected from the group consisting of: a CD79A protein, a CD79B protein, the third, i.e. CD79A-like, polypeptide and the fourth, i.e. CD79B-like, polypeptide. Also preferably, the membrane domain of the third, i.e. CD79A-like, polypeptide and/or the membrane domain of the fourth, i.e. CD79B-like, polypeptide is able to interact with and/or bind to the membrane domain of at least one polypeptide selected from the group consisting of: a membrane-bound immunoglobulin and the first polypeptide of the invention.

Furthermore, the fifth, i.e. CD16-like, polypeptide may comprise a membrane domain. In particular, the membrane domain is located within the membrane of the modified cell of the invention. The membrane domain of the fifth polypeptide may comprise the sequence motif “FXXDT” or “FXXNT”, i.e. “FXX(D/N)T”. Furthermore, the membrane domain of the fifth polypeptide may comprise a sequence that has at least 80% sequence identity to the sequence set forth in SEQ ID NO: 34. Furthermore, the membrane domain of the fifth polypeptide may comprise a sequence that has at least 50% sequence identity to the membrane domain of a CD16 protein and/or the sequence set forth in SEQ ID NO: 36.

Furthermore, the fifth, i.e. CD16-like, polypeptide according to the invention may comprise an extracellular domain. In particular, the extracellular domain is located at the external side of the modified cell of the invention. Sequence motifs of CD16 that are able to interact with antibodies have been identified; see, e.g., Sondermann (2000), Nature 406. In particular, it is known that the extracellular domain of antibodies, i.e. the constant region or Fc region, and the extracellular domain of CD16 can interact.

Thus, the extracellular domain of the fifth, i.e. CD16-like, polypeptide may comprise the sequence motif set forth in SEQ ID NO: 39, or a sequence that has at least 80% sequence identity to SEQ ID NO: 39. Furthermore, the extracellular domain of the fifth polypeptide may comprise a sequence that has at least 50% sequence identity to the sequence set forth in SEQ ID NO: 41. Furthermore, the extracellular domain of the fifth polypeptide may comprise a sequence that has at least 50% sequence identity to the extracellular domain of a CD16 protein, and/or the sequence set forth in SEQ ID NO: 43 or 45, e.g. a sequence that has a sequence identity of at least 80%, preferably at least 90% to SEQ ID NO: 43.

Furthermore, herein and in the context of the invention, e.g. when CD16 and/or the fifth polypeptide of the invention is involved, the constant region of the first polypeptide may comprise the sequence motif set forth in SEQ ID NO: 50, or a sequence that has at least 80% sequence identity to SEQ ID NO: 50. Furthermore, the constant region of the first polypeptide may comprise a sequence that has at least 50% sequence identity to the sequence set forth in SEQ ID NO: 52. Furthermore, the constant region of the first polypeptide of the invention may comprise a sequence that has at least 50% sequence identity to a constant domain of an immunoglobulin, e.g. C_{H}1, C_{H}2, C_{H}3 or C_{H}4, and/or the sequence set forth in SEQ ID NO: 62, 64, 66 or 68. Furthermore, the constant region of the first polypeptide may comprise a sequence that has at least 50% sequence identity to the constant region of an immunoglobulin and/or the sequence set forth in SEQ ID NO: 54.

Furthermore, in the context of the present invention, the constant region and/or membrane domain of the first, i.e. antibody heavy chain-like, polypeptide may be able to interact with and/or bind to the extracellular domain and/or membrane domain of at least one polypeptide selected from the group consisting of: a Fc-receptor, a CD16 protein, and the fifth, i.e. CD16-like, polypeptide of the invention. Furthermore, the extracellular domain and/or membrane domain of the fifth polypeptide of the invention may be able to interact with and/or bind to the constant region and/or membrane domain of at least one polypeptide selected from the group consisting of: a membrane-bound immunoglobulin or at least one constant domain thereof, e.g. C_{H}1, C_{H}2, C_{H}3 or C_{H}4, and the first polypeptide of the invention.

Preferably, the constant region of the first polypeptide of the invention is able to interact with and/or bind to the extracellular domain of at least one polypeptide selected from the group consisting of: a Fc-receptor protein, a CD16 protein, and the fifth polypeptide of the invention. Also preferably, the extracellular domain of the fifth polypeptide of the invention is able to interact with and/or bind to the constant region of at least one polypeptide selected from the group consisting of: a membrane-bound immunoglobulin or at least one constant domain thereof, e.g. C_{H}1, C_{H}2, C_{H}3 or C_{H}4, and the first polypeptide according to the invention.

Furthermore, herein and in the context of the invention, e.g. when the fifth polypeptide and/or CD16 is involved, the third, i.e. CD3 zeta-like, polypeptide may comprise a sequence that has at least 50% sequence identity to a CD3 zeta protein and/or the sequence set forth in SEQ ID NO: 56. Furthermore, herein and in the context of the invention, e.g. when the fifth polypeptide and/or CD16 is involved, the fourth, i.e. FceRIg-like, polypeptide may comprise a sequence that has at least 50% sequence identity to a FceRIg protein and/or the sequence set forth in SEQ ID NO: 58.

In the context of the invention, the membrane domain and/or extracellular domain of the fifth, i.e. CD16-like, polypeptide may be able to interact with and/or bind to the membrane domain and/or extracellular domain of at least one polypeptide selected from the group consisting of: a CD3 zeta protein, a FceRIg protein, the third, i.e. CD3 zeta-like, polypeptide, and the fourth, i.e. FceRIg-like, polypeptide. Furthermore, the membrane domain and/or extracellular domain of the third, i.e. CD3 zeta-like, polypeptide and/or the membrane domain and/or extracellular domain of the fourth, i.e. FceRIg-like, polypeptide may be able to interact with and/or bind to the membrane domain and/or extracellular domain of at least one polypeptide selected from the group consisting of: a CD16 protein, and the fifth polypeptide according to the invention. Preferably, the membrane domain of the fifth polypeptide is able to interact with and/or bind to the membrane domain of at least one polypeptide selected from the group consisting of: a CD3 zeta protein, a FceRIg protein, the third, i.e. CD3 zeta-like, polypeptide, and the fourth, i.e. FceRIg-like, polypeptide. Also preferably, the membrane domain of the third, i.e. CD3 zeta-like, polypeptide and/or the membrane domain of the fourth, i.e. FceRIg-like, polypeptide is able to interact with and/or bind to the membrane domain of at least one polypeptide selected from the group consisting of: a CD16 protein, and the fifth polypeptide according to the invention.

In certain embodiments of the invention, the modified cell comprises the third, i.e. CD79A-like, polypeptide and the fourth, i.e. CD79B-like, polypeptide, as described herein, as well as the first and second polypeptide of the invention. In particular, the first, second, third and fourth polypeptide may form a protein complex, i.e., a BCR-like complex and/or antigen-receptor complex as described herein, i.e., in the modified cell of the invention.

Furthermore, i.e. in the context of these embodiments, it is possible that the modified cell does not comprise a CD16 protein. Furthermore, i.e. in the context of these embodiments, it is possible that the modified cell does not comprise a polypeptide that has at least 90% sequence identity to the sequence set forth in SEQ ID NO: 47.

In certain embodiments of the invention, the modified cell comprises the third, i.e. CD3 zeta-like, polypeptide and/or the fourth, i.e. FceRIg-like, polypeptide as described herein, as well as the first and second polypeptide of the invention.

Furthermore, it is possible in the context of the invention, that the modified cell does not comprise a CD79A protein, a CD79B protein or a polypeptide that has at least 90% sequence identity to the sequence set forth in SEQ ID NO: 16 or 18. It is also possible that the modified cell does not comprise a polypeptide that has at least 90% sequence identity to the sequence set forth in SEQ ID NO: 175 or 178.

Herein, and in the context of the present invention, the second polypeptide may further comprise a constant region comprising a sequence that has at least 50% sequence identity to the constant region of a light chain of an antibody and/or the sequence set forth in SEQ ID NO: 60. In particular, the constant region of the second polypeptide is located at the external side of the modified cell of the invention. Furthermore, the second polypeptide may be located in its entirety at the external side of the cell.

Furthermore, herein, and in the context of the invention, the first and the second polypeptide may be able to form a Y-shaped protein comprising two first polypeptide chains that are connected to each other, for example by a disulfide bond, and two second polypeptide chains, wherein each of the first polypeptide chains is connected to a second polypeptide chain, for example, by a disulfide bond.

Furthermore, the first polypeptide according to the invention may comprise a dimerization domain. In particular, the first polypeptide according to the invention may form a homodimer, i.e., via the dimerization domain. Preferably, the dimerization domain is comprised in the extracellular and/or membrane domains of the first polypeptide, preferably in the extracellular domain. In particular, the dimerization domain may be comprised in the constant region of the first polypeptide of the invention. Furthermore, the dimerization domain may be, or may be derived from, the constant region or Fc-part of an antibody. Dimerization domains are well known in the art, and any of them may be employed in the context of the invention.

Furthermore, herein and in the context of the present invention, the first polypeptide may further comprise a linker region between the variable region and the membrane domain. In particular, the linker region forms a flexible linker. In particular, the first polypeptide of the invention may comprise a linker region between the constant region and the membrane domain. Furthermore, the linker region according to the invention may comprise about 10 to about 100 amino acids, preferably about 50 amino acids. Preferably, the linker is a glycine-serine linker. For example, the linker region may comprise 2 to 20 repeats of the amino acid sequence GGGGS (SEQ ID NO: 69). Furthermore, the linker region may have at its N-terminus the sequence SGGGGS (SEQ ID NO: 70), for example, as set forth in SEQ ID NO: 72. Furthermore, the linker may have a length of about 5 to 50 nm, preferably about 20 nm.

In particular herein, and in the context of the invention, the first polypeptide, the third polypeptide and/or the fourth polypeptide may comprise an intracellular domain comprising at least one signaling domain, wherein the signaling domain(s) may be the same or different between the first, third and/or fourth polypeptides. In particular, the at least one signaling domain is located at the internal side of the modified cell of the invention.

In context of the present invention and as described herein, a signaling domain, e.g. in the first, third and/or fourth polypeptide, may comprise at least one signaling or activation motif or region, e.g. an ITAM, ITAM region or ITSM, of a protein selected from the group consisting of: CD3 zeta, FcεRly (FceRIg), CD16A, CD16B, NKp30, NKp46, KIR2DS1-2, KIR2DS3-6, KIR3DS1, NKG2C, NKG2D, 2B4 (CD244), CD2, CRACC, NTB-A (SLAMF6), DNAM-1 (CD226), CD7, CD59, BY55, KIR2DL4 (CD158d), CD44, TNFRSF9 (4-1BB), SLAMF1 (CD150), CD28, TMIGD2 (CD28H), SLAMF7 (CD319), TNFRSF18 (CD357), CD84, HCST (DAP10), TYROB (DAP12), FCRL3, TNFRSF13C (BAFF), and a polypeptide that that at least 50% sequence identity to any of said proteins.

For example, a signaling domain according to the invention, e.g. in the first, third and/or fourth polypeptide, may comprise at least one signaling or activation motif or region, e.g. an ITAM or ITAM region, of a protein selected from the group consisting of: CD3 zeta, FcεRly (FceRIg), CD16A, CD16B, NKp30, NKp46, KIR2DS1-2, KIR2DS3-6, KIR3DS1, NKG2C, NKG2D, 2B4, CD2, CRACC, NTB-A, DNAM-1, CD7, CD59, BY55, KIR2DL4, CD44, and a polypeptide that that at least 50% sequence identity to any of said proteins.

Furthermore, a signaling domain according to the invention, e.g. in the first, third and/or fourth polypeptide, may comprise an ITAM consensus motif, i.e., the sequence motif “Y-XX-I or L-X_{(6 to 12)}-Y-XX-I or L”, e.g., as set forth in SEQ ID NO: 73, 74, 48 or 49, or a sequence that has at least 80% sequence identity to SEQ ID NO: 73, 74, 48 or 49. Of note, X means “any amino acid” and X_{(6 to 12)} means 6 to 12 amino acids.

Furthermore, a signaling domain according to the invention, e.g. in the first, third and/or fourth polypeptide, may comprise at least one immunoreceptor tyrosine-based activation motif (ITAM) of a CD3 zeta protein, a sequence that has at least 50% sequence identity to the sequence set forth in SEQ ID NO: 76, a sequence that has at least 50% sequence identity to the sequence set forth in SEQ ID NO: 78, and/or a sequence that has at least 50% sequence identity to the sequence set forth in SEQ ID NO: 80.

Furthermore, a signaling domain according to the invention, e.g. in the first, third and/or fourth polypeptide, may comprise at least one ITAM region of a CD3 zeta protein, and/or a sequence that has at least 50% sequence identity to the sequence set forth in SEQ ID NO: 82.

Furthermore, herein and in the context of the invention, the intracellular domain of the first, third and/or fourth polypeptide may comprise the intracellular domain of a CD3 zeta protein, and/or a sequence that has at least 50% sequence identity to the sequence set forth in SEQ ID NO: 84.

Furthermore, a signaling domain according to the invention, e.g. in the first, third and/or fourth polypeptide, may comprise at least one immunoreceptor tyrosine-based activation motif (ITAM) of a FceRIg protein, and/or a sequence that has at least 50% sequence identity to the sequence set forth in SEQ ID NO: 86.

Furthermore, herein and in the context of the invention, the intracellular domain of the first, third and/or fourth polypeptide may comprise the intracellular domain of a FceRIg protein, and/or a sequence that has at least 50% sequence identity to the sequence set forth in SEQ ID NO: 88, or 183, preferably SEQ ID NO: 88.

Furthermore, herein and in the context of the invention, the intracellular domain of the first, third and/or fourth polypeptide, in particular the first polypeptide, may comprise the intracellular domain of a membrane-bound immunoglobulin, e.g. an IgG1, and/or a sequence that has at least 50% sequence identity to the sequence set forth in SEQ ID NO: 90.

Furthermore, a signaling domain according to the invention, e.g. in the first, third and/or fourth polypeptide, may comprise at least one ITAM of a CD79A protein, and/or a sequence that has at least 50% sequence identity to the sequence set forth in SEQ ID NO: 92.

Furthermore, herein and in the context of the invention, the intracellular domain of the first, third and/or fourth polypeptide, in particular the third polypeptide, may comprise the intracellular domain of a CD79A protein, and/or a sequence that has at least 50% sequence identity to the sequence set forth in SEQ ID NO: 96.

Furthermore, a signaling domain according to the invention, e.g. in the first, third and/or fourth polypeptide, may comprise at least one ITAM of a CD79B protein, and/or a sequence that has at least 50% sequence identity to the sequence set forth in SEQ ID NO: 94.

Furthermore, herein and in the context of the invention, the intracellular domain of the first, third and/or fourth polypeptide, in particular the fourth polypeptide, may comprise the intracellular domain of a CD79B protein, and/or a sequence that has at least 50% sequence identity to the sequence set forth in SEQ ID NO: 98.

In following further signaling domains and intracellular domains according to the invention that may be contained, in particular, in the first, third and/or fourth polypeptide of the invention are described:

Furthermore, herein and in the context of the invention, a signaling domain may comprise an immunoreceptor tyrosine-based switch motif (ITSM) consensus motif, i.e. TXYXX(V/I), e.g. “TXYXXV” or “TXYXXI”.

Furthermore, a signaling domain according to the invention may comprise an ITSM from SLAMF1, in particular the motif “TIYAQV” (SEQ ID NO: 187) or a sequence that has at least 50% sequence identity thereto. Furthermore, a signaling domain according to the invention may comprise an extended motif from SLAMF1, in particular the motif set forth in SEQ ID NO: 188, or a sequence that has at least 50% sequence identity thereto. Furthermore, an intracellular domain according to the invention may comprise an intracellular domain from SLAMF1, in particular a sequence that has at least 50% sequence identity to SEQ ID NO: 207.

Furthermore, a signaling domain according to the invention may comprise at least one ITSM from SLAMF6, in particular the motif “TVYASV” (SEQ ID NO: 200) or a sequence that has at least 50% sequence identity thereto, and/or the motif “ITIYSTI” (SEQ ID NO: 201) or a sequence that has at least 50% sequence identity thereto. Furthermore, an intracellular domain according to the invention may comprise an intracellular domain from SLAMF6, in particular a sequence that has at least 50% sequence identity to SEQ ID NO: 235.

Furthermore, a signaling domain according to the invention may comprise at least one ITSM from CD244, in particular the motif “TLYSLI” (SEQ ID NO: 190) or a sequence that has at least 50% sequence identity thereto, and/or the motif “TIYEVI” (SEQ ID NO: 191) or a sequence that has at least 50% sequence identity thereto. Furthermore, an intracellular domain according to the invention may comprise an intracellular domain from CD244, in particular a sequence that has at least 50% sequence identity to SEQ ID NO: 213.

Furthermore, an signaling domain according to the invention may comprise at least one ITSM from CD244, in particular the motif “TLYSLI” (SEQ ID NO: 190) or a sequence that has at least 50% sequence identity thereto, and/or the motif “TIYEVI” (SEQ ID NO: 191) or a sequence that has at least 50% sequence identity thereto.

Furthermore, an intracellular domain according to the invention may comprise an intracellular domain from CD244, in particular a sequence that has at least 50% sequence identity to SEQ ID NO: 213.

Furthermore, an intracellular domain according to the invention may comprise an intracellular domain from TNFRSF9, in particular a sequence that has at least 50% sequence identity to SEQ ID NO: 205.

Furthermore, an intracellular domain according to the invention may comprise an intracellular domain from KIR2DL4, in particular a sequence that has at least 50% sequence identity to SEQ ID NO: 209.

Furthermore, a signaling domain according to the invention may comprise a motif, especially an immunoreceptor tyrosine tail (ITT)-like motif, from CD226, in particular the motif “EDIYVN” (SEQ ID NO: 189) or a sequence that has at least 50% sequence identity thereto. Furthermore, an intracellular domain according to the invention may comprise an intracellular domain from CD226, in particular a sequence that has at least 50% sequence identity to SEQ ID NO: 211.

Furthermore, a signaling domain according to the invention may comprise at least one motif from CD28, in particular the motif “YMNM” (SEQ ID NO: 192) or a sequence that has at least 50% sequence identity thereto, and/or the motif “PYAP” (SEQ ID NO: 193) or a sequence that has at least 50% sequence identity thereto. Furthermore, an intracellular domain according to the invention may comprise an intracellular domain from CD28, in particular a sequence that has at least 50% sequence identity to SEQ ID NO: 215.

Furthermore, a signaling domain according to the invention may comprise at least one motif from TMIGD2, in particular the motif “YXN”, e.g., “YSN”, and/or the proline rich motif “PSPRPCPSPRPGHP” (SEQ ID NO: 194) or a sequence that has at least 50% sequence identity thereto. Furthermore, an intracellular domain according to the invention may comprise an intracellular domain from TMIGD2, in particular a sequence that has at least 50% sequence identity to SEQ ID NO: 217.

Furthermore, an intracellular domain according to the invention may comprise an intracellular domain from TNFRSF18, in particular a sequence that has at least 50% sequence identity to SEQ ID NO: 221.

Furthermore, an intracellular domain according to the invention may comprise an intracellular domain from CD44, in particular a sequence that has at least 50% sequence identity to SEQ ID NO: 223.

Furthermore, a signaling domain according to the invention may comprise a motif from CD7, in particular the motif “YEDM” (SEQ ID NO: 197) or a sequence that has at least 50% sequence identity thereto. Furthermore, an intracellular domain according to the invention may comprise an intracellular domain from CD7, in particular a sequence that has at least 50% sequence identity to SEQ ID NO: 225.

Furthermore, an intracellular domain according to the invention may comprise an intracellular domain from CD84, in particular a sequence that has at least 50% sequence identity to SEQ ID NO: 227.

Furthermore, a signaling domain according to the invention may comprise at least one motif from HCST (DAP10), in particular the motif “YXXM”, e.g., “YINM” (SEQ ID NO: 198). Furthermore, an intracellular domain according to the invention may comprise an intracellular domain from HCST, in particular a sequence that has at least 50% sequence identity to SEQ ID NO: 229.

Furthermore, a signaling domain according to the invention may comprise a motif, in particular an ITAM, from TYROB, in particular the motif “YQELQGQRSDVYSDL” (SEQ ID NO: 197) or a sequence that has at least 50% sequence identity thereto.

Furthermore, an intracellular domain according to the invention may comprise an intracellular domain from TYROB, in particular a sequence that has at least 50% sequence identity to SEQ ID NO: 231.

Furthermore, an intracellular domain according to the invention may comprise an intracellular domain from FCRL3, in particular a sequence that has at least 50% sequence identity to SEQ ID NO: 233.

Furthermore, an intracellular domain according to the invention may comprise an intracellular domain from TNFRSF13C, in particular a sequence that has at least 50% sequence identity to SEQ ID NO: 237.

As described herein, the present invention also relates to chimeric polypeptides. In particular, the first, third and/or fourth polypeptide of the present invention may be also chimeric polypeptides. For example, a chimeric polypeptide according to the invention (e.g. the third CD79A-like polypeptide) may have (i) an extracellular domain and/or membrane domain derived from a certain protein (e.g. CD79A or CD79B) and (ii) an intracellular domain that derived from another protein (e.g. CD3 zeta) and/or an intracellular domain containing a signaling domain comprising one or more motifs from another protein (e.g. one or more ITAMs from CD3 zeta). As described herein and as illustrated in the appended examples, chimeric polypeptides may be advantageous in context of the present invention. For example, chimeric polypeptides of the present invention may enhance the efficacy, in particular the killing activity, of the modified cell of the invention.

In some embodiments of the invention, e.g., in the context of chimeric polypeptides according to the invention, the first polypeptide comprises a constant region as described herein, and a membrane domain as defined described herein, for example, the first polypeptide may comprise a sequence as set forth in SEQ ID NO: 106, or positions 1 to 371 of SEQ ID NO: 100. Furthermore, in particular in the context of these embodiments, the first polypeptide may comprise between the constant region and the membrane domain a linker region as described herein. Furthermore, in particular in the context of these embodiments, the first polypeptide may further comprise an intracellular domain as described herein, for example, the first polypeptide may comprise a sequence as set forth in SEQ ID NO: 100 or 102. Furthermore, e.g., in the context of these embodiments, the first polypeptide may comprise an intracellular domain comprising an ITAM as described herein in the context of FceRIg and/or an intracellular domain as described herein in the context of FceRIg, for example, as set forth in SEQ ID NO: 114. Furthermore, e.g., in the context of these embodiments, the first polypeptide may comprise an intracellular domain comprising at least one ITAM, or an ITAM region as described herein in the context of CD3 zeta, and/or an intracellular domain as described herein in the context of CD3 zeta.

In certain embodiments of the invention, e.g., in the context of chimeric polypeptides according to the invention, the third polypeptide comprises an extracellular domain as described herein in the context of the third CD79A-like polypeptide, and a membrane domain as described herein in the context of the CD79A-like third polypeptide. Furthermore, e.g., in the context of these embodiments, the third CD79-like polypeptide may comprise an intracellular domain comprising an ITAM as described herein in the context of CD79A and/or an intracellular domain as described herein in the context of CD79A, for example, as set forth in SEQ ID NO: 16 or 175. Furthermore, e.g. in the context of these embodiments, the third CD79-like polypeptide may comprise an intracellular domain comprising at least one ITAM, or ITAM region as described herein in the context of CD3 zeta and/or an intracellular domain, as described herein in the context of CD3 zeta, for example, as set forth in SEQ ID NO: 110 or 184. Furthermore, e.g. in the context of these embodiments, the third CD79-like polypeptide may comprise an intracellular domain comprising an ITAM as described herein in the context of FceRIg and/or an intracellular domain as described herein in the context of FceRIg.

In certain embodiments of the invention, e.g., in the context of chimeric polypeptides according to the invention, the fourth CD79B-like polypeptide comprises an extracellular domain as described in the context for the fourth CD79B-like polypeptide, and a membrane domain as described herein in the context of the fourth CD79B-like polypeptide. Furthermore, e.g. in the context of these embodiments, the fourth CD79B-like polypeptide may further comprise an intracellular domain comprising an ITAM and/or an intracellular domain as described herein in the context of CD79B, for example, as set forth in SEQ ID NO: 18 or 178. Furthermore, e.g. in the context of these embodiments, the fourth CD79B-like polypeptide may comprise an intracellular domain comprising at least one ITAM or ITAM region as described herein in the context of CD3 zeta, and/or an intracellular domain as described herein in the context of CD3 zeta, for example, as set forth in SEQ ID NO: 112 or 185. Furthermore, e.g. in the context of these embodiments, the fourth polypeptide may comprise an intracellular domain comprising an ITAM as described herein in the context of FceRIg and/or an intracellular domain as described herein in the context of FceRIg.

Furthermore, a chimeric polypeptide of the invention (e.g. the third CD79A-like polypeptide of the invention and/or the fourth CD79B-like polypeptide of the invention) may comprise (i) an extracellular domain and a membrane domain from CD79A or CD79B, in particular a sequence that has at least 50%, preferably at least 80%, sequence identity to SEQ ID NO: 202 or SEQ ID NO: 203, and (ii) a signaling domain (e.g. comprising one or more motifs such as ITAMs) and/or a intracellular domain from another protein as described herein, e.g. from CD3 zeta, FceRIg, TNFRSF9, SLAMF1, KIR2DL4, CD226, CD244, CD28, TMIGD2, SLAMF7, TNFRSF18, CD44, CD7, CD84, HCST, TYROB, FCRL3, SLAMF6, TNFRSF13C.

In particular, the intracellular domain from CD3 zeta may have a sequence identity of at least 50% to SEQ ID NO: 84, the intracellular domain from FceRIg may have a sequence identity of at least 50% to SEQ ID NO: 88, the intracellular domain from TNFRSF9 may have a sequence identity of at least 50% to SEQ ID NO: 205, the intracellular domain from SLAMF1 may have a sequence identity of at least 50% to SEQ ID NO: 207, the intracellular domain from KIR2DL4 may have a sequence identity of at least 50% to SEQ ID NO: 209, the intracellular domain from CD226 may have a sequence identity of at least 50% to SEQ ID NO: 211, the intracellular domain from CD244 may have a sequence identity of at least 50% to SEQ ID NO: 213, the intracellular domain from CD28 may have a sequence identity of at least 50% to SEQ ID NO: 215, the intracellular domain from TMIGD2 may have a sequence identity of at least 50% to SEQ ID NO: 217, the intracellular domain from SLAMF7 may have a sequence identity of at least 50% to SEQ ID NO: 219, the intracellular domain from TNFRSF18 may have a sequence identity of at least 50% to SEQ ID NO: 221, the intracellular domain from CD44 may have a sequence identity of at least 50% to SEQ ID NO: 223, the intracellular domain from CD7 may have a sequence identity of at least 50% to SEQ ID NO: 225, the intracellular domain from CD84 may have a sequence identity of at least 50% to SEQ ID NO: 227, the intracellular domain from HCST may have a sequence identity of at least 50% to SEQ ID NO: 229, the intracellular domain from TYROB may have a sequence identity of at least 50% to SEQ ID NO: 231, the intracellular domain from FCRL3 may have a sequence identity of at least 50% to SEQ ID NO: 233, the intracellular domain from SLAMF6 may have a sequence identity of at least 50% to SEQ ID NO: 235, and the intracellular domain from TNFRSF13C may have a sequence identity of at least 50% to SEQ ID NO: 237.

Corresponding signaling domains, in particular within said intracellular domains, e.g., containing one or more motifs such as ITAMs or ITSMs are described herein as well and may be used for defining the chimeric polypeptides of the invention, including also corresponding embodiments of the first, third and/or fourth polypeptide of the present invention.

The extracellular domain from CD79A or CD79B may or may not contain an N-terminal leader sequence, in particular a sequence as shown in SEQ ID NO: 173 and 176, respectively, or a sequence that has at least 50% sequence identity thereto. Thus, the extracellular domain and a membrane domain from CD79A may also refer to a composed sequence defined by SEQ ID NO: 12 directly followed at the C-terminus by SEQ ID NO: 8, or a sequence that has at least 50% sequence identity to said composed sequence. Furthermore, the extracellular domain and a membrane domain from CD79B may also refer to a composed sequence defined by SEQ ID NO: 14 directly followed at the C-terminus by SEQ ID NO: 10, or a sequence that has at least 50% sequence identity to said composed sequence.

Moreover, two or more chimeric polypeptides of the invention may be combined, e.g. in a modified cell of the invention, e.g. the first, third and/or fourth polypetide described herein in context of chimeric polypeptides.

Accordingly, two or more signaling domains and/or intracellular domains may be combined, e.g., by employing multiple chimeric polypeptides. A preferred combination herein and in context of the present invention is (i) a signaling domain and/or intracellular domain from FceRIg as described herein, e.g. in context of the first polypeptide of the invention and (ii) a signaling domain and/or intracellular domain from CD3 zeta as described herein, e.g. in context of the third or fourth polypeptide of the invention.

Furthermore, two or more signaling domains and/or intracellular domains may be combined by employing a chimeric CD79A-like polypeptide as described herein and a chimeric CD79B-like polypeptide as described herein. Particular combinations are shown in SEQ ID NO: 238 to 431. These sequences are designated by a name that has a structure as the following illustrative example: “CD79A(EC-TM)-CD357(CYT)-CD79B(EC-TM)-CD44(CYT)”. The term “CD79A(EC-TM)” denotes the extracellular (EC) domain and membrane domain (TM; i.e. transmembrane domain) from CD79A, and the term “CD79B” (EC-TM)” denotes the extracellular (EC) domain and membrane domain (TM) from CD79B. The term “CYT” refers to the intracellular domain (i.e. the cytoplasmic domain). Accordingly, in this example, the term “CD357(CYT)” refers to the intracellular domain of CD357 (i.e. TNFRSF18) and the term “CD44(CYT)” refers to the intracellular domain of CD44. Therefore, in this exemplary sequence, the intracellular domains from TNFRSF18 and CD44 are combined.

In the sequences shown in SEQ ID NO: 238 to 431, the sequence of the first chimeric polypeptide (in the above example: CD79A(EC-TM)-CD357(CYT)) and the sequence of the second chimeric polypeptide (in the above example: CD79B(EC-TM)-CD44(CYT)) are separated by a 2A sequence, i.e. as shown in SEQ ID NO: 439 and 440 for DNA and the polypeptide, respectively. Since translation is skipped at the 2A sequence, two separate polypeptides (e.g. a third polypeptide of the invention and a fourth polypeptide of the invention) are produced. Therefore, in the sequences shown in SEQ ID NO: 238 to 431, the N-terminal polypeptide (or corresponding DNA sequence) extends, in particular, from position 1 to the last position prior to the 2A sequence shown in SEQ ID NO: 439 and 440. Moreover, in these sequences, the C-terminal polypeptide (or corresponding DNA sequence) extends, in particular, from the first position after the 2A sequence shown in SEQ ID NO: 439 and 440 to the last position of the entire sequence.

The combinations of chimeric CD79-like polypeptides shown in SEQ ID NO: 238 to 431 are further indicated in the following:

CD79A(EC-TM)-4-1BB(CYT)-CD79B(EC-TM)-CD3z(CYT)
CD79A(EC-TM)-CD28(CYT)-CD79B(EC-TM)-CD3z(CYT)
CD79A(EC-TM)-CD150(CYT)-CD79B(EC-TM)-CD3z(CYT)
CD79A(EC-TM)-CD226(CYT)-CD79B(EC-TM)-CD3z(CYT)
CD79A(EC-TM)-CD84(CYT)-CD79B(EC-TM)-CD3z(CYT)
CD79A(EC-TM)-CD244(CYT)-CD79B(EC-TM)-CD3z(CYT)
CD79A(EC-TM)-DR3(CYT)-CD79B(EC-TM)-CD3z(CYT)
CD79A(EC-TM)-CD7(CYT)-CD79B(EC-TM)-CD3z(CYT)
CD79A(EC-TM)-CD357(CYT)-CD79B(EC-TM)-CD3z(CYT)
CD79A(EC-TM)-FceR1g(CYT)-CD79B(EC-TM)-CD3z(CYT)
CD79A(EC-TM)-DAP12(CYT)-CD79B(EC-TM)-CD3z(CYT)
CD79A(EC-TM)-DAP10(CYT)-CD79B(EC-TM)-CD3z(CYT)
CD79A(EC-TM)-TNFRSF13C(CYT)-CD79B(EC-TM)-CD3z(CYT)
CD79A(EC-TM)-CD158d(CYT)-CD79B(EC-TM)-CD3z(CYT)
CD79A(EC-TM)-CD44(CYT)-CD79B(EC-TM)-CD3z(CYT)
CD79A(EC-TM)-NTB-A(CYT)-CD79B(EC-TM)-CD3z(CYT)
CD79A(EC-TM)-CD319(CYT)-CD79B(EC-TM)-CD3z(CYT)
CD79A(EC-TM)-CD28H(CYT)-CD79B(EC-TM)-CD3z(CYT)
CD79A(EC-TM)-BAFF(CYT)-CD79B(EC-TM)-CD44(CYT)
CD79A(EC-TM)-BAFF(CYT)-CD79B(EC-TM)-NTB-A(CYT)
CD79A(EC-TM)-BAFF(CYT)-CD79B(EC-TM)-CD28H(CYT)
CD79A(EC-TM)-CD44(CYT)-CD79B(EC-TM)-NTB-A(CYT)
CD79A(EC-TM)-CD44(CYT)-CD79B(EC-TM)-CD28H(CYT)
CD79A(EC-TM)-NTB-A(CYT)-CD79B(EC-TM)-CD28H(CYT)
CD79A(EC-TM)-CD226(CYT)-CD79B(EC-TM)-CD244(CYT)
CD79A(EC-TM)-CD226(CYT)-CD79B(EC-TM)-CD7(CYT)
CD79A(EC-TM)-CD226(CYT)-CD79B(EC-TM)-CD357(CYT)
CD79A(EC-TM)-CD226(CYT)-CD79B(EC-TM)-DAP12(CYT)
CD79A(EC-TM)-CD226(CYT)-CD79B(EC-TM)-DAP10(CYT)
CD79A(EC-TM)-CD226(CYT)-CD79B(EC-TM)-BAFF(CYT)
CD79A(EC-TM)-CD226(CYT)-CD79B(EC-TM)-CD44(CYT)
CD79A(EC-TM)-CD226(CYT)-CD79B(EC-TM)-NTB-A(CYT)
CD79A(EC-TM)-CD226(CYT)-CD79B(EC-TM)-CD28H(CYT)
CD79A(EC-TM)-CD244(CYT)-CD79B(EC-TM)-BAFF(CYT)
CD79A(EC-TM)-CD244(CYT)-CD79B(EC-TM)-Dap10(CYT)
CD79A(EC-TM)-CD7(CYT)-CD79B(EC-TM)-CD28H(CYT)
CD79A(EC-TM)-CD7(CYT)-CD79B(EC-TM)-Dap10(CYT)
CD79A(EC-TM)-BAFF(CYT)-CD79B(EC-TM)-CD28H(CYT)
CD79A(EC-TM)-CD226(CYT)-CD79B(EC-TM)-CD84(CYT)
CD79A(EC-TM)-CD244(CYT)-CD79B(EC-TM)-CD7(CYT)
CD79A(EC-TM)-CD244(CYT)-CD79B(EC-TM)-CD357(CYT)
CD79A(EC-TM)-CD244(CYT)-CD79B(EC-TM)-DAP12(CYT)
CD79A(EC-TM)-CD244(CYT)-CD79B(EC-TM)-CD44(CYT)
CD79A(EC-TM)-CD244(CYT)-CD79B(EC-TM)-NTB-A(CYT)
CD79A(EC-TM)-CD244(CYT)-CD79B(EC-TM)-CD28H(CYT)
CD79A(EC-TM)-CD7(CYT)-CD79B(EC-TM)-CD357(CYT)
CD79A(EC-TM)-CD7(CYT)-CD79B(EC-TM)-DAP12(CYT)
CD79A(EC-TM)-CD7(CYT)-CD79B(EC-TM)-BAFF(CYT)
CD79A(EC-TM)-CD7(CYT)-CD79B(EC-TM)-CD44(CYT)
CD79A(EC-TM)-CD7(CYT)-CD79B(EC-TM)-NTB-A(CYT)
CD79A(EC-TM)-CD7(CYT)-CD79B(EC-TM)-CD28H(CYT)
CD79A(EC-TM)-CD357(CYT)-CD79B(EC-TM)-DAP12(CYT)
CD79A(EC-TM)-CD357(CYT)-CD79B(EC-TM)-DAP10(CYT)
CD79A(EC-TM)-CD357(CYT)-CD79B(EC-TM)-BAFF(CYT)
CD79A(EC-TM)-CD357(CYT)-CD79B(EC-TM)-CD44(CYT)
CD79A(EC-TM)-DAP12(CYT)-CD79B(EC-TM)-DAP10(CYT)
CD79A(EC-TM)-DAP12(CYT)-CD79B(EC-TM)-BAFF(CYT)
CD79A(EC-TM)-DAP12(CYT)-CD79B(EC-TM)-CD44(CYT)
CD79A(EC-TM)-DAP12(CYT)-CD79B(EC-TM)-NTB-A(CYT)
CD79A(EC-TM)-DAP12(CYT)-CD79B(EC-TM)-CD28H(CYT)
CD79A(EC-TM)-DAP10(CYT)-CD79B(EC-TM)-CD44(CYT)
CD79A(EC-TM)-DAP10(CYT)-CD79B(EC-TM)-NTB-A(CYT)
CD79A(EC-TM)-DAP10(CYT)-CD79B(EC-TM)-CD28H(CYT)
CD79A(EC-TM)-41BB(CYT)-CD79B(EC-TM)-CD28(CYT)
CD79A(EC-TM)-41BB(CYT)-CD79B(EC-TM)-CD266(CYT)
CD79A(EC-TM)-41BB(CYT)-CD79B(EC-TM)-CD84(CYT)
CD79A(EC-TM)-41BB(CYT)-CD79B(EC-TM)-CD244(CYT)
CD79A(EC-TM)-41BB(CYT)-CD79B(EC-TM)-CD7(CYT)
CD79A(EC-TM)-41BB(CYT)-CD79B(EC-TM)-CD357(CYT)
CD79A(EC-TM)-41BB(CYT)-CD79B(EC-TM)-DAP12(CYT)
CD79A(EC-TM)-41BB(CYT)-CD79B(EC-TM)-DAP10(CYT)
CD79A(EC-TM)-41BB(CYT)-CD79B(EC-TM)-BAFF(CYT)
CD79A(EC-TM)-41BB(CYT)-CD79B(EC-TM)-CD44(CYT)
CD79A(EC-TM)-41BB(CYT)-CD79B(EC-TM)-NTB-A(CYT)
CD79A(EC-TM)-41BB(CYT)-CD79B(EC-TM)-CD28H(CYT)
CD79A(EC-TM)-CD28(CYT)-CD79B(EC-TM)-CD266(CYT)
CD79A(EC-TM)-CD28(CYT)-CD79B(EC-TM)-CD84(CYT)
CD79A(EC-TM)-CD28(CYT)-CD79B(EC-TM)-CD244(CYT)
CD79A(EC-TM)-CD28(CYT)-CD79B(EC-TM)-CD7(CYT)
CD79A(EC-TM)-CD28(CYT)-CD79B(EC-TM)-CD357(CYT)
CD79A(EC-TM)-CD28(CYT)-CD79B(EC-TM)-DAP12(CYT)
CD79A(EC-TM)-CD28(CYT)-CD79B(EC-TM)-DAP10(CYT)
CD79A(EC-TM)-CD28(CYT)-CD79B(EC-TM)-BAFF(CYT)
CD79A(EC-TM)-CD28(CYT)-CD79B(EC-TM)-CD44(CYT)
CD79A(EC-TM)-CD28(CYT)-CD79B(EC-TM)-NTB-A(CYT)
CD79A(EC-TM)-CD28(CYT)-CD79B(EC-TM)-CD28H(CYT)
CD79A(EC-TM)-CD84(CYT)-CD79B(EC-TM)-CD244(CYT)
CD79A(EC-TM)-CD84(CYT)-CD79B(EC-TM)-CD7(CYT)
CD79A(EC-TM)-CD84(CYT)-CD79B(EC-TM)-CD357(CYT)
CD79A(EC-TM)-CD84(CYT)-CD79B(EC-TM)-DAP12(CYT)
CD79A(EC-TM)-CD84(CYT)-CD79B(EC-TM)-DAP10(CYT)
CD79A(EC-TM)-CD84(CYT)-CD79B(EC-TM)-BAFF(CYT)
CD79A(EC-TM)-CD84(CYT)-CD79B(EC-TM)-CD44(CYT)
CD79A(EC-TM)-CD84(CYT)-CD79B(EC-TM)-NTB-A(CYT)
CD79A(EC-TM)-CD84(CYT)-CD79B(EC-TM)-CD28H(CYT)
CD79A(EC-TM)-CD357(CYT)-CD79B(EC-TM)-NTB-A(CYT)
CD79A(EC-TM)-CD357(CYT)-CD79B(EC-TM)-CD28H(CYT)

Moreover, e.g in context of chimeric polypeptides, the CD79A(EC-TM) and CD79B(EC-TM) may be exchanged with each other. Furthermore, the amino acid sequences of the individual domains, e.g. the extracellular and/or membrane domain of CD79A or CD79B, and the various signaling domains and/or intracellular domains may have a sequence identity of at least 50% to the corresponding amino acid sequences described herein in context of the domains indicated in the above list and in SEQ ID NO: 238 to 431.

Herein, and in the context of the present invention, the process in the cell, in particular the modified cell of the invention, that enables the cell to promote death of a target cell may comprise activation of at least one signaling pathway. In particular, upon binding of the antigen-binding site to a corresponding antigen, at least one of the signaling domains may activate at least one signaling pathway in the cell that enables the cell to promote death of a target cell comprising said antigen on the cell surface. For example, the signaling pathway(s) in the context of the invention may comprise or involve Ca2+ signaling, and/or at least one protein selected from the group consisting of: at least one Src family kinase, at least one Syk family kinase, PLCG1, PI3K, Vav1, at least one Rho family GTPase, ERK1/2, and NFAT. In particular, the modified cell of the invention is able to kill a target cell comprising the corresponding antigen on the cell surface, when the death-promoting process according to the invention is triggered and/or said at least one signaling pathway according to the invention is activated.

It has been further reported that the signaling domains or intracellular domains of the following proteins can have the following effects in cells, in particular NK cells (or T cells where indicated):
TNFRSF9: co-stimulation/proliferation; SLAMF1: co-activation/co-stimulation;
KIR2DL4: activation/inhibition; CD226: activation/cell adhesion; CD244: strong co-activation; CD28 (commonly used in the field): co-stimulation; TMIGD2: co-activation;
SLAMF7: co-activation; TNFRSF18: activation, proliferation, cytokine production (in T cells); CD44: activation, recirculation and homing; CD7: co-activation; CD84: co-activation/cell adhesion; HCST: one of the most common NK cell activating signals/strong activation; TYROB: one of the most common NK activating signals/strong activation; FCRL3: co-activation/co-inhibition; SLAMF6: co-activation;
TNFRSF13C: very potent activator in T cells.

Of note, it is possible that also certain inhibitory effects in a cell (e.g. reduction of overstimulation) may lead to a better overall activation of the cell such that the cell has an increased efficiency of promoting death of a target cell.

Hence, a combination of multiple signaling domains in multiple polypeptides in the multi-chain antigen receptor of the present invention (e.g. in the first, third and/or fourth polypeptide of the invention) may further increase the efficiency of promoting death of a target cell, as also illustrated in the appended Examples, e.g. Example 5. In particular, combinations of multiple signaling domains may promote or enhance the overall activation of the modified cell which may entail an enhanced efficiency of promoting death of a target cell. Furthermore, an increased activation of the cell may be, inter alia, characterized by an increased proliferation, increased secretion of cytokines (e.g. immunostimulatory cytokines), and/or an increased antigen-specific killing activity.

Moreover, the presence of certain signaling domains (or active motifs thereof) may have further effects on the cell upon antigen-binding, e.g., an altered or increased co-stimulation, altered cell adhesion, or altered recirculation and/or homing, as indicated above.

The modified cell of the invention may promote death of the target cell or kill the target cell by secreting a cytotoxic compound and/or contacting the target cell with a cytotoxic compound upon binding of the antigen-binding site to a corresponding antigen, in particular, upon binding of an antigen on the surface of the target cell. Furthermore, the modified cell may secrete a granzyme and/or a perforin upon binding of the antigen-binding site to a corresponding antigen, in particular, upon binding of an antigen on the surface of the target cell. Furthermore, the modified cell of the invention may secrete at least one cytokine upon binding of the antigen-binding site to a corresponding antigen.

Furthermore, herein and in the context of the invention, the modified cell may express and/or secrete IL-2 and/or IL-15.

Furthermore, herein and in the context of the invention, the modified cell may express a kill switch protein that kills the modified cell upon binding of a small molecule. Kill switch proteins and corresponding small molecules are well known in the art, e.g. a Caspase 9 kill switch.

Furthermore, the modified cell of the invention may be used for treating a disease as described herein, and/or in a method of treatment.

As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

Diseases that may be treated in the context of the invention include, inter alia, cancer, metabolic diseases, cardiovascular diseases, infectious diseases, respiratory diseases, hematologic disorders, immunological diseases, autoimmune diseases, neurological diseases, muscular diseases, or skeletal diseases.

Accordingly, the present invention further relates to the modified cell of the invention for use in treating a disease in a mammalian subject. Preferably, the subject herein and in the context of the invention is a human. However, the subject may be also any other mammal, e.g. a horse, dog, cat, cow, pig, sheep, goat, monkey, or polar bear etc.

Furthermore, the present invention relates to the modified cell of the invention for use in treating a disease that is caused and/or associated with a pathogenic target cell, wherein said modified cell promotes death of said pathogenic target cell, in particular, upon binding of the antigen-binding site to a corresponding antigen, e.g., upon binding of an antigen at the surface of the target cell. In particular, the pathogenic cell expresses, i.e. at the cell surface, an antigen that is recognized by the antigen-binding site of the modified cell of the invention. Herein, and in the context of the invention, the pathogenic target cell may be a tumor cell, or a pathogenic lymphocyte that is associated with and/or causes an autoimmune disease.

Accordingly, the present invention also relates to the modified cell of the invention for use in treating a cancer in a mammalian subject, preferably a human. The cancer is not particularly limited and may be any cancer, including liquid and solid tumors. For example, the cancer may be, inter alia, a breast cancer, a liver cancer, e.g. a hepatocellular carcinoma, a skin cancer, e.g. a melanoma, a prostate cancer, a blood cancer or leukemia, a brain cancer, e.g. a glioblastoma, a lung cancer, etc.

Furthermore, the present invention also relates to the modified cell of the invention for use in treating an autoimmune disease in a mammalian subject, preferably a human. For example, the autoimmune disease may be, inter alia, Rhumatoid arthritis, Lupus, Inflammatory bowel disease, Multiple sclerosis, Diabetes mellitus, Guillan barre syndrome, Psoriasis, Chronic inflammatory demyelinating polyneuropathy, Graves' disease, Hashimoto's thyroiditis, Myasthenia gravis, or Vasculitis.

Furthermore, the present invention relates to the modified cell of the invention for use in an immunotherapy in a mammalian subject, preferably a human.

Herein, and in the context of the invention, e.g., in context of the medical uses, the modified cell may be an allogenic or autologous cell. Preferably the modified cell of the invention is an allogenic cell.

Furthermore, the kit of the present invention may comprise:
(i) at least one nucleic acid molecule, each comprising the coding sequence of either the first, second, third, or fourth polypeptide, or optionally the fifth polypeptide;
(ii) at least one nucleic acid molecule, each comprising the coding sequence of two of the first, second, third, and fourth polypeptide, and optionally the fifth polypeptide, for example, wherein one nucleic acid molecule comprises the coding sequence of the first and second polypeptide, and another nucleic acid molecule comprises the coding sequence of the third and fourth polypeptide; and/or
(iii) at least one nucleic acid molecule comprising the coding sequence of at least three, four or all of the first, second, third, and fourth polypeptide, and optionally the fifth polypeptide.

In said options (ii) and/or (iii), the at least two coding sequences may be separated by at least one 2A or IRES sequence, and not separated by stop codons.

Furthermore, the kit of the invention may comprise at least one plasmid or viral vector, each comprising a nucleic acid molecule according to said options (i), (ii) or (iii). Furthermore, a plasmid or viral vector comprising a nucleic acid molecule according to said options (ii) or (iii) may comprise a promoter that is capable of producing an mRNA comprising the at least two coding sequences, in particular in a mammalian cell. In particular, said mRNA can be translated into the at least two polypeptides, in particular in a mammalian cell such as the modified cell of the invention.

Furthermore, a plasmid or viral vector comprising a nucleic acid molecule according to said options (ii) or (iii) may comprise a plurality of promoters, each being capable of producing an mRNA comprising one of the at least two coding sequences, in particular in a mammalian cell such as the modified cell of the invention.

Herein, and in the present invention, e.g. in the context of the kit of the invention, the nucleic acid molecule(s) may be DNA molecules or RNA molecules.

Furthermore, the viral vector of the invention may be a lentiviral vector, preferably a baboon pseudotyped lentivirus.

Furthermore, the present invention relates to a method of producing a modified cell of the invention, wherein the method comprises a step of introducing the nucleic acid molecule(s) as described herein, e.g. in the context of the kit of the invention, or the plasmid or viral vector described herein, into a mammalian cell.

As regards the mammalian cell, the same applies as is described herein, e.g., in the context of the modified cell of the invention. Accordingly, the mammalian cell may be, inter alia, an NK cell or a T cell.

Furthermore, the production method of the invention may further comprise a step of activating the mammalian cell, for example, by contacting the mammalian cell with a cytokine such as IL-2.

In certain embodiments of the invention, e.g. in the context of the modified cell of the invention, the first and second polypeptide of the invention are covalently linked, preferably by a peptide bond. Preferably, in the context of these embodiments, the modified cell of the invention further comprises the third CD79A-like polypeptide of the invention, the fourth CD79B-like polypeptide of the invention and/or the fifth CD16-like polypeptide of the invention.

Therefore, the present invention further relates to a modified mammalian comprising
(I) the first and second polypeptide of the invention, and
(II) (i) the third CD79A-like and/or the fourth CD79B-like polypeptide of the invention, and/or
   (ii) the fifth CD16-like polypeptide of the invention, optionally, wherein the first and second polypeptide of the invention are covalently linked, e.g. by a peptide bond.

Furthermore, said modified mammalian cell may have the properties of modified cells of the invention as described herein in general, e.g. with respect to the promotion of death of a target cell.

Accordingly, in certain embodiments of the invention, the first and second coding sequences form a contiguous nucleic acid sequence encoding a polypeptide comprising the amino acid sequences of the first and second polypeptide of the present invention.

Therefore, the present invention further relates to a kit comprising at least one nucleic acid molecule comprising
(I) a first coding sequence encoding the first polypeptide according to the invention, and a second coding sequence encoding the second polypeptide according to the invention, and
(II) (i) a third and/or fourth coding sequence encoding the third CD79A-like and/or the fourth CD79B-like polypeptide according to the invention, and/or
   (ii) a fifth coding sequence encoding the fifth CD16-like polypeptide of the invention,
optionally, wherein the first and second coding sequences form a contiguous nucleic acid sequence encoding a polypeptide comprising the amino acid sequences of the first and second polypeptide of the present invention.

Furthermore, the present invention relates to chimeric polypeptides, e.g., as described herein above and in the following. Moreover, the invention relates to a nucleic acid molecule, e.g. a DNA or RNA, as described herein encoding a chimeric polypeptide of the invention, as well as a plasmid or viral vector comprising said nucleic acid molecule as described herein.

Accordingly, the present invention relates to a polypeptide comprising
(a)
   (I) an extracellular domain comprising a sequence that has at least 50% sequence identity to the extracellular domain of a CD79A protein, and/or to the sequence set forth in SEQ ID NO: 12 or 174, e.g, a sequence that has at least about 70% sequence identity to SEQ ID NO: 12; and/or a membrane domain comprising
   (i) a sequence that has at least 50% sequence identity to the sequence set forth in SEQ ID NO: 4, and/or
   (ii) a sequence that has at least 50% sequence identity to the membrane domain of a CD79A protein and/or the sequence set forth in SEQ ID NO: 8;
and
(II) an intracellular domain comprising
   (i) at least one immunoreceptor tyrosine-based activation motif (ITAM) of a CD3 zeta protein, a sequence that has at least 50% sequence identity to the sequence set forth in SEQ ID NO: 76, a sequence that has at least 50% sequence identity to the sequence set forth in SEQ ID NO: 78, and/or a sequence that has at least 50% sequence identity to the sequence set forth in SEQ ID NO: 80,
   (ii) at least one ITAM region of a CD3 zeta protein, and/or a sequence that has at least 50% sequence identity to the sequence set forth in SEQ ID NO: 82, and/or
   (iii) the intracellular domain of a CD3 zeta protein, and/or a sequence that has at least 50% sequence identity to the sequence set forth in SEQ ID NO: 84;
and/or
(b) a sequence that has at least 50% sequence identity to the sequence set forth in SEQ ID NO: 110 or 184.

Furthermore, said polypeptide, in particular the membrane domain thereof, may be able to interact with and/or bind to the membrane domain of a membrane-bound immunoglobulin in a mammalian cell. Furthermore, the invention relates to a nucleic acid molecule comprising a coding sequence encoding said polypeptide. Said nucleic acid molecule may DNA or RNA. Furthermore, the invention relates to a viral vector comprising said nucleic acid molecule

Furthermore, the invention relates to a polypeptide comprising
(a)
   (I) an extracellular domain comprising a sequence that has at least 50% sequence identity to the extracellular domain of a CD79B protein, and/or the sequence set forth in SEQ ID NO: 14 or 177, e.g, a sequence that has at least about 70% sequence identity to SEQ ID NO: 14; and/or a membrane domain comprising
      (i) a sequence that has at least 50% sequence identity to the sequence set forth in SEQ ID NO: 6, and/or
      (ii) a sequence that has at least 50% sequence identity to the membrane domain of a CD79B protein and/or the sequence set forth in SEQ ID NO: 10;
   and
   (II) an intracellular domain comprising
      (i) at least one immunoreceptor tyrosine-based activation motif (ITAM) of a CD3 zeta protein, a sequence that has at least 50% sequence identity to the sequence set forth in SEQ ID NO: 76, a sequence that has at least 50% sequence identity to the sequence set forth in SEQ ID NO: 78, and/or a sequence that has at least 50% sequence identity to the sequence set forth in SEQ ID NO: 80,
      (ii) at least one ITAM region of a CD3 zeta protein, and/or a sequence that has at least 50% sequence identity to the sequence set forth in SEQ ID NO: 82, and/or
      (iii) the intracellular domain of a CD3 zeta protein, and/or a sequence that has at least 50% sequence identity to the sequence set forth in SEQ ID NO: 84;
and/or
(b) a sequence that has at least 50% sequence identity to the sequence set forth in SEQ ID NO: 112 or 185.

Furthermore, said polypeptide, i.e. the membrane domain thereof, may be able to interact with and/or bind to the membrane domain of a membrane-bound immunoglobulin in a mammalian cell. Furthermore, the invention relates to a nucleic acid molecule comprising a coding sequence encoding said polypeptide. Said nucleic acid molecule may be DNA or RNA. Furthermore, the invention relates to a viral vector comprising said nucleic acid molecule.

Furthermore, the invention relates to a polypeptide comprising
(a)
   (I) a constant region comprising
      (i) a sequence that has at least 50% sequence identity to the sequence set forth in SEQ ID NO: 52,
      (ii) a sequence that has at least 50% sequence identity to a constant domain of an immunoglobulin, e.g. C_{H}1, C_{H}2, C_{H}3 or C_{H}4, and/or the sequence set forth in SEQ ID NO: 62, 64, 66 or 68, and/or
      (iii) the constant region of an immunoglobulin and/or the sequence set forth in SEQ ID NO: 54; and/or
      a membrane domain comprising a sequence that has at least 50% sequence identity to the membrane domain of a membrane-bound immunoglobulin and/or a sequence set forth in SEQ ID NO: 20, 21, 22, 23, 24, 25, 26, 27, 29, or 31, preferably SEQ ID NO: 20, 21, 22, 29 or 31; and
   (II) an intracellular domain comprising
      (i) at least one immunoreceptor tyrosine-based activation motif (ITAM) of a FceRIg protein, and/or a sequence that has at least 50% sequence identity to the sequence set forth in SEQ ID NO: 86, and/or
      (ii) the intracellular domain of a FceRIg protein, and/or a sequence that has at least 50% sequence identity to the sequence set forth in SEQ ID NO: 88;
   and/or
   (b) a sequence that has at least 50% sequence identity to the sequence set forth in SEQ ID NO: 114.

Furthermore, said polypeptide, i.e. the membrane domain thereof, may be able to interact with and/or bind to the membrane domain of CD79A and/or CD79B in a mammalian cell. Furthermore, said polypeptide, i.e. the constant region thereof, may be able to interact with and/or bind to the extracellular domain of a Fc-receptor, and/or a CD16 protein in a mammalian cell. Furthermore, the invention relates to a nucleic acid molecule comprising a coding sequence encoding said polypeptide. Said nucleic acid molecule may be DNA or RNA. Furthermore, the invention relates to a viral vector comprising said nucleic acid molecule.

Furthermore, the invention relates to a mammalian cell comprising at least one of the inventive polypeptides provided herein, e.g. at least one of the chimeric polypeptides of the invention, and/or at least one nucleic acid molecule encoding at least one of said polypeptides, e.g. at least one of the chimeric polypeptides of the invention.

### REFERENCES

The following detailed references relate to the short references indicated herein above and below and in the appended Examples.
Abel, A. M., et al. 2018 Natural Killer Cells: Development, Maturation, and Clinical Utilization. Front Immunol 9:1869.
Al Qaraghuli, Mohammed M., et al. 2020 Antibody-protein binding and conformational changes: identifying allosteric signalling pathways to engineer a better effector response. Scientific Reports 10(1).
Baeuerle, Patrick A., et al. 2019 Synthetic TRuC receptors engaging the complete T cell receptor for potent anti-tumor response. Nature Communications 10(1).
Bendle, Gavin M., et al. 2010 Lethal graft-versus-host disease in mouse models of T cell receptor gene therapy. Nature Medicine 16(5):565-570.
Blszquez-Moreno, Alfonso, et al. 2017 Transmembrane features governing Fc receptor CD16A assembly with CD16A signaling adaptor molecules. Proceedings of the National Academy of Sciences 114(28):E5645-E5654.
Bloemberg, Darin, et al. 2020 A High-Throughput Method for Characterizing Novel Chimeric Antigen Receptors in Jurkat Cells. Molecular Therapy—Methods & Clinical Development 16:238-254.
Brown, Christine E., et al. 2005 Biophotonic cytotoxicity assay for high-throughput screening of cytolytic killing. Journal of Immunological Methods 297(1):39-52.
Bryceson, Yenan T., et al. 2006 Synergy among receptors on resting NK cells for the activation of natural cytotoxicity and cytokine secretion. Blood 107(1):159-166.
Caballero, Adriana, et al. 2006 Functional and structural requirements for the internalization of distinct BCR-ligand complexes. European Journal of Immunology 36(12):3131-3145.
Cambier, J. C., and K. S. Campbell 1989 Membrane Immunoglobulins Are Associated with a Family of Membrane Phosphoproteins. Pp. 651-658: Springer Berlin Heidelberg.
Capuano, Cristina, et al. 2021 Harnessing CD16-Mediated NK Cell Functions to Enhance Therapeutic Efficacy of Tumor-Targeting mAbs. Cancers 13(10):2500.
Chang, Yu-Hsiang, et al. 2013 A Chimeric Receptor with NKG2D Specificity Enhances Natural Killer Cell Activation and Killing of Tumor Cells. Cancer Research 73(6):1777-1786.
Chavez, Kathryn J., Sireesha V. Garimella, and Stanley Lipkowitz 2011 Triple negative breast cancer cell lines: One tool in the search for better treatment of triple negative breast cancer. Breast Disease 32(1-2):35-48.
Chen, Xiaoying, Jennica L. Zaro, and Wei-Chiang Shen 2013 Fusion protein linkers: Property, design and functionality. Advanced Drug Delivery Reviews 65(10):1357-1369.
Chu, P. G., and D. A. Arber 2001 CD79: a review. Appl Immunohistochem Mol Morphol 9(2):97-106.
Clémenceau, Béatrice, et al. 2015 In Vitro and In Vivo Comparison of Lymphocytes Transduced with a Human CD16 or with a Chimeric Antigen Receptor Reveals Potential Off-Target Interactions due to the IgG2 C_{H}2-C_{H}3 CAR-Spacer. Journal of Immunology Research 2015:e482089.
Clémenceau, Béatrice, et al. 2013 The human natural killer cytotoxic cell line NK-92, once armed with a murine CD16 receptor, represents a convenient cellular tool for the screening of mouse mAbs according to their ADCC potential. mAbs 5(4):587-594.
Colamartino, Aurelien B. L., et al. 2019 Efficient and Robust NK-Cell Transduction With Baboon Envelope Pseudotyped Lentivector. Frontiers in Immunology 10.
Cooper, Megan A., Todd A. Fehniger, and Michael A. Caligiuri 2001 The biology of human natural killer-cell subsets. Trends in Immunology 22(11):633-640.
Correia, Ana Luisa, et al. 2021 Hepatic stellate cells suppress NK cell-sustained breast cancer dormancy. Nature 594(7864):566-571.
Dal Porto, Joseph M., et al. 2004 B cell antigen receptor signaling 101. Molecular Immunology 41(6):599-613.
Dodev, Tihomir S., et al. 2014 A tool kit for rapid cloning and expression of recombinant antibodies. Scientific Reports 4(1):5885.
Doshi, Jiten, et al. 2020 Multiple Alternative Promoters and Alternative Splicing Enable Universal Transcription-Based Logic Computation in Mammalian Cells. Cell Reports 33(9):108437.
Dull, Tom, et al. 1998 A Third-Generation Lentivirus Vector with a Conditional Packaging System. Journal of Virology 72(11):8463-8471.
Dylke, Janis, et al. 2007 Role of the extracellular and transmembrane domain of Ig-α/β in assembly of the B cell antigen receptor (BCR). Immunology Letters 112(1):47-57.
Eshhar, Z., et al. 1993 Specific activation and targeting of cytotoxic lymphocytes through chimeric single chains consisting of antibody-binding domains and the gamma or zeta subunits of the immunoglobulin and T-cell receptors. Proceedings of the National Academy of Sciences 90(2):720-724.
Feucht, Judith, et al. 2019 Calibration of CAR activation potential directs alternative T cell fates and therapeutic potency. Nature Medicine 25(1):82-88.
Gauthier, Mélanie, et al. 2021 Natural Killer cells and monoclonal antibodies: Two partners for successful antibody dependent cytotoxicity against tumor cells. Critical Reviews in Oncology/Hematology 160:103261.
Gómez Román, Victor Rául, Joseph C. Murray, and Louis M. Weiner 2014 Antibody-Dependent Cellular Cytotoxicity (ADCC). Pp. 1-27: Elsevier.
Gong, J. H., G. Maki, and H. G. Klingemann 1994 Characterization of a human cell line (NK-92) with phenotypical and functional characteristics of activated natural killer cells. Leukemia 8(4):652-658.
Gross, G., T. Waks, and Z. Eshhar 1989 Expression of immunoglobulin-T-cell receptor chimeric molecules as functional receptors with antibody-type specificity. Proceedings of the National Academy of Sciences 86(24):10024-10028.
Hombach, Andreas A., et al. 2007 T Cell Activation by Antibody-Like Immunoreceptors: The Position of the Binding Epitope within the Target Molecule Determines the Efficiency of Activation of Redirected T Cells. The Journal of Immunology 178(7):4650-4657.
Huang, Ruihao, et al. 2020 Recent advances in CAR-T cell engineering. Journal of Hematology & Oncology 13(1):86.
Iannello, A., and D. H. Raulet 2013 Immune Surveillance of Unhealthy Cells by Natural Killer Cells. Cold Spring Harbor Symposia on Quantitative Biology 78(0):249-257.
Jayaraman, Jayapriya, et al. 2020 CAR-T design: Elements and their synergistic function. EBioMedicine 58.
Kim, Dae In, et al. 2016 An improved smaller biotin ligase for BiolD proximity labeling. Molecular Biology of the Cell 27(8):1188-1196.
Kuwana, Yoshihisa, et al. 1987 Expression of chimeric receptor composed of immunoglobulin-derived V resions and T-cell receptor-derived C regions. Biochemical and Biophysical Research Communications 149(3):960-968.
Lee, Soo-Chin, et al. 2020 Phase I Trial of Expanded, Activated Autologous NK-cell Infusions with Trastuzumab in Patients with HER2-positive Cancers. Clinical Cancer Research 26(17):4494-4502.
Li, Liang-Ping, et al. 2010 Transgenic mice with a diverse human T cell antigen receptor repertoire. Nature Medicine 16(9):1029-1034.
Liang, S., et al. 2018 Cetuximab combined with natural killer cells therapy: an alternative to chemoradiotherapy for patients with advanced non-small cell lung cancer (NSCLC). Am J Cancer Res 8(5):879-891.
Liu, Ziqing, et al. 2017 Systematic comparison of 2A peptides for cloning multi-genes in a polycistronic vector. Scientific Reports 7(1):2193.
Lois, C. 2002 Germline Transmission and Tissue-Specific Expression of Transgenes Delivered by Lentiviral Vectors. Science 295(5556):868-872.
Muntasell, A., et al. 2017 Interplay between Natural Killer Cells and Anti-HER2 Antibodies: Perspectives for Breast Cancer Immunotherapy. Front Immunol 8:1544.
Nimmerjahn, Falk, and Jeffrey V. Ravetch 2006 Fcγ Receptors: Old Friends and New Family Members. Immunity 24(1):19-28.
Park, Song Y., et al. 2021 How we treat Merkel cell carcinoma: within and beyond current guidelines. Future Oncology 17(11):1363-1377.
Patel, Kashyap R., Jacob T. Roberts, and Adam W. Barb 2019 Multiple Variables at the Leukocyte Cell Surface Impact Fc γ Receptor-Dependent Mechanisms. Frontiers in Immunology 10(223).
Pech, Matthew F, et al. 2019 Systematic identification of cancer cell vulnerabilities to natural killer cell-mediated immune surveillance. eLife 8:e47362.
Pesch, T., et al. 2019 Molecular Design, Optimization, and Genomic Integration of Chimeric B Cell Receptors in Murine B Cells. Front Immunol 10:2630.
Pfefferle, Aline, and Nicholas D. Huntington 2020 You Have Got a Fast CAR: Chimeric Antigen Receptor NK Cells in Cancer Therapy. Cancers 12(3).
Prochazka, Laura, et al. 2014 Highly modular bow-tie gene circuits with programmable dynamic behaviour. Nature Communications 5(1):1-12.
Ryan, M. D., A. M. Q. King, and G. P. Thomas 1991 Cleavage of foot-and-mouth disease virus polyprotein is mediated by residues located within a 19 amino acid sequence. Journal of General Virology 72(11):2727-2732.
Schroeder, Harry W., and Lisa Cavacini 2010 Structure and function of immunoglobulins. Journal of Allergy and Clinical Immunology 125(2, Supplement 2):S41-S52.
Stelzer, Christoph, and Yaakov Benenson 2020 Precise determination of input-output mapping for multimodal gene circuits using data from transient transfection. PLOS Computational Biology 16(11):e1008389.
Sun, Yue, et al. 2021 Antibody-receptor interactions mediate antibody-dependent cellular cytotoxicity. Journal of Biological Chemistry 297(1):100826.
Szöör, Árpád, et al. 2020 Trastuzumab derived HER2-specific CARs for the treatment of trastuzumab-resistant breast cancer: CAR T cells penetrate and eradicate tumors that are not accessible to antibodies. Cancer Letters 484:1-8.
Thurber, Greg M., Michael M. Schmidt, and K. Dane Wittrup 2008 Antibody tumor penetration: Transport opposed by systemic and antigen-mediated clearance. Advanced Drug Delivery Reviews 60(12):1421-1434.
Tiscornia, Gustavo, Oded Singer, and Inder M. Verma 2006 Production and purification of lentiviral vectors. Nature Protocols 1(1):241-245.
Trempe, G. L. 1976 Human Breast Cancer in Culture. Pp. 33-41: Springer Berlin Heidelberg.
Tsourkas, P. K., et al. 2007 Mechanisms of B-cell synapse formation predicted by Monte Carlo simulation. Biophys J 92(12):4196-208.
Venkitaraman, Ashok R., et al. 1991 The B-cell antigen receptor of the five immunoglobulin classes. Nature 352(6338):777-781.
Vivier, Eric, et al. 2008 Functions of natural killer cells. Nature Immunology 9(5):503-510.
Wang, Enxiu, et al. 2015a Generation of Potent T-cell Immunotherapy for Cancer Using DAP12-Based, Multichain, Chimeric Immunoreceptors. Cancer Immunology Research 3(7):815-826.
Wang, W., et al. 2015b NK Cell-Mediated Antibody-Dependent Cellular Cytotoxicity in Cancer Immunotherapy. Front Immunol 6:368.
Weinkove, Robert, et al. 2019 Selecting costimulatory domains for chimeric antigen receptors: functional and clinical considerations. Clinical & Translational Immunology 8(5).
Xie, Guozhu, et al. 2020 CAR-NK cells: A promising cellular immunotherapy for cancer. EBioMedicine 59:102975.
Yáñez-Muñoz, Rafael J., and Stephan A. Grupp 2018 CAR-T in the clinic: drive with care. Gene Therapy 25(3):157-161.
Zajc, Charlotte U., et al. 2021 Driving CARs with alternative navigation tools—the potential of engineered binding scaffolds. The FEBS Journal 288(7):2103-2118.

The invention is also characterized by the following figures, figure legends and the following non-limiting examples.

### BRIEF DESCRIPTION OF THE DRAWINGS

FIG. 1: Canonical ADCC versus cis-ADCC.

a, Schematics of canonical ADCC elicited by a CD16^{POS} NK cell supplemented with a soluble IgG1 antibody against a target antigen-positive cancer cell. b, Schematics of cis-ADCC against a target antigen-positive cancer cell according to the invention. The legend shows the visual elements.

FIG. 2: Implementation of cis-ADCC against Her2-positive target cells.

a, Schematics of the constructs used to manufacture the lentiviral vectors for NK-92 cells transduction. Displayed are the constructs for EF1A driven constitutive expression of CD16, membrane bound anti-Her2 immunoglobulin γ 1 (mIgG1), and CD79 expression, carrying fluorescent markers SBFP2, mScarlet, and mCerulean, respectively. Color codes (better visible in the priority application EP21217757.0) of various building blocks are used throughput the rest of the visuals in the Examples for consistency. b, Schematics and description of the proteins and protein domains used for the implementation of cis-ADCC. The term “CD79A/B” used here and throughout the Figures means “CD79A and CD79B”. The term “CD3z” has the same meaning as “CD3ζ-CD247”, the term “FCERIG” has the same meaning as “FcεRly”, the term “CD16” has the same meaning as “CD16:FCGR3A”, the term “SK-BR-3” has the same meaning as “SK-BR-3-Luc-Cit” and the term “MDA-MB-468” has the same meaning as “MDA-MB-468-Luc-Cit”, here and throughout the Figures. c-l, Specific lysis quantified using an LCA co-incubation assay after 4 h of NK-92 cell line variants. The cell line names (See Table 1) and the schematics of the cell surface modifications are displayed above the respective dose-response charts. The charts show the degree of specific cell lysis (y axis) as a function of increasing effector to target cell ratios (E:T cell ratios) (x axis). Red color indicates the effect on Her2-positive SK-BR-3 cells and blue color, the effect on Her2-negative MDA-MB-468 cells. Displayed are means of biological triplicates +−SD (extrapolated as a shaded area between discrete E:T ratios). *** represents p-values <0.001 of a statistical significance between the cytotoxic effects towards Her2-positive and Her2-negative target cells for a given E:T ratio.

FIG. 3: Characterization of NK-92 derivatives encoding an Her2-tIgG1.

a, Schematics of an anti-Her2 immunoglobulin tethered from the cell membrane with a (GGGGS)₁₁ₓ linker, CD79 heterodimers and a CD16 CD3ζ complex. All domains within the respective proteins are labeled with the precise protein fragments used. b, Schematics of lentiviral vectors. Displayed are constructs for EF1A driven constitutive expression of CD16, membrane tethered anti-Her2 immunoglobulin γ 1 (tIgG1), and CD79 expression, carrying fluorescent markers SBFP2 (of note: mTagBFP2 could be used as an equivalent), mScarlet, and mCerulean, respectively. Color codes (better visible in the priority application EP21217757.0) in (a), (b), correspond to cartoons in panels (c), (e), and (g). c, e, g, Fluorescent microscopy images taken after a 4 h co-incubation (i.e. a 4 h killing assay) of NK-92 variants (depicted schematically in the cartoons above) and Her2-positive SK-BR-3 cells at an E:T cell ratio of 10:1. mScarlet (red pseudocolor), SBFP2 (blue pseudocolor), mCerulean (turquois pseudocolor) indicate the expression of the antibody, the CD16 and the CD79 proteins, respectively. mCitrine is an indicator for SK-BR-3 target cells (LUTs for mScarlet were adjusted between (c), (e) 300-8000, and (g) 300-3000 due to lower NK-92 clustering in (g)). d, f, Specific lysis quantified using the LCA co-incubation assay after 4 h of NK-92 variants depicted respectively in panels (c) and (e) at different E:T cell ratios (x-axis) against Her2-positive SK-BR-3 red color and Her2-negative MDA-MB-468 cells blue color. Displayed are means of biological triplicates +−SD (extrapolated as a shaded area between discrete E:T ratios). h,i, The summary of specific data obtained with all NK-92 variants triggering ADCC in the presence of overexpressed CD16 (different cell lines shown by different colors according to the legend), at different E:T cell ratios (x-axis) against Her2-positive SK-BR-3 (i) and Her2-negative MDA-MB-468 cells (h). *** represents p-values <0.001 of a statistical significance between the cytotoxic effects towards Her2-positive and Her2-negative cell lines. Of note, “Trastuzumab” is the same as “Herceptin”.

FIG. 4: CD79-CD3 zeta chimeric polypeptide fusions in a multi-chain receptor

a, d, g, Schematics surface expressed membrane-bound immunoglobulins in complex with CD79-CD3 zeta chimeric polypeptides. All domains within the respective proteins are labeled to indicate the precise protein fragments. b, e, h, Assessment of expression and surface localization of stably integrated constructs displayed, respectively, in (a), (d), and (g). Surface expression of the membrane-bound Immunoglobulins was determined by immunostaining against the immunoglobulin constant domains followed by confocal microscopy and flow cytometry. One cell is shown per picture. The histograms show the expression level in a cell population (right-shifted distribution) compared to an unstained control (left-shifted distribution). c, f, i Specific lysis calculated from a LCA co-incubation assay after 4 h of the NK-92 variant displayed in, respectively, panels (a), (d) and (g) at different E:T cell ratios (x-axis) against Her2-positive SK-BR-3 red color and Her2-negative MDA-MB-468 cells blue color. Displayed are means of biological triplicates +−SD (extrapolated as a shaded area between discrete E:T ratios). *** represents p-values <0.001 of a statistical significance between the cytotoxic effects towards Her2-positive and Her2-negative cell lines.

FIG. 5: Incorporating two ADCC signaling domains into the receptor

a, d, g, Schematics of a surface expressed c-terminally modified membrane-bound immunoglobulin γ 1 (mIgG1) with FcεRIγ in complex with CD79-CD3 zeta chimeric polypeptides and as is. In the schematics, all domains within the respective proteins are labeled with the precise protein fragments used. b, e, h, Assessment of expression and surface localization of stably integrated constructs displayed, respectively, in (a), (d), and (g). Surface expression of the membrane-bound immunoglobulin was determined by immunostaining against the immunoglobulin constant domains followed confocal microscopy and flow cytometry. One cell is shown per picture. The histograms show the expression level in a cell population (right-shifted distribution) compared to an unstained control (left-shifted distribution). c, f, i, Specific lysis calculated from a LCA co-incubation assay after 4 h of the NK-92 variant displayed in (a), (d), (g) at different E:T cell ratios (x-axis) against Her2-positive SK-BR-3 red color and Her2-negative MDA-MB-468 cells blue color. Displayed are means of biological triplicates +−SD (extrapolated as a shaded area between discrete E:T ratios). *** represents p-values <0.001 of a statistical significance between the cytotoxic effects towards Her2-positive and Her2-negative cell lines.

FIG. 6: Surface expression of Trastuzumab-derived mIgG1 in HeLa cells.

In panels a-c, the schematics on the left illustrate the receptor structure and localization. The schemes in the middle show the transfected constructs. The micrographs on the right show fixed HeLa cells with Brightfield 10× labels indicating a brightfield channel, red pseudocolor representing the expression of the transfection control mCherry (in panels a and b) or the expression of the antibody chains (in panel c), the green pseudocolor reflecting the intensity of an anti-IgG staining and antibody surface expression, and the turquoise pseudocolor indicating mCerulean, the proxy for CD79 expression (panel c only). The scale bar is 100 μm. a, Plasmid transfection of constitutively driven heavy and light chains of the antibody alongside a transfection control. b, Plasmid transfection of constructs encoding the heavy and light antibody chains on a contiguous scaffold and the CD79A and CD79B proteins encoded on two separate plasmids. c, The transfection of polycistronic constructs adapted for lentiviral packaging and encoding the antibody chains with the mScarlet fluorescent reporter, and the lentiviral-adapted polycistronic construct encoding CD79A and CD79B with an mCerulean fluorescent reporter. Note that the green pseudocolor intensity cannot be directly compared between panels (a) and (b) on one hand, and panel (c) on the other, because the primary antibody used to stain samples in panel c originated from a different lot with stronger staining compared to the lot used in panels (a) and (b).

FIG. 7: Generation of stably transduced NK-92 cells.

a, SBFP2-mCerulean flow cytometry scatter plots of NK-92 cells stably transduced with Iv-EF1A-CD79 (mCerulean) and/or Iv-EF1A-CD16 (SBFP2) showing cell sorting gates and population frequencies of pre-sorted cells. NK-92-WT cells (plot on the left) were used as control and not sorted. Names of the cell lines resulting from the sorts are indicated above the scatter plots. b, mScarlet-FCS flow cytometry scatter plots of cell lines whose sorting is described in panel a, stably transduced with Iv-EF1A-mIgG1/Her2 or Iv-EF1A-mIgG1/Pollen with indicated sorting windows and population frequencies. Each plot in this panel shows the result of transducing the cells sorted beforehand according to their BFP, i.e. SBFP2, and Cerulean expression as shown in the plot right above it in panel a. Names of the cell lines resulting from the sort are indicated above the scatter plots. c-k, Antibody surface staining of membrane-bound immunoglobulin and CD16 in transduced NK-92 cell lines. Shown are histograms of membrane-bound immunoglobulin (light) and CD16 (dark) surface expression. Cell line names (Table 1) and the illustrations of cell surface modifications are shown above the histograms. I, Median expression intensity (i.e. median fluorescence) of membrane-bound immunoglobulin (light) and CD16 (dark) surface expressions corresponding to histograms in (d)-(k) depicted as a bar chart. m, Percent lysis of SK-BR-3 cells in co-incubation (i.e 4 h incubation) with NK-92 cells transduced with the viral vectors indicated below the bar chart at an E:T cell ratio of 5:1 or, alternatively, supplied with 10 μg/m L Herceptin (canonical ADCC) or 2% TritonX-100 (positive control for cell lysis) where indicated. Displayed are the means of biological triplicates +−SD ***, **, *represent p-values of, respectively, <0.001, <0.01, or <0.05, of statistical significance of the difference in the effects magnitudes between the compared conditions. For plasmid and viral vector details refer to FIG. 12 and for cell line details refer to Table 1.

FIG. 8: Target cell generation and characterization.

In panels (a) and (b) the green pseudocolor represents mCitrine and the red pseudocolor indicates APC, a measure of surface expression of Her2/ErbB2 (see Methods). a, Characterization of MDA-MB-468-LUC-CIT cell line. b, Characterization of SK-BR-3-LUC-CIT cell line. The scale bar is 50 μm. c, Correlation between the number of SK-BR-3-LUC-CIT cells and the total luminescent signal. Shown are means of biological triplicates +−S.D. d, Total luminescence of SK-BR-3-LUC-CIT cells supplied with increasing amounts of TritonX-100 for 30 min. Displayed are six independent biological replicates.

FIG. 9: Sorting of NK-92 cells stably transduced with tIgG1:

a, mScarlet-FSC flow cytometry scatter plots show the mScarlet expression in cell lines described in FIG. 7a after transduction with Iv-EF1A-tigG1/Her2 showing cell sorting gates and population frequencies of pre-sorted cells. Names of the cell lines resulting from the sorts are indicated above the scatter plots. b-e, Antibody surface staining against immunoglobulin and CD16 in transduced and sorted NK-92 cell lines. Shown are histograms of membrane-bound immunoglobulin (light) and CD16 (dark) surface expression. Cell line names (Table 1) and the illustrations of cell surface modifications are shown above the histograms. f, Medians of membrane-bound immunoglobulin (light) and CD16 (dark) surface expressions corresponding to histograms in (b)-(e) depicted as a bar chart. g, Percent lysis of SK-BR-3 cells in co-incubation (i.e. 4 h incubation) with NK-92 cells transduced with the viral vectors indicated below the bar chart at an E:T cell ratio of 5:1 or, alternatively, supplied with 10 μg/m L Herceptin (canonical ADCC) or 2% TritonX-100 (positive control for cell lysis) where indicated. Displayed are the means of biological triplicates +−SD ***, **, *represent p-values of, respectively, <0.001, <0.01, or <0.05, of statistical significance in the difference of the effects magnitudes between the compared conditions. For plasmid and viral vector details refer to FIG. 12 and for cell line details refer to Table 1.

FIG. 10: Sorting of NK-92 cells stably transduced with CD79-CD3 fusion.

a-b, mScarlet over mCerulean flow cytometry scatter plots showing cell sorting gates and population frequencies of pre-sorted cells a, of NK-92-WT and NK-92-mIgG1/Her2 cells stably transduced with Iv-EF1A-CD79-CD3. NK-92-WT cells (plot on the left) were used as control and not sorted. b, of NK-92-CD79-CD3 transduced with Iv-EF1A-mIgG1/Pollen. Names of the cell lines resulting from the sorts are indicated above the scatter plots. c, Percent lysis of SK-BR-3 cells in co-incubation with NK-92 cells transduced with the viral vectors indicated below the bar chart at an E:T cell ratio of 5:1 or, alternatively, supplied with 10 μg/mL Herceptin (canonical ADCC) or 2% TritonX-100 (positive control for cell lysis) where indicated. Displayed are the means of biological triplicates +−SD ***, **, *represent p-values of, respectively, <0.001, <0.01, or <0.05, of statistical significance between the effects magnitudes between the compared conditions. For plasmid and viral vector details refer to FIG. 12 and for cell line details refer to Table 1. d, mScarlet over mCerulean flow cytometry scatter plot of NK-92-CD79-CD3 additionally transduced with Iv-EF1A-mIgM/Her2 showing cell sorting gates and population frequencies of pre-sorted cells. e, Antibody surface staining against the membrane-bound immunoglobulin (light green) in NK-92 cells transduced with Iv-EF1A-mIgM/Her2 or Iv-EF1A-CD79-CD3. f, Medians of membrane-bound immunoglobulin (light green) surface expressions of histograms in (e) are summarized in a bar chart. For plasmid and viral vector details refer to FIG. 12 and for cell line details refer to Table 1.

FIG. 11: Sorting NK-92 cells stably transduced with mIgG1-FcεRIγ.

a, mScarlet over mCerulean flow cytometry scatter plots of NK-92-WT or NK-92-CD79-CD3 transduced with Iv-EF1A-mIgG1/Her2-FceRIg or Iv-EF1A-mIgG1/Pollen-FceRIg showing cell sorting gates and population frequencies of pre-sorted cells. b, Percent lysis of SK-BR-3 cells in co-incubation with NK-92 cells transduced with the viral vectors indicated below the bar chart at an E:T cell ratio of 5:1 or, alternatively, supplied with 10 μg/mL Herceptin (canonical ADCC) or 2% TritonX-100 (positive control for cell lysis) where indicated. Displayed are the means of biological triplicates +−SD ***, **, *represent p-values of, respectively, <0.001, <0.01, or <0.05, of statistical significance between the effects magnitudes between the compared conditions. For plasmid and viral vector details refer to FIG. 12 and for cell line details refer to Table 1.

FIG. 12. Constructs and lentiviral vector notations

FIG. 13. Receptor target specificity exchange.

a, b, c, and d, Specific lysis (y-axis) of CD19 and CD20 positive Raji target cells (black line) after a 4 h co-incubation with NK-92 cells that were modified with ASIMut receptors targeting HER2 (a), CD19 (b,c), and CD20 (d) at different E:T cell ratios (x-axis). The corresponding receptor description and structure are shown above each panel. Displayed are the means of biological triplicates +−SD.

### EXAMPLES

Methods and materials are described herein for use in the present disclosure other, suitable methods and materials known in the art can also be used. The materials, methods, and examples are illustrative only and not intended to be limiting.

### Example 1: Materials and Methods

### Materials

| Bacterial strains | Source | Identifier |
|---|---|---|
| Top10 | ThermoFisher | Cat#C404010 |
| Mach1 | ThermoFisher | Cat#C862003 |
| Stbl3 | ThermoFisher | Cat#C737303 |

| Antibodies | Source | Identifier/Lot |
|---|---|---|
| Anti-human IgG | SouthernBiotech | IgG1Cat#2040-08; |
| (Biotinylated) | | Lot#C1316-PM87D |
| Streptavidin-FITC | Southernbiotech | Cat#7100-02S; |
| | | Lot#D1017-TL27D |
| Anti-human IgG | Invitrogen | Cat# 13-4998-83; |
| (Biotinylated) | | Lot# 2311211 |
| Anti-human Ig | Invitrogen | Cat# 31782; |
| (Biotinylated) | | Lot# WE3278964 |
| Anti-human CD16 | Invitrogen | Cat# 56-0168-41; |
| (Alexa Fluor 700) | | Lot#2072513 |
| Streptavidin-BB515 | BD Bioscience | Cat# 564453; |
| | | Lot# 1025848 |
| Trastuzumab | MedChemExpress | Cat# HY-P9907/CS-7821; |
| | | Lot# 31861 |

| Software | Source | Identifier |
|---|---|---|
| Snapgene 4.3.11 | Snapgene | RRID: SCR_015052 |
| Fiji 2.0.0-rc-69/1.52p | https://imagej.net/ | RRID: SCR_002285 |
| Flowjo 10.6.1 | BD Biosciences | RRID: SCR_008520 |
| Prism 8 | GraphPad Prism | RRID: SCR_002798 |
| Instruments | Manufacturer | Identifier |
| BD LSR Fortessa II | BD Biosciences | RRID: SCR_002159 |
| Analyzer | | |
| Nikon Eclipse Ti2 - | Nikon | N/A |
| Microscope | | |
| Nikon A1 | Nikon | N/A |
| Leica SP8-Falcon | Leica | N/A |
| BD FACSMelody | BD Biosciences | N/A |
| BD Aria fusion | BD Biosciences | N/A |
| Tecan Infinite pro | Tecan | N/A |
| M1000 | | |

TABLE 1
List of cell line notations.

| Cell Line | Starting | transduced |
|---|---|---|
| shorthand notation | Cell line | lentiviral vectors |
| NK-92-m Scarlet | NK-92 | Iv-EF1A-mScarlet |
| NK-92-CD16 | NK-92 | Iv-EF1A-CD16 |
| NK-92-CD79 | NK-92 | Iv-EF1A-CD79 |
| NK-92-mIgG1/Her2 | NK-92 | Iv-EF1A-mIgG1/Her2 |
| NK-92-mIgG1/Her2- | NK-92 | Iv-EF1A-mIgG1/Her2 |
| CD79 | | Iv-EF1A-CD79 |
| NK-92-mIgG1/Her2- | NK-92 | Iv-EF1A-mIgG1/Her2 |
| CD16 | | Iv-EF1A-CD16 |
| NK-92-mIgG1/Her2- | NK-92 | Iv-EF1A-mIgG1/Her2 |
| CD16-CD79 | | Iv-EF1A-CD16 |
| | | Iv-EF1A-CD79 |
| NK-92-mIgG1/Pollen- | NK-92 | Iv-EF1A-mIgG1/Pollen |
| CD16-CD79 | | Iv-EF1A-CD16 |
| | | Iv-EF1A-CD79 |
| NK-92-tIgG1/Her2 | NK-92 | Iv-EF1A-tIgG1/Her2 |
| NK-92-tIgG1/Her2- | NK-92 | Iv-EF1A-tIgG1/Her2 |
| CD79 | | Iv-EF1A-CD79 |
| NK-92-tIgG1/Her2- | NK-92 | Iv-EF1A-tIgG1/Her2 |
| CD16 | | Iv-EF1A-CD16 |
| NK-92-tIgG1/Her2- | NK-92 | Iv-EF1A-tIgG1/Her2 |
| CD16-CD79 | | Iv-EF1A-CD16 |
| | | Iv-EF1A-CD79 |
| NK-92- mIgM/Her2 | NK-92 | Iv-EF1A-mIgM/Her2 |
| NK-92- mIgM/Her2- | NK-92 | Iv-EF1A-mIgM/Her2 |
| CD79-CD3 | | Iv-EF1A-CD79-CD3 |
| NK-92-mIgG1/Her2- | NK-92 | Iv-EF1A-mIgG1/Her2-FceRIg |
| FceRIg | | |
| NK-92-mIgG1/Her2- | NK-92 | Iv-EF1A-mIgG1/Her2-FceRIg |
| FceRIg-CD79-CD3 | | Iv-EF1A-CD79-CD3 |
| NK-92-mIgG1/Pollen- | NK-92 | Iv-EF1A-mIgG1/Pollen-FceRIg |
| FceRIg-CD79-CD3 | | Iv-EF1A-CD79-CD3 |
| SK-BR-3/ | SK-BR-3 | Iv-EF1A- Luc-Cit |
| SK-BR-3-LUC-CIT | | |
| MDA-MB-468/ | MDA-MB-468 | Iv-EF1A- Luc-Cit |
| MDA-MB-468-LUC- | | |
| CIT | | |

For further details about lentiviral vectors please refer to FIG. 12.

TABLE 2
Primers

| | Sequences are as shown in the attached | |
|---|---|---|
| Primer ID | sequence listing pursuant to WIPO St. 26 | SEQ ID NO: |
| PR5184 | [symbol] | 115 |
| PR5185 | [symbol] | 116 |
| PR5857 | [symbol] | 117 |
| PR5858 | [symbol] | 118 |
| PR5693 | [symbol] | 119 |
| PR5856 | [symbol] | 120 |
| PR5859 | [symbol] | 121 |
| PR5860 | [symbol] | 122 |
| PR8371 | [symbol] | 123 |
| PR8372 | [symbol] | 124 |
| PR8563 | [symbol] | 125 |
| PR8564 | [symbol] | 126 |
| PR3894 | [symbol] | 127 |
| PR8672 | [symbol] | 128 |
| PR8673 | [symbol] | 129 |
| PR8674 | [symbol] | 130 |
| PR8675 | [symbol] | 131 |
| PR8676 | [symbol] | 132 |
| PR8742 | [symbol] | 133 |
| PR8700 | [symbol] | 134 |
| PR8701 | [symbol] | 135 |
| PR8702 | [symbol] | 136 |
| PR8703 | [symbol] | 137 |
| PR8704 | [symbol] | 138 |
| PR8705 | [symbol] | 139 |
| PR8667 | [symbol] | 140 |
| PR8668 | [symbol] | 141 |
| PR8669 | [symbol] | 142 |
| PR6868 | [symbol] | 143 |
| PR8867 | [symbol] | 144 |
| PR8868 | [symbol] | 145 |
| PR8869 | [symbol] | 146 |
| PR8870 | [symbol] | 147 |
| PR8871 | [symbol] | 148 |
| PR8875 | [symbol] | 149 |
| PR8876 | [symbol] | 150 |
| PR6702 | [symbol] | 151 |
| PR8982 | [symbol] | 152 |
| PR8983 | [symbol] | 153 |
| PR8862 | [symbol] | 154 |
| PR8860 | [symbol] | 155 |
| PR4413 | [symbol] | 156 |
| PR8857 | [symbol] | 157 |
| PR4412 | [symbol] | 158 |
| PR9004 | [symbol] | 159 |
| PR9005 | [symbol] | 160 |
| PR9006 | [symbol] | 161 |
| PR9007 | [symbol] | 162 |
| PR9275 | [symbol] | 163 |
| PR4426 | [symbol] | 164 |
| PR4427 | [symbol] | 165 |

[symbol]

indicates data missing or illegible when filed

TABLE 3
gBlocks

| | Sequences are as shown in the attached | |
|---|---|---|
| gBlock ID | sequence listing pursuant to WIPO St. 26 | SEQ ID NO: |
| gBlock330 | [symbol] | 166 |
| gBlock331 | [symbol] | 167 |
| gBlock332 | [symbol] | 168 |
| gBlock339 | [symbol] | 169 |
| gBlock357 | [symbol] | 170 |
| gBlock359 | [symbol] | 171 |
| gBlock365 | [symbol] | 172 |

[symbol]

indicates data missing or illegible when filed

### Methods

### Cell Culture

SK-BR-3 cells (American Type Culture Collection, Cat #HTB-30, LOT #70022931), HEK293T (ATCC, Cat #CRL-11268), and HeLa cells (ATCC, Cat #CCL-2, Lot #58930571) were cultured in DMEM medium (Gibco, Cat #41966-029) supplemented with 10% fetal bovine serum (Gibco, Cat #10270-106), penicillin (100 U/mL), and streptomycin (100 μg/mL) (Gibco, Cat #15140-148) at 37° C. and 5% CO2. MDA-MB-468 cells (Deutsche Sammlung von Mikroorganismen und Zellkulturen GmbH; Cat #ACC738, Lot #5) were cultured in Leibovitz medium (Gibco, Cat #11415-064) supplemented with 10% FBS, penicillin (100 U/mL), and streptomycin (100 μg/mL) at 37° C. and atmospheric CO2 concentrations. NK-92 cells (Deutsche Sammlung von Mikroorganismen und Zellkulturen GmbH; Cat #ACC488, Lot #11) were cultured in Alpha MEM without ribonucleosides (Thermofisher, Cat #12000-063) solubilized in H2O (Gibco, Cat #10977-035) supplemented with 2.2 g/L of sodium bicarbonate (Sigma, Cat #S5761), 0.2 mM Myo-Inositol (Sigma, Cat #1-7508), 0.1 mM 2-Mercaptoethanol (Gibco, Cat #21985-023), 0.02 mM folic acid (Sigma, Cat #F-8758), 12.5% horse serum (Gibco, Cat #16050122), 12.5% fetal bovine serum, (100 U/mL), and streptomycin (100 μg/mL), and 10 ng/mL recombinant human IL-2 (Gibco, Cat #PHC0026) at 37° C. and 5% CO2. All media were sterile filtered using 0.22 μm (TPP, Cat #99950, Lot #20210129). Adherent cell lines were cultured in filter cap T-75 flasks (Greiner bio one; Cat #658175) and suspension culture cell lines were cultured in filter cap T-75 flasks (Greiner bio one; Cat #658195). All cell lines were tested negative for mycoplasma contamination.

### Lentiviral Vector Production

HEK293T cells were seeded at 5.5*10⁶ cells per T75 plate (Greiner Bio One, Cat #658175) and incubated at 37° C., 5% CO2 for 20 hours. DMEM supplemented with 10% FBS and no antibiotic was used in culturing cells for lentivirus production. DNA-Opti-MEM mix was prepared by mixing the following components: 34.2 μg of transfer plasmid (pFS312, pFS322, pFS331, pFS335, pFS349, pFS350, pFS353, pFS354, pFS355, pFS357, and pBA1037; see FIG. 12 and Table 4), 22.9 μg of pJD14 (See Table 4), 4.5 μg of either pJD15 for VSV-G pseudotype or pFS295 (See Table 4) for baboon pseudotype, and 2.3 μg of pJD16 (See Table 4) in a final volume of 500 μL in Opti-MEM. On the side, 500 μL of Lipofectamine 2000-Opti-MEM mix was prepared by adding 128 μl of Lipofectamine2000 (Invitrogen, Cat #11668019) to Opti-MEM such that the DNA (μg): Lipofectamine2000 (μl) ratio remained 1:2. Then, Lipofectamine2000-Opti-MEM mix was gently added dropwise to DNA-Opti-MEM mix and incubated at room temperature for 15 minutes. The transfection mix was added dropwise to the HEK293T packaging cells and incubated for 10 hours. Then, the media was gently aspirated and supplied with 15 mL pre-warmed fresh media. The lentivirus present in the supernatant (media) was harvested at 48 hours, stored at 4° C., and the cells were supplied with 15 mL pre-warmed fresh media. The same was repeated at 72 hours post transfection. The lentiviral harvests from 48 and 72 hours were pooled together. The pooled lentivirus was centrifuged at 500×g for 5 minutes and then filtered using 0.45 mm filter (Sartorius, Cat #16555-K). The viral supernatant was loaded on Amicon Ultra-15 centrifugal filter units (Merck Millipore, Cat #UFC910096) for concentration and buffer exchange by following manufacturer's instructions. The buffer was exchanged to sterile PBS-MK buffer. Lentiviral titration was adapted from Tiscornia et al. 2006 (Tiscornia, et al. 2006). 5.0*105 HEK293T cells were seeded in a 24 well plate (Thermo Scientific, Cat #142475), in a final volume of 500 μl of DMEM media per well. The plate was incubated at 37° C.; 5% CO2. After 24 h tenfold serial dilutions of the virus stock were made in PBS-MK from undiluted to a dilution of 10⁻³. 20 μl of each viral dilution to the cells, mixed thoroughly but gently and incubated the cells at 37° C. Cells were grown for 48 hours. The media was removed and discarded. Cells were resuspended in 150 μl of Accutase (ThermoFisher, Cat #A11105-0) and incubated for 5 min at RT.

Flow cytometry analysis was performed to determine the percentage of fluorescent reporter positive cells.

Calculate biological titer (BT=TU/ml, transducing units) according to the following formula: TU/μl=(P×N/100×V)×1/DF, where P=% Fluorophore+cells, N=number of cells at time of transduction=1*105, V=volume of dilution added to each well=20 μl and DF=dilution factor=1 (undiluted), 10⁻¹ (diluted 1/10), 10⁻² (diluted 1/100) using cells of a dilution that resulted in less than 40% transduction efficiency (if available).

TABLE 4
Plasmid cloning strategies.

| Recombinant DNA | Source | Identifier |
|---|---|---|
| pJD13 (5′LTR-UbC-GFP- | (Lois 2002) | Addgene Plasmid #14883 |
| WPRE-3′LTR) | | |
| pJD14 (CMV-Gag/Pol) | (Dull, et al. | Addgene Plasmid #12251 |
| | 1998) | |
| pJD15 (CMV-VSV-G) | (Dull, et al. | Addgene Plasmid #12259 |
| | 1998) | |
| pJD16 (REV-Rev) | (Dull, et al. | Addgene Plasmid #12253 |
| | 1998) | |
| pKH026 (EF1A-mCherry) | (Prochazka, | N/A |
| | et al. 2014) | |
| pPMT048 (EF1A-mScarlet) | | N/A |
| pJD146 (EF1A-mCerulean) | (Doshi, et | N/A |
| | al. 2020) | |
| pCS187 (EF1A- SBFP2) | (Stelzer and | N/A |
| | Benenson | |
| | 2020) | |
| pBA1037(5′LTR-EF1A- | | pJD17 was opened using EcoRI and |
| FFLuciferase-P2A-mCitrine- | | Hpal. FFLuciferase-P2A-m Citrine |
| WPRE-3′LTR) | | were amplified from pIK014 using |
| | | PR4426 and PR4427. The backbone |
| | | and PCR product were Gibson |
| | | assembled to obtain pBA1037. |
| pFS122 (EF1A-V_{L}Trastuzuab- | (Dodev, et | Addgene Plasmid# 61883 |
| κCL-pA_EF1A- | al. 2014) | |
| V_{H}Trastuzumab-γC_{H}-pA) | | |
| pFS190 (CMV- MCS-13X | (Kim, et al. | Addgene Plasmid #80899 |
| Linker-BioID2-HA) | 2016) | |
| pFS186 (EF1A-V_{L}Trastuzuab- | This work | The pFS122 backbone was digested |
| κCL-pA_EF1A- | | with PacI followed by gel purification |
| V_{H}Trastuzumab-γC_{H}-pA) | | (7843 bp). The AMP resistance |
| | | cassette, as well as the |
| | | ColE1/pMB1/pBR322/pUC origin of |
| | | replication were PCR apliefied from |
| | | pJD13 using PR5184 and PR5185 |
| | | followed by gel purification (1907 |
| | | bp). Gibson assembly was |
| | | performed of the PCR fragment and |
| | | the backbone to clone pFS186 |
| pFS188 (EF1A-V_{L}Trastuzuab- | This work | The backbone pFS186 was opened |
| κCL-pA_EF1A- | | using BsrGI followed by gel |
| V_{H}Trastuzumab-mγC_{H}-pA) | | purification (9376 bp). Oligos |
| | | PR5857 and PR5858 were annealed |
| | | and extended. The resulting |
| | | fragment was PCR amplified using |
| | | oligos PR5859 and PR5860 and gel |
| | | purified (256 bp). A fragment of the |
| | | IgG1 HC was PCR ampliefied using |
| | | oligos PR5693 and 5856 followed by |
| | | gel purification (385 bp) to allow |
| | | gibson assembly. Gibson assembly |
| | | was performed for all fragments and |
| | | the backbone to clone pFS188 |
| pFS273 (EF1A-CD16) | This work | The backone pJD146 was opened |
| | | using EcoRI and NotI followed by gel |
| | | purification (4279 bp). The oligos |
| | | PR8371 and PR8372 were used to |
| | | PCR amplify gBlock330 containing |
| | | CD16. The resulting PCR product |
| | | was digested using EcoRI and NotI |
| | | followed by gel extraction (829 bp). |
| | | T4 ligase was used to ligate the |
| | | Backbone with the Insert to create |
| | | pFS273 |
| pFS274 (EF1A-CD79A) | This work | The backbone pJD146 was opened |
| | | using EcoRI and NotI followed by gel |
| | | purification (4279 bp). The oligos |
| | | PR8371 and PR8372 were used to |
| | | PCR amplify gBlock331 containing |
| | | CD79A. The resulting PCR product |
| | | was digested using EcoRI and NotI |
| | | followed by gel extraction (745 bp). |
| | | T4 ligase was used to ligate the |
| | | Backbone with the Insert to create |
| | | pFS274 |
| pFS275 (EF1A-CD79B) | This work | The backone pJD146 was opened |
| | | using EcoRI and NotI followed by gel |
| | | purification (4279 bp). The oligos |
| | | PR8371 and PR8372 were used to |
| | | PCR amplify gBlock332 containing |
| | | CD79A. The resulting PCR product |
| | | was digested using EcoRI and NotI |
| | | followed by gel extraction (754 bp). |
| | | T4 ligase was used to ligate the |
| | | Backbone with the Insert to create |
| | | pFS275 |
| pFS281 (5′LTR-EF1A- | This work | The backbone pJD17 was opened |
| mScarlet-WPRE-3′LTR) | | using AgeI and BsrGI followed by gel |
| | | purification (8105 bp). mScarlet was |
| | | extracted from pPMT048 using AgeI |
| | | and BsrGI. T4 ligase was used to |
| | | ligate insert and backbone to form |
| | | pFS281 |
| pFS295 (CMV-BaEVTR) | This work | The backbone pJD15 was amplified |
| | | using oligos PR8563 and PR8564 |
| | | followed by gel purification (4331 |
| | | bp). Gibson assembly was |
| | | performed for the backbone and the |
| | | BaEVTR containing gBlock339 to |
| | | obtain pFS295 |
| pFS309 (5′LTR-EF1A- | This work | The backbone pFS281 was opened |
| mCerulean-P2A-CD79A-T2A- | | using EcoRI and BstBI followed by |
| CD79B-WPRE-3′LTR) | | gel purification (9117 bp). mCerulean |
| | | was PCR amplified from pJD146 |
| | | using oligos PR3894 and PR8667 |
| | | followed by gel purification (798 bp). |
| | | CD79A was PCR amplified from |
| | | pFS274 using oligos PR8668 and |
| | | PR8669 followed by gel purification |
| | | (767 bp). CD79B was PCR amplified |
| | | from pFS275 using oligos PR8670 |
| | | and PR8671 followed by gel |
| | | purification (771 bp). Gibson |
| | | assembly was performed for the |
| | | backbone and all PCR fragments to |
| | | obtain pFS309 |
| pFS312 (5′LTR-EF1A- | This work | The backbone pFS281 was opened |
| mScarlet-T2A-V_{L}Trastuzuab- | | using BstBI and EcoRI followed by |
| κC_{L}-P2A-V_{H}Trastuzumab- | | gel purification (9117 bp). mScarlet |
| mγC_{H}-WPRE-3′LTR) | | was PCR amplified from pFS281 |
| | | using oligos PR3894 and PR8672 |
| | | followed by gel purification (779 bp). |
| | | The Trastuzumab kappa light chain |
| | | was PCR amplified from pFS188 |
| | | using oligos PR8673 and PR8674. |
| | | followed by gel purification (782 bp). |
| | | The Trastuzumab heavy chain with |
| | | human transmembrane and |
| | | cytosolic domain was PCR amplified |
| | | from pFS188 using oligos PR8675 |
| | | and PR8677 followed by gel |
| | | purification (1694 bp). Gibson |
| | | assembly was performed for the |
| | | backbone and all PCR fragments to |
| | | form pFS312 |
| pFS317 (EF1A-V_{L}Trastuzuab- | | The backbone pFS188 was opened |
| κCL-pA_EF1A- | | using BsrGI and gel purified (9885 |
| V_{H}Trastuzumab-m-linker-γC_{H}- | | bp). A fragment of IgG1 was PCR |
| pA) | | amplified from pFS188 to allow |
| | | Gibson assembly using PR8700 and |
| | | PR87001 followed by gel purification |
| | | (330 bp). The 11xGGGGS linker was |
| | | PCR ampliefied from pFS190 using |
| | | oligos PR8702 and PR8703 followed |
| | | by gel purification (240 bp). The |
| | | human mIgG1 transmembrane and |
| | | cytosolic domain were PCR |
| | | amplified from pFS188 using oligos |
| | | PR8704 and PR8705 to allow |
| | | Gibson assembly and gel purified |
| | | (256 bp). Gibson assembly was |
| | | performed for the backbone and all |
| | | inserts to form pFS317 |
| pFS322 (5′LTR-EF1A- | This work | The backbone pFS312 was opened |
| mScarlet-T2A-V_{L}Trastuzuab- | | using BstBI and BamHI followed by |
| κC_{L}-P2A-V_{H}Trastuzumab-m- | | gel purification (10651 bp). The |
| linker-γC_{H}-WPRE-3′LTR) | | Trastuzumab heavy chain containing |
| | | a 11xGGGGS linker and human |
| | | transmembrane and cytosolic |
| | | domain was PCR amplified from |
| | | pFS317 using oligos PR8675 and |
| | | PR8742 followed by gel purification |
| | | (1906 bp). Gibson assembly was |
| | | performed for the backbone and |
| | | Insert to form pFS322 |
| pFS331 (5′LTR-EF1A- | This work | The backbone pFS309 was opened |
| mCerulean-T2A-CD79A-P2A- | | using AsiSI and EcoRI followed by |
| CD79B-WPRE-3′LTR) | | gel purification (9112 bp). mCerulean |
| | | was PCR amplified from pJD146 |
| | | using oligos PR6868 and PR8667 |
| | | followed by gel purification (798 bp). |
| | | CD79A was PCR amplified from |
| | | pFS274 using oligos PR8868 and |
| | | PR8869 followed by gel purification |
| | | (767 bp). CD79B was PCR amplified |
| | | from pFS275 using oligos PR8870 |
| | | and PR8871 followed by gel |
| | | purification (771 bp). Gibson |
| | | assembly was performed for the |
| | | backbone and all PCR fragments to |
| | | obtain pFS331 |
| pFS335 (5′LTR-EF1A- SBFP2- | This work | The backbone pFS309 was opened |
| T2A-CD16-WPRE-3′LTR) | | using AsiSI and EcoRI followed by |
| | | gel purification (9112 bp). SBFP2 |
| | | was PCR amplified from pCS187 |
| | | using oligos PR8871 and PR8867 |
| | | followed by gel purification (808 bp). |
| | | CD16was PCR amplified from |
| | | pFS273 using oligos PR8875 and |
| | | PR8876 followed by gel purification |
| | | (846 bp). Gibson assembly was |
| | | performed for the backbone and all |
| | | PCR fragments to obtain pFS335 |
| pFS341 (CMV-CD19-CAR- | (Bloemberg, | Addgene Plasmid #135992 |
| BBz) | et al. 2020) | |
| pFS343 (5′LTR-EF1A- | This work | The backbone pFS331 was liniarized |
| mCerulean-T2A- | | with two PCRs using oligos PR8857 |
| CD79A:CD3z-P2A-CD79B- | | and PR4413 (5651 bp), as well as |
| WPRE-3′LTR) | | oligos PR8862 and PR8860 (5584 |
| | | bp). The CD3zeta cytosolic domain |
| | | was PCR amplified from pFS341 |
| | | using oligos PR8660 and PR8862 |
| | | followed by gel purification (383 bp). |
| | | Gibson assembly was performed for |
| | | the backbone and all PCR fragments |
| | | to obtain pFS343 |
| pFS349 (5′LTR-EF1A- | This work | The backbone pFS312 was opened |
| mScarlet-T2A-V_{L}Trastuzuab- | | using AvrII and EcoRI followed by gel |
| κCL-P2A-V_{H}Trastuzumab- | | purification (6742 bp). A part of the |
| mγC_{H}-FceRIg-WPRE-3′LTR) | | pFS312 backbone was PCR |
| | | amplified using oligos PR6702 and |
| | | PR8982 followed by gel purification |
| | | (2504 bp) to allow Gibson assembly. |
| | | mScarlet, Trastuzumab kappa light |
| | | chain as well as Trastuzumab heavy |
| | | chain containing the human mIgG1 |
| | | transmembrane and cytosolic |
| | | domain were amplified from pFS312 |
| | | using oligos PR8983 and PR6868 |
| | | followed by gel purification (3179 |
| | | bp). FceRIg was introduced via the |
| | | overhangs of oligos PR8982 and |
| | | PR8983. Gibson assembly was |
| | | performed for the backbone and all |
| | | PCR fragments to obtain pFS349. |
| pFS350 (5′LTR-EF1A- | This work | The backbone pFS343 was liniarized |
| mCerulean-T2A- | | with two PCRs using oligos PR9004 |
| CD79A:CD3z-P2A- | | and PR4413 (6520 bp), as well as |
| CD79B:CDE3z-WPRE-3′LTR) | | oligos PR8862 and PR4413 (5584 |
| | | bp). An additional CD3zeta cytosolic |
| | | domain was PCR amplified |
| | | gBlock365 using oligos PR9006 and |
| | | PR9007 followed by gel purification |
| | | (348 bp). Gibson assembly was |
| | | performed for the backbone and all |
| | | PCR fragments to obtain pFS350. |
| pFS354 (5′LTR-EF1A- | This work | The backbone pFS312 was opened |
| mScarlet-T2A-V_{L}Trastuzuab- | | using BstBi and Esp3I followed by |
| κC_{L}-P2A-V_{H}Trastuzumab- | | gel purification (11048 bp). Gibson |
| mμC_{H}-WPRE-3′LTR) | | assembly was performed for the |
| | | backbone and gBlock357 containing |
| | | the mIgM constant domain to obtain |
| | | pFS354 |
| pFS355 (5′LTR-EF1A- | This work | The backbone pFS312 was opened |
| mScarlet-T2A-V_{L}Pollen-κC_{L}- | | using EcoRI and Esp3I followed by |
| P2A-V_{H}Pollen-mγC_{H}-WPRE- | | gel purification (10322 bp). The |
| 3′LTR) | | Pollen FVs were amplified from |
| | | gBlock359 using oligos PR6868 and |
| | | PR9275 followed by gel purificaiton |
| | | (2021 bp). Gibson assembly was |
| | | performed for the backbone and |
| | | insert to obtain pFS355 |
| pFS357 (5′LTR-EF1A- | This work | Plasmids pFS349 and pFS355 were |
| mScarlet-T2A-V_{L}Pollen-κC_{L}- | | digested using EcoRI and Esp3I. |
| P2A-V_{H}Pollen-mγC_{H}-FceRIg- | | Fragments were gel purified. |
| WPRE-3′LTR) | | Backbone from pFS349 (10382 bp) |
| | | containing the mIgG1 with cytosolic |
| | | FceRIg chain and Insert from |
| | | pFS355 (1943 bp) containing the |
| | | Pollen FV fragments were ligated to |
| | | obtain pFS357. |

Of note, mTagBFP2 could be used herein and in context of the invention, e.g. in the Examples, as an equivalent in place of SBFP2.

### Lentiviral Transduction of Target Cells

3 million MDA-MB-468 or SK-BR-3 cells were seeded in a T-75 flask (Greiner bio one; Cat #658175) and supplied with a VSV-G pseudotyped lentivirus carrying the EF1A-Luciferase-P2A-mCitrine gene (pBA1037) at an MOI of 3. Cells were incubated at corresponding culturing conditions (see cell culture methods) for 3 days. For sorting the cells were detached using 2 mL Trypsin-EDTA (Gibco; Cat #25200072) for 5 min. The reaction was stopped by the addition of 8 mL the appropriate culture medium. The cells were centrifuged at 350×g for 5 min. The supernatant was discarded and the cells were resuspended in sterile filtered 2 mL PBS+5% FBS. The cells were sorted using the FACSMelody (BD Biosciences) (Ex: 488 nm, Em: 527/32 nm).

### Lentiviral Transduction of NK Cells

NK-92 cells were activated by adding 10 ng/mL fresh IL-2 (Gibco, Cat #PHC0026) two hours prior to transduction. After the incubation time elapsed, the cells were counted using a Neubauer counting chamber. 100000 cells were transferred to a sterile 1.5 mL Eppendorf tube. Cells were centrifuged at 350×g for 5 min at RT. The supernatant was discarded and the cells were resuspended in an MOI of 100 of the baboon pseudotyped lentivirus. The volume of the cell mix was adjusted to 1 mL using the appropriate medium. The cell mixes where then spinfected at 1000×g for 30 min at RT. Following spinfection the transduced NK cells were transferred to a 12 well plate and supplied with 1 mL of additional NK-cell medium (see cell culture). Cells were then incubated at 37° C. and 5% CO₂. After 2 days the cells were expanded for sorting by transferring them to a T75 flask (Greiner bio one; Cat #658195) and supplying them with 15 mL of the NK-cell medium. The cells were expanded for 7 additional days prior to sorting. The cells were sorted using a BD Aria sorter (BD bioscience) to sort SBFP2 (Ex: 405 nm, Em: 450/50) and mCerulean (Ex: 405 nm, Em: 510/50) transduced cells and using a BD FACSMelody (BD bioscience) to sort mScarlet transduced cells (Ex: 561 nm, Em: 613/18 nm).

### Staining and Imaging of Antibody Surface Expression in HeLa Cells

HeLa cells were transfected using Lipofectamine 2000 transfection reagent (Invitrogen, Cat #11668-027) in 8-well p-slides (ibidi, Cat #80827). Cells were seeded 24 hours prior to transfection at a density of 2.5*10⁴ per well to obtain around 80-90% of confluency at the time of transfection. Up to 175 ng Plasmids were mixed with Opti-MEM (ThermoFisher, Cat #31985-062) to obtain a final volume of 12.5 μL. The appropriate volume of lipofectamine2000 was mixed with Opti-MEM to make final volume of 12.5 μL with a DNA:Lipofectamine 2000 ratio of 1:2, incubated for 20 min and added dropwise to the sample. 48 h post transfection the medium was removed. Cells were washed three times with 300 μl PBS (Gibco, Cat #10010-023) and fixed using 200 μl Image-iT (Invitrogen, Cat #FB002) for 15 min. After the removal of the fixing solution the cells were washed three times with 300 μl PBS. The primary antibody targeting human IgG1 (SouthernBiotech; Cat #2040-08; Lot #C1316-PM87D) was diluted 1:500 in PBS+5% FBS (Gibco, Cat #10270-106). 250 μl of this dilution were added per well and incubated for 30 min at RT. After removal of the antibody mixture the cells were washed three times with 300 μl PBS+5% FBS. Streptavidin-FITC (SouthernBiotech; Cat #7100-02S; Lot #D1017-TL27D) was diluted 1:500 in PBS+5% FBS. 250 μl of this dilution were added per well and incubated for 30 min at RT in the dark. After removal of the staining mixture the cells were washed three times with 300 μl PBS+5% FBS. 200 μl PBS+5% FBS were added per well for storage until imaging. Cells were imaged using a Nikon Eclipse Ti microscope (see Methods: Fluorescent microscopy).

### Recombinant DNA Methods

For different kits used, manufacturer's instructions were followed unless indicated otherwise. Standard cloning techniques were used to generate plasmids. DNA amplification was performed using Phusion High Fidelity DNA Polymerase (NEB, Cat #M0530). De-salted primers/oligonucleotides (Table 2) were ordered from IDT/Sigma Aldrich. Gene fragments and gBlocks were ordered from IDT or Twist Biosciences (Table 3). Digestion fragments were purified using MinElute PCR purification kit (QIAGEN, Cat #28006) or Qiaquick PCR purification kit (QIAGEN, Cat #28106). Gel extraction and purification was performed using MinElute Gel purification kit (QIAGEN, Cat #28606) or Qiaquick Gel Extraction kit (QIAGEN, Cat #28706). Restriction digestion was performed for BstBI at 65° C., Sfil at 50° C., BtgZI at 70C and for all other enzymes at 37° C. Ligation reaction was performed using T4 DNA ligase (NEB, Cat #M0202). Mix and Go E. coli transformation kit (Zymo, Cat #T3001) was used for preparing chemically-competent cells—Top10 (ThermoFisher, Cat #C404010). In-house prepared Machi electro-competent cells (ThermoFisher, Cat #C862003) and chemically competent Stbl3 cells (ThermoFisher, Cat #C737303) were also used for cloning. Screening of positive clones was either performed using restriction digestion or performing colony PCR with Quick-Load Taq 2× Master Mix (NEB, Cat #M0271). Plasmid isolation from positive clones was performed using GenElute Plasmid Mini-prep kit (Sigma Aldrich, Cat #PLN350-1KT). All the plasmids were verified using Sanger sequencing service provided by Microsynth AG (Switzerland). Transformed bacteria were cultured in Difco LB broth, Miller (BD, Cat #244610) supplemented with Ampicillin 100 mg/mL (Sigma Aldrich, Cat #A9518). PureYield Plasmid Midi-prep System (Promega, Cat #A2495) was used for plasmid isolation and purification. Endotoxin Removal kit (Norgen, Cat #52200) was used for removing endotoxins from purified plasmids. Gibson et al. (2009) assembly was performed at 50° C. for 1 hour in 20 mL final volume by mixing vector (50 ng) and inserts (5 molar equivalent) in 1× Gibson assembly buffer (0.1 M Tris-HCl, pH 7.5, 0.01 M MgCl2, 0.2 mM dGTP, 0.2 mM dATP, 0.2 mM dTTP, 0.2 mM dCTP, 0.01 M DTT, 5% (w/v) PEG-8000, 1 mM NAD), 0.04 units of T5 exonuclease (NEB, Cat #M0363), 0.25 units of Phusion DNA polymerase (NEB, Cat #M0530) and 40 units of Taq DNA ligase (NEB, Cat #M0208). Negative controls for Gibson assemblies included vectors alone.

### Staining of Mlgs and CD16 on NK-92 Cells

NK cells were counted using a Neubauer counting chamber. 3 million cells per condition were transferred to a 15 mL falcon tube (Greiner bio one; Cat #188261) and centrifuged at 350×g for 5 min at 4° C. After centrifugation the supernatant was discarded and the cells were washed three times with 3 mL ice cold PBS. Afterwards the cells were resuspended in 250 μl of a 1:500 dilution of biotinylated goat anti human IgG antibody (Invitrogen, Cat #13-4998-83, Lot #2311211) in cold PBS+5% FBS to stain IgG and in a 1:500 dilution of biotinylated donkey anti human Ig antibody (Invitrogen; Cat #31782, Lot #WE3278964) in cold PBS+5% FBS to stain IgM. The mixes were incubated in the dark on ice for 45 min. Afterwards, the mixes were centrifuged at 350×g for 5 min. The supernatant was removed and the cells were washed in 3 mL ice cold PBS+5% FBS. The cell pallet was resuspended in 250 μl of a 1:500 dilution of anti-human CD16 antibody (Invitrogen; Cat #56-0168-41, Lot #2072513) and 1:500 Streptavidin-BB515 (BD Bioscience; Cat #564453; Lot #1025848) in cold PBS+5% FBS. The cells were incubated on ice in the dark for 45 min. Afterwards the cells were centrifuged at 350×g for 5 min at 4° C. After centrifugation the supernatant was discarded and the cells were washed 3 times with 3 mL ice cold PBS. Following the last washing step, the supernatant was discarded and the cells were resuspended in 200 μl Image-iT fixation solution (Invitrogen, Cat #FB002) for 15 min. After the removal of the fixing solution the cells were washed three times with 1 mL RT PBS. Stained NK cells were either used for Flow cytometry or confocal microscopy.

### Flow Cytometry

To analyze stained NK cells (see Staining of migs and CD16 on NK-92 cells) on a flow cytometer, samples were taken, transferred to a 1.5 mL Eppendorf LoBind Tubes (Eppendorf, Cat #022431021), and centrifuged at 350×g for 3 min at RT. Supernatant was discarded and cells were resuspended in PBS and kept on ice until measuring. For adherent cells, the medium was removed, cells were washed with 500 μl PBS and detached with Accutase (ThermoFisher, Cat #A11105-01) in a total volume of 150 μL. Cells were then re-suspended and transferred to micro-dilution tubes (Cat #02-1412-0000, Life Systems Design). Following this, cells were analyzed using BD LSR Fortessa II Cell Analyzer (BD Biosciences). The machine was calibrated with Sphero Rainbow Calibration Particles 8-peak beads (Spherotech, Cat #PCP-30-5A) prior to use. The excitation lasers (Ex) and emission filters (Em) used for respective fluorescent protein measurements are as follows: SBFP2 (Ex: 405 nm, Em: 450/50 nm), mCerulean/CFP (Ex: 445 nm, Em: 473/10 nm), FITC (Ex: 488 nm, Em: 530/30 nm, longpass filter 505 nm), mScarlet (Ex: 561 nm, Em: 610/20 nm, longpass filter 600 nm), and AlexaFlour 700 (Ex: 640, Em: 730/45. Photomultiplier mV values of FSC-A: 450, SSC-A: 270, SBFP2: 550, mCerulean: 1000, mScarlet: 600 were used. FITC: 650, and Alexafluor 700: 500.

### Fluorescence Microscopy

When not specified as “confocal imaging” images were acquired utilizing Nikon Eclipse Ti microscope equipped with a mechanized stage and temperature control chamber held at 37° C. The excitation light was generated by a Nikon IntensiLight C-HGFI mercury lamp or LED source and filtered through a set of optimized Semrock filter cubes. The resulting images were collected by a Hammamatsu, ORCA R2, Flash4, or Prime BSI Express camera using a 10× objective. The following optimal excitation (Ex), emission (Em) and dichroic (Dc) filter sets were used to minimize the cross-talk between different fluorescent channels: mScarlet (Ex 562/40 nm or 575 nm LED with 10% intensity, Em 624/40 nm, Dc 593 nm), mCitrine (Ex 500/24 nm or 475 nm LED with 10% intensity, Em 542/27 nm, Dc 520 nm), GFP (Ex 500/24 nm or 475 nm LED with 10% intensity, Em 542/27 nm, Dc 520 nm) CFP/mCerulean (Ex 438/24 or 438 nm LED with 10% intensity, Em 483/32 nm, Dc 458 nm) and SBFP2 (Ex 370/36 nm or 390 nm LED with 10% intensity, Em 483/32 nm, Dc 458 nm). Image processing for figure preparation was performed using Fiji software (https://imagej.net/).

### Confocal Microscopy

2 μl of previously stained and fixed NK-92 cell lines (see methods: staining of migs and CD16 on NK-92 cells) were transferred to microscope slides and covered with a cover slip. Images were taken using a Leica SP8-Falcon point-scanning confocal microscope with a Leica DMI 8 base, Leica TCS Tandem scanner, 2 PMT+2 HyD detectors and a HC PL APO CS2 63×/1.40 oil immersion objective (Leica). As light sources a 442 nm diode laser, an Argon laser (run at 30% power) and a white light laser (run at 85% power, 80 MHz) were used. Images were acquired in sequential mode: Sequence 1: mCerulean (Ex: 442 nm, AOBS at 10%; Em: HyD SMD2 448-483 nm) and mScarlet (Ex: white light laser at 561 nm, AOBS at 10%; Em: HyD SMD4 571-674 nm). Sequence 2: BD BB515 (Ex: Argon laser 488 nm line, AOBS at 5%; Em: HyD SMD2 495-553 nm). Images were taken with a view field of 2048×2048 pixels, unidirectionally scanning at a speed of 400 Hz, with a pixel size of 90 nm, and a pixel dwell time of 0.79 μs. Confocal pinhole was set to 95.5 μm, and z-step was 0.3 μm. No averaging or summation of frames was applied.

Stained target cells on an ibidi cover slip were used directly (See methods: staining of target cells). Images were taken using a Nikon A1 Microscope with a Nikon Eclipse Ti2-E base, a Nikon A1 H25 scan head with Galvano scanner, 2× GaAsP+3×PMT detectors using an S Plan Fluor ELWD 20×ph ADM objective (NA 0.45, Nikon). Images were acquired in sequential channel mode: Sequence 1: mCitrine (Ex: 488 nm laser at 8%; Em: 525/50 nm, Gain: 32 mV), Sequence 2: APC (Ex: 640 nm laser at 10%; Em: 700/75 nm, Gain: 70 mV). Images were taken with a view field of 1024×1024 pixels at a zoom of 1.821, unidirectionally scanning with a pixel size of 340 nm, and a pixel dwell time of 10.2 μs. Confocal pinhole was set to 46 μm for both channels, and a line integration of 8× was applied. Image processing for figure preparation was performed using Fiji software (https://imagej.net/).

### Killing Assay

We assessed the killing capacity of the NK-92 cell array by luminescence-based cytolytic assay (LCA) (Brown, et al. 2005), using luciferase-modified target cells and luciferase activity as a measure of cell viability. The luminescent signal resulting from processing Luciferin with ATP by the Firefly Luciferase expressing target cells is used as assay readout. Dead cells, or cells with compromised membrane integrity cannot maintain their intracellular ATP level. This reduction leads to a decrease in observed luminescent signal. To this end, SK-BR-3 and MDA-MB-468 target cells were seeded at 20*105 per well in a 96 well plate (Greiner bio one, Cat #655094) in 100 μl of the cell line appropriate medium 24 h prior to the experiment. NK-92 derived cell lines were activated 24 h prior to the experiment by supplying them with fresh 10 ng/mL IL-2. On the day of the experiment viable cells of the target and NK-92 cell lines were counted using a Neubauer counting chamber. The number of NK-92 cells to obtain 10:1, 5:1, 2:5:1, and 1.25:1 effector to target cell ratios was transferred to a 15 mL Falcon tube and centrifuged at 350×g for 5 min. The supernatant was removed, and the NK-cells were resuspended in the culture medium of the target cell. The medium of the target cells was then replaced with 100 μl the appropriate effector cell mix. For the 2% Triton X100 control, target cells were supplied with 100 μl cell line appropriate medium containing 2% Triton X 100 (Carl Roth GmbH+Co. KG, Cat #9002-93-1). No killing controls were supplemented with 100 μl of the cell line appropriate medium. The killing assay was either incubated in a cell culture incubator in the dark for 4 h at 37° C. and 5% CO₂, or for 4 h at 37° C. and 5% CO₂ in the incubation chamber of a Nikon Ti2 microscope (see Methods: Fluorescent microscopy) in a dark room. If the plate was imaged, an image was taken every 30 min. mScarlet 500 ms exposure time, mCitrine 500 ms exposure time, mCerulean: 500 ms exposure time, and SBFP2 500 ms exposure time. 15 min prior to the end of the incubation time 5 μl of 15 mg/mL Luciferin (Promega, E1605) in PBS was added to each well. After the incubation was finished, the Luminescence was measured using a Tecan Infinite pro M1000 plate reader. Specific lysis values were calculated as follows:

% specific lysis=(1−Luminescence of sample of interest−Luminescence of 2% Triton X100 sample/Luminescence of untreated sample−Luminescence of 2% Triton X100 sample)*100

Specific lysis values that were below 0% were assumed to be 0%.

### Target Cell Death—Luminescence Correlation

35,000 SK-BR-3-LUC-CIT cells in 100 μl DMEM medium (Gibco, Cat #41966-029) supplemented with 10% fetal bovine serum (Gibco, Cat #10270-106), penicillin (100 U/mL), and streptomycin (100 μg/mL) (Gibco, Cat #15140-148) were seeded per well of a 96 well plate (Greiner bio one, Cat #655094) and incubated for 24 h at 37° C. and 5% CO₂. The next day dilutions of TritonX-100 (SigmaAldrich; Cat #X100-100ML) were prepared in DMEM. The cell medium of the cells in the 96 well plate was replaced with 100 μl of the TritonX-100 dilutions (6 replicates each) and incubated at 37° C. and 5% CO₂ for 15 min. Afterwards 5 μl of 15 mg/mL Luciferin (Promega, E1605) in PBS were added to each well and the plate was incubated for another 15 min at 37° C. and 5% CO₂. Subsequently the Luminescence of the samples were measured with a Tecan Infinite pro M1000 plate reader.

### Target Cell Number—Luminescence Correlation

SK-BR-3-LUC-CIT cells were seeded in wells of a 96 well plate (Greiner bio one, Cat #655094) at indicated cell numbers in DMEM medium (Gibco, Cat #41966-029) supplemented with 10% fetal bovine serum (Gibco, Cat #10270-106), penicillin (100 U/mL), and streptomycin (100 μg/mL) (Gibco, Cat #15140-148). After 4 h of attachment time at 37° C. and 5% CO₂, the cells were supplied with 5 μl of 15 mg/mL Luciferin (Promega, E1605) in PBS. Subsequently the Luminescence of the samples were measured with a Tecan Infinite pro M1000 plate reader. Afterwards, the cell medium was removed, the cells were washed with 200 μl PBS (Gibco, Cat #10010-023) and detached for 10 min at RT with 250 μl Accutase (ThermoFisher, Cat #A11105-01). The full volume containing all cells of each well was transferred to a BD Trucount tube (BD, Cat #340334), and run on a BD LSR Fortessa II Cell Analyzer (BD Biosciences) and counted according to the manufacturer's protocol. SK-BR-3 cells were identified using mCitrine (Ex: 488 nm, Em: 530/30 nm, Iongpass filter 505 nm).

### Staining of Her2 on Target Cells

SK-BR-3-LUC-CIT and MDA-MB-468-LUC-CIT cells were seeded in 8-well p-slides (ibidi, Cat #80827) 24 hours prior to staining and incubated at 37° C. and 5% CO₂. Cells were washed three times with 300 μl PBS (Gibco, Cat #10010-023) and fixed using 200 μl Image-iT (Invitrogen, Cat #FB002) for 15 min. After the removal of the fixing solution the cells were washed three times with 300 μl PBS. The antibody targeting human Her2 (Invitrogen; Cat #2040-08; Lot #C1316-PM87D) was diluted 1:500 in PBS+5% FBS (Gibco, Cat #10270-106). 250 μl of this dilution were added per well and incubated for 30 min at RT. After removal of the antibody mixture the cells were washed three times with 300 μl PBS. 200 μl PBS+5% FBS were added per well for storage until imaging. Cells were imaged using a Nikon A1 point scanning microscope (see confocal microscopy).

### Statistical Analyses

Statistical analyses were performed in Microsoft Excel. Student's t-test was used to compare quantitative differences (mean±SD) between samples; P-values were two-sided and P<0.05 was considered significant.

### Example 2: Surface Expression of a Membrane Bound IgG1 Immunoglobulin from a Polycistronic Construct

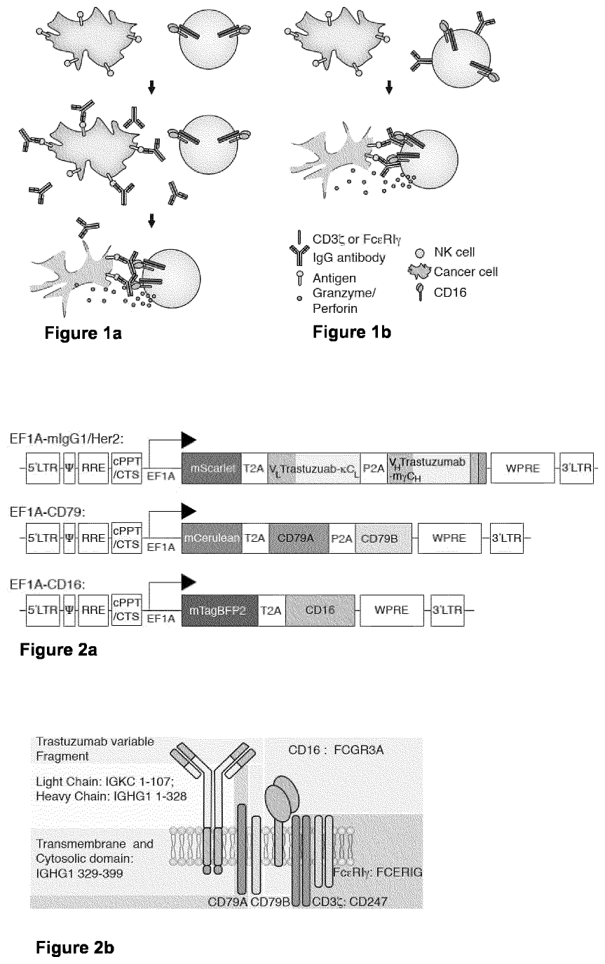

Conventional antibody dependent cellular cytotoxicity (ADCC) involves a signaling receptor CD16 expressed at the surface of an effector cell which recognizes the Fc domain of a soluble antibody bound to an antigen at a target cell. Therefore, ADCC conventionally depends on the presence of a soluble antibody (Gauthier, et al. 2021; Gómez Román, et al. 2014) (FIG. 1a). According to the present invention, however, ADCC can be triggered, inter alia, by a cis interaction between a membrane-bound/tethered antibody and a CD16 receptor on the cell surface of an effector cell such as an NK cell (FIG. 1b). The present inventors used (i) a cell line NK-92, which does not naturally express CD16 but can be supplemented with exogenous CD16 via lentiviral transduction; (ii) HER2/neu (Her2; ErbB2) as a model antigen on the target cell surface; (iii) Her2-expressing and non-expressing breast cancer cell lines to represent potential target cells; and (iv) a Her2 monoclonal antibody Trastuzumab as the basis for antigen-specific recognition.

Surprisingly, the inventors were able to tether an antibody to the cell surface of NK-92 cells. Initially, the inventors evaluated a number of tethering approaches in HeLa cells due to their easier genetic manipulation. Trastuzumab is a soluble antibody, and like any other antibody it is derived from a membrane-bound immunoglobulin (mIg) via alternative splicing event that removes the transmembrane domain. Accordingly, the inventors recreated an mIg version of trastuzumab heavy chain by fusing the constant, the transmembrane, and the cytosolic domains of the human genomic IGHG1 locus to the antigen-binding heavy chain variable fragment (V_{H}) of Trastuzumab (Dodev, et al. 2014). However, transfection of HeLa cells with a construct expressing this modified heavy chain and the Trastuzumab K light chain (Dodev, et al. 2014), each under the control of a constitutive EF1A promoter, failed to generate surface expression of trastuzumab as evidenced by the lack of surface staining against the IgG1 region (FIG. 6a). It was previously shown that some classes of Igs require the presence of the BCR co-factors CD79A and CD79B for efficient surface expression (Dylke, et al. 2007; Venkitaraman, et al. 1991). While these studies indicate that surface localization of mIgG1 does not depend on CD79, they also show that co-expression of CD79 with mIgG1 results in mIgG1-CD79 BCR-complex formation (Venkitaraman, et al. 1991). Thus, it was unclear, based on the findings in the prior art, whether this dimerization might boost mIgG1 surface expression. Surprisingly, the inventors found that cotransfection of the mIgG1 heavy- and light chain-encoding genes together with CD79A- and CD79B-encoding genes into HeLa cells resulted in substantial increase in the surface localization of mIgG1 (FIG. 6b).

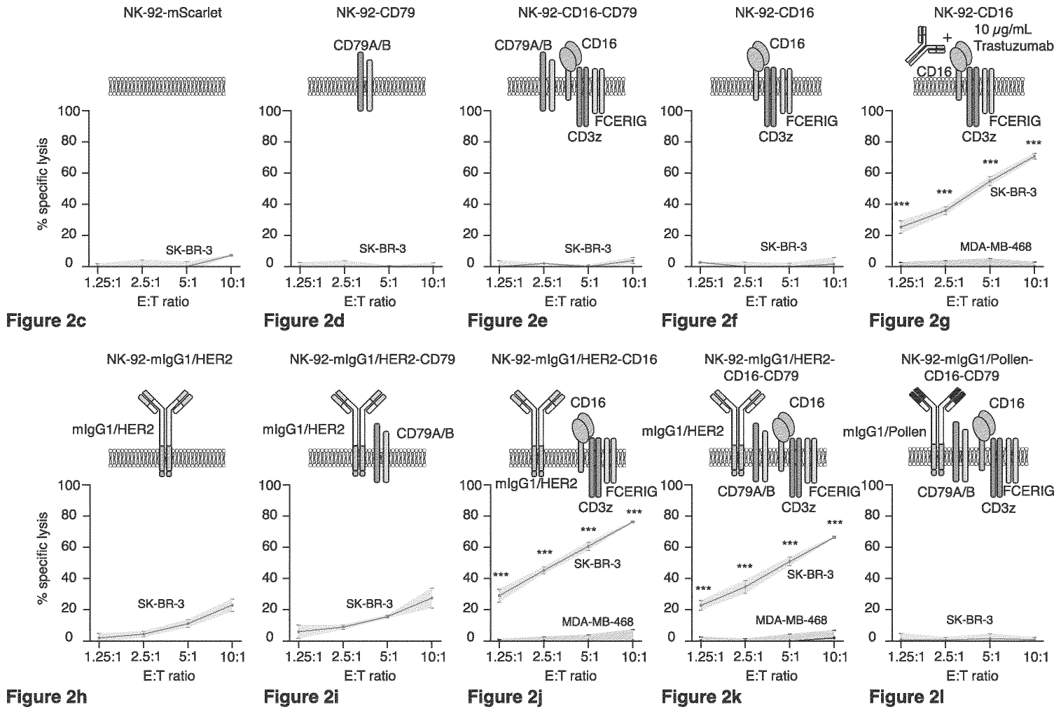

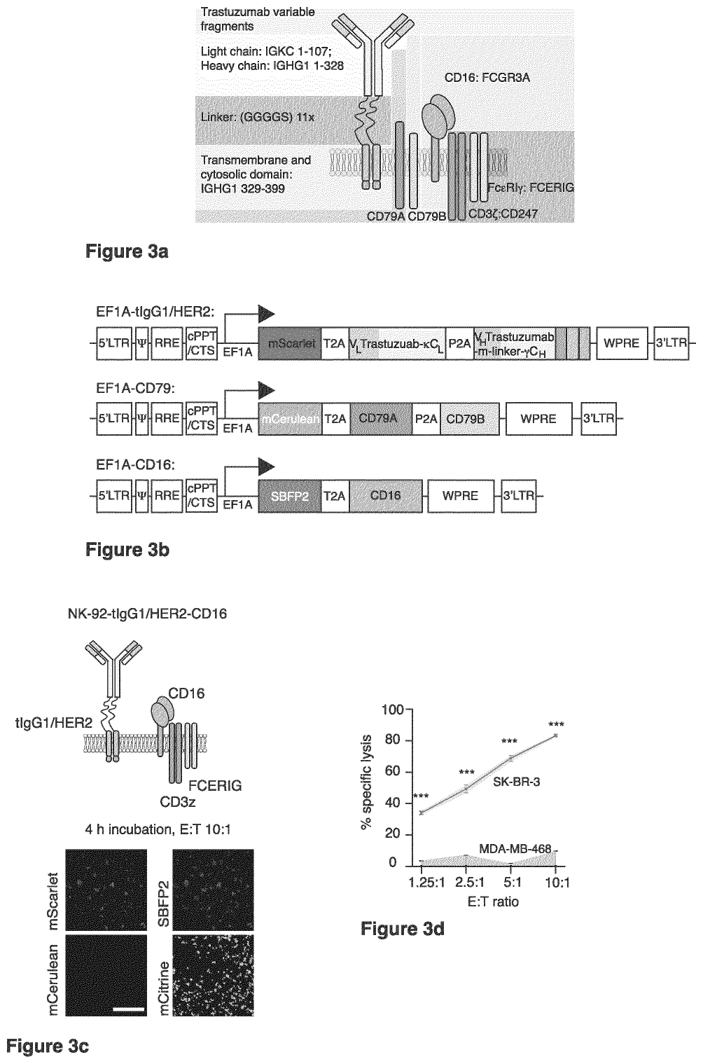

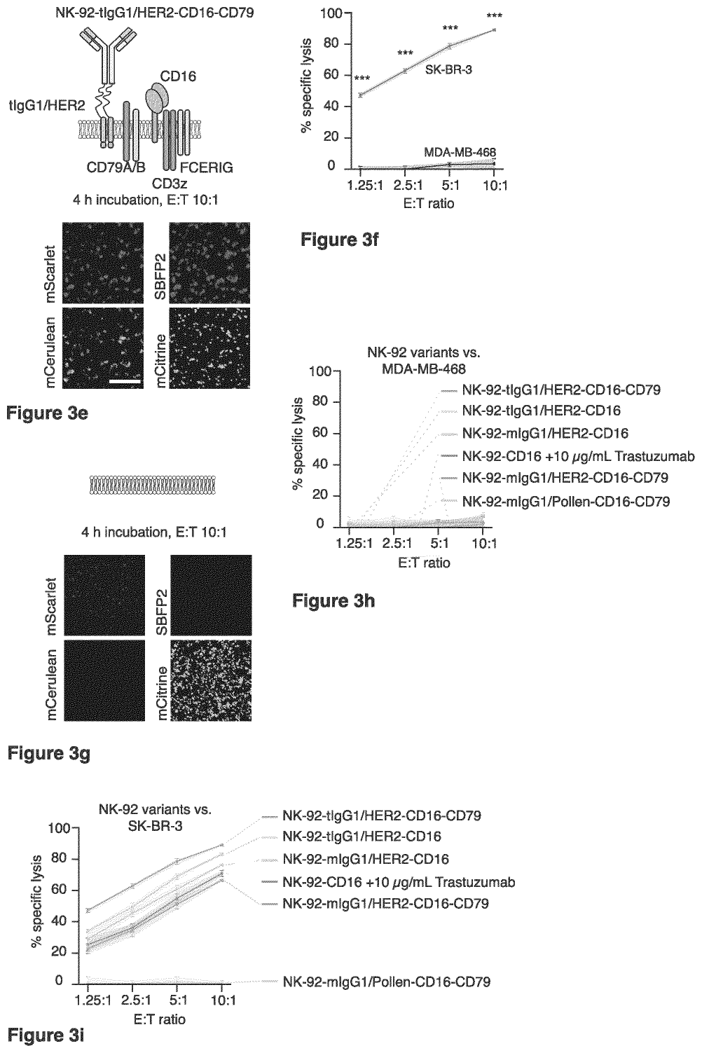

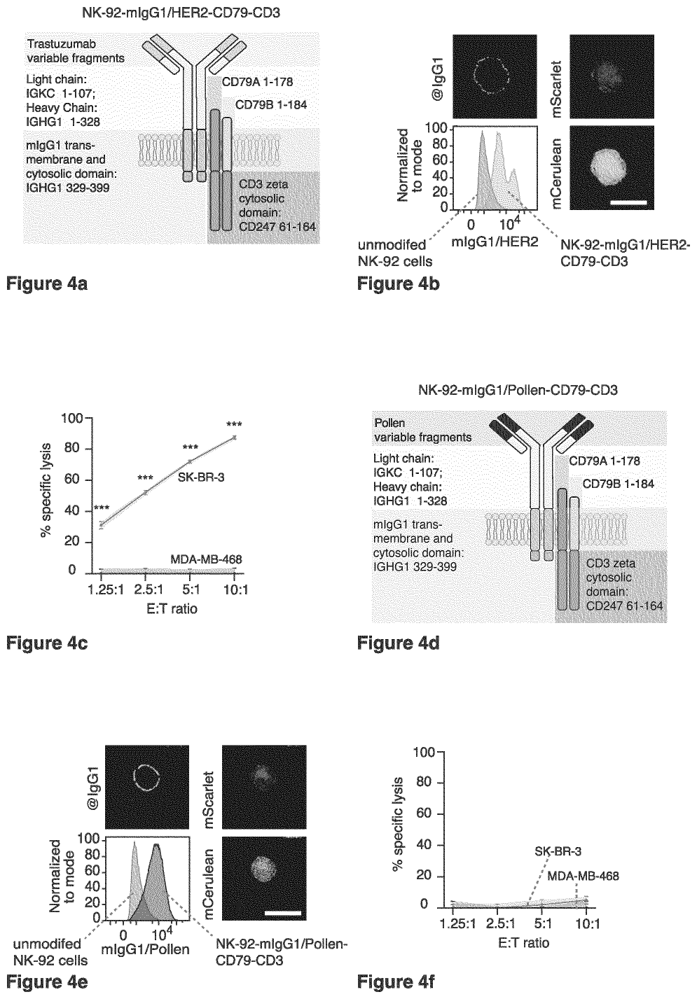

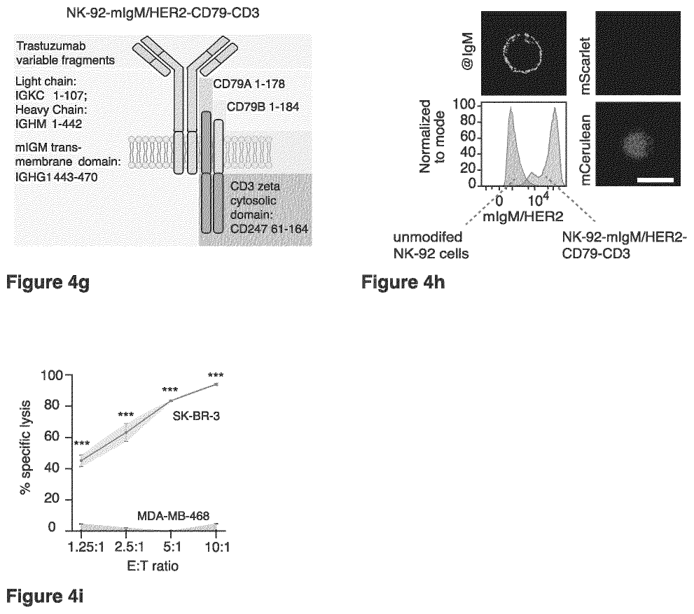

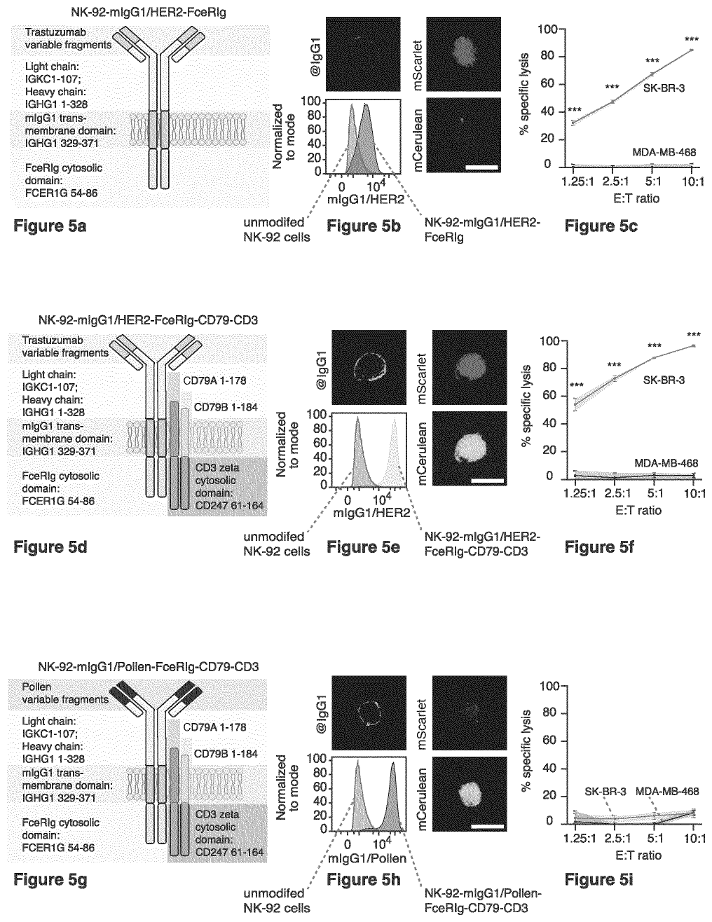

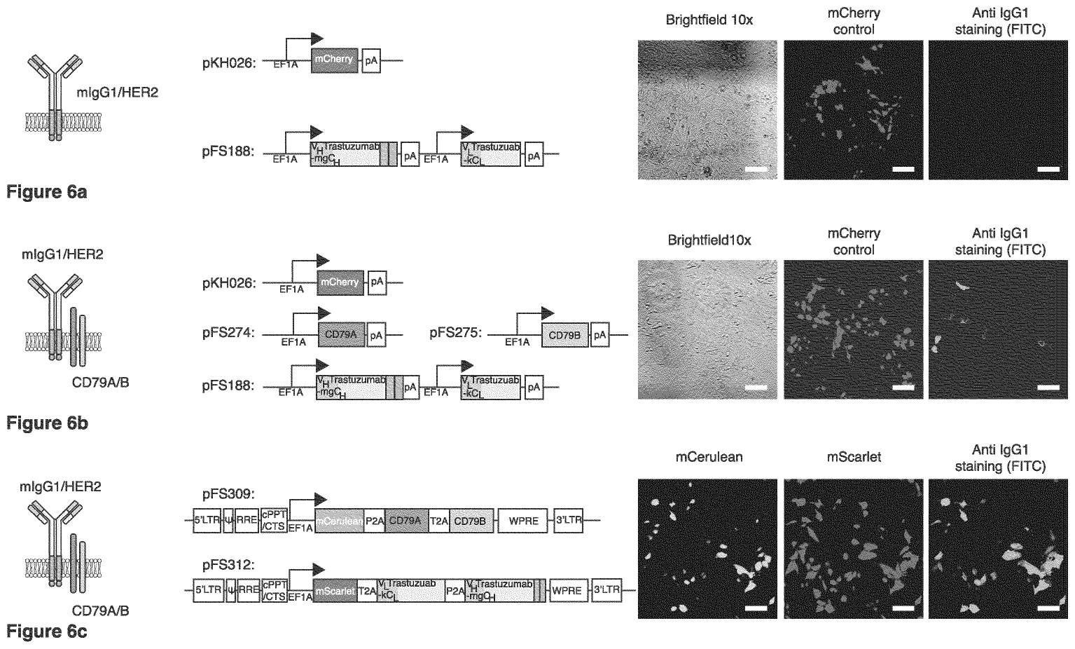

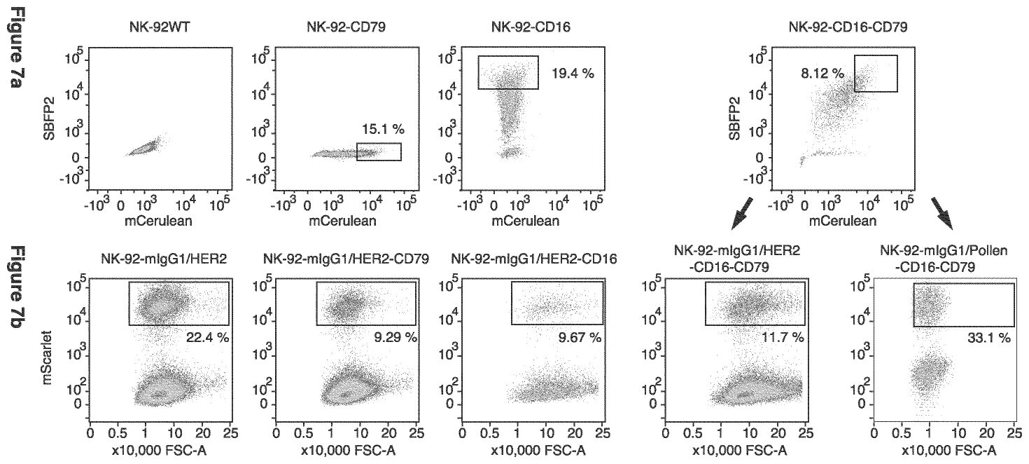

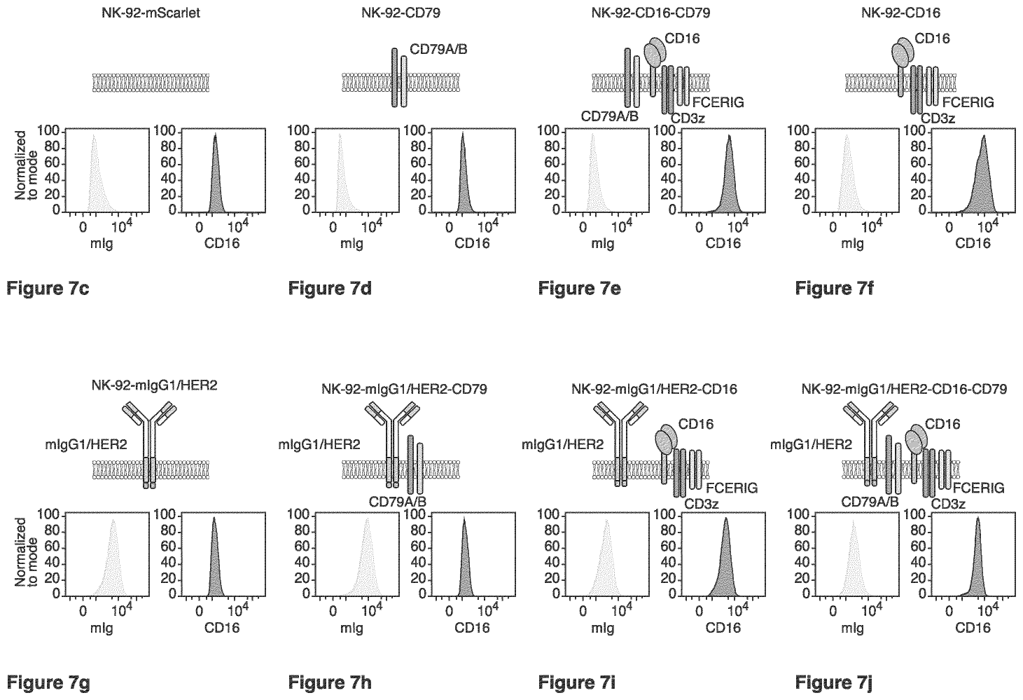

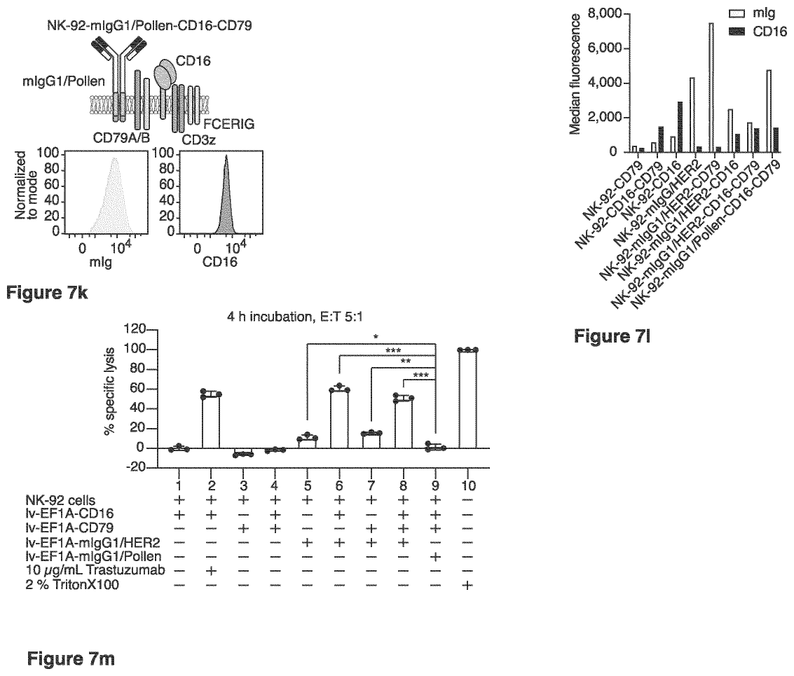

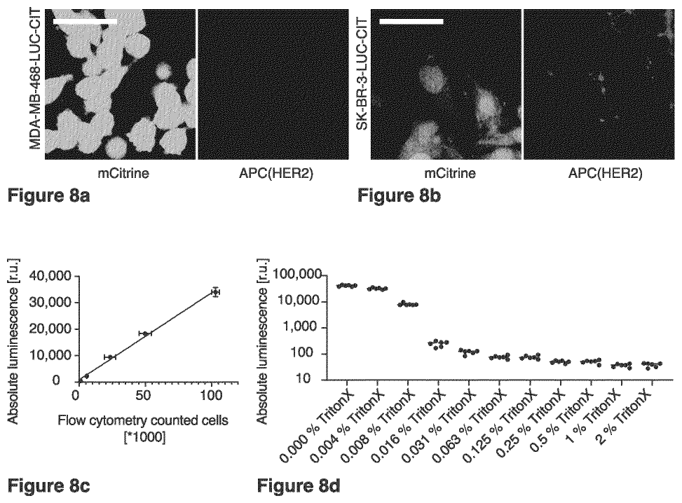

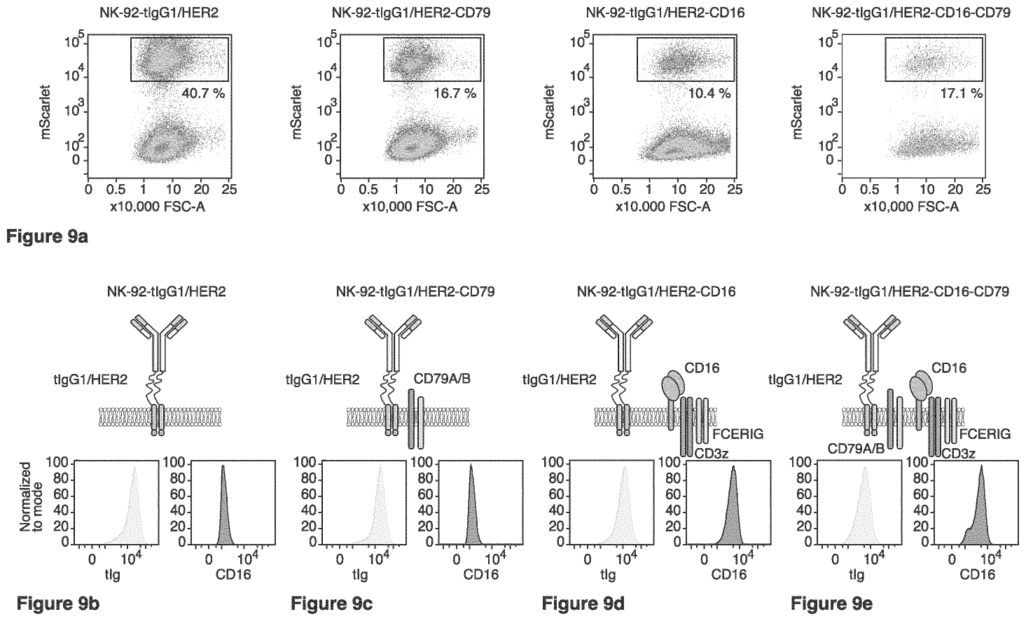

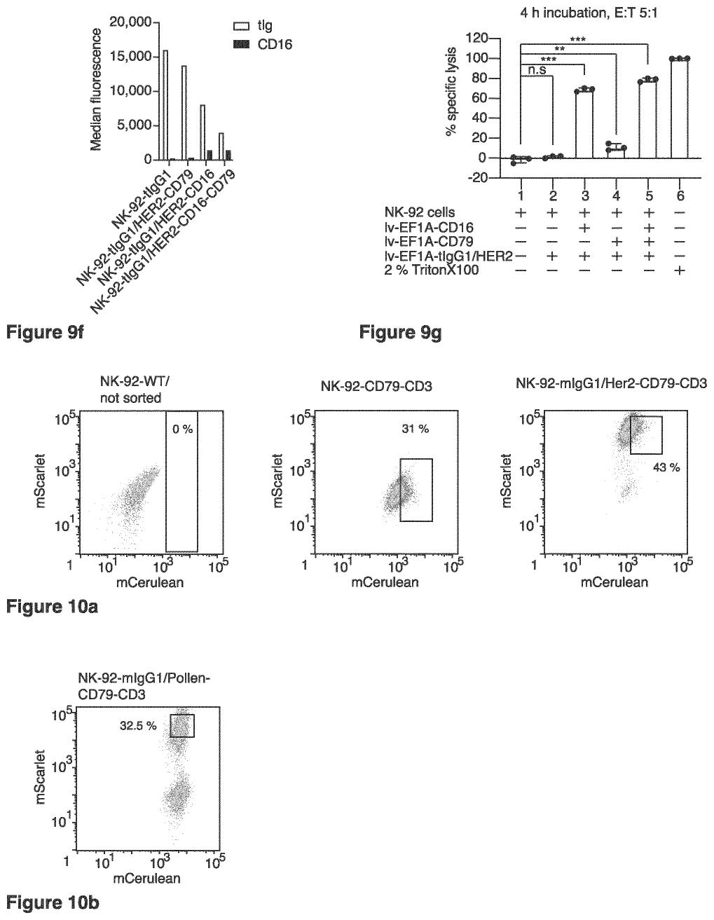

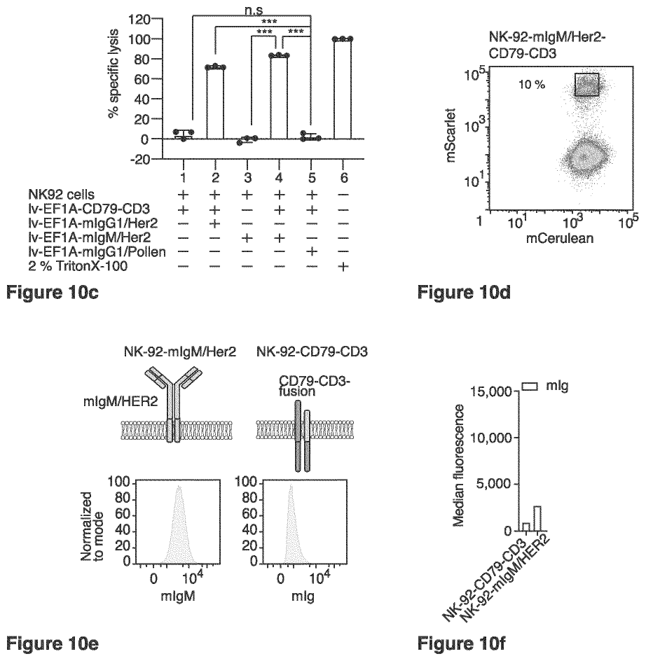

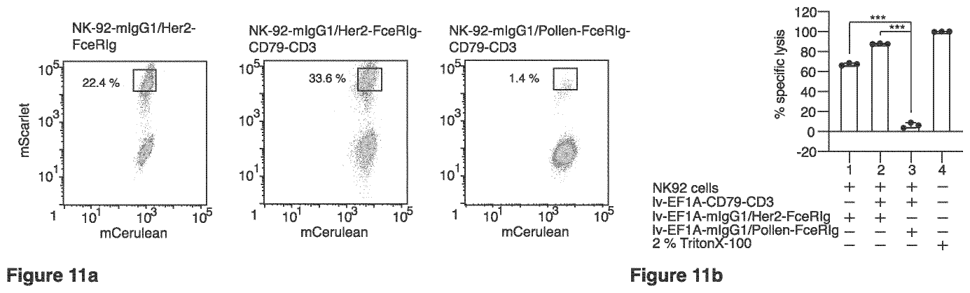

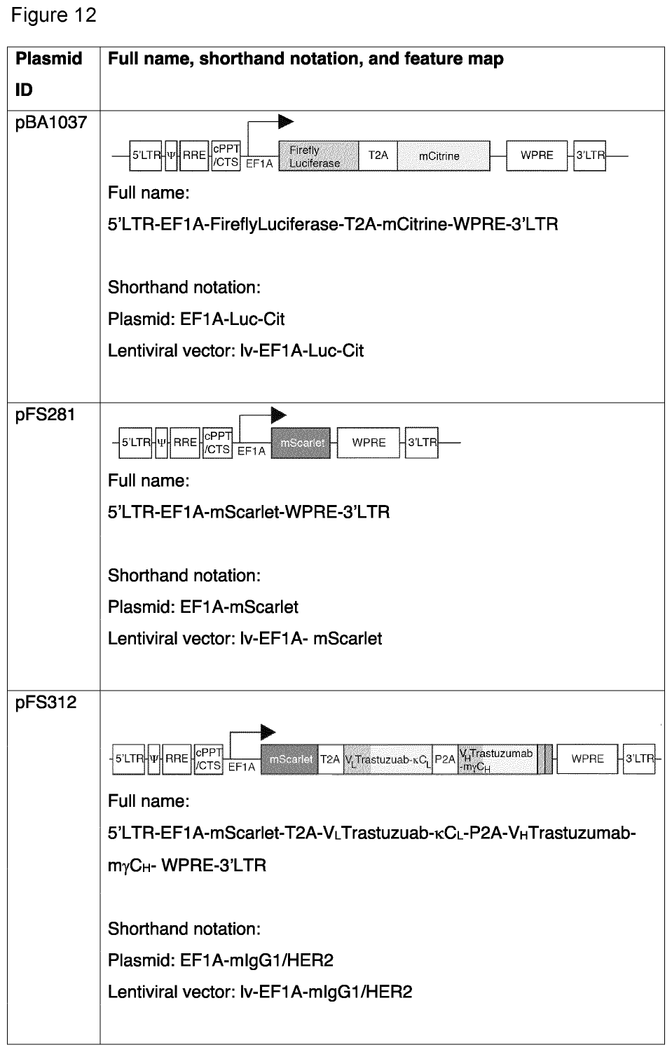

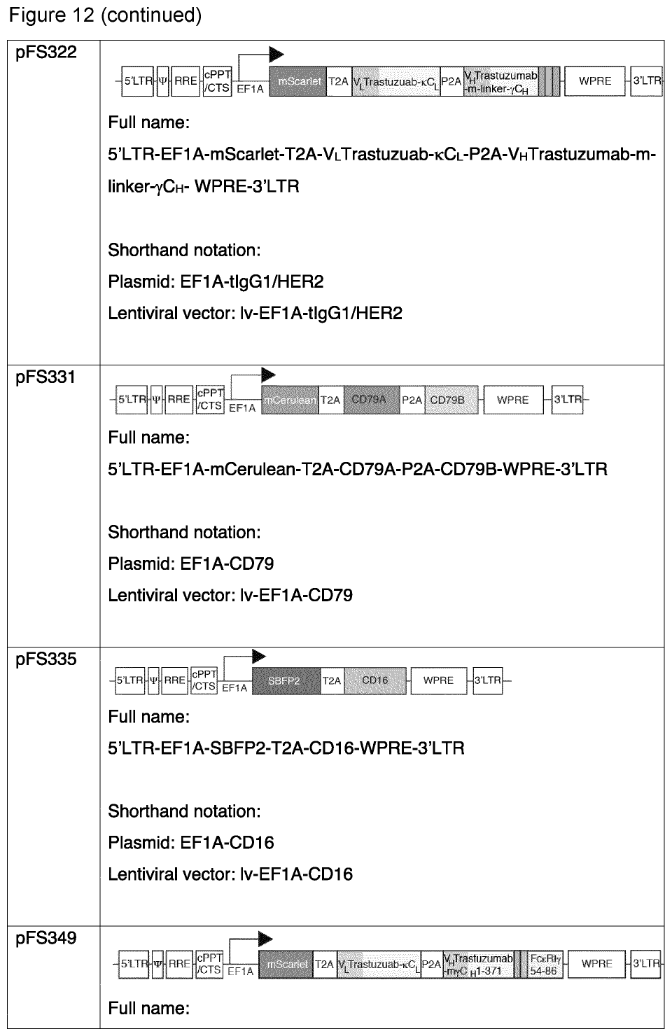

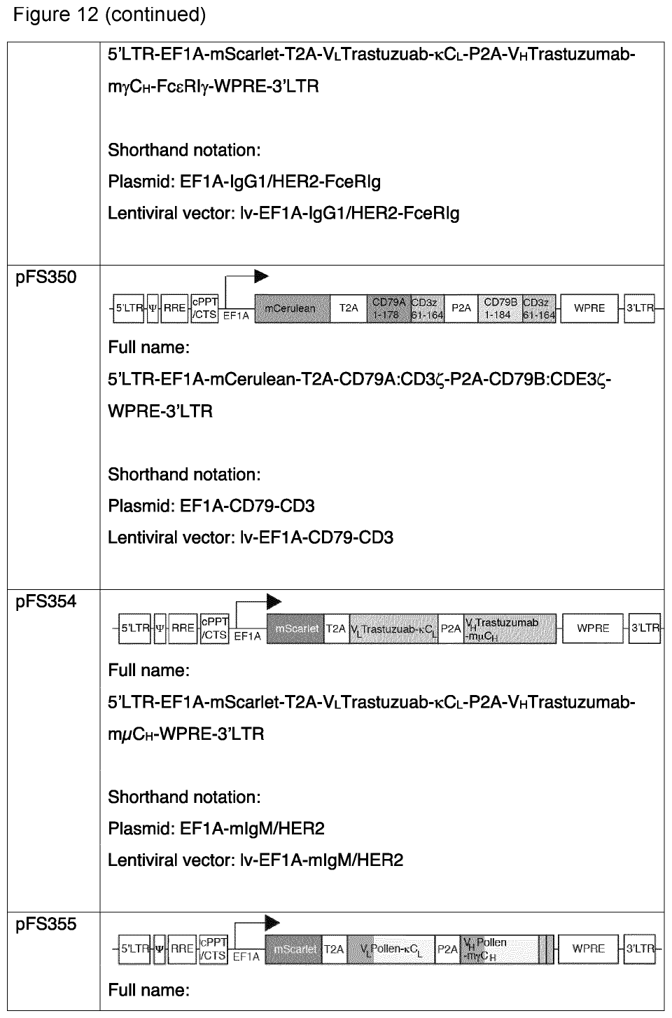

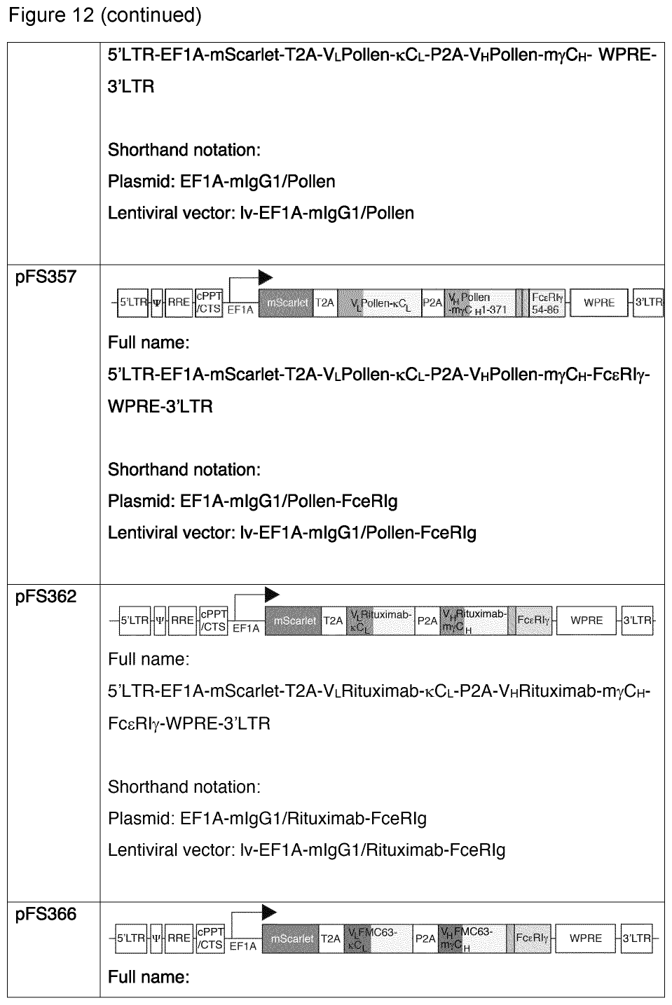

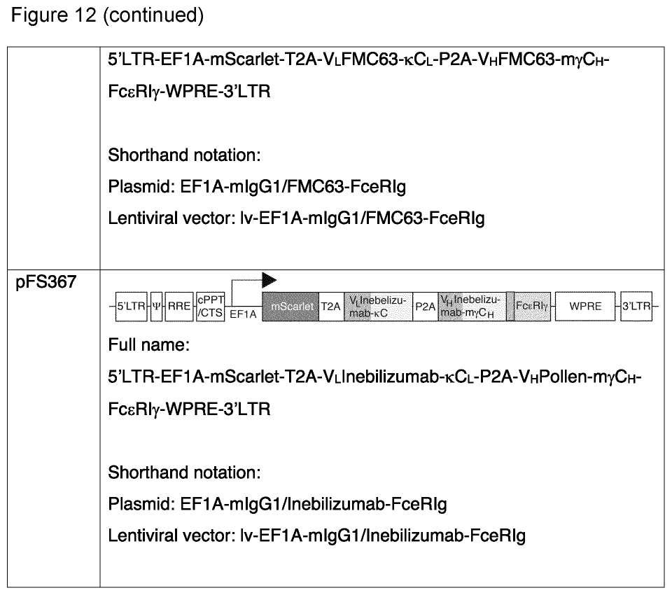

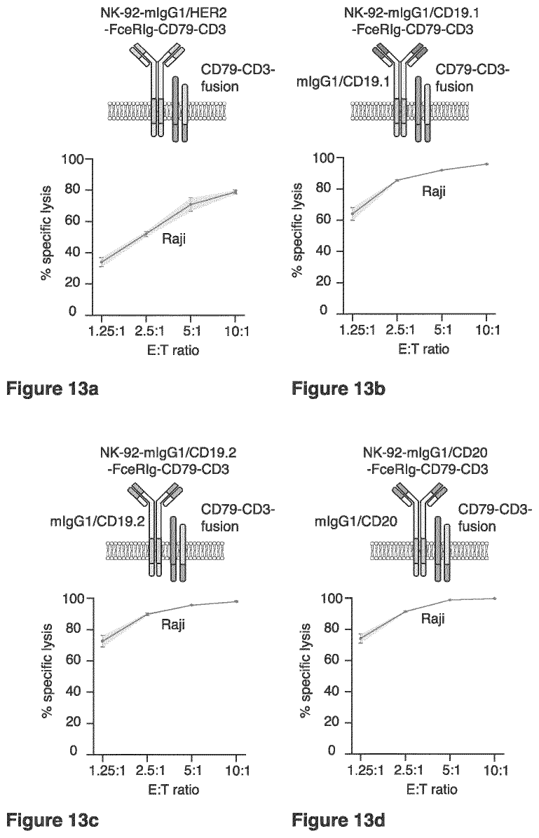

While the above experiments were done using plasmids and transient transfections, the inventors reasoned that clinical applications may require stable modifications of the NK cells. These modifications are usually implemented with the help of retroviral vectors (Colamartino, et al. 2019) and there is a need to reduce the number of vectors to simplify the manufacturing process. The inventors have solved this problem by adapting the constructs to lentiviral vector encoding, while minimizing the number of vectors. To this end, the inventors constringed the transcription of the anti-Her2 mIgG1 heavy and K light chain, on one hand, and the transcription of CD79A and CD79B, on the other, to single open reading frames in which the protein coding sequences were separated with 2A ribosome-skipping sites (Liu, et al. 2017; Ryan, et al. 1991). Merely to simplify the selection of transduced NK cells, the inventors added the coding sequences for fluorescent reporter proteins mScarlet and mCerulean at the 5′-end of anti-Her2 κlight chain/mIgG1 heavy chain construct, and the CD79A/CD79B construct, respectively. A third construct was built to encode CD16 in combination with the fluorescent reporter SBFP2 for CD16 overexpression in NK-92 cells (FIG. 2a). Unexpectedly, transient transfection of the mIgG1- and CD79A/CD79B-encoding constructs into HeLa cells resulted in mIgG1 surface expression (FIG. 6c), suggesting that the polycistronic cassettes embedded in lentiviral backbone were functional.

Overall, these surprising results show that a soluble monoclonal antibody can be modified to become a membrane-bound immunoglobulin, and suggest that the degree of its surface expression, at least in HeLa cells, can be increased by the co-expression of CD79A and CD79B (for details about the constructs and lentiviral vectors reference is made to FIG. 12).

### Example 3: Stable NK-92 Cell Line Transduction

After the surprising finding that an antibody can be immobilized to the cell membrane of HeLa cells, the inventors considered the NK cell model, namely the NK-92 cell line, a benchmark cell line for ADCC testing (Clémenceau, et al. 2013). All constructs were packaged into baboon-pseudotyped lentiviral vectors that transduce NK-92 cells with high efficiency (Colamartino, et al. 2019). As the NK-92 cells do not endogenously express the Fcγ receptor CD16 (Gong, et al. 1994) required for ADCC, it was initially unclear whether exogenous CD16 overexpression would be required in addition to the antibody and the CD79A/CD79B components (FIG. 2b) or not, in order to recapitulate cis ADCC (Colamartino, et al. 2019). To investigate this question, the inventors generated seven variants of NK-92 cells transduced with all possible subsets of the three constructs. NK-92 cells transduced with a vector expressing constitutive mScarlet were used as a negative control. An additional negative control cell line was created by exchanging the variable fragment of the antibody in NK-92-mIgG1/Her2-CD16-CD79 with an anti-pollen antigen variable Fragment (Fv) (Dodev, et al. 2014). To generate the stable cells lines, the inventors first transduced NK-92 cells with the CD79 and CD16-encoding lentiviruses (where needed) and sorted them for high expression of mCerulean and/or SBFP2, respectively (FIG. 7a). In the second step, the wild-type NK-92 cells and the three sorted cell lines were either transduced with the antibody-encoding constructs and sorted for high mScarlet expression (FIG. 7b), or used as is (for cell line details reference is made to Table 1).

To evaluate the functionality of the lentiviral vectors, the inventors measured surface expression of mIgG1 and CD16 on the transduced NK-92 cell lines (FIG. 7c-l). Based on the mIgG1 expression tests in HeLa cells (Figure B), surface expression of mIgG1 was expected only upon co-expression with CD79 co-factors. Very surprisingly, however, the inventors found a population of cells positive for mIgG1 surface expression among the NK-92 cells transduced with only the mIgG1-encoding vector (FIG. 7g), even though NK-92 cells are not known to express either CD79A or CD79B endogenously (Chu and Arber 2001). With that, the inventors still observed an increase in surface expression of mIgG1 upon co-expression of CD79 (FIG. 7h). As a side remark, simultaneous co-expression of CD16 and mIgG1 led to a decrease in surface localization of the mIgG1, indicating interference between the immunoglobulin and CD16 (FIG. 7i). However, all NK-92 cells that were transduced with an mIgG1 and/or a CD16 encoding vectors, had detectable surface expression of these proteins, and cis-ADCC was always observed, indicating that such potential interference is no problem.

### Example 3: Efficient Cis-ADCC by NK-92 Cells Transduced with mIgG1 and CD16

The inventors assessed the cytotoxicity of the modified NK-92 cell lines by luminescence-based cytolytic assay (LCA) (Brown, et al. 2005), using luciferase-modified target cells and luciferase activity as a measure of cell viability. To this end, NK-92 resistant breast cancer cell lines SK-BR-3 (Her2 positive) (Trempe 1976) and MDA-MB-468 (Her2 negative) (Chavez, et al. 2011) were stably transduced with a VSV-G lentiviral vector encoding a constitutively driven firefly luciferase and mCitrine coding sequences connected by the 2A linker, sorted for Citrine expression, and stained to verify Her2 surface expression (FIG. 8a, b). Linear correlation between cell number and luciferase signal was likewise confirmed (FIG. 8c, d). Cytotoxic activity of modified NK-92 cells was evaluated after a 4 hour co-incubation between the target and the NK-92 cells at effector to target cell ratios (E:T ratios) of 10:1, 5:1, 2.5:1 and 1.25:1 (FIG. 2c-1). At a 10:1 E:T ratio, NK-92-mIgG1/Her2-CD16 and NK-92-mIgG1/Her2-CD16-CD79 cells reached lysis levels of 76% and 67%, respectively (FIG. 2j,k). Surprisingly, these results were comparable to the 71% lysis measured for the control condition of the canonical ADCC where NK-92-CD16 cells were supplied with 10 μg/mL of soluble Trastuzumab at an E:T ratio of 10:1 (FIG. 2g). For all cytotoxic cell lines, lysis increased in an effector cell number-dependent manner. None of the mIgG1-negative control cell lines (NK92-mScarlet, NK-92-CD79, NK-92-CD16, nor NK-92-CD16-CD79) (FIG. 2c-f), nor the control cell line NK-92-mIgG1/Pollen-CD16-CD79 (FIG. 2l), triggered any substantial lysis of SK-BR-3 cells at any tested effector to target cell ratio. The cell lines that elicited cytotoxicity against Her2-positive cells were nevertheless inactive against Her2-negative cell line MDA-MB-468 with less than 10% lysis even at the highest E:T ratio (FIG. 2g, j, k), confirming antigen specificity of the effector cells. Remarkably and unexpectedly, NK-92-mIgG1/Her2 and NK-92-mIgG1/Her2-CD79 cells that express mIgG1/Her2 but lack CD16, eliminated up to 27% of Her2 positive target cells (FIG. 2h, i), indicating that mIgG1 may function on its own.

These surprising results show that mIgG1/Her2 can be expressed on the surface of NK-92 cells and, in cooperation with CD16, induce strong antigen-specific cis ADCC against target cells. It is likely that the strongly-responding cell lines rely on CD3ζ and FcεRIγ signaling domains, associated with the over-expressed CD16, for their cytotoxicity. However, the surprising findings of the inventors also suggest that these domains may not be engaged in CD16-negative cells, and therefore, those cells may rely on signaling domains typically active in B-cells, such as mIgG1 cytosolic tail and CD79 ITAMs present in the transduced constructs and able to signal with the help of the associated Syk kinase (Dal Porto, et al. 2004), resulting in a less pronounced but still considerable and measurable cytotoxicity.

### Example 4: Increased Immunoglobulin Membrane Distance Using a Tethering GS Linker

Conventionally, under physiological conditions ADCC is triggered upon the contact initiation of an NK cell with an antibody-coated target cell via the interaction of the membrane-bound antibody and CD16 (FIG. 1a). In the context of the present invention, the inventors observed reduced surface localization of mIgG1 in NK-92 cells when cotransduced with CD16. Thus, the inventors asked whether this might be caused by non-specific interactions between mIgG1 and CD16 that could lead to mIgG1 internalization (Al Qaraghuli, et al. 2020; Caballero, et al. 2006; Sun, et al. 2021).

However, it was completely unclear whether by increasing the distance between the mIgG1 constant domain and CD16, the inventors would be able to increase mIg surface localization, and facilitate efficient effector target cell interaction and thus, increase target cell lysis. To address this question, the inventors chose a flexible (GGGGS)₁₁ linker of almost 21 nm, which is comparable to twice the size of extracellular vertical protrusion of a Fcγ-IgG complex (Chen, et al. 2013; Patel, et al. 2019). In the context of the invention, this construct is also called “tethered IgG1” (tIgG1, FIG. 3a, b). In view of the surprising finding that all four mIgG1/Her2-expressing NK-92 cell lines showed cytotoxic activity (FIG. 2h-k), the inventors built analogous cell lines substituting mIgG1/Her2 with tIgG1/Her2 using similar cell transduction and sorting strategy (FIG. 9a). Surprisingly, all four NK-92 cell lines transduced with tIgG1 (NK-92-tIgG1/Her2, NK-92-tIgG1/Her2-CD16, NK-92-tIgG1/Her2-CD79, and NK-92-tIgG1/Her2-CD16-CD79) showed strong surface expression of tIgG1 (FIG. 9b-f). Additionally, and also surprisingly, CD16 and tIgG1 positive NK-92 cell lines displayed high cellular cytotoxicity (FIG. 3c-f, and FIG. 9g). NK-92-tIgG1/Her2-CD16 cells elicited 84% lysis at an E:T ratio of 10:1 (FIG. 3c, d) and NK-92-tIgG1/Her2-CD16-CD79 cells eliminated 91% of SK-BR-3 cells at the same E:T ratio (FIG. 3e, f). Both of these cell lines only showed background activity against MDA-MB-468 (FIG. 3d, f). When the inventors analyzed surface expressions in NK-92-tIgG1/Her2-CD16 and NK-92-mIgG1/Her2-CD16 with similar mScarlet expression modes of 6747 relative fluorescent units (r.f.u) and 6516 r.f.u. (data not shown), respectively, a tIgG1 surface expression mode of 8045 r.f.u. and an mIgG1 surface expression mode of 2514 r.f.u. was observed (compare FIG. 7i and FIG. 9d). This difference in surface expression translated into an increase in cytotoxicity of 10% (p-value=0.00015) (FIGS. 3h, i). In case of NK-92-mIgG1/Her2-CD16-CD79 and NK-92-tIgG1/Her2-CD16-CD79, similar comparison may be not entirely unproblematic, due to lower mScarlet levels in NK-92-mIgG1/Her2; nevertheless, the tIgG1-encoding cells resulted in 89% cell lysis compared to 67% lysis in mIgG1-encoding cells. Notably, and unexpectedly, in NK-92-tIgG1/Her2-CD16-CD79 the inventors measured a target cell lysis of 45% at an E:T of 1.25:1, which surpassed canonical ADCC with NK92-CD16 and 10 μg/mL Trastuzumab by 2-fold (FIG. 3i), although the mode tIgG1 surface expression was only 4075 r.f.u. For NK-92 cells without CD16 (NK-92-tIgG1/Her2 and NK-92-tIgG1/Her2-CD79), adding the linker did not provide an increase in cytotoxic responses compared to the non-linker IgG1 versions (compare FIGS. 2h-l at E:T 5:1 and FIG. 9g bars 2 and 4).

These surprising results show that a flexible GS-linker can be added between the transmembrane and the constant domains of an IgG1 antibody. Unexpectedly, this linker-tethered IgG1 results in stronger immunoglobulin surface expression, and at least for NK-92-tigG1-CD16, triggers a significantly stronger cytotoxic response than the mIgG1/Her2 counterpart at comparable antibody expression levels. Furthermore, and also unexpectedly, Her2-specific cytotoxicity obtained with a cis-ADCC on NK-92-tIgG1-CD16-CD79 dramatically surpassed canonical ADCC (FIGS. 3h, i).

### Example 5: From Recapitulating ADCC to a Modular Receptor Architecture

The inventors have observed a moderate cytotoxic effect with NK-92-mIgG1/Her2-CD79 cells (FIG. 2i). A combination of an mIgG1 and CD79A/CD79B reconstitutes a B-cell receptor (BCR) complex. In view of the surprising findings shown in Examples 1 to 4, the inventors thought that BCR-like complexes may be able to trigger cytotoxicity when expressed in NK cells without the need for CD16 (Gong, et al. 1994). Therefore, the inventors addressed the question whether it would be possible to transform these complexes into a multi-chain antigen receptor architecture that does not rely on CD16 yet elicits strong cytotoxicity comparable to CD16-expressing NK cells. The inventors envisioned this Antigen-specific Synthetic Immunoglobulin-based Multi-chain (ASIMut) receptor platform fulfilling two requirements: i) modular control over the signaling domains, to fine tune the downstream response; and ii) high modularity of the immunoglobulin constant module, in order to be able to control possible interactions with Fc receptors like CD16. As a proof of concept, the inventors decided to endow this receptor scaffold with the singling domains that are normally associated with CD16, in order to induce ADCC-like cytotoxicity in NK-92 cells without overexpressing CD16 per se.

First, the inventors asked whether it would be possible to replace CD79 cytosolic domains with the CD3ζ-derived ITAMs to trigger strong cytotoxic responses without the need for CD16. To this end, the inventors replaced the CD79A¹⁷⁹⁻²²⁶ and CD79B¹⁸⁵⁻²²⁹ cytosolic domains with the CD3ζ⁶¹⁻¹⁶⁴ domain containing three ITAM motifs, resulting in the fusion constructs CD79¹⁻¹⁷⁹::CD3ζ⁶¹⁻¹⁶⁴ and CD79⁶¹⁻¹⁸⁵::CD3ζ⁶¹⁻¹⁶⁴ (CD79-CD3), i.e. CD79-CD3 zeta chimeric polypeptides (FIG. 4a).

Modifying the CD79A and CD79B cytosolic tails did not interfere with mIgG1/Her2 surface localization, namely, NK-92 cells transduced with mIgG1/Her2, and CD79-CD3ζ (CD3 zeta) chimeric polypeptides (FIG. 10a) exhibited an mIgG1 surface expression (FIG. 4b) that was similar to NK-92-mIgG1-CD79 cells (FIG. 7h). Of note, in these experiments, the inventors relied on mIgG1 instead of tigG1 to guarantee correct formation of the immunological synapse (Tsourkas, et al. 2007). Surprisingly, the cells expressing the hybrid receptor triggered 87% lysis of SK-BR3 cells at E:T 10:1 (FIG. 4c), comparable to NK-92-tIgG1/Her2-CD16-CD79 (compare FIG. 4c to FIG. 3i), and cytotoxicity levels against MDA-MB-468 below 5% (FIG. 4c). This cytotoxic response shows that CD79A and CD79B cytosolic chains can be exchanged with other signaling domains in order to increase cytotoxicity while eliminating the need for CD16 overexpression, and thus fulfilling our first requirement. To validate Her2 antigen specificity of this receptor, an identical receptor containing an anti-pollen variable fragment was built and transduced into NK-92 cells (FIG. 4d-e, FIG. 10b). Although the cell line exhibited comparable surface expression of mIgG1 (compare FIG. 4b to FIG. 4e), it lysed less than 5% of SK-BR-3 cells (FIG. 4f). A cell line expressing CD79-CD3 zeta fusions alone lysed less than 10% of target cells (FIG. 10c).

Next, the inventors assessed the modularity of the constant immunoglobulin domain. All previous NK-92 variants used in the experiments described above relied on CD16 and therefore were, possibly, limited to an IgG class immunoglobulin due to a presumed ADCC dependency on an IgG constant domain for CD16 binding. The inventors wondered whether removal of CD16 component may have rendered the requirement for an IgG constant domain obsolete. Hence, the inventors asked whether this would enable the use of non-IgG constant domains without compromising receptor functionality while increasing design flexibility and avoiding receptor CD16 interactions in CD16^{POS} NK cells. As a proof of concept, the inventors exchanged the heavy chain constant domain of the mIgG1 in mIgG1/Her2 construct with an IgM class domain (mIgM/Her2) (FIG. 4g). Another reason for testing IgM was to assess whether increasing the distance between the effector and target cell due to a longer protein would still preserve the effect (Schroeder and Cavacini 2010), in particular because a reduction was observed under similar circumstances in the context of other synthetic immune receptors (Hombach, et al. 2007). Lastly, IgM class immunoglobulins are known to have a higher dependency on CD79 co-factors for efficient surface expression (Venkitaraman, et al. 1991) compared to mIgG1. However, it was unclear whether this might lead to a better controllability of immunoglobulin surface expression via the CD79 component.

First, the inventors transduced NK-92 cells with a vector encoding a constitutive mIgM/Her2 (FIG. 10d). In an immunostaining assay against the IgM in these cells, only 15% were positive for immunoglobulin surface expression (FIG. 10e-f). Surprisingly, however, when these cells were co-transduced with Iv-EF1A-CD79-CD3 zeta chimeric polypeptides, surface localization of IgM increased to 93% (FIG. 4h). Further surprisingly, in a cell killing assay using these NK-92-mIgM/Her2-CD79-CD3 zeta cells, 92% lysis of SK-BR-3 was measured at a 10:1 E:T cell ratio (FIG. 4i). Moreover, lysis of 43% at E:T of 1.25:1 was among the strongest responses measured for any of the receptors.

Similar to the use of CD79 cytoplasmic domains as an attachment point for signaling domains, the inventors further inquired whether the cytoplasmic domains of the antibody constructs could be used as “slots” to introduce additional domains, potentially strengthening the overall effect. Mindful of the fact that ADCC and CD16 also rely on FcεRIγ (sometimes in synergy with a CD3 zeta domain), the inventors decided to address that question by attaching FcεRIγ signaling domain to the antibody.

While this domain was used as a part of a CAR in NK-92 cells by Clemensau et al. (Clëmenceau, et al. 2015), the combination of FcεRIγ and CD3ζ within one antigen-specific receptor in NK-92 cells has not been assessed previously. To this end, the inventors introduced the FcεRIγ ITAM domain immediately after the −KVK motif at the transition of the mIgG1-Her2-transmembrane to cytosolic domain (FIG. 5a). The inventors kept the −KVK motif intact, in particular, to any potential avoid mIgG1 surface expression issues, as it is the conserved minimal cytosolic tail of various mIg classes (Cambier and Campbell 1989). The inventors first transduced NK-92 cells with an Iv-EF1A-mIgG1-FceRIg/Her2 chimeric polypeptide (FIG. 11a, b) and quantified the surface expression of the fusion mIgG1. Surprisingly, 24% of NK-92 cells transduced with the FcεRIγ-fused mIgG1 chimeric polypeptide were positive for mIgG1 surface localization without co-expression of CD79 (FIG. 5b). Moreover, and also surprisingly, the expression of this FcεRIγ-fused antibody in NK-92 cells was sufficient to induce a substantial cytotoxic response against target cells (FIG. 5c). With 84% lysis at a 10:1 E:T ratio and 32% at a 1.25:1 ratio, the cell death remained lower than for NK-92-tIgG1/Her2-CD16-CD79 (compare FIG. 5c to FIG. 3i), but, unexpectedly, still higher than canonical ADCC (compare to FIG. 3i). Remarkably, and also unexpectedly, additional transduction of this cell line with CD79-CD3 zeta chimeric polypeptides (FIG. 11a) further increased the immunoglobulin surface expression to 95% (FIG. 5d, e) and increased the lysis to 96% at a 10:1 E:T ratio (FIG. 5f). This was by far the strongest response seen for any of the receptor designs, especially when considering the cytotoxic capacity at 1.25:1 E:T ratio, with lysis of 54% of the target cells (FIG. 5f). Her2 specificity was preserved, since co-culturing with MDA-MB-468 did not result in target cell lysis above background levels (FIG. 5f). A Fab fragment exchange from anti-Her2 to an anti-Pollen Fab fragment (FIG. 11a) resulted in similar surface expression (FIG. 5g, h), but led to the loss of target cell lysis in co-cultures with SK-BR-3 (FIG. 5i), suggesting antigen specificity.

### Example 6. Alteration of Target Specificity of Modular Multi-Chain Receptors

To further confirm that the antigen-specificity of the multi-chain receptors of the invention, e.g., receptors based on the ASIMut receptor platform described in Example 5, can be switched to kill different target cells as desired, the inventors replaced the variable fragments of the receptor shown in FIG. 5d by the variable fragments of antibodies against CD19.1 (i.e. FMC63), CD19.2 (i.e. Inebilizumab) or CD20 (i.e. Rituximab); see SEQ ID NO: 433, 435 and 437 and FIG. 12. To this end, the inventors targeted CD19 and CD20 positive Raji cells which originate from a B-cell malignancy with the original anti-HER2 ASIMut receptor shown in FIG. 5d (see FIG. 13a) or these altered receptors (having specificity against CD19.1, CD19.2 or CD20; see FIG. 13b-d). The experiments have been performed the same way as described herein above, in particular in context of FIG. 5, c, f and i.

It has been found that the multi-chain antigen receptors which had a variable domain from an anti-CD19.1 antibody, an anti-CD19.2 antibody or an anti-CD20 antibody killed the target cells, i.e. Raji cells, very efficiently and more efficiently than the anti-HER2 receptor which had a variable domain from trastuzumab. Of note, the observed killing activity of the anti-HER2 multi-chain antigen receptor against Raji cells (FIG. 13a) is likely not mediated by antigen-specific binding (i.e. HER2 binding) but may rather relate to the intrinsic killing activity of NK cells against these cells, i.e. the background target cell lysis in this experiment.

Hence, these experiments confirm that the specificity of multi-chain antigen receptors, e.g. ASIMut receptors, can be changed and other antigen expressing tumors or cancer cells can be killed by modified cells (e.g. NK cells) expressing altered receptors containing corresponding antigen binding sites.

Sequence listing (partial)
For the complete sequence listing, please see the attached
sequence listing pursuant to WIPO St. 26.

| SEQ | Organ- | | | |
|---|---|---|---|---|
| ID NO. | ism | Type | Name | Sequence |
| 1 | Art | Prt | CD79A - Motif of | EXXXXXXXXXXP |
| | | | interface region of | |
| | | | membrane domain | |
| 2 | Art | Prt | CD79B - Motif of | QXXXXXXXXXXP |
| | | | interface region of | |
| | | | membrane domain | |
| 3 | Human | DNA | CD79A - Interface | GAAGGCATTATCCTCTTGTT |
| | | | region of membrane | TTGCGCAGTTGTGCCG |
| | | | domain | |
| 4 | Human | Prt | CD79A - Interface | EGIILLFCAVVP |
| | | | region of membrane | |
| | | | domain | |
| 5 | Human | DNA | CD79B - Interface | CAAACTCTCTTGATCATTTT |
| | | | region of membrane | GTTTATCATCGTCCCT |
| | | | domain | |
| 6 | Human | Prt | CD79B - Interface | QTLLIILFIIVP |
| | | | region of membrane | |
| | | | domain | |
| 7 | Human | DNA | CD79A - Membrane | see attached sequence listing |
| | | | domain | pursuant to WIPO St. 26 |
| 8 | Human | Prt | CD79A - Membrane | IITAEGIILLFCAVVPGTLLLF |
| | | | domain | |
| 9 | Human | DNA | CD79B - Membrane | see attached sequence listing |
| | | | domain | pursuant to WIPO St. 26 |
| 10 | Human | Prt | CD79B - Membrane | GIIMIQTLLIILFIIVPIFLL |
| | | | domain | |
| 11 | Human | DNA | CD79A - | see attached sequence listing |
| | | | Extracellular domain | pursuant to WIPO St. 26 |
| 12 | Human | Prt | CD79A - | see attached sequence listing |
| | | | Extracellular domain | pursuant to WIPO St. 26 |
| 13 | Human | DNA | CD79B - | see attached sequence listing |
| | | | Extracellular domain | pursuant to WIPO St. 26 |
| 14 | Human | Prt | CD79B - | see attached sequence listing |
| | | | Extracellular domain | pursuant to WIPO St. 26 |
| 15 | Human | DNA | CD79A - full length | see attached sequence listing |
| | | | | pursuant to WIPO St. 26 |
| 16 | Human | Prt | CD79A - full length | see attached sequence listing |
| | | | | pursuant to WIPO St. 26 |
| 17 | Human | DNA | CD79B - full length | see attached sequence listing |
| | | | | pursuant to WIPO St. 26 |
| 18 | Human | Prt | CD79B - full length | see attached sequence listing |
| | | | | pursuant to WIPO St. 26 |
| 19 | Human | Prt | mlg - Large motif of | WXXXXXFXXLFXLXXXYSXXX |
| | | | interface region of | T |
| | | | membrane domain | |
| 20 | Human | Prt | mlg - Consensus | WTTITIFITLFLLSVCYSATVTF |
| | | | sequence of interface | F |
| | | | region of membrane | |
| | | | domain | |
| 21 | Human | Prt | mlgM - Fragment of | WATASTFIVLFLLSLFYSTTVT |
| | | | membrane domain | LF |
| 22 | Human | Prt | mlgG1 - Fragment of | WTTITIFITLFLLSVCYSATVTF |
| | | | membrane domain | F |
| 23 | Human | Prt | mlgG2 - Fragment of | WTTITIFITLFLLSVCYSATITFF |
| | | | membrane domain | |
| 24 | Human | Prt | mlgG3 - Fragment of | WTTITIFITLFLLSVCYSATVTF |
| | | | membrane domain | F |
| 25 | Human | Prt | mlgG4 - Fragment of | WTTITIFITLFLLSVCYSATVTF |
| | | | membrane domain | F |
| 26 | Human | Prt | mlgD - Fragment of | WTTLSTFVALFILTLLYSGIVTF |
| | | | membrane domain | I |
| 27 | Human | Prt | mlgE - Fragment of | WTWTGLCIFAALFLLSVSYSA |
| | | | membrane domain | AITLLMV |
| 28 | Human | DNA | mlgG1- Membrane | see attached sequence listing |
| | | | domain | pursuant to WIPO St. 26 |
| 29 | Human | Prt | mlgG1- Membrane | ELQLEESCAEAQDGELDGLW |
| | | | domain | TTITIFITLFLLSVCYSATVTFF |
| 30 | Human | DNA | mlgM- Membrane | see attached sequence listing |
| | | | domain | pursuant to WIPO St. 26 |
| 31 | Human | Prt | mlgM- Membrane | GFENLWATASTFIVLFLLSLFY |
| | | | domain | STTVTLF |
| 32 | Art | Prt | CD16 - Motif | FXXDT |
| | | | interface region of | |
| | | | membrane domain | |
| | | | (interaction with | |
| | | | FceRlg and CD3z) | |
| 33 | Human | DNA | CD16A - Interface | TTCGCAGTAGACACT |
| | | | region of membrane | |
| | | | domain (interaction | |
| | | | with FceRlg and | |
| | | | CD3z) | |
| 34 | Human | Prt | CD16A - Interface | FAVDT |
| | | | region of membrane | |
| | | | domain (interaction | |
| | | | with FceRlg and | |
| | | | CD3z) | |
| 35 | Human | DNA | CD16A - Membrane | see attached sequence listing |
| | | | domain | pursuant to WIPO St. 26 |
| 36 | Human | Prt | CD16A - Membrane | VSFCLVMVLLFAVDTGLYFSV |
| | | | domain | |
| 37 | Human | Prt | mlgA1 membrane | WPTTITFLTLFLLSLFYSTALT |
| | | | domain | VT |
| 38 | Human | Prt | mlgA2 membrane | WPTTITFLTLFLLSLFYSTALT |
| | | | domain | VT |
| 39 | Art | Prt | CD16 - Motif interface | IGWXXXXXXXXXXXXXXXXX |
| | | | region of extracellular | XXXXXWXXTAXHKXTXXXXX |
| | | | domain (interaction | XXRKYXHHXXXXXXXXXXXX |
| | | | with lg) | XXXXXXXRXXXGXK |
| 40 | Human | DNA | CD16A - Interface | see attached sequence listing |
| | | | region of extracellular | pursuant to WIPO St. 26 |
| | | | domain (interaction | |
| | | | with lg) | |
| 41 | Human | Prt | CD16A - Interface | IGWLLLQAPRWVFKEEDPIHL |
| | | | region of extracellular | RCHSWKNTALHKVTYLQNGK |
| | | | domain (interaction | GRKYFHHNSDFYIPKATLKDS |
| | | | with lg) | GSYFCRGLFGSK |
| 42 | Human | DNA | CD16A - Extracellular | see attached sequence listing |
| | | | domain (partial) | pursuant to WIPO St. 26 |
| 43 | Human | Prt | CD16A - Extracellular | see attached sequence listing |
| | | | domain | pursuant to WIPO St. 26 |
| 44 | Human | DNA | CD16A - Extracellular | see attached sequence listing |
| | | | domain (version 2) | pursuant to WIPO St. 26 |
| 45 | Human | Prt | CD16A - Extracellular | see attached sequence listing |
| | | | domain (version 2) | pursuant to WIPO St. 26 |
| 46 | Human | DNA | CD16A - Full length | see attached sequence listing |
| | | | | pursuant to WIPO St. 26 |
| 47 | Human | Prt | CD16A - Full length | see attached sequence listing |
| | | | | pursuant to WIPO St. 26 |
| 48 | Art | Prt | ITAM consensus | YXX(I/L)XXXXXXXYXX(I/L) |
| | | | motif 1, variant 1 | |
| 49 | Art | Prt | ITAM consensus | YXX(I/L)XXXXXXXXYXX(I/L) |
| | | | motif 1, variant 2 | |
| 50 | Art | Prt | IgG1 - Motif interface | LLGGPSXXXXXXXXXXXXXX |
| | | | region of constant | XXXXXXXXXXXDXSXEXXXX |
| | | | region (interaction | XXXXXXXXXXXXXXXXXXXX |
| | | | with CD16) | XXXNSTXXXXXXXXXXXXXX |
| | | | | XXXXXXXXXXXXXALPAXI |
| 51 | Human | DNA | lgG1 - Interface | see attached sequence listing |
| | | | region of constant | pursuant to WIPO St. 26 |
| | | | region (interaction | |
| | | | with CD16) | |
| 52 | Human | Prt | lgG1 - Interface | see attached sequence listing |
| | | | region of constant | pursuant to WIPO St. 26 |
| | | | region (interaction | |
| | | | with CD16) | |
| 53 | Human | DNA | lgG1 - heavy chain | see attached sequence listing |
| | | | constant region | pursuant to WIPO St. 26 |
| 54 | Human | Prt | lgG1 - heavy chain | see attached sequence listing |
| | | | constant region | pursuant to WIPO St. 26 |
| 55 | Human | DNA | CD3zeta - full length | see attached sequence listing |
| | | | | pursuant to WIPO St. 26 |
| 56 | Human | Prt | CD3zeta - full length | see attached sequence listing |
| | | | | pursuant to WIPO St. 26 |
| 57 | Human | DNA | FceRlg - full length | see attached sequence listing |
| | | | | pursuant to WIPO St. 26 |
| 58 | Human | Prt | FceRlg - full length | see attached sequence listing |
| | | | | pursuant to WIPO St. 26 |
| 59 | Human | DNA | Kappa light chain | see attached sequence listing |
| | | | constant region | pursuant to WIPO St. 26 |
| 60 | Human | Prt | Kappa light chain | see attached sequence listing |
| | | | constant region | pursuant to WIPO St. 26 |
| 61 | Human | DNA | lgG1 heavy chain | see attached sequence listing |
| | | | constant domain CH1 | pursuant to WIPO St. 26 |
| 62 | Human | Prt | lgG1 heavy chain | see attached sequence listing |
| | | | constant domain CH1 | pursuant to WIPO St. 26 |
| 63 | Human | DNA | lgG1 heavy chain | see attached sequence listing |
| | | | constant domain CH2 | pursuant to WIPO St. 26 |
| 64 | Human | Prt | lgG1 heavy chain | see attached sequence listing |
| | | | constant domain CH2 | pursuant to WIPO St. 26 |
| 65 | Human | DNA | lgG1 heavy chain | see attached sequence listing |
| | | | constant domain CH3 | pursuant to WIPO St. 26 |
| 66 | Human | Prt | lgG1 heavy chain | see attached sequence listing |
| | | | constant domain CH3 | pursuant to WIPO St. 26 |
| 67 | Human | DNA | lgM heavy chain | see attached sequence listing |
| | | | constant domain CH4 | pursuant to WIPO St. 26 |
| 68 | Human | Prt | lgM heavy chain | see attached sequence listing |
| | | | constant domain CH4 | pursuant to WIPO St. 26 |
| 69 | Art | Prt | Glycine-serine linker | GGGGS |
| | | | motif | |
| 70 | Art | Prt | Glycine-serine linker | SGGGGS |
| | | | N-terminus | |
| 71 | Art | DNA | Glycine-serine linker | see attached sequence listing |
| | | | | pursuant to WIPO St. 26 |
| 72 | Art | Prt | Glycine-serine linker | see attached sequence listing |
| | | | | pursuant to WIPO St. 26 |
| 73 | Art | Prt | ITAM consensus | YXX(I/L)XXXXXXYXX(I/L) |
| | | | motif 1 | |
| 74 | Art | Prt | ITAM consensus | YXX(I/L)XXXXXXXXXXXXYXX |
| | | | motif 2 | (I/L) |
| 75 | Human | DNA | CD3 zeta - ITAM 1 | tataacgagctcaatctaggacgaagag |
| | | | | aggagtacgatgttttg |
| 76 | Human | Prt | CD3 zeta - ITAM 1 | YNELNLGRREEYDVL |
| 77 | Human | DNA | CD3 zeta - ITAM 2 | tacaatgaactgcagaaagataagatg |
| | | | | gcggaggcctacagtgagatt |
| 78 | Human | Prt | CD3 zeta - ITAM 2 | YNELQKDKMAEAYSEI |
| 79 | Human | DNA | CD3 zeta - ITAM 3 | taccagggtctcagtacagccaccaagg |
| | | | | acacctacgacgccctt |
| 80 | Human | Prt | CD3 zeta - ITAM 3 | YQGLSTATKDTYDAL |
| 81 | Human | DNA | CD3 zeta - ITAM | see attached sequence listing |
| | | | region | pursuant to WIPO St. 26 |
| 82 | Human | Prt | CD3 zeta - ITAM | see attached sequence listing |
| | | | region | pursuant to WIPO St. 26 |
| 83 | Human | DNA | CD3 zeta - | see attached sequence listing |
| | | | Intracellular domain | pursuant to WIPO St. 26 |
| 84 | Human | Prt | CD3 zeta - | see attached sequence listing |
| | | | Intracellular domain | pursuant to WIPO St. 26 |
| 85 | Human | DNA | FceRlg - ITAM | see attached sequence listing |
| | | | | pursuant to WIPO St. 26 |
| 86 | Human | Prt | FceRlg - ITAM | YTGLSTRNQETYETL |
| 87 | Human | DNA | FceRlg - Intracellular | see attached sequence listing |
| | | | domain | pursuant to WIPO St. 26 |
| 88 | Human | Prt | FceRlg - Intracellular | AITSYEKSDGVYTGLSTRNQE |
| | | | domain | TYETLKHEKPPQ |
| 89 | Human | DNA | mlgG1 - Intracellular | see attached sequence listing |
| | | | domain | pursuant to WIPO St. 26 |
| 90 | Human | Prt | mlgG1 - Intracellular | KVKWIFSSVVDLKQTIIPDYRN |
| | | | domain | MIGQGA |
| 91 | Human | DNA | CD79A - ITAM | see attached sequence listing |
| | | | | pursuant to WIPO St. 26 |
| 92 | Human | Prt | CD79A - ITAM | YEGLNLDDCSMYEDI |
| 93 | Human | DNA | CD79B - ITAM | see attached sequence listing |
| | | | | pursuant to WIPO St. 26 |
| 94 | Human | Prt | CD79B - ITAM | YEGLDIDQTATYEDI |
| 95 | Human | DNA | CD79A - Intracellular | see attached sequence listing |
| | | | domain | pursuant to WIPO St. 26 |
| 96 | Human | Prt | CD79A - Intracellular | see attached sequence listing |
| | | | domain | pursuant to WIPO St. 26 |
| 97 | Human | DNA | CD79B - Intracellular | see attached sequence listing |
| | | | domain | pursuant to WIPO St. 26 |
| 98 | Human | Prt | CD79B - Intracellular | see attached sequence listing |
| | | | domain | pursuant to WIPO St. 26 |
| 99 | Human | DNA | lgG1 heavy chain - | see attached sequence listing |
| | | | constant region, | pursuant to WIPO St. 26 |
| | | | membrane domain, | |
| | | | and intracellular | |
| | | | domain | |
| 100 | Human | Prt | lgG1 heavy chain - | see attached sequence listing |
| | | | constant region, | pursuant to WIPO St. 26 |
| | | | membrane domain, | |
| | | | and intracellular | |
| | | | domain | |
| 101 | Human | DNA | lgG1 heavy chain - | see attached sequence listing |
| | | | constant region, | pursuant to WIPO St. 26 |
| | | | linker, membrane | |
| | | | domain, and | |
| | | | intracellular domain | |
| 102 | Human | Prt | lgG1 heavy chain - | see attached sequence listing |
| | | | constant region, | pursuant to WIPO St. 26 |
| | | | linker, membrane | |
| | | | domain, and | |
| | | | intracellular domain | |
| 103 | Human | DNA | mlgM- constant | see attached sequence listing |
| | | | region | pursuant to WIPO St. 26 |
| 104 | Human | Prt | mlgM- constant | see attached sequence listing |
| | | | region | pursuant to WIPO St. 26 |
| 105 | Human | DNA | mlgM- constant | see attached sequence listing |
| | | | region and | pursuant to WIPO St. 26 |
| | | | membrane domain | |
| | | | and cytosolic tail | |
| 106 | Human | Prt | mlgM- constant | see attached sequence listing |
| | | | region and | pursuant to WIPO St. 26 |
| | | | membrane domain | |
| | | | and cytosolic tail | |
| 107 | Human | DNA | mlgG1 membrane | see attached sequence listing |
| | | | domain_FceRlg | pursuant to WIPO St. 26 |
| | | | intracellular domain | |
| | | | fusion | |
| 108 | Human | Prt | mlgG1 membrane | see attached sequence listing |
| | | | domain_FceRlg | pursuant to WIPO St. 26 |
| | | | intracellular domain | |
| | | | fusion | |
| 109 | Human | DNA | CD79A extracellular | see attached sequence listing |
| | | | and membrane | pursuant to WIPO St. 26 |
| | | | domain_CD3 zeta | |
| | | | intracellular domain | |
| | | | fusion | |
| 110 | Human | Prt | CD79A extracellular | see attached sequence listing |
| | | | and membrane | pursuant to WIPO St. 26 |
| | | | domain_CD3 zeta | |
| | | | intracellular domain | |
| | | | fusion | |
| 111 | Human | DNA | CD79B extracellular | see attached sequence listing |
| | | | and membrane | pursuant to WIPO St. 26 |
| | | | domain_CD3 zeta | |
| | | | intracellular domain | |
| | | | fusion | |
| 112 | Human | Prt | CD79B extracellular | see attached sequence listing |
| | | | and membrane | pursuant to WIPO St. 26 |
| | | | domain_CD3 zeta | |
| | | | intracellular domain | |
| | | | fusion | |
| 113 | Human | DNA | mlgG1 constant | see attached sequence listing |
| | | | region and | pursuant to WIPO St. 26 |
| | | | membrane | |
| | | | domain_FceRlg | |
| | | | intracellular domain | |
| | | | fusion | |
| 114 | Human | Prt | mlgG1 constant | see attached sequence listing |
| | | | region and | pursuant to WIPO St. 26 |
| | | | membrane | |
| | | | domain_FceRlg | |
| | | | intracellular domain | |
| | | | fusion | |

Herein, an amino acid sequence with a designated name may be encoded by a DNA sequence having the same designated name herein. Furthermore, any amino acid sequence, i.e. polypeptide, that is encoded by a DNA sequence disclosed herein, is also disclosed herein, in particular in the context of the present invention.

Further reference to the sequences set forth in SEQ ID NO: 115 to 172 can be found in Tables 2 and 3 herein.

1. A modified mammalian cell comprising the following (I) and (II):
(I) a first and second polypeptide, each comprising a variable region, wherein the variable region of the first polypeptide and the variable region of the second polypeptide form an antigen-binding site at the external side of the cell,
wherein the first polypeptide further comprises a membrane domain located within the membrane of the cell, and
wherein the first and second polypeptide are not, preferably do not comprise:
(i) an alpha and beta chain of a T cell receptor (TCR), or
(ii) a gamma and delta chain of a TCR; and
(II) at least one polypeptide, e.g. said first polypeptide and/or at least one further polypeptide, comprising an intracellular domain containing at least one signaling domain; and
wherein, upon binding of the antigen-binding site to a corresponding antigen, at least one of the signaling domains triggers a process in the cell that enables the cell to promote death of a target cell comprising said antigen on the cell surface; preferably, wherein the modified cell is not a B cell that is required to interact with another immune cell type to promote death of the target cell.

2. The modified cell of claim 1, wherein the cell comprises one or more nucleic acid molecules, from which the first and second polypeptide are expressed.

3. A kit comprising one or more nucleic acid molecules, wherein said nucleic acid molecule(s) comprise the following (I) and (II):
(I) a first coding sequence encoding a first polypeptide, and a second coding sequence encoding a second polypeptide,
wherein the first and second polypeptide each comprise a variable region, and the first polypeptide further comprises a membrane domain, wherein the variable region of the first polypeptide and the variable region of the second polypeptide are able to form an antigen-binding site when expressed together in a modified mammalian cell,
and wherein the first and second polypeptide are not, preferably do not comprise:
(i) an alpha and beta chain of a T cell receptor (TCR), or
(ii) a gamma and delta chain of a TCR; and
(II) at least one coding sequence encoding at least one polypeptide, e.g. said first polypeptide and/or at least one further polypeptide, comprising an intracellular domain containing at least one signaling domain; and
wherein, in a modified cell comprising the first and second polypeptide and at least one polypeptide comprising an intracellular domain containing at least one signaling domain, upon binding of the antigen-binding site to a corresponding antigen, at least one of the signaling domains is able to trigger a process in the cell that enables the cell to promote death of a target cell comprising said antigen on the cell surface.

4. The modified cell of claim 1 or 2, or the kit of claim 3, wherein the variable regions of the first and second polypeptide contain at least one, preferably at least three, preferably all, complementary determining region(s) (CDR) of an antibody, preferably wherein the first and second polypeptide contain each at least one, preferably three, CDR(s) of the antibody.

5. The modified cell of any one of claims 1, 2, or 4, or the kit of claim 3 or 4, wherein the first polypeptide contains the complementary determining regions (CDRs) of a heavy chain of an antibody, i.e., CDR-H1, CDR-H2 and CDR-H3, and/or
the variable region of the second polypeptide contains the CDRs of a light chain of said antibody, i.e., CDR-L1, CDR-L2 and CDR-L3.

6. The modified cell of any one of claims 1, 2, or 4, or the kit of claim 3 or 4, wherein the first polypeptide contains the complementary determining regions (CDRs) of a light chain of an antibody, i.e., CDR-L1, CDR-L2 and CDR-L3, and/or
the variable region of the second polypeptide contains the CDRs of a heavy chain of said antibody, i.e., CDR-H1, CDR-H2 and CDR-H3.

7. The modified cell of any one of claims 1, 2 or 4 to 6, or the kit of any one of claims 3 to 6, wherein the first polypeptide comprises the variable region of a heavy chain of the antibody, and/or the second polypeptide comprises the variable region of a light chain of the antibody.

8. The modified cell of any one of claims 1, 2 or 4 to 7, or the kit of any one of claims 3 to 7, wherein the modified cell is a natural killer (NK) cell.

9. The modified cell of any one of claims 1, 2 or 4 to 7, or the kit of any one of claims 3 to 7, wherein the modified cell is a T cell, in particular a CD8+ T cell or a CD4+ cell such as a helper or a regulatory T cell, preferably a CD8+ T cell.

10. The modified cell of any one of claims 1, 2 or 4 to 9, or the kit of any one of claims 3 to 9, wherein the modified cell is a primary cell or a cell line.

11. The modified cell of any one of claims 1, 2 or 4 to 10, or the kit of any one of claims 3 to 10, wherein the modified cell is derived from cord blood or peripheral blood, obtained by differentiation of a pluripotent cell, e.g. an induced pluripotent stem cell, or obtained by reprogramming of another cell type.

12. The modified cell of any one of claims 1, 2 or 4 to 10, or the kit of any one of claims 3 to 10, wherein
(a) the first polypeptide comprises an intracellular domain containing at least one signaling domain, and/or
(b) the modified cell comprises a third and/or a fourth polypeptide, and the nucleic acid molecule(s) comprise a third coding sequence encoding a third polypeptide and/or a fourth coding sequence encoding a fourth polypeptide, respectively,
wherein at least one of the third and fourth polypeptides comprises an intracellular domain containing at least one signaling domain, and wherein
(i) the third and/or fourth polypeptide comprising an intracellular domain containing at least one signaling domain is able to interact with and/or bind to the first polypeptide, i.e., in the modified cell, and/or
(ii) the modified cell comprises a fifth polypeptide, wherein the fifth polypeptide is able to interact with and/or bind to the first polypeptide, and the third and/or fourth polypeptide comprising an intracellular domain containing at least one signaling domain, i.e., in the modified cell; wherein the nucleic acid molecule(s) in the kit may further comprise a fifth coding sequence encoding the fifth polypeptide.

13. The modified cell or the kit of claim 12, wherein the first polypeptide further comprises a constant region, in particular, wherein the constant region is located at the external side of the cell.

14. The modified cell or the kit of claims 12 to 13, wherein the third polypeptide and/or the fourth polypeptide comprise a membrane domain, in particular, wherein the membrane domain is located within the membrane of the cell.

15. The modified cell or the kit of claim 14, wherein the membrane domain of the third polypeptide comprises the sequence motif “E-X₍₁₀₎-P” (i.e. EXXXXXXXXXXP), and/or the membrane domain of the fourth polypeptide comprises the sequence motif “Q-X₍₁₀₎-P” (i.e. QXXXXXXXXXXP).

16. The modified cell or the kit of claim 14 or 15, wherein the membrane domain of the third polypeptide comprises a sequence that has at least 50% sequence identity to the sequence set forth in SEQ ID NO: 4; and/or wherein the membrane domain of the fourth polypeptide comprises a sequence that has at least 50% sequence identity to the sequence set forth in SEQ ID NO: 6.

17. The modified cell or the kit of any one of claims 14 to 16, wherein the membrane domain of the third polypeptide comprises a sequence that has at least 50% sequence identity to the membrane domain of a CD79A protein and/or the sequence set forth in SEQ ID NO: 8, and/or wherein the membrane domain of the fourth polypeptide comprises a sequence that has at least 50% sequence identity to the membrane domain of a CD79B protein and/or the sequence set forth in SEQ ID NO: 10.

18. The modified cell or the kit of any one of claims 12 to 17, wherein the third polypeptide and/or the fourth polypeptide comprises an extracellular domain, in particular, wherein the extracellular domain is located at the external side of the cell.

19. The modified cell or the kit of claim 18, wherein the extracellular domain of the third polypeptide comprises a sequence that has at least 50% sequence identity to the extracellular domain of a CD79A protein and/or to the sequence set forth in SEQ ID NO: 12 or SEQ ID NO: 174; and/or the extracellular domain of the fourth polypeptide comprises a sequence that has at least 50% sequence identity to the extracellular domain of a CD79B protein and/or to the sequence set forth in SEQ ID NO: 14 or SEQ ID NO: 177.

20. The modified cell or the kit of any one of claims 12 to 19, wherein the third polypeptide comprises a sequence that has at least 50% sequence identity to a CD79A protein and/or to the sequence set forth in SEQ ID NO: 16 or SEQ ID NO: 175; and/or wherein the fourth polypeptide comprises a sequence that has at least 50% sequence identity to a CD79B protein, and/or to the sequence set forth in SEQ ID NO: 18 or SEQ ID NO: 178.

21. The modified cell or the kit of any one of claims 12 to 20, wherein the third and fourth polypeptide are able to interact with and/or bind to each other, i.e., in the modified cell.

22. The modified cell of any one of claims 1, 2 or 4 to 21, or the kit of any one of claims 3 to 21, wherein the membrane domain of the first polypeptide comprises the sequence motif “YS”.

23. The modified cell of any one of claims 1, 2 or 4 to 22, or the kit of any one of claims 3 to 22, wherein the membrane domain of the first polypeptide comprises the sequence motif “WXXXXXFXXLFXLXXXYSXXXT” (SEQ ID NO: 19).

24. The modified cell of any one of claims 1, 2 or 4 to 23, or the kit of any one of claims 3 to 23, wherein the membrane domain of the first polypeptide comprises a sequence that has at least 50% sequence identity to the membrane domain of a membrane-bound immunoglobulin and/or to a sequence set forth in SEQ ID NO: 20, 21, 22, 23, 24, 25, 26, 27, 29, 31, 37 or 38, preferably SEQ ID NO: 20, 21, 22, 29 or 31.

25. The modified cell or the kit of any one of claims 14 to 24, wherein
the membrane domain and/or constant region of the first polypeptide is able to interact with and/or bind to the membrane domain and/or extracellular domain of at least one polypeptide selected from the group consisting of: a CD79A protein, a CD79B protein, the third polypeptide and the fourth polypeptide; and/or
the membrane domain and/or extracellular domain of the third polypeptide and/or the membrane domain and/or extracellular domain of the fourth polypeptide is able to interact with and/or bind to the membrane domain and/or constant region of at least one polypeptide selected from the group consisting of: a membrane-bound immunoglobulin and the first polypeptide.

26. The modified cell or the kit of any one of claims 14 to 25, wherein
the membrane domain of the first polypeptide is able to interact with and/or bind to the membrane domain of at least one polypeptide selected from the group consisting of: a CD79A protein, a CD79B protein, the third polypeptide and the fourth polypeptide; and/or
the membrane domain of the third polypeptide and/or the membrane domain of the fourth polypeptide is able to interact with and/or bind to the membrane domain of at least one polypeptide selected from the group consisting of: a membrane-bound immunoglobulin and the first polypeptide.

27. The modified cell or the kit of any one of claims 12 to 26, wherein the fifth polypeptide comprises a membrane domain, in particular, wherein the membrane domain is located within the membrane of the cell.

28. The modified cell or the kit of claim 27, wherein the membrane domain of the fifth polypeptide comprises the sequence motif “FXXDT” or the sequence motif “FXXNT”.

29. The modified cell or the kit of claim 27 or 28, wherein the membrane domain of the fifth polypeptide comprises a sequence that has at least 80% sequence identity to the sequence set forth in SEQ ID NO: 34.

30. The modified cell or the kit of any one of claims 27 to 29, wherein the membrane domain of the fifth polypeptide comprises a sequence that has at least 50% sequence identity to the membrane domain of a CD16 protein and/or to the sequence set forth in SEQ ID NO: 36.

31. The modified cell or the kit of any one of claims 12 to 30, wherein the fifth polypeptide comprises an extracellular domain, in particular, wherein the extracellular domain is located at the external side of the cell.

32. The modified cell or the kit of any one of claims 12 to 31, wherein the extracellular domain of the fifth polypeptide comprises the sequence motif set forth in SEQ ID NO: 39.

33. The modified cell or the kit of claim 31 or 32, wherein the extracellular domain of the fifth polypeptide comprises a sequence that has at least 50% sequence identity to the sequence set forth in SEQ ID NO: 41.

34. The modified cell or the kit of any one of claims 31 to 33, wherein the extracellular domain of the fifth polypeptide comprises a sequence that has at least 50% sequence identity to the extracellular domain of a CD16 protein, and/or to the sequence set forth in SEQ ID NO: 43 or 45.

35. The modified cell or the kit of any one of claims 13 to 34, wherein the constant region of the first polypeptide comprises the sequence motif set forth in SEQ ID NO: 50.

36. The modified cell or the kit of any one of claims 13 to 35, wherein the constant region of the first polypeptide comprises a sequence that has at least 50% sequence identity to the sequence set forth in SEQ ID NO: 52.

37. The modified cell or the kit of any one of claims 13 to 36, wherein the constant region of the first polypeptide comprises a sequence that has at least 50% sequence identity to a constant domain of an immunoglobulin, e.g. C_{H}1, C_{H}2, C_{H}3 or C_{H}4, and/or to the sequence set forth in SEQ ID NO: 62, 64, 66 or 68, or to the constant region of an immunoglobulin and/or to the sequence set forth in SEQ ID NO: 54.

38. The modified cell or the kit of any one of claims 27 to 37, wherein
the constant region and/or membrane domain of the first polypeptide is able to interact with and/or bind to the extracellular domain and/or membrane domain of at least one polypeptide selected from the group consisting of: a Fc-receptor, a CD16 protein, and the fifth polypeptide; and/or
the extracellular domain and/or membrane domain of the fifth polypeptide is able to interact with and/or bind to the constant region and/or membrane domain of at least one polypeptide selected from the group consisting of: a membrane-bound immunoglobulin or at least one constant domain thereof, e.g. C_{H}1, C_{H}2, C_{H}³ or C_{H}4, and the first polypeptide.

39. The modified cell or the kit of any one of claims 31 to 38, wherein
the constant region of the first polypeptide is able to interact with and/or bind to the extracellular domain of at least one polypeptide selected from the group consisting of: a Fc-receptor protein, a CD16 protein, and the fifth polypeptide; and/or
the extracellular domain of the fifth polypeptide is able to interact with and/or bind to the constant region of at least one polypeptide selected from the group consisting of: a membrane-bound immunoglobulin or at least one constant domain thereof, e.g. C_{H}1, C_{H}2, C_{H}3 or C_{H}4, and the first polypeptide.

40. The modified cell or the kit of any one of claims 12 to 14 or 27 to 39, wherein the third polypeptide comprises a sequence that has at least 50% sequence identity to a CD3 zeta protein and/or to the sequence set forth in SEQ ID NO: 56; and/or the fourth polypeptide comprises a sequence that has at least 50% sequence identity to a FceRIg protein and/or to the sequence set forth in SEQ ID NO: 58.

41. The modified cell or the kit of any one of claims 27 to 40, wherein
the membrane domain and/or extracellular domain of the fifth polypeptide is able to interact with and/or bind to the membrane domain and/or extracellular domain of at least one polypeptide selected from the group consisting of: a CD3 zeta protein, a FceRIg protein, the third polypeptide, and the fourth polypeptide; and/or
the membrane domain and/or extracellular domain of the third polypeptide and/or the membrane domain and/or extracellular domain of the fourth polypeptide is able to interact with and/or bind to the membrane domain and/or extracellular domain of at least one polypeptide selected from the group consisting of: a CD16 protein, and the fifth polypeptide.

42. The modified cell or the kit of any one of claims 27 to 41, wherein
the membrane domain of the fifth polypeptide is able to interact with and/or bind to the membrane domain of at least one polypeptide selected from the group consisting of: a CD3 zeta protein, a FceRIg protein, the third polypeptide, and the fourth polypeptide; and/or
the membrane domain of the third polypeptide and/or the membrane domain of the fourth polypeptide is able to interact with and/or bind to the membrane domain of at least one polypeptide selected from the group consisting of: a CD16 protein, and the fifth polypeptide.

43. The modified cell or the kit of any one of claims 12 to 42, wherein the modified cell comprises the third and fourth polypeptide as defined in any one of claims 14 to 26, and the first and second polypeptide, preferably wherein the first, second, third and fourth polypeptide form a protein complex, i.e. in the modified cell.

44. The modified cell or the kit of any one of claims 12 to 26, 35 to 37, or 43, wherein the modified cell does not comprise a CD16 protein, or a polypeptide that has at least 90% sequence identity to the sequence set forth in SEQ ID NO: 47.

45. The modified cell or the kit of any one of claims 12 to 42, wherein the modified cell comprises the third and/or fourth polypeptide as defined in any one of claims 40 to 42, and the first and second polypeptide.

46. The modified cell or the kit of any one of claims 12 to 14, 22 to 24, 27 to 42, or 45, wherein the modified cell does not comprise a CD79A protein, a CD79B protein or a polypeptide that has at least 90% sequence identity to the sequence set forth in SEQ ID NO: 16, 18, 175 or 178.

47. The modified cell of any one of claims 1, 2 or 4 to 46, or the kit of any one of claims 3 to 46, wherein the second polypeptide further comprises a constant region comprising a sequence that has at least 50% sequence identity to the constant region of a light chain of an antibody and/or a the sequence set forth in SEQ ID NO: 60; in particular, wherein the constant region of the second polypeptide is located at the external side of the cell.

48. The modified cell of any one of claims 1, 2 or 4 to 47, or the kit of any one of claims 3 to 47, wherein the second polypeptide is located in its entirety at the external side of the cell.

49. The modified cell of any one of claims 1, 2 or 4 to 48, or the kit of any one of claims 3 to 48, wherein the first and the second polypeptide are able to form an Y-shaped protein comprising two first polypeptide chains that are connected to each other, for example by a disulfide bond, and two second polypeptide chains, wherein each of the first polypeptide chains is connected to a second polypeptide chain, for example, by a disulfide bond.

50. The modified cell of any one of claims 1, 2 or 4 to 49, or the kit of any one of claims 3 to 49, wherein the first polypeptide further comprises a linker region between the variable region and the membrane domain, in particular, wherein the linker region forms a flexible linker.

51. The modified cell or the kit of any one of claims 13 to 50, wherein the first polypeptide comprises a linker region between the constant region and the membrane domain, in particular, wherein the linker region forms a flexible linker.

52. The modified cell or the kit of claim 50 or 51, wherein the linker region comprises about 10 to about 100 amino acids, preferably about 50 amino acids.

53. The modified cell or the kit of any one of claims 50 to 52, wherein the linker is a glycine-serine linker.

54. The modified cell or the kit of any one of claims 50 to 53, wherein the linker region comprises 2 to 20 repeats of the amino acid sequence GGGGS (SEQ ID NO: 69) and/or the linker region has at its N-terminus the sequence SGGGGS (SEQ ID NO: 70), for example, as set forth in SEQ ID NO: 72.

55. The modified cell or the kit of any one of claims 50 to 54, wherein the linker has a length of about 5 to 50 nm, preferably about 20 nm.

56. The modified cell or the kit of any one of claims 12 to 55, wherein the first polypeptide, the third polypeptide and/or the fourth polypeptide comprises an intracellular domain comprising at least one signaling domain, wherein the signaling domain(s) may be the same or different between the first, third and/or fourth polypeptides; in particular, wherein the at least one signaling domain is located at the internal side of the cell.

57. The modified cell of any one of claims 1, 2 or 4 to 56, or the kit of any one of claims 3 to 56, wherein a signaling domain, e.g. in the first, third and/or fourth polypeptide, comprises at least one signaling or activation motif or region, e.g. an ITAM, ITAM region or ITSM, of a protein selected from the group consisting of: CD3 zeta, FcεRly (FceRIg), CD16A, CD16B, NKp30, NKp46, KIR2DS1-2, KIR2DS3-6, KIR3DS1, NKG2C, NKG2D, 2B4 (CD244), CD2, CRACC, NTB-A (SLAMF6), DNAM-1 (CD226), CD7, CD59, BY55, KIR2DL4 (CD158d), CD44, TNFRSF9 (4-1BB), SLAMF1 (CD150), CD28, TMIGD2 (CD28H), SLAMF7 (CD319), TNFRSF18 (CD357), CD84, HCST (DAP10), TYROB (DAP12), FCRL3, TNFRSF13C (BAFF), and a polypeptide that that at least 50% sequence identity to any of said proteins.

58. The modified cell of any one of claims 1, 2 or 4 to 57, or the kit of any one of claims 3 to 57, wherein a signaling domain, e.g. in the first, third and/or fourth polypeptide, comprises the sequence motif “Y-XX-I or L-X_{(6 to 12)}-Y-XX-I or L”, e.g., as set forth in SEQ ID NO: 73, 48, 49, or 74, or the sequence motif set forth in SEQ ID NO: 186.

59. The modified cell of any one of claims 1, 2 or 4 to 58, or the kit of any one of claims 3 to 58, wherein a signaling domain, e.g. in the first, third and/or fourth polypeptide, comprises at least one immunoreceptor tyrosine-based activation motif (ITAM) of a CD3 zeta protein, a sequence that has at least 50% sequence identity to the sequence set forth in SEQ ID NO: 76, a sequence that has at least 50% sequence identity to the sequence set forth in SEQ ID NO: 78, and/or a sequence that has at least 50% sequence identity to the sequence set forth in SEQ ID NO: 80.

60. The modified cell of any one of claims 1, 2 or 4 to 59, or the kit of any one of claims 3 to 59, wherein a signaling domain, e.g. in the first, third and/or fourth polypeptide, comprises at least one ITAM region of a CD3 zeta protein, and/or a sequence that has at least 50% sequence identity to the sequence set forth in SEQ ID NO: 82.

61. The modified cell of any one of claims 1, 2 or 4 to 59, or the kit of any one of claims 3 to 59, wherein the intracellular domain of the first, third and/or fourth polypeptide comprises the intracellular domain of a CD3 zeta protein, and/or a sequence that has at least 50% sequence identity to the sequence set forth in SEQ ID NO: 84.

62. The modified cell of any one of claims 1, 2 or 4 to 61, or the kit of any one of claims 3 to 61, wherein a signaling domain, e.g. in the first, third and/or fourth polypeptide, comprises at least one immunoreceptor tyrosine-based activation motif (ITAM) of a FceRIg protein, and/or a sequence that has at least 50% sequence identity to the sequence set forth in SEQ ID NO: 86.

63. The modified cell of any one of claims 1, 2 or 4 to 62, or the kit of any one of claims 3 to 62, wherein the intracellular domain of the first, third and/or fourth polypeptide comprises the intracellular domain of a FceRIg protein, and/or a sequence that has at least 50% sequence identity to the sequence set forth in SEQ ID NO: 88.

64. The modified cell of any one of claims 1, 2 or 4 to 63, or the kit of any one of claims 3 to 63, wherein the intracellular domain of the first, third and/or fourth polypeptide, in particular the first polypeptide, comprises the intracellular domain of a membrane-bound immunoglobulin, e.g. an IgG1, and/or a sequence that has at least 50% sequence identity to the sequence set forth in SEQ ID NO: 90.

65. The modified cell of any one of claims 1, 2 or 4 to 64, or the kit of any one of claims 3 to 64, wherein a signaling domain, e.g. in the first, third and/or fourth polypeptide, comprises at least one ITAM of a CD79A protein, and/or a sequence that has at least 50% sequence identity to the sequence set forth in SEQ ID NO: 92.

66. The modified cell of any one of claims 1, 2 or 4 to 65, or the kit of any one of claims 3 to 65, wherein the intracellular domain of the first, third and/or fourth polypeptide, in particular the third polypeptide, comprises the intracellular domain of a CD79A protein, and/or a sequence that has at least 50% sequence identity to the sequence set forth in SEQ ID NO: 96.

67. The modified cell of any one of claims 1, 2 or 4 to 66, or the kit of any one of claims 3 to 66, wherein a signaling domain, e.g. in the first, third and/or fourth polypeptide, comprises at least one ITAM of a CD79B protein, and/or a sequence that has at least 50% sequence identity to the sequence set forth in SEQ ID NO: 94.

68. The modified cell of any one of claims 1, 2 or 4 to 67, or the kit of any one of claims 3 to 67, wherein the intracellular domain of the first, third and/or fourth polypeptide, in particular the fourth polypeptide, comprises the intracellular domain of a CD79B protein, and/or a sequence that has at least 50% sequence identity to the sequence set forth in SEQ ID NO: 98.

69. The modified cell of any one of claims 1, 2 or 4 to 68, or the kit of any one of claims 3 to 68, wherein the first polypeptide comprises a constant region as defined in any one of claims 35 to 39, and a membrane domain as defined in any one of claims 22 to 26, for example, the first polypeptide comprises a sequence as set forth in SEQ ID NO: 106, or positions 1 to 371 of SEQ ID NO: 100.

70. The modified cell or the kit of claim 69, wherein the first polypeptide comprises between the constant region and the membrane domain a linker region as defined in any one of claims 50 to 55.

71. The modified cell or the kit of claim 69 or 70, wherein the first polypeptide further comprises an intracellular domain as defined in claim 64, for example, the first polypeptide comprises a sequence as set forth in SEQ ID NO: 100 or 102.

72. The modified cell or the kit of claim 69 or 70, wherein the first polypeptide comprises an intracellular domain comprising an ITAM as defined in claim 62 and/or an intracellular domain as defined in claim 63, for example, the first polypeptide comprises a sequence as set forth in SEQ ID NO: 114.

73. The modified cell or the kit of claim 69 or 70, wherein the first polypeptide comprises an intracellular domain comprising at least one ITAM as defined in claim 59, an ITAM region as defined in claim 60, and/or an intracellular domain as defined in claim 61.

74. The modified cell or the kit of any one of claims 12 to 73, wherein the third polypeptide comprises an extracellular domain as defined in claim 19 for the third polypeptide, and a membrane domain as defined in any one of claims 15 to 17 for the third polypeptide.

75. The modified cell or the kit of claim 74, wherein the third polypeptide further comprises an intracellular domain comprising an ITAM as defined in claim 65, and/or an intracellular domain as defined in claim 66, for example, the third polypeptide comprises a sequence as set forth in SEQ ID NO: 16 or 175.

76. The modified cell or the kit of claim 74, wherein the third polypeptide comprises an intracellular domain comprising at least one ITAM as defined in claim 59, an ITAM region as defined in claim 60, and/or an intracellular domain as defined in claim 61, for example, the third polypeptide comprises a sequence as set forth in SEQ ID NO: 110.

77. The modified cell or the kit of claim 74, wherein the third polypeptide comprises an intracellular domain comprising an ITAM as defined in claim 62 and/or an intracellular domain as defined in claim 63.

78. The modified cell or the kit of any one of claims 12 to 77, wherein the fourth polypeptide comprises an extracellular domain as defined in claim 19 for the fourth polypeptide, and a membrane domain as defined in any one of claims 15 to 17 for the fourth polypeptide.

79. The modified cell or the kit of claim 78, wherein the fourth polypeptide further comprises an intracellular domain comprising an ITAM as defined in claim 67, and/or an intracellular domain as defined in claim 68, for example, the third polypeptide comprises a sequence as set forth in SEQ ID NO: 18 or 178.

80. The modified cell or the kit of claim 78, wherein the fourth polypeptide comprises an intracellular domain comprising at least one ITAM as defined in claim 59, an ITAM region as defined in claim 60, and/or an intracellular domain as defined in claim 61, for example, the fourth polypeptide comprises a sequence as set forth in SEQ ID NO: 112.

81. The modified cell or the kit of claim 78, wherein the fourth polypeptide comprises an intracellular domain comprising an ITAM as defined in claim 62 and/or an intracellular domain as defined in claim 63.

82. The modified cell of any one of claims 1, 2 or 4 to 81, or the kit of any one of claims 3 to 81, wherein the process in the cell that enables the cell to promote death of a target cell comprises activation of at least one signaling pathway.

83. The modified cell of any one of claims 1, 2 or 4 to 82, or the kit of any one of claims 3 to 82, wherein, upon binding of the antigen-binding site to a corresponding antigen, at least one of the signaling domains activates at least one signaling pathway in the cell that enables the cell to promote death of a target cell comprising said antigen on the cell surface.

84. The modified cell or the kit of claim 82 or 83, wherein the signaling pathway(s) comprise or involve Ca2+ signaling, and/or at least one protein selected from the group consisting of: at least one Src family kinase, at least one Syk family kinase, PLCG1, PI3K, Vav1, at least one Rho family GTPase, ERK1/2, and NFAT.

85. The modified cell of any one of claims 1, 2 or 4 to 84, or the kit of any one of claims 3 to 84, wherein the modified cell is able to kill a target cell comprising said antigen on the cell surface, when said process is triggered and/or said at least one signaling pathway is activated.

86. The modified cell of any one of claims 1, 2 or 4 to 85, or the kit of any one of claims 3 to 85, wherein the modified cell promotes death of the target cell or kills the target cell by secreting a cytotoxic compound and/or contacting the target cell with a cytotoxic compound upon binding of the antigen-binding site to a corresponding antigen, in particular, on the surface of the target cell.

87. The modified cell of any one of claims 1, 2 or 4 to 86, or the kit of any one of claims 3 to 86, wherein the modified cell secretes a granzyme and/or a perforin upon binding of the antigen-binding site to a corresponding antigen, in particular, on the surface of the target cell.

88. The modified cell of any one of claims 1, 2 or 4 to 87, or the kit of any one of claims 3 to 87, wherein the modified cell secretes at least one cytokine upon binding of the antigen-binding site to a corresponding antigen.

89. The modified cell of any one of claims 1, 2 or 4 to 88, or the kit of any one of claims 3 to 88, wherein the modified cell expresses and/or secretes IL-2 and/or IL-15.

90. The modified cell of any one of claims 1, 2 or 4 to 89, or the kit of any one of claims 3 to 90, wherein the modified cell expresses a kill switch protein that kills the modified cell upon binding of a small molecule.

91. The modified cell of any one of claims 1, 2 or 4 to 90 for use in treating a disease in a mammalian subject, preferably a human.

92. The modified cell of any one of claims 1, 2 or 4 to 91 for use in treating a disease that is caused and/or associated with a pathogenic target cell, wherein said modified cell promotes death of said pathogenic target cell, in particular, upon binding of the antigen-binding site to a corresponding antigen, e.g., at the surface of the target cell.

93. The modified cell for use according to claim 92, wherein the pathogenic cell expresses, in particular at the cell surface, an antigen that is recognized by the antigen-binding site of said modified cell.

94. The modified cell for use according to claim 91 or 92, wherein said pathogenic target cell is a tumor cell, or a pathogenic lymphocyte that is associated with and/or causes an autoimmune disease.

95. The modified cell of any one of claims 1, 2 or 4 to 94 for use in treating a cancer in a mammalian subject, preferably a human.

96. The modified cell of any one of claims 1, 2 or 4 to 94 for use in treating an autoimmune disease in a mammalian subject, preferably a human.

97. The modified cell of any one of claims 1, 2 or 4 to 94 for use in an immunotherapy in a mammalian subject, preferably a human.

98. The modified cell for use according to any one of claims 91 to 97, wherein the modified cell is an allogenic or autologous cell, preferably an allogenic cell.

99. The kit of any one of claims 12 to 90, wherein the kit comprises
(i) at least one nucleic acid molecule, each comprising the coding sequence of either the first, second, third, or fourth polypeptide, or optionally the fifth polypeptide;
(ii) at least one nucleic acid molecule, each comprising the coding sequence of two of the first, second, third, and fourth polypeptide, and optionally the fifth polypeptide, for example, wherein one nucleic acid molecule comprises the coding sequence of the first and second polypeptide, and another nucleic acid molecule comprises the coding sequence of the third and fourth polypeptide; and/or
(iii) at least one nucleic acid molecule comprising the coding sequence of at least three, four or all of the first, second, third, and fourth polypeptide, and optionally the fifth polypeptide.

100. The kit of claim 99, wherein in options (ii) and/or (iii), the at least two coding sequences are separated by at least one 2A or IRES sequence, and not separated by stop codons.

101. The kit of claim 99 or 100, wherein the kit comprises at least one plasmid or viral vector, each comprising a nucleic acid molecule according to (i), (ii) or (iii).

102. The kit of claim 101, wherein a plasmid or viral vector comprising a nucleic acid molecule according to (ii) or (iii) comprises a promoter that is capable of producing an mRNA comprising the at least two coding sequences, in particular in a mammalian cell.

103. The kit of claim 102, wherein said mRNA can be translated into the at least two polypeptides, in particular in a mammalian cell.

104. The kit of claim 101, wherein a plasmid or viral vector comprising a nucleic acid molecule according to (ii) or (iii) comprises a plurality of promoters, each being capable of producing an mRNA comprising one of the at least two coding sequences, in particular in a mammalian cell.

105. The kit of any one of claims 3 to 90, or 99 to 104, wherein the nucleic acid molecule(s) are DNA molecules or RNA molecules.

106. The kit of any one of claims 101 to 105, wherein the viral vector is a lentiviral vector, preferably a baboon pseudotyped lentivirus.

107. A method of producing the modified cell according to any one of claims 1 to 90, wherein the method comprises a step of introducing the nucleic acid molecule(s) as defined in the kit of any one of claims 3 to 90, 99, 100 or 105, or the plasmid or viral vector of any one of claims 101 to 106 into a mammalian cell.

108. The method of claim 107, wherein the mammalian cell is an NK cell or a T cell.

109. The method of claim 107 or 108, further comprising a step of activating the mammalian cell, for example, by contacting the mammalian cell with a cytokine such as IL-2.

110. The modified cell of any one of claims 1, 2 or 4 to 90, the kit of any one of claims 3 to 90 or 99 to 106, the modified cell for use according to any one of claims 91 to 98, or the method of claim 108 or 109, wherein the first and second polypeptide are covalently linked, preferably by a peptide bond, and/or the first and second coding sequence form a contiguous nucleic acid sequence encoding a polypeptide comprising the amino acid sequence of the first and second polypeptide.

111. A polypeptide comprising
(a)
(I) an extracellular domain comprising a sequence that has at least 50% sequence identity to the extracellular domain of a CD79A protein, and/or to the sequence set forth in SEQ ID NO: 12 or 174; and/or
a membrane domain comprising
(i) a sequence that has at least 50% sequence identity to the sequence set forth in SEQ ID NO: 4, and/or
(ii) a sequence that has at least 50% sequence identity to the membrane domain of a CD79A protein and/or to the sequence set forth in SEQ ID NO: 8;
and
(II) an intracellular domain comprising
(i) at least one immunoreceptor tyrosine-based activation motif (ITAM) of a CD3 zeta protein, a sequence that has at least 50% sequence identity to the sequence set forth in SEQ ID NO: 76, a sequence that has at least 50% sequence identity to the sequence set forth in SEQ ID NO: 78, and/or a sequence that has at least 50% sequence identity to the sequence set forth in SEQ ID NO: 80,
(ii) at least one ITAM region of a CD3 zeta protein, and/or a sequence that has at least 50% sequence identity to the sequence set forth in SEQ ID NO: 82, and/or
(iii) the intracellular domain of a CD3 zeta protein, and/or a sequence that has at least 50% sequence identity to the sequence set forth in SEQ ID NO: 84;
and/or
(b) a sequence that has at least 50% sequence identity to the sequence set forth in SEQ ID NO: 110 or 184.

112. The polypeptide of claim 111, wherein the polypeptide, i.e. the membrane domain, is able to interact with and/or bind to the membrane domain of a membrane-bound immunoglobulin in a mammalian cell.

113. A nucleic acid molecule comprising a coding sequence encoding the polypeptide of claim 112.

114. The nucleic acid molecule of claim 113 which is DNA or RNA.

115. A viral vector comprising the nucleic acid molecule of claim 113 or 114.

116. A polypeptide comprising
(a)
(I) an extracellular domain comprising a sequence that has at least 50% sequence identity to the extracellular domain of a CD79B protein, and/or to the sequence set forth in SEQ ID NO: 14 or 177; and/or
a membrane domain comprising
(i) a sequence that has at least 50% sequence identity to the sequence set forth in SEQ ID NO: 6, and/or
(ii) a sequence that has at least 50% sequence identity to the membrane domain of a CD79B protein and/or to the sequence set forth in SEQ ID NO: 10;
and
(II) an intracellular domain comprising
(i) at least one immunoreceptor tyrosine-based activation motif (ITAM) of a CD3 zeta protein, a sequence that has at least 50% sequence identity to the sequence set forth in SEQ ID NO: 76, a sequence that has at least 50% sequence identity to the sequence set forth in SEQ ID NO: 78, and/or a sequence that has at least 50% sequence identity to the sequence set forth in SEQ ID NO: 80,
(ii) at least one ITAM region of a CD3 zeta protein, and/or a sequence that has at least 50% sequence identity to the sequence set forth in SEQ ID NO: 82, and/or
(iii) the intracellular domain of a CD3 zeta protein, and/or a sequence that has at least 50% sequence identity to the sequence set forth in SEQ ID NO: 84;
and/or
(b) a sequence that has at least 50% sequence identity to the sequence set forth in SEQ ID NO: 112.

117. The polypeptide of claim 116, wherein the polypeptide, i.e. the membrane domain, is able to interact with and/or bind to the membrane domain of a membrane-bound immunoglobulin in a mammalian cell.

118. A nucleic acid molecule comprising a coding sequence encoding the polypeptide of claim 116 or 117.

119. The nucleic acid molecule of claim 118 which is DNA or RNA.

120. A viral vector comprising the nucleic acid molecule of claim 118 or 119.

121. A polypeptide comprising
(a)
(I) a constant region comprising
(i) a sequence that has at least 50% sequence identity to the sequence set forth in SEQ ID NO: 52,
(ii) a sequence that has at least 50% sequence identity to a constant domain of an immunoglobulin, e.g. C_{H}1, C_{H}2, C_{H}3 or C_{H}4, and/or to the sequence set forth in SEQ ID NO: 62, 64, 66 or 68, and/or
(iii) the constant region of an immunoglobulin and/or the sequence set forth in SEQ ID NO: 54; and/or
a membrane domain comprising a sequence that has at least 50% sequence identity to the membrane domain of a membrane-bound immunoglobulin and/or to a sequence set forth in SEQ ID NO: 20, 21, 22, 23, 24, 25, 26, 27, 29, or 31, preferably SEQ ID NO: 20, 21, 22, 29 or 31;
and
(II) an intracellular domain comprising
(i) at least one immunoreceptor tyrosine-based activation motif (ITAM) of a FceRIg protein, and/or a sequence that has at least 50% sequence identity to the sequence set forth in SEQ ID NO: 86, and/or
(ii) the intracellular domain of a FceRIg protein, and/or a sequence that has at least 50% sequence identity to the sequence set forth in SEQ ID NO: 88;
and/or
(b) a sequence that has at least 50% sequence identity to the sequence set forth in SEQ ID NO: 114.

122. The polypeptide of claim 116, wherein the polypeptide, i.e. the membrane domain, is able to interact with and/or bind to the membrane domain of CD79A and/or CD79B in a mammalian cell; and/or the polypeptide, i.e. the constant region, is able to interact with and/or bind to the extracellular domain of a Fc-receptor, and/or CD16 protein in a mammalian cell.

123. A nucleic acid molecule comprising a coding sequence encoding the polypeptide of claim 121 or 122.

124. The nucleic acid molecule of claim 123 which is DNA or RNA.

125. A viral vector comprising the nucleic acid molecule of claim 123 or 125.

126. A mammalian cell comprising the polypeptide of any one of claim 111, 112, 116, 117, 121 or 122, and/or the nucleic acid molecule of any one of claims 113, 114, 118, 119, 123 or 124.
